(19) United States
(12) Patent Application Publication
(10) Pub. No.: US 2025/0230159 A1
(43) Pub. Date: Jul. 17, 2025

(54) FUSED HETEROCYCLIC COMPOUNDS AS MODULATORS OF RAS SIGNALLING

(71) Applicants: Shenzhen Ionova Life Science Co., Ltd., Shenzhen (CN); Foshan Ionova Biotherapeutics Co., Inc., Foshan (CN)

(72) Inventors: Jing Su, Shenzhen (CN); Hongjian Yang, Shenzhen (CN); Yexing Cao, Shenzhen (CN); Jianming Bao, Shenzhen (CN); Weiguo Quan, Shenzhen (CN); Jing Zhang, Shenzhen (CN); Xuxiang Zhang, Shenzhen (CN); Yingduo Gao, Shenzhen (CN); Kan Ho, Shenzhen (CN); Weiping Ma, Shenzhen (CN); Yongkui Sun, Shenzhen (CN)

(21) Appl. No.: 18/730,747

(22) PCT Filed: Jan. 28, 2023
(86) PCT No.: PCT/CN2023/073511

§ 371 Date: Jul. 19, 2024

Related U.S. Application Data
(60) Provisional application No. 63/310,765, filed on Feb. 16, 2022.

(30) Foreign Application Priority Data
Jan. 21, 2022 (WO) PCT/CN2022/073235

(51) Int. Cl. C07D 471/14; A61K 31/519; A61K 31/5377; A61K 31/5386; A61K 31/541; A61K 31/551; A61K 31/553; C07D 519/00

(52) U.S. Cl. CPC C07D 471/14; A61K 31/519; A61K 31/5377; A61K 31/5386; A61K 31/541; A61K 31/551; A61K 31/553; C07D 519/00

(57) ABSTRACT

Among others, the present invention provides fused heterocyclic compounds of Formula (I) or tautomers, stereoisomer, or pharmaceutically acceptable salts thereof. These compounds and compositions containing them are useful in the treatment of cancers and other disorders associated with RAS dysfunction in a subject.

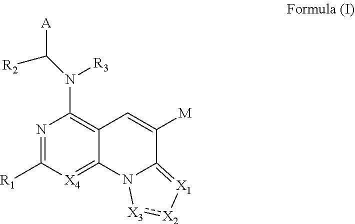

### CROSS-REFERENCE TO RELATED APPLICATIONS

This application claims priority to international application No. PCT/CN2022/073235 filed on Jan. 21, 2022, and U.S. application No. 63/310,765 filed on Feb. 16, 2022, the contents of which are incorporated herein by reference in their entirety.

### BACKGROUND OF THE INVENTION

The Rat Sarcoma Virus (RAS) is one of the most highly validated targets in cancer. However, despite the clinical significance of targeting RAS, the discovery of potent inhibitors of RAS has been difficult to achieve. RAS proteins have earned a well-deserved reputation as being “undruggable” for years.

In human cells, three closely related RAS genes, termed HRAS, KRAS, and NRAS, encode four highly related protein isoforms (H-Ras, N-Ras, K-Ras4A, and K-Ras4B). These RAS proteins are guanosine triphosphatases (GTPases) involved in a broad spectrum of key molecular and cellular activities, including proliferation, differentiation and cell death among others (Front. Oncol., Oct. 18, 2019). The RAS proteins continually cycle between an inactive, guanosine diphosphate (GDP)-bound state and an active guanosine triphosphate (GTP)-bound state, to relay cellular signals in response to extracellular stimuli. Activation of Ras proteins is also regulated by guanine nucleotide exchange factors (GEFs), which catalyze nucleotide exchange, and GTPase-activating proteins (GAP), which aid in GTP hydrolysis. On activation, RAS directly interacts with and activates several downstream effector pathways including the mitogen-activated protein kinase (MAPK) and phosphatidylinositol 3-kinase (PI3K) pathways. However, mutations in RAS disrupt the guanine exchange cycle, typically by becoming GAP-independent and ‘locking’ RAS in the active, GTP-bound state, thereby activating downstream signaling pathways resulting in tumor cell growth. (PNAS, Mar. 4, 2014 111 (9) 3401-3406; Nat Rev. Drug Discov., 2020 August; 19(8): 533-552).

Mutations in RAS genes occur frequently in solid and hematological malignancies with around 20% of all tumors harboring a mutation in at least one isoform. Most oncogenic mutations in RAS isoforms are missense gain-of-function mutations in codons G12, G13 or Q61, although different RAS isoforms differ in their frequency of such alterations (Genes 2021 June; 12(6): 899). Mutation at amino acids G12, G13, Q61 and A146 are found in a variety of human cancers including lung cancer, colorectal cancer and pancreatic cancer (Cox et al., Nat. Rev. Drug Discov., 2014, 13(1): 828-51). Recent biochemical analyses, however, demonstrated these mutated proteins still require nucleotide cycling for activation based on their intrinsic GTPase activity and/or partial sensitivity to extrinsic GTPases. As such, mutant RAS proteins are sensitive to inhibition of upstream factors such as SOS or SHP2, which are upstream signaling proteins required for RAS activation (Nat Rev Drug Discov. 2020 August; 19(8): 533-552).

Son of Sevenless (SOS) exists as two homologs, SOS1 and SOS2. SOS1 encodes a protein that is a guanine nucleotide exchange factor (GEF) for RAS proteins. The product of this gene is critically involved in the activation of RAS proteins by mediating the exchange of GDP for GTP. SOS2 is a homolog of SOS1 in mammalian cells. It also acts as a GEF for the activation of RAS-family proteins. SOS1 and SOS2 share about 70% amino acid sequence identity. The mouse SOS2 knockout is viable whereas the SOS1 knockout is embryonically lethal. A tamoxifen-inducible SOS1 knockout mouse model was used to interrogate the role of SOS1 and SOS2 in adult mice and demonstrated the SOS1 knockout mouse was viable but the SOS1 and SOS2 (SOS 1/2) double knockout died, suggesting functional redundancy and that selective inhibition of SOS1 may have a sufficient therapeutic index for the treatment of SOS1-RAS activated diseases (Mol Cell Biol., 2013 November; 33(22): 4562-4578).

SOS proteins bind to RAS through a catalytic binding site that promotes nucleotide exchange as well as through an allosteric site that binds GTP-bound RAS-family proteins which increases the catalytic function of SOS (PNAS, Nov. 7, 2006, 103 (45) 16692-16697). Binding to the allosteric site relieves steric occlusion of the catalytic site and is therefore required for full activation of the catalytic site. Retention of the active conformation at the catalytic site following interaction with the allosteric site is maintained in isolation due to strengthened interactions of key domains in the activated state. Selective pharmacological inhibition of the interaction of the SOS1 to RAS is expected to prevent SOS1 mediated activation of RAS signaling.

In this invention, Applicants provide a novel compound that inhibits the interaction between SOS1 and RAS protein, thereby, preventing the recycling of KRAS into the active GTP-bound form. These novel compounds provide therapeutic benefits for a wide range of cancers, particularly RAS protein-associated cancers.

### BRIEF SUMMARY OF THE INVENTION

In one aspect, the present invention provides a fused heterocyclic compound of Formula (I), and its tautomer or stereoisomer thereof, or a pharmaceutically acceptable salt thereof. The Formula (I) represents:

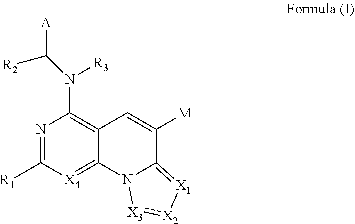

wherein
X₁ is N or CR₁₁;
X₂ and X₃ are each, independently, N, CR₁₁ or O, but are not both O at the same time; and when X₂ or X₃ is O, X₂ and X₃ are bonded together by a single bond;
R₁₁ is H, halo, alkyl, haloalkyl, cycloalkyl, heterocycloalkyl, hydroxyl, hydroxyalkyl, alkoxyl, alkoxyalkyl, amine, cyano, alkynyl or alkenyl;
X₄ is N or CR₁₂; R₁₂ is H, halo, haloalkyl or alkyl;
R₁ is H, halo, alkyl, haloalkyl, cycloalkyl, heterocycloalkyl, hydroxyl, hydroxyalkyl, alkoxyl, alkoxyalkyl, amine, cyano, alkynyl or alkenyl;
each of R₂ and R₃ is, independently, H, halo, alkyl, haloalkyl, alkoxyl, alkoxyalkyl, hydroxyl, hydroxyalkyl, acyl, alkoxycarbonyl, or amine;
A is cycloalkyl, heterocycloalkyl, cycloalkenyl, heterocycloalkenyl, aryl, heteroaryl or 8- to 13-membered fused bicyclic ring, and A is optionally substituted with one or more substituents, each of which is independently selected from halo, alkyl, haloalkyl, alkoxy, alkoxyalkyl, hydroxy, hydroxyalkyl, acyl, alkoxycarbonyl, alkoxyalkcarbonyl, cyano, amine, alkylamino or dialkylamino; the 8- to 13-membered fused bicyclic ring contains at least one aromatic ring;
M is amine, alkyl, halo, haloalkyl, hydroxy, hydroxyalkyl, alkoxy, alkoxyalkyl, acyl, alkoxycarbonyl, aminocarbonyl, carboxyl, alkenyl, alkynyl, alkylthio, aryl, heteroaryl, cycloalkyl, heterocycloalkyl, cycloalkenyl, heterocycloalkenyl, fused bicyclic cycloalkyl or heterocyloalkyl, spirocyclic cycloalkyl or heterocyloalkyl, bridged cycloalkyl or heterocyloalkyl; and M is optionally substituted with 1-5 R₁₀ groups; each R₁₀ group is independently selected from oxo, acyl, hydroxylcarbonyl, hydroxyalkyl-carbonyl, alkoxycarbonyl, alkoxyalkcarbonyl, aminocarbonyl, alkylaminocarbonyl, dialkylaminocarbonyl, —C(O)—(CH₂)ₙ—OR, —S(O)₂—R, alk-S(O)₂—R, alk-C(O)—R, alk-C(O)—NRR′, —(CH₂)ₙ—OR, CD₃-O—, cyano, alkoxyl, alkoxyalkyl, hydroxyl, hydroxyalkyl, alkyl, halo, haloalkyl, cycloalkyl, heterocycloalkyl, cycloalkenyl or heterocycloalkenyl; optionally, two R₁₀ groups together with the atom to which they are both bonded form cycloalkyl or heterocycloalkyl;
optionally, M is connected to the fused tri-heterocyclic moiety in Formula (I) via a linker, which is —(CH)₂—, —(CH₂)ₙ—O—(CH₂)ₙ—, —(CH₂)ₙ—NR—(CH₂)ₙ—, —(CH₂)ₙ—C(O)—O—(CH₂)ₙ—, —(CH₂)ₙ—O—C(O)—(CH₂)ₙ—, —(CH₂)ₙ—C(O)—(CH₂)ₙ—, —NR—C(O)— or —C(O)—NR—;
each R is independently H or alkyl;
each R′ is independently H or alkyl;
n is 0, 1, 2, or 3;
wherein each substituent is optionally further substituted;
a ring-forming carbon atom or heteroatom in cycloalkyl, heterocycloalkyl, cycloalkenyl and heterocycloalkenyl is optionally substituted by one or two oxo groups;
any hydrogen (H) can be optionally replaced by deuterium (D).

In Formula (I), a dash line and a solid line together (shown as “--”) between X₂ and X₃ means that the connection between X₂ and X₃ can be a double bond (═) or a single bond (—), depending on the definitions of X₂ and X₃.

In some embodiments, A in formula (I) is aryl, heteroaryl, or 8- to 13-membered fused bicyclic ring, and is optionally substituted with one or more substituents each of which is independently halo, alkyl, haloalkyl, alkoxy, alkoxyalkyl, hydroxy, hydroxyalkyl, acyl, alkoxycarbonyl, alkoxyalkcarbonyl, cyano, amine, alkylamino, or dialkylamino.

In some embodiments, A is phenyl or fused phenyl; and A is optionally substituted with 1-5 substituents selected from halo, haloalkyl, alkyl, and amine. Preferably, the halo is F.

Examples of a suitable A include, but are not limited to,

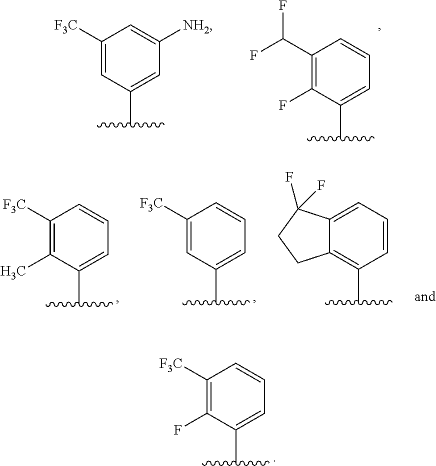

In some embodiments, X₁ is N.

In some embodiments, X₄ is N.

In some embodiments, X₂ and X₃ are each independently CH or N.

In some embodiments, M in Formula (I) is alkoxycarbonyl, alkoxyalkyl-carbonyl, aminocarbonyl, alkylaminocarbonyl, dialkylaminocarbonyl, acyl,

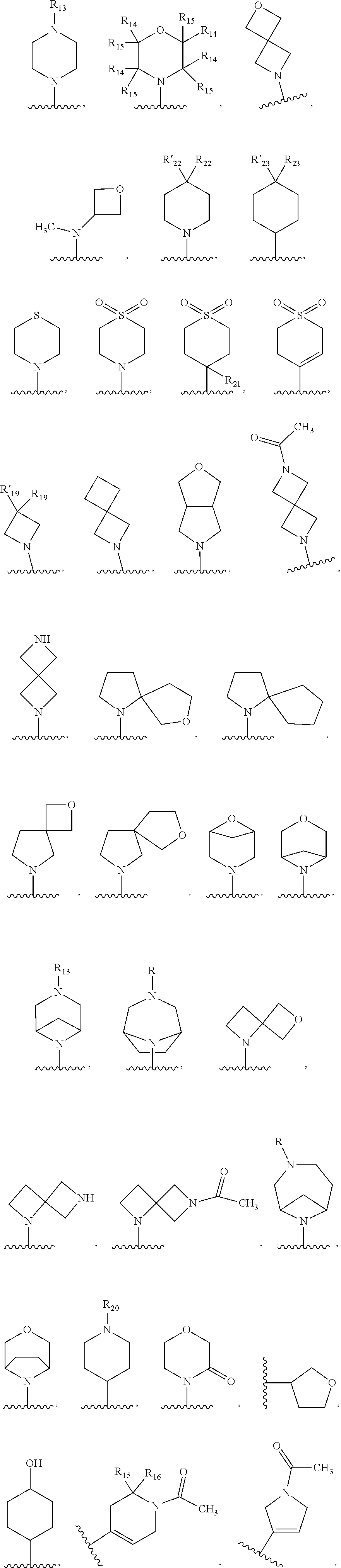

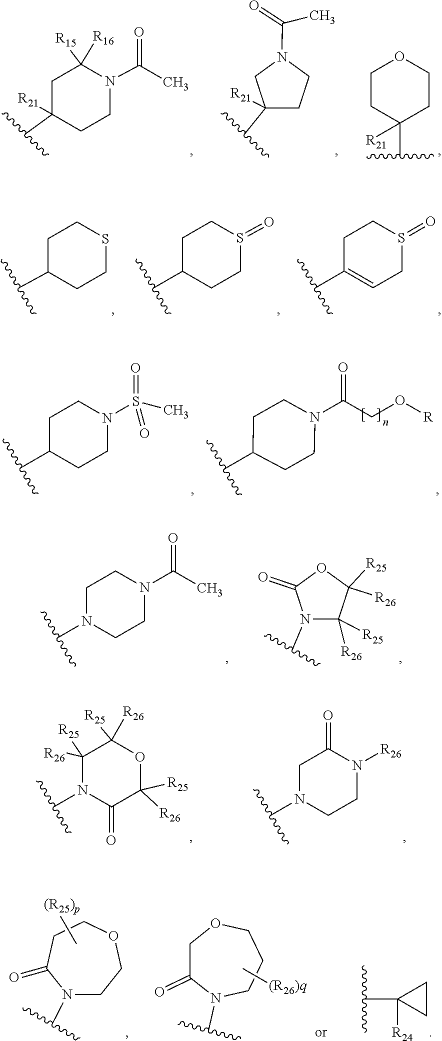

R₁₃ is H, hydroxyl, hydroxyalkyl, alkoxyl, alkoxyalkyl, acyl, —C(O)—NR₁₇R₁₈, —S(O)₂—R, alk-S(O)₂—R, hydroxylcarbonyl, alkoxycarbonyl, hydroxyalkyl-carbonyl, alkoxyalkyl-carbonyl, oxo, alkyl, cycloalkenyl, heterocycloalkenyl, cycloalkyl or heterocycloalkyl; R₁₃ can be optionally further substituted with halo, alkyl, hydroxyl, haloalkyl, or alkoxy;
each of R₁₄, R₁₅, and R₁₆ is, independently, H, halo, hydroxyl, hydroxyalkyl, haloalkyl, alkyl, amine, alkoxyalkyl or alkoxy; optionally, R₁₄ and R₁₅ together with the atom to which they are both bonded form cycloalkyl or heterocycloalkyl; or optionally, R₁₅ and R₁₆ together with the atom to which they are both bonded form cycloalkyl or heterocycloalkyl.
each of R₁₇ and R₁₈ is, independently, H, cycloalkyl, heterocycloalkyl, or alkyl; optionally, R₁₇ and R₁₈ together with the nitrogen atom (N) to which they are both bonded form heterocycloalkyl;
each of R₁₉ and R′₁₉ is independently H, alkyl, alkoxy, alkoxyalkyl, hydroxyl, hydroxyalkyl, halo, haloalkyl, oxo, acyl, hydroxylcarbonyl, alkoxycarbonyl, hydroxyalkyl-carbonyl, alkoxyalkyl-carbonyl, cyano, —S(O)₂—R, alk-S(O)₂—R, —C(O)—NR₁₇R₁₈, cycloalkenyl, heterocycloalkenyl, cycloalkyl or heterocycloalkyl; optionally, R₁₉ and R′₁₉ together with the atom to which they are both bonded form cycloalkyl or heterocycloalkyl;
R₂₀ is H, alkyl, halo, haloalkyl, alkoxyl, alkoxyalkyl, acyl, oxo, —S(O)₂—R, alk-S(O)₂—R, hydroxylcarbonyl, alkoxycarbonyl, hydroxyalkyl-carbonyl, alkoxyalkyl-carbonyl, —C(O)—NR₁₇R₁₈—, cycloalkenyl, heterocycloalkenyl, cycloalkyl, heterocycloalkyl, or

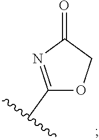 [SMILES: *C1=NC(=O)CO1]

R₂₁ is H, alkyl, hydroxyl, hydroxyalkyl, alkoxyl, alkoxyalkyl, halo, haloalkyl, haloalkyloxy, CF₃O—, CHF₂O—, CD₃-O—, —NR—C(O)—R, or —(CH₂)ₙ—C(O)—NRR′;
each of R₂₂, R′₂₂, R₂₃ and R′₂₃ is, independently, H, alkyl, alkoxyalkyl, alkoxy, hydroxyl, hydroxyalkyl, halo, haloalkyl, acyl, hydroxylcarbonyl, alkoxycarbonyl, hydroxyalkyl-carbonyl, alkoxyalkyl-carbonyl, amine, oxo, —S(O)₂—R, alk-S(O)₂—R, —NR—C(O)—R, —C(O)—NR₁₇R₁₈, or —(CH₂)ₙ—C(O)—NR; optionally, R₂₂ and R′₂₂ together with the atom to which they are both bonded form cycloalkyl or heterocycloalkyl; or optionally, R₂₃ and R′₂₃ together with the atom to which they are both bonded form cycloalkyl or heterocycloalkyl; R₂₄ is H, alkyl, haloalkyl, haloalkyloxy, hydroxy, hydroxyalkyl, alkoxyl, alkoxyalkyl, or cyano;
each of R₂₅ and R₂₆ is, independently, H, alkyl, halo, haloalkyl, alkoxyalkyl, alkoxy, hydroxyl, hydroxyalkyl, cycloalkyl, or heterocycloalkyl; optionally, R₂₅ and R₂₆ together with the atom to which they are both bonded form cycloalkyl or heterocycloalkyl;
each R is independently H or alkyl;
each R′ is independently H or alkyl;
n is 0, 1, 2, or 3;
p is 0, 1, 2, 3, or 4;
q is 0, 1, 2, 3, 4, 5, or 6;
optionally, M is connected to the fused tri-heterocyclic moiety in Formula (I) via a linker which is —(CH₂)ₙ—O—(CH₂)ₙ—, —NR—, —(CH₂)ₙ—C(O)—O—(CH₂)ₙ—, —(CH₂)ₙ—O—C(O)—(CH₂)ₙ—, —(CH₂)ₙ—C(O)—(CH₂)ₙ—, —NR—C(O)— or —C(O)—NR—.

Preferably, M in Formula (I) is

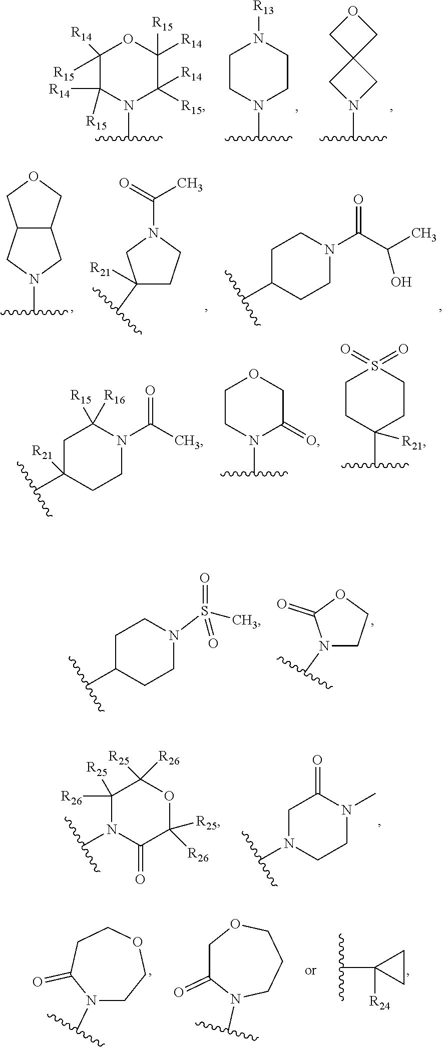

optionally, M is connected to the fused tri-heterocyclic moiety in Formula (I) via the linker C(O)—.

In some further embodiments, R₁ is H or alkyl, R₂ is alkyl and R₃ is H.

Examples of a compound of Formula (I) include:

| Cpd # | Structure |
|---|---|
| 1 | 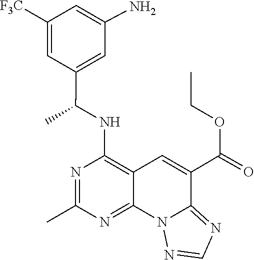 [SMILES: CCOC(=O)c1cc2c(N[C@H](C)c3cc(N)cc(C(F)(F)F)c3)nc(C)nc2n2ncnc12] |
| 2 | 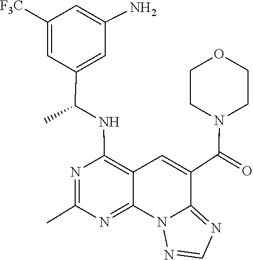 [SMILES: Cc1nc(N[C@H](C)c2cc(N)cc(C(F)(F)F)c2)c2cc(C(=O)N3CCOCC3)c3ncnn3c2n1] |
| 3 | 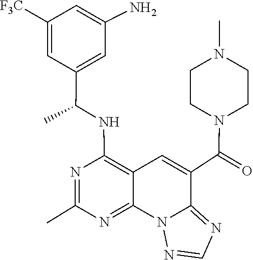 [SMILES: Cc1nc(N[C@H](C)c2cc(N)cc(C(F)(F)F)c2)c2cc(C(=O)N3CCN(C)CC3)c3ncnn3c2n1] |
| 4 | 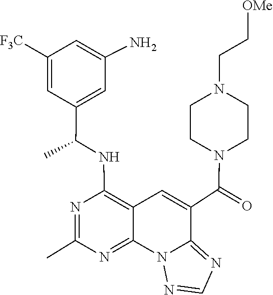 [SMILES: COCCN1CCN(C(=O)c2cc3c(N[C@H](C)c4cc(N)cc(C(F)(F)F)c4)nc(C)nc3n3ncnc23)CC1] |
| 5 | 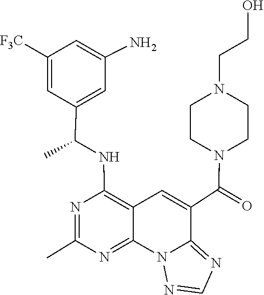 [SMILES: Cc1nc(N[C@H](C)c2cc(N)cc(C(F)(F)F)c2)c2cc(C(=O)N3CCN(CCO)CC3)c3ncnn3c2n1] |
| 6 | 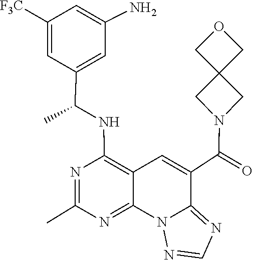 [SMILES: Cc1nc(N[C@H](C)c2cc(N)cc(C(F)(F)F)c2)c2cc(C(=O)N3CC4(COC4)C3)c3ncnn3c2n1] |
| 7 | 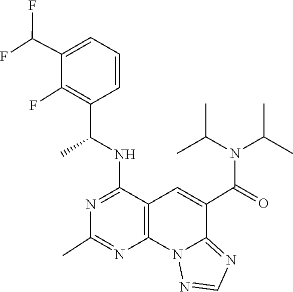 [SMILES: Cc1nc(N[C@H](C)c2cccc(C(F)F)c2F)c2cc(C(=O)N(C(C)C)C(C)C)c3ncnn3c2n1] |
| 8 | 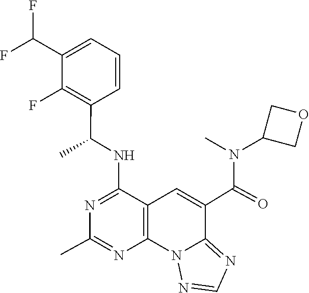 [SMILES: Cc1nc(N[C@H](C)c2cccc(C(F)F)c2F)c2cc(C(=O)N(C)C3COC3)c3ncnn3c2n1] |
| 9 | 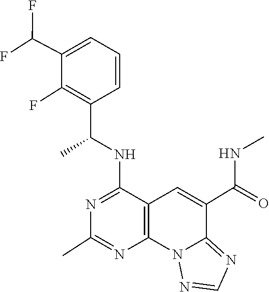 [SMILES: CNC(=O)c1cc2c(N[C@H](C)c3cccc(C(F)F)c3F)nc(C)nc2n2ncnc12] |
| 10 | 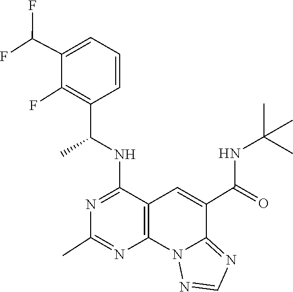 [SMILES: Cc1nc(N[C@H](C)c2cccc(C(F)F)c2F)c2cc(C(=O)NC(C)(C)C)c3ncnn3c2n1] |
| 11 | 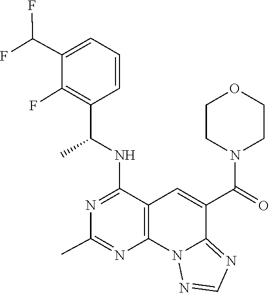 [SMILES: Cc1nc(N[C@H](C)c2cccc(C(F)F)c2F)c2cc(C(=O)N3CCOCC3)c3ncnn3c2n1] |
| 12 | 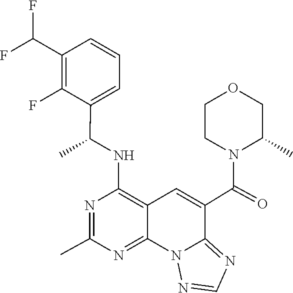 [SMILES: Cc1nc(N[C@H](C)c2cccc(C(F)F)c2F)c2cc(C(=O)N3CCOC[C@@H]3C)c3ncnn3c2n1] |
| 13 | 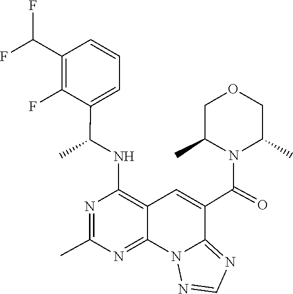 [SMILES: Cc1nc(N[C@H](C)c2cccc(C(F)F)c2F)c2cc(C(=O)N3[C@@H](C)COC[C@@H]3C)c3ncnn3c2n1] |
| 14 | 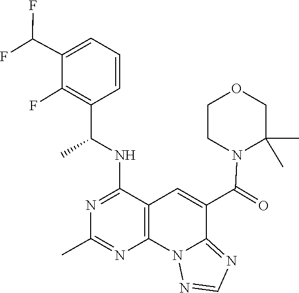 [SMILES: Cc1nc(N[C@H](C)c2cccc(C(F)F)c2F)c2cc(C(=O)N3CCOCC3(C)C)c3ncnn3c2n1] |
| 15 | 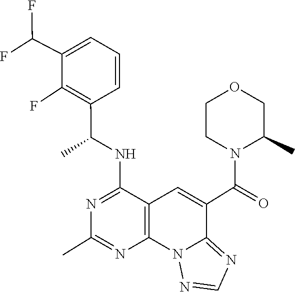 [SMILES: Cc1nc(N[C@H](C)c2cccc(C(F)F)c2F)c2cc(C(=O)N3CCOC[C@H]3C)c3ncnn3c2n1] |
| 16 | 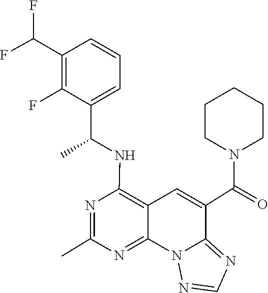 [SMILES: Cc1nc(N[C@H](C)c2cccc(C(F)F)c2F)c2cc(C(=O)N3CCCCC3)c3ncnn3c2n1] |
| 17 | 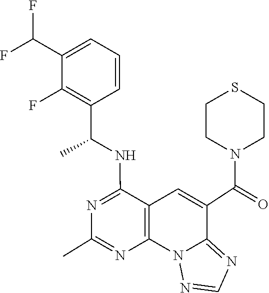 [SMILES: Cc1nc(N[C@H](C)c2cccc(C(F)F)c2F)c2cc(C(=O)N3CCSCC3)c3ncnn3c2n1] |
| 18 | 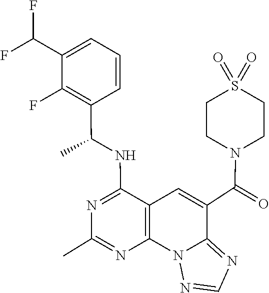 [SMILES: Cc1nc(N[C@H](C)c2cccc(C(F)F)c2F)c2cc(C(=O)N3CCS(=O)(=O)CC3)c3ncnn3c2n1] |
| 19 | 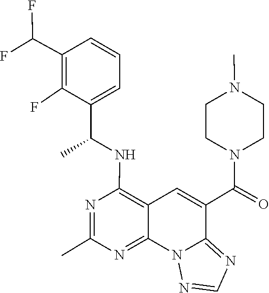 [SMILES: Cc1nc(N[C@H](C)c2cccc(C(F)F)c2F)c2cc(C(=O)N3CCN(C)CC3)c3ncnn3c2n1] |
| 20 | 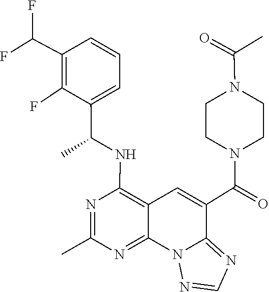 [SMILES: CC(=O)N1CCN(C(=O)c2cc3c(N[C@H](C)c4cccc(C(F)F)c4F)nc(C)nc3n3ncnc23)CC1] |
| 21 | 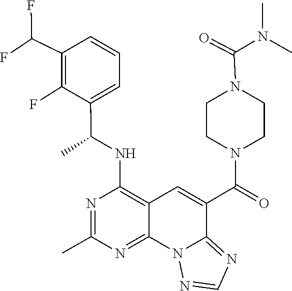 [SMILES: Cc1nc(N[C@H](C)c2cccc(C(F)F)c2F)c2cc(C(=O)N3CCN(C(=O)N(C)C)CC3)c3ncnn3c2n1] |
| 22 | 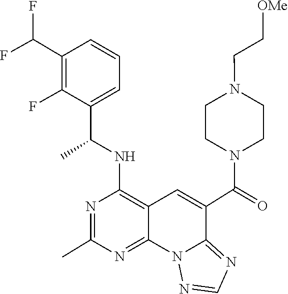 [SMILES: COCCN1CCN(C(=O)c2cc3c(N[C@H](C)c4cccc(C(F)F)c4F)nc(C)nc3n3ncnc23)CC1] |
| 23 | 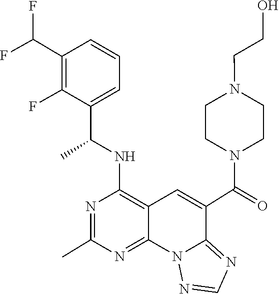 [SMILES: Cc1nc(N[C@H](C)c2cccc(C(F)F)c2F)c2cc(C(=O)N3CCN(CCO)CC3)c3ncnn3c2n1] |
| 24 | 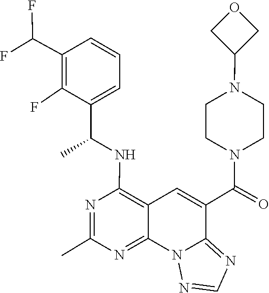 [SMILES: Cc1nc(N[C@H](C)c2cccc(C(F)F)c2F)c2cc(C(=O)N3CCN(C4COC4)CC3)c3ncnn3c2n1] |
| 25 | 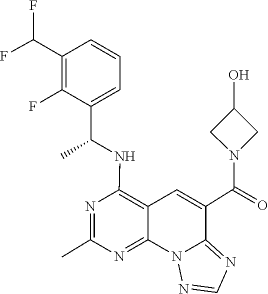 [SMILES: Cc1nc(N[C@H](C)c2cccc(C(F)F)c2F)c2cc(C(=O)N3CC(O)C3)c3ncnn3c2n1] |
| 26 | 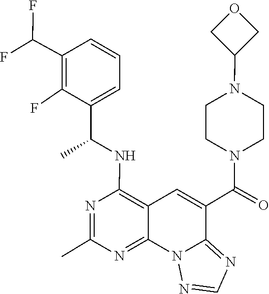 [SMILES: Cc1nc(N[C@H](C)c2cccc(C(F)F)c2F)c2cc(C(=O)N3CCN(C4COC4)CC3)c3ncnn3c2n1] |
| 27 | 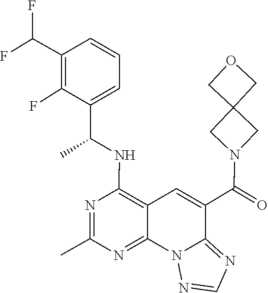 [SMILES: Cc1nc(N[C@H](C)c2cccc(C(F)F)c2F)c2cc(C(=O)N3CC4(COC4)C3)c3ncnn3c2n1] |
| 28 | 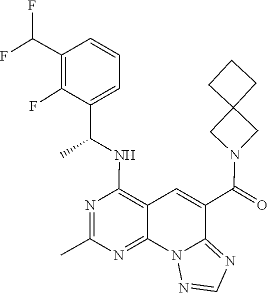 [SMILES: Cc1nc(N[C@H](C)c2cccc(C(F)F)c2F)c2cc(C(=O)N3CC4(CCC4)C3)c3ncnn3c2n1] |
| 29 | 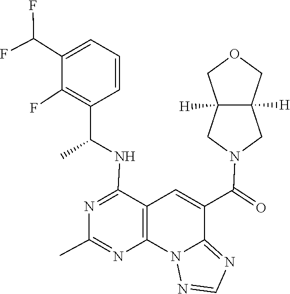 [SMILES: Cc1nc(N[C@H](C)c2cccc(C(F)F)c2F)c2cc(C(=O)N3C[C@H]4COC[C@H]4C3)c3ncnn3c2n1] |
| 30 | 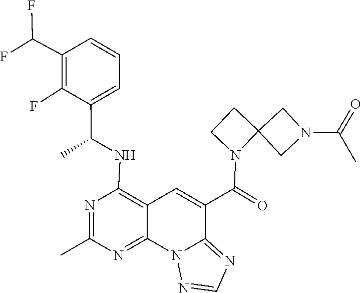 [SMILES: CC(=O)N1CC2(CCN2C(=O)c2cc3c(N[C@H](C)c4cccc(C(F)F)c4F)nc(C)nc3n3ncnc23)C1] |
| 31 | 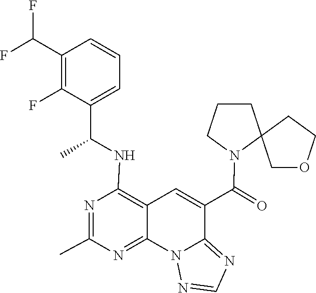 [SMILES: Cc1nc(N[C@H](C)c2cccc(C(F)F)c2F)c2cc(C(=O)N3CCCC34CCOC4)c3ncnn3c2n1] |
| 32 | 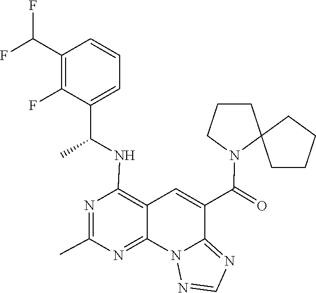 [SMILES: Cc1nc(N[C@H](C)c2cccc(C(F)F)c2F)c2cc(C(=O)N3CCCC34CCCC4)c3ncnn3c2n1] |
| 33 | 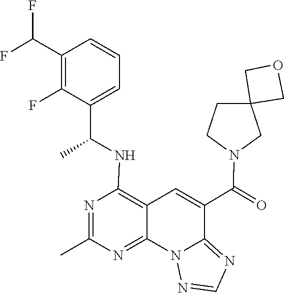 [SMILES: Cc1nc(N[C@H](C)c2cccc(C(F)F)c2F)c2cc(C(=O)N3CCC4(COC4)C3)c3ncnn3c2n1] |
| 34 | 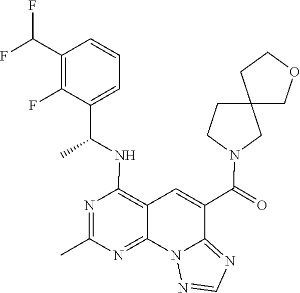 [SMILES: Cc1nc(N[C@H](C)c2cccc(C(F)F)c2F)c2cc(C(=O)N3CCC4(CCOC4)C3)c3ncnn3c2n1] |
| 35 | 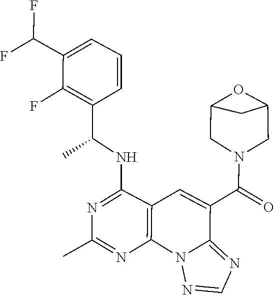 [SMILES: Cc1nc(N[C@H](C)c2cccc(C(F)F)c2F)c2cc(C(=O)N3CC4CC(C3)O4)c3ncnn3c2n1] |
| 36 | 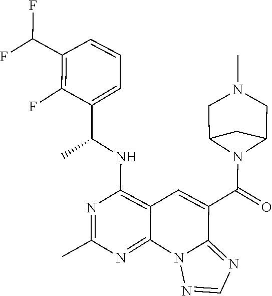 [SMILES: Cc1nc(N[C@H](C)c2cccc(C(F)F)c2F)c2cc(C(=O)N3C4CC3CN(C)C4)c3ncnn3c2n1] |
| 37 | 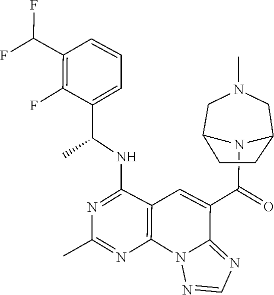 [SMILES: Cc1nc(N[C@H](C)c2cccc(C(F)F)c2F)c2cc(C(=O)N3C4CCC3CN(C)C4)c3ncnn3c2n1] |
| 38 | 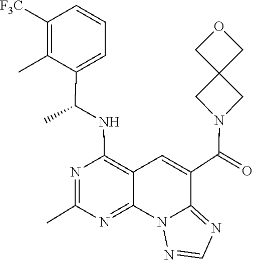 [SMILES: Cc1nc(N[C@H](C)c2cccc(C(F)(F)F)c2C)c2cc(C(=O)N3CC4(COC4)C3)c3ncnn3c2n1] |
| 39 | 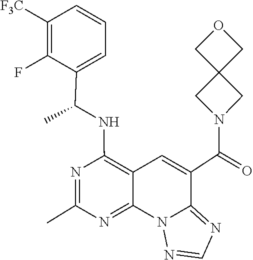 [SMILES: Cc1nc(N[C@H](C)c2cccc(C(F)(F)F)c2F)c2cc(C(=O)N3CC4(COC4)C3)c3ncnn3c2n1] |
| 40 | 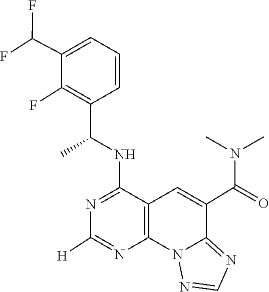 [SMILES: C[C@@H](Nc1ncnc2c1cc(C(=O)N(C)C)c1ncnn12)c1cccc(C(F)F)c1F] |
| 41 | 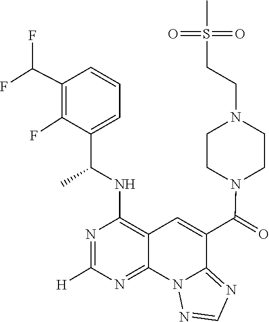 [SMILES: C[C@@H](Nc1ncnc2c1cc(C(=O)N1CCN(CCS(C)(=O)=O)CC1)c1ncnn12)c1cccc(C(F)F)c1F] |
| 42 | 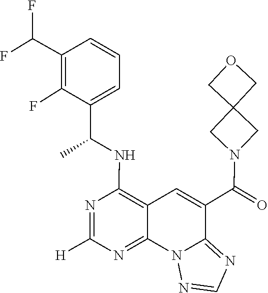 [SMILES: C[C@@H](Nc1ncnc2c1cc(C(=O)N1CC3(COC3)C1)c1ncnn12)c1cccc(C(F)F)c1F] |
| 43 | 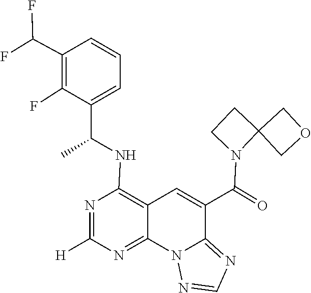 [SMILES: C[C@@H](Nc1ncnc2c1cc(C(=O)N1CCC13COC3)c1ncnn12)c1cccc(C(F)F)c1F] |
| 44 | 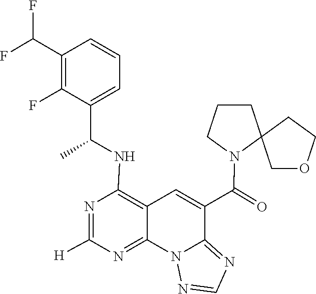 [SMILES: C[C@@H](Nc1ncnc2c1cc(C(=O)N1CCCC13CCOC3)c1ncnn12)c1cccc(C(F)F)c1F] |
| 45 | 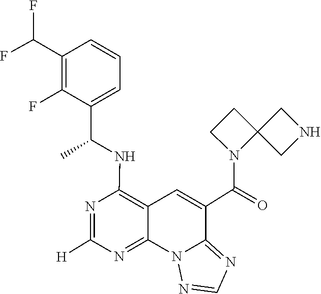 [SMILES: C[C@@H](Nc1ncnc2c1cc(C(=O)N1CCC13CNC3)c1ncnn12)c1cccc(C(F)F)c1F] |
| 46 | 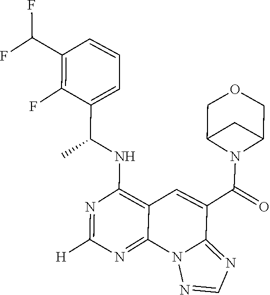 [SMILES: C[C@@H](Nc1ncnc2c1cc(C(=O)N1C3COCC1C3)c1ncnn12)c1cccc(C(F)F)c1F] |
| 47 | 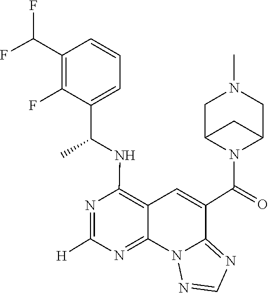 [SMILES: C[C@@H](Nc1ncnc2c1cc(C(=O)N1C3CC1CN(C)C3)c1ncnn12)c1cccc(C(F)F)c1F] |
| 48 | 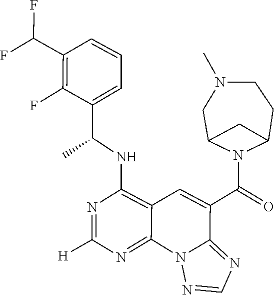 [SMILES: C[C@@H](Nc1ncnc2c1cc(C(=O)N1C3CCN(C)CC1C3)c1ncnn12)c1cccc(C(F)F)c1F] |
| 49 | 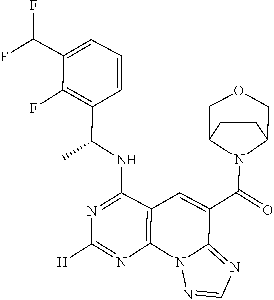 [SMILES: C[C@@H](Nc1ncnc2c1cc(C(=O)N1C3CCC1COC3)c1ncnn12)c1cccc(C(F)F)c1F] |
| 50 | 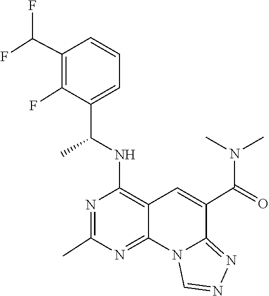 [SMILES: Cc1nc(N[C@H](C)c2cccc(C(F)F)c2F)c2cc(C(=O)N(C)C)c3nncn3c2n1] |
| 51 | 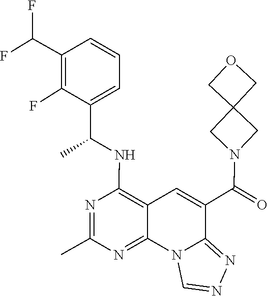 [SMILES: Cc1nc(N[C@H](C)c2cccc(C(F)F)c2F)c2cc(C(=O)N3CC4(COC4)C3)c3nncn3c2n1] |
| 52 | 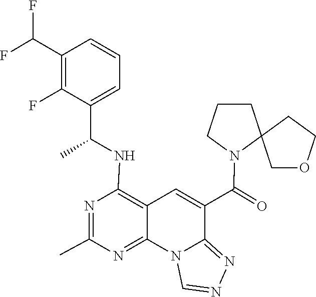 [SMILES: Cc1nc(N[C@H](C)c2cccc(C(F)F)c2F)c2cc(C(=O)N3CCCC34CCOC4)c3nncn3c2n1] |
| 53 | 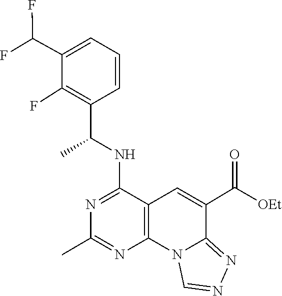 [SMILES: CCOC(=O)c1cc2c(N[C@H](C)c3cccc(C(F)F)c3F)nc(C)nc2n2cnnc12] |
| 54 | 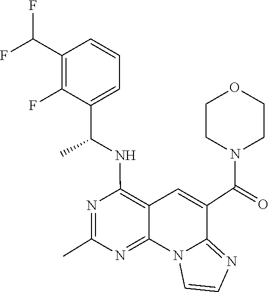 [SMILES: Cc1nc(N[C@H](C)c2cccc(C(F)F)c2F)c2cc(C(=O)N3CCOCC3)c3nccn3c2n1] |
| 55 | 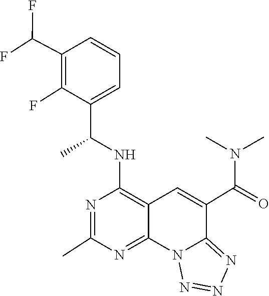 [SMILES: Cc1nc(N[C@H](C)c2cccc(C(F)F)c2F)c2cc(C(=O)N(C)C)c3nnnn3c2n1] |
| 56 | 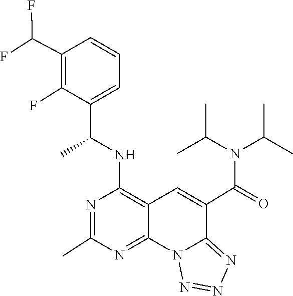 [SMILES: Cc1nc(N[C@H](C)c2cccc(C(F)F)c2F)c2cc(C(=O)N(C(C)C)C(C)C)c3nnnn3c2n1] |
| 57 | 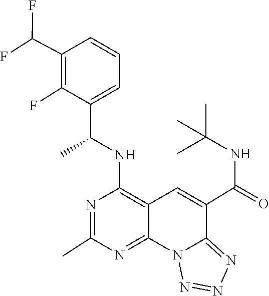 [SMILES: Cc1nc(N[C@H](C)c2cccc(C(F)F)c2F)c2cc(C(=O)NC(C)(C)C)c3nnnn3c2n1] |
| 58 | 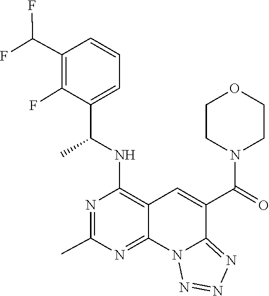 [SMILES: Cc1nc(N[C@H](C)c2cccc(C(F)F)c2F)c2cc(C(=O)N3CCOCC3)c3nnnn3c2n1] |
| 59 | 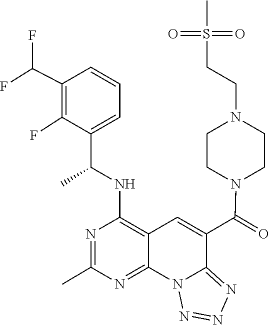 [SMILES: Cc1nc(N[C@H](C)c2cccc(C(F)F)c2F)c2cc(C(=O)N3CCN(CCS(C)(=O)=O)CC3)c3nnnn3c2n1] |
| 60 | 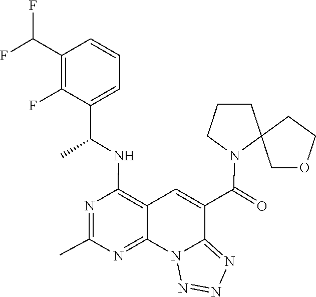 [SMILES: Cc1nc(N[C@H](C)c2cccc(C(F)F)c2F)c2cc(C(=O)N3CCCC34CCOC4)c3nnnn3c2n1] |
| 61 | 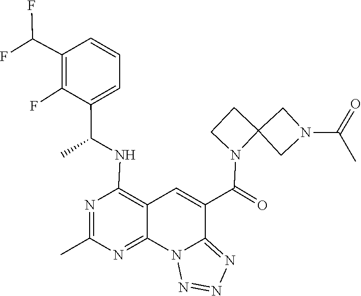 [SMILES: CC(=O)N1CC2(CCN2C(=O)c2cc3c(N[C@H](C)c4cccc(C(F)F)c4F)nc(C)nc3n3nnnc23)C1] |
| 62 | 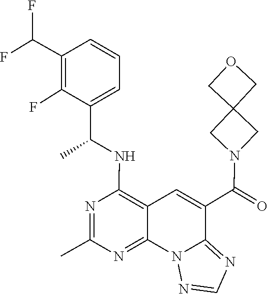 [SMILES: Cc1nc(N[C@H](C)c2cccc(C(F)F)c2F)c2cc(C(=O)N3CC4(COC4)C3)c3ncnn3c2n1] |
| 63 | 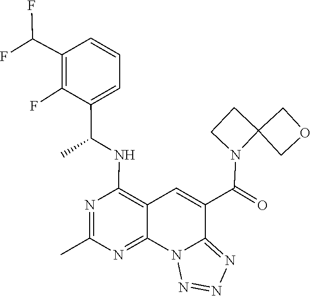 [SMILES: Cc1nc(N[C@H](C)c2cccc(C(F)F)c2F)c2cc(C(=O)N3CCC34COC4)c3nnnn3c2n1] |
| 64 | 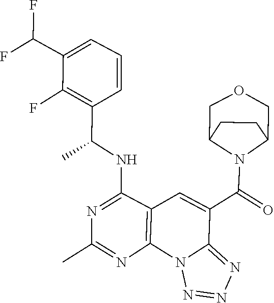 [SMILES: Cc1nc(N[C@H](C)c2cccc(C(F)F)c2F)c2cc(C(=O)N3C4CCC3COC4)c3nnnn3c2n1] |
| 65 | 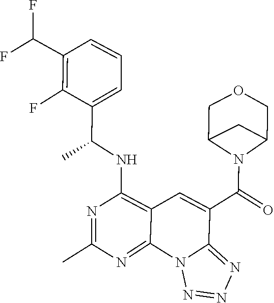 [SMILES: Cc1nc(N[C@H](C)c2cccc(C(F)F)c2F)c2cc(C(=O)N3C4COCC3C4)c3nnnn3c2n1] |
| 66 | 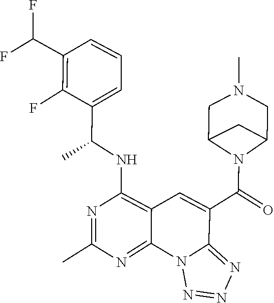 [SMILES: Cc1nc(N[C@H](C)c2cccc(C(F)F)c2F)c2cc(C(=O)N3C4CC3CN(C)C4)c3nnnn3c2n1] |
| 67 | 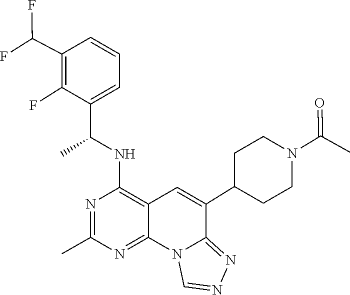 [SMILES: CC(=O)N1CCC(c2cc3c(N[C@H](C)c4cccc(C(F)F)c4F)nc(C)nc3n3cnnc23)CC1] |
| 68 | 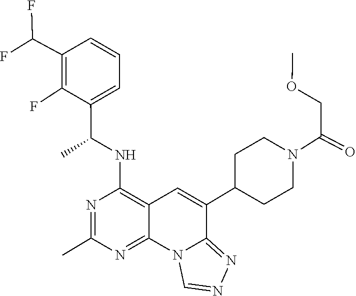 [SMILES: COCC(=O)N1CCC(c2cc3c(N[C@H](C)c4cccc(C(F)F)c4F)nc(C)nc3n3cnnc23)CC1] |
| 69 | 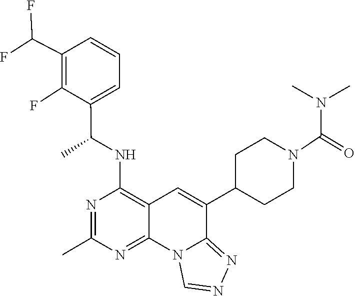 [SMILES: Cc1nc(N[C@H](C)c2cccc(C(F)F)c2F)c2cc(C3CCN(C(=O)N(C)C)CC3)c3nncn3c2n1] |
| 70 | 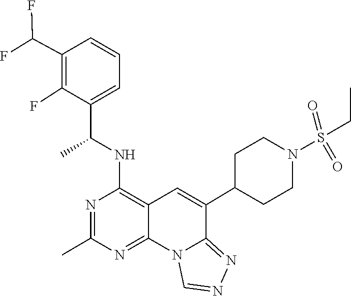 [SMILES: CCS(=O)(=O)N1CCC(c2cc3c(N[C@H](C)c4cccc(C(F)F)c4F)nc(C)nc3n3cnnc23)CC1] |
| 71 | 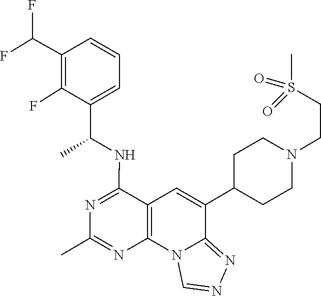 [SMILES: Cc1nc(N[C@H](C)c2cccc(C(F)F)c2F)c2cc(C3CCN(CCS(C)(=O)=O)CC3)c3nncn3c2n1] |
| 72 | 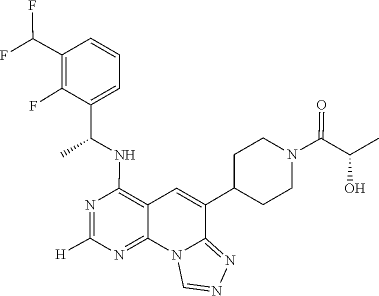 [SMILES: C[C@H](O)C(=O)N1CCC(c2cc3c(N[C@H](C)c4cccc(C(F)F)c4F)ncnc3n3cnnc23)CC1] |
| 73 | 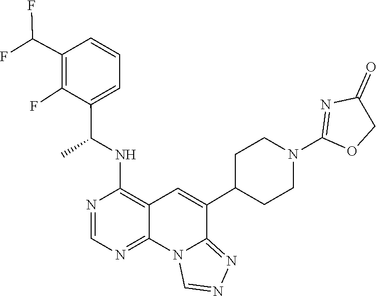 [SMILES: C[C@@H](Nc1ncnc2c1cc(C1CCN(C3=NC(=O)CO3)CC1)c1nncn12)c1cccc(C(F)F)c1F] |
| 74 | 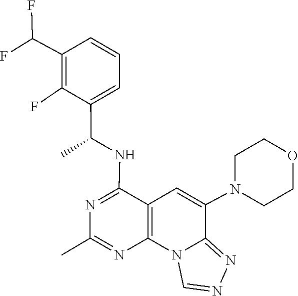 [SMILES: Cc1nc(N[C@H](C)c2cccc(C(F)F)c2F)c2cc(N3CCOCC3)c3nncn3c2n1] |
| 75 | 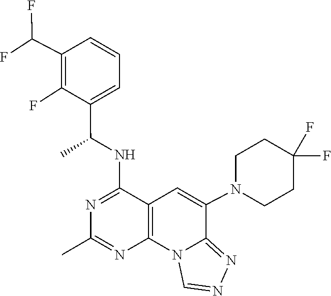 [SMILES: Cc1nc(N[C@H](C)c2cccc(C(F)F)c2F)c2cc(N3CCC(F)(F)CC3)c3nncn3c2n1] |
| 76 | 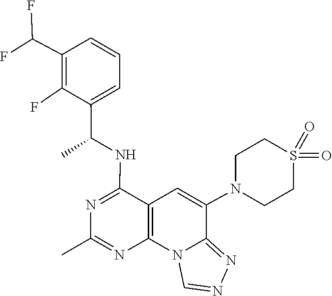 [SMILES: Cc1nc(N[C@H](C)c2cccc(C(F)F)c2F)c2cc(N3CCS(=O)(=O)CC3)c3nncn3c2n1] |
| 77 | 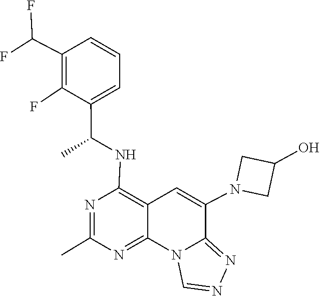 [SMILES: Cc1nc(N[C@H](C)c2cccc(C(F)F)c2F)c2cc(N3CC(O)C3)c3nncn3c2n1] |
| 78 | 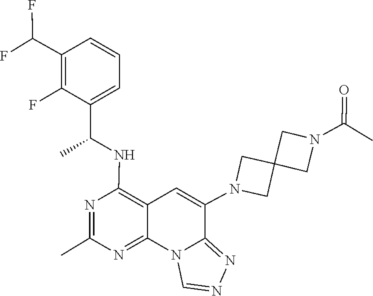 [SMILES: CC(=O)N1CC2(C1)CN(c1cc3c(N[C@H](C)c4cccc(C(F)F)c4F)nc(C)nc3n3cnnc13)C2] |
| 79 | 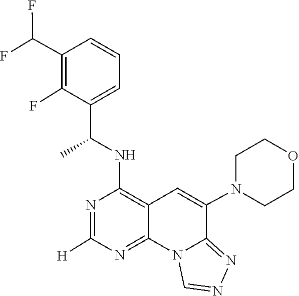 [SMILES: C[C@@H](Nc1ncnc2c1cc(N1CCOCC1)c1nncn12)c1cccc(C(F)F)c1F] |
| 80 | 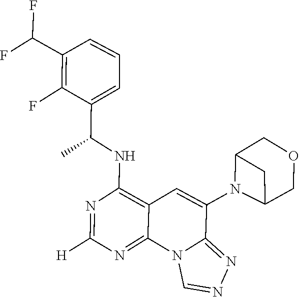 [SMILES: C[C@@H](Nc1ncnc2c1cc(N1C3COCC1C3)c1nncn12)c1cccc(C(F)F)c1F] |
| 81 | 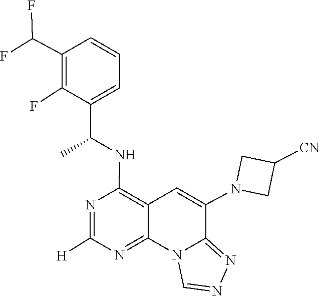 [SMILES: C[C@@H](Nc1ncnc2c1cc(N1CC(C#N)C1)c1nncn12)c1cccc(C(F)F)c1F] |
| 82 | 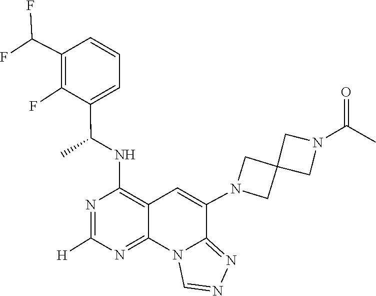 [SMILES: CC(=O)N1CC2(C1)CN(c1cc3c(N[C@H](C)c4cccc(C(F)F)c4F)ncnc3n3cnnc13)C2] |
| 83 | 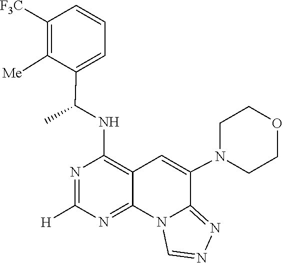 [SMILES: Cc1c([C@@H](C)Nc2ncnc3c2cc(N2CCOCC2)c2nncn23)cccc1C(F)(F)F] |
| 84 | 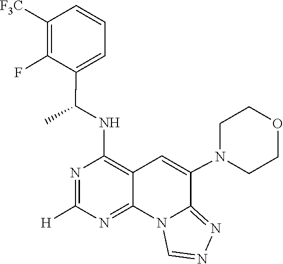 [SMILES: C[C@@H](Nc1ncnc2c1cc(N1CCOCC1)c1nncn12)c1cccc(C(F)(F)F)c1F] |
| 85 | 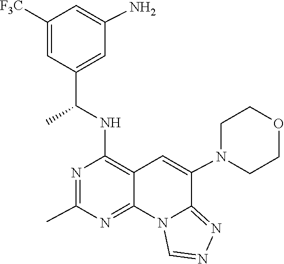 [SMILES: Cc1nc(N[C@H](C)c2cc(N)cc(C(F)(F)F)c2)c2cc(N3CCOCC3)c3nncn3c2n1] |
| 86 | 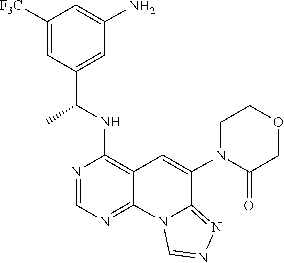 [SMILES: C[C@@H](Nc1ncnc2c1cc(N1CCOCC1=O)c1nncn12)c1cc(N)cc(C(F)(F)F)c1] |
| 87 | 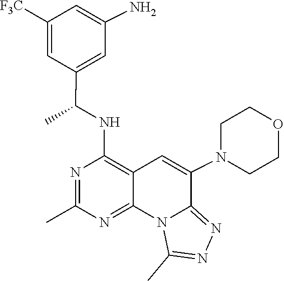 [SMILES: Cc1nc(N[C@H](C)c2cc(N)cc(C(F)(F)F)c2)c2cc(N3CCOCC3)c3nnc(C)n3c2n1] |
| 88 | 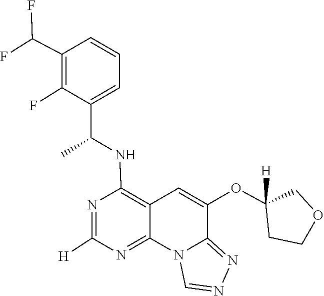 [SMILES: C[C@@H](Nc1ncnc2c1cc(O[C@H]1CCOC1)c1nncn12)c1cccc(C(F)F)c1F] |
| 89 | 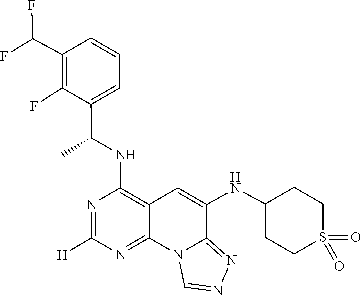 [SMILES: C[C@@H](Nc1ncnc2c1cc(NC1CCS(=O)(=O)CC1)c1nncn12)c1cccc(C(F)F)c1F] |
| 90 | 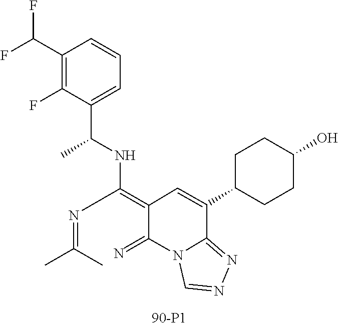 [SMILES: CC(C)=N/C(N[C@H](C)c1cccc(C(F)F)c1F)=c1/cc([C@H]2CC[C@@H](O)CC2)c2nncn2c1=N] |
| | 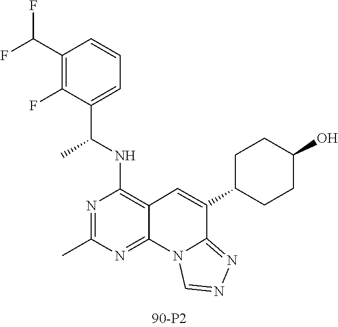 [SMILES: Cc1nc(N[C@H](C)c2cccc(C(F)F)c2F)c2cc([C@H]3CC[C@H](O)CC3)c3nncn3c2n1] |
| 91 | 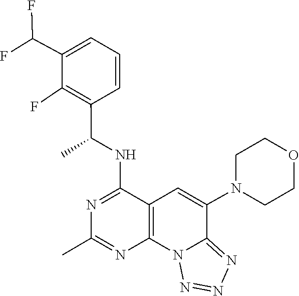 [SMILES: Cc1nc(N[C@H](C)c2cccc(C(F)F)c2F)c2cc(N3CCOCC3)c3nnnn3c2n1] |
| 92 | 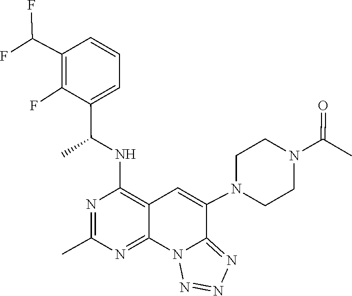 [SMILES: CC(=O)N1CCN(c2cc3c(N[C@H](C)c4cccc(C(F)F)c4F)nc(C)nc3n3nnnc23)CC1] |
| 93 | 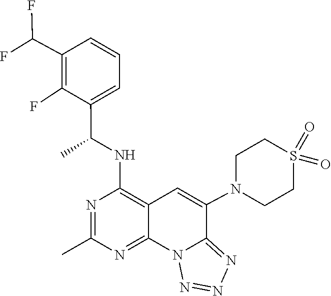 [SMILES: Cc1nc(N[C@H](C)c2cccc(C(F)F)c2F)c2cc(N3CCS(=O)(=O)CC3)c3nnnn3c2n1] |
| 94 | 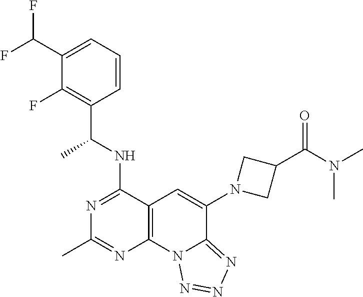 [SMILES: Cc1nc(N[C@H](C)c2cccc(C(F)F)c2F)c2cc(N3CC(C(=O)N(C)C)C3)c3nnnn3c2n1] |
| 95 | 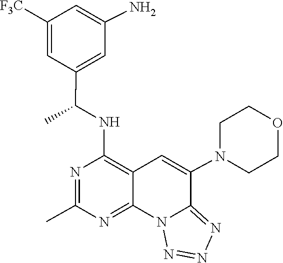 [SMILES: Cc1nc(N[C@H](C)c2cc(N)cc(C(F)(F)F)c2)c2cc(N3CCOCC3)c3nnnn3c2n1] |
| 96 | 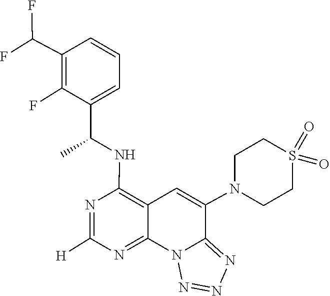 [SMILES: C[C@@H](Nc1ncnc2c1cc(N1CCS(=O)(=O)CC1)c1nnnn12)c1cccc(C(F)F)c1F] |
| 97 | 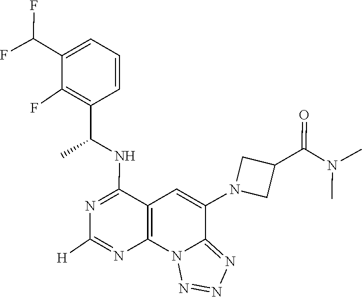 [SMILES: C[C@@H](Nc1ncnc2c1cc(N1CC(C(=O)N(C)C)C1)c1nnnn12)c1cccc(C(F)F)c1F] |
| 98 | 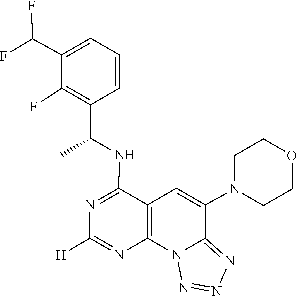 [SMILES: C[C@@H](Nc1ncnc2c1cc(N1CCOCC1)c1nnnn12)c1cccc(C(F)F)c1F] |
| 99 | 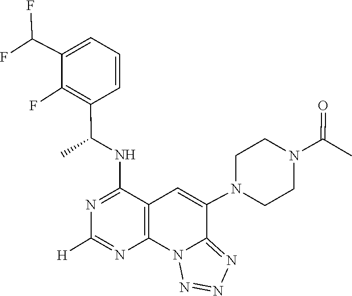 [SMILES: CC(=O)N1CCN(c2cc3c(N[C@H](C)c4cccc(C(F)F)c4F)ncnc3n3nnnc23)CC1] |
| 100 | 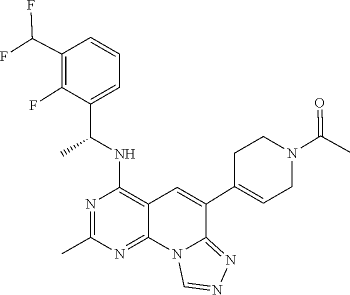 [SMILES: CC(=O)N1CC=C(c2cc3c(N[C@H](C)c4cccc(C(F)F)c4F)nc(C)nc3n3cnnc23)CC1] |
| 101 | 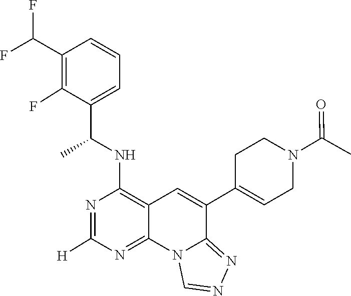 [SMILES: CC(=O)N1CC=C(c2cc3c(N[C@H](C)c4cccc(C(F)F)c4F)ncnc3n3cnnc23)CC1] |
| 102 | 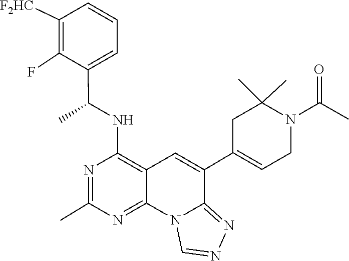 [SMILES: CC(=O)N1CC=C(c2cc3c(N[C@H](C)c4cccc(C(F)F)c4F)nc(C)nc3n3cnnc23)CC1(C)C] |
| 103 | 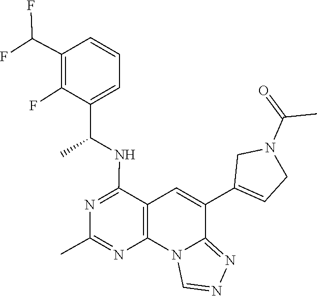 [SMILES: CC(=O)N1CC=C(c2cc3c(N[C@H](C)c4cccc(C(F)F)c4F)nc(C)nc3n3cnnc23)C1] |
| 104 | 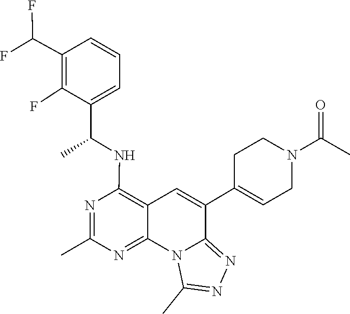 [SMILES: CC(=O)N1CC=C(c2cc3c(N[C@H](C)c4cccc(C(F)F)c4F)nc(C)nc3n3c(C)nnc23)CC1] |
| 105 | 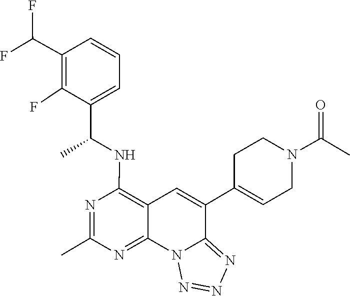 [SMILES: CC(=O)N1CC=C(c2cc3c(N[C@H](C)c4cccc(C(F)F)c4F)nc(C)nc3n3nnnc23)CC1] |
| 106 | 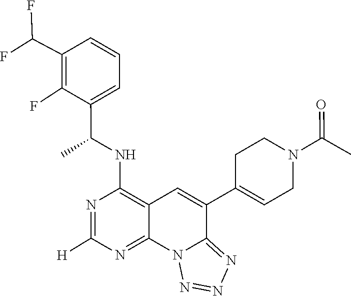 [SMILES: CC(=O)N1CC=C(c2cc3c(N[C@H](C)c4cccc(C(F)F)c4F)ncnc3n3nnnc23)CC1] |
| 107 | 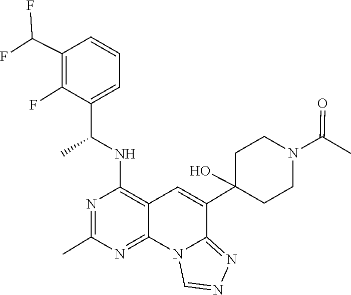 [SMILES: CC(=O)N1CCC(O)(c2cc3c(N[C@H](C)c4cccc(C(F)F)c4F)nc(C)nc3n3cnnc23)CC1] |
| 108 | 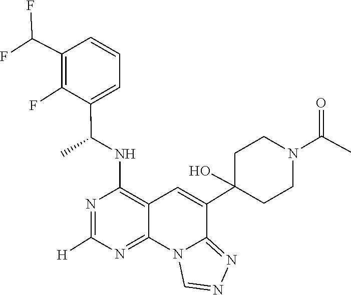 [SMILES: CC(=O)N1CCC(O)(c2cc3c(N[C@H](C)c4cccc(C(F)F)c4F)ncnc3n3cnnc23)CC1] |
| 109 | 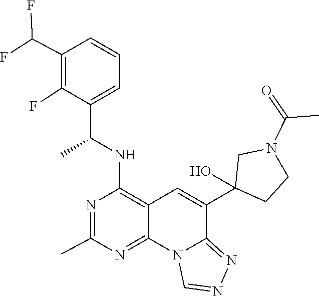 [SMILES: CC(=O)N1CCC(O)(c2cc3c(N[C@H](C)c4cccc(C(F)F)c4F)nc(C)nc3n3cnnc23)C1] |
| 110 | 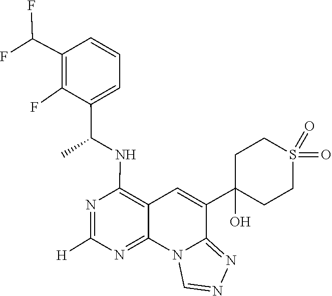 [SMILES: C[C@@H](Nc1ncnc2c1cc(C1(O)CCS(=O)(=O)CC1)c1nncn12)c1cccc(C(F)F)c1F] |
| 111 | 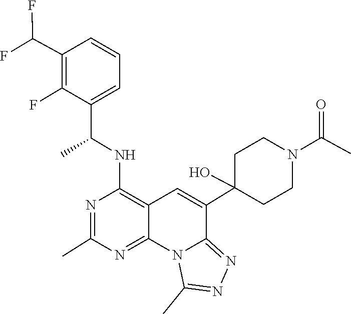 [SMILES: CC(=O)N1CCC(O)(c2cc3c(N[C@H](C)c4cccc(C(F)F)c4F)nc(C)nc3n3c(C)nnc23)CC1] |
| 112 | 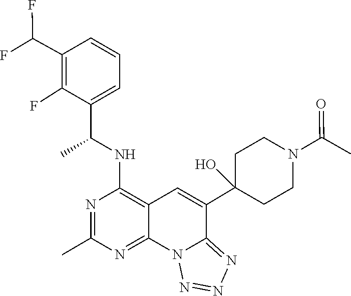 [SMILES: CC(=O)N1CCC(O)(c2cc3c(N[C@H](C)c4cccc(C(F)F)c4F)nc(C)nc3n3nnnc23)CC1] |
| 113 | 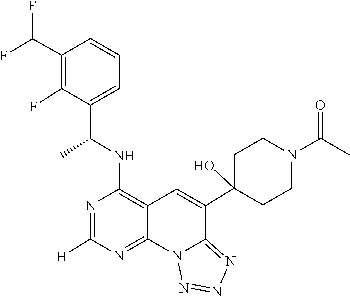 [SMILES: CC(=O)N1CCC(O)(c2cc3c(N[C@H](C)c4cccc(C(F)F)c4F)ncnc3n3nnnc23)CC1] |
| 114 | 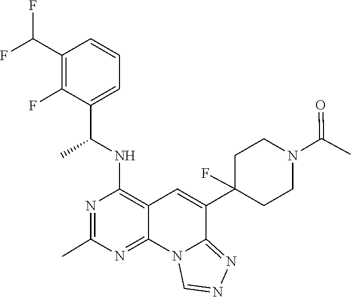 [SMILES: CC(=O)N1CCC(F)(c2cc3c(N[C@H](C)c4cccc(C(F)F)c4F)nc(C)nc3n3cnnc23)CC1] |
| 115 | 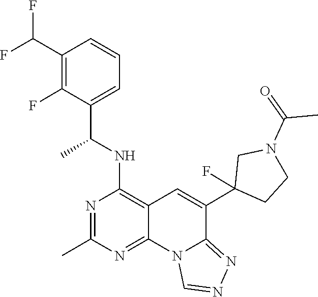 [SMILES: CC(=O)N1CCC(F)(c2cc3c(N[C@H](C)c4cccc(C(F)F)c4F)nc(C)nc3n3cnnc23)C1] |
| 116 | 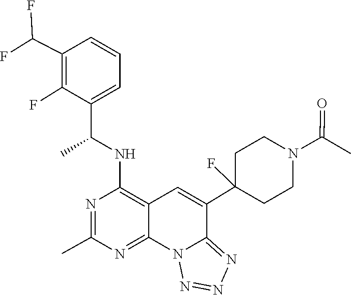 [SMILES: CC(=O)N1CCC(F)(c2cc3c(N[C@H](C)c4cccc(C(F)F)c4F)nc(C)nc3n3nnnc23)CC1] |
| 117 | 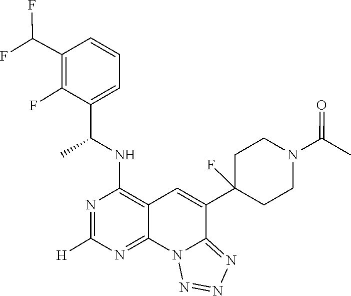 [SMILES: CC(=O)N1CCC(F)(c2cc3c(N[C@H](C)c4cccc(C(F)F)c4F)ncnc3n3nnnc23)CC1] |
| 118 | 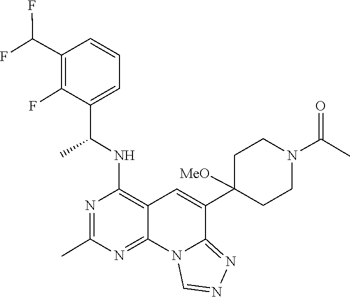 [SMILES: COC1(c2cc3c(N[C@H](C)c4cccc(C(F)F)c4F)nc(C)nc3n3cnnc23)CCN(C(C)=O)CC1] |
| 119 | 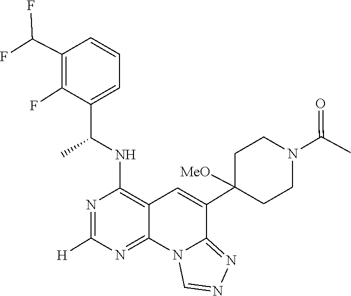 [SMILES: COC1(c2cc3c(N[C@H](C)c4cccc(C(F)F)c4F)ncnc3n3cnnc23)CCN(C(C)=O)CC1] |
| 120 | 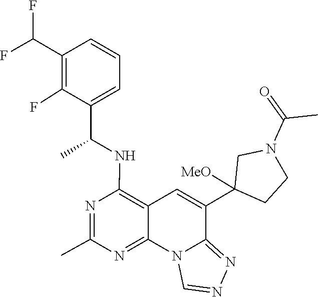 [SMILES: COC1(c2cc3c(N[C@H](C)c4cccc(C(F)F)c4F)nc(C)nc3n3cnnc23)CCN(C(C)=O)C1] |
| 121 | 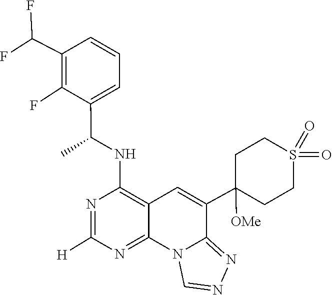 [SMILES: COC1(c2cc3c(N[C@H](C)c4cccc(C(F)F)c4F)ncnc3n3cnnc23)CCS(=O)(=O)CC1] |
| 122 | 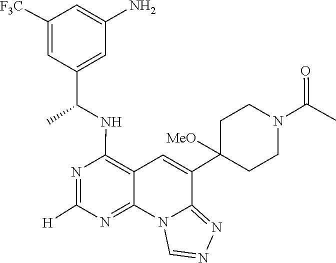 [SMILES: COC1(c2cc3c(N[C@H](C)c4cc(N)cc(C(F)(F)F)c4)ncnc3n3cnnc23)CCN(C(C)=O)CC1] |
| 123 | 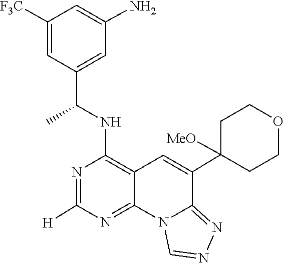 [SMILES: COC1(c2cc3c(N[C@H](C)c4cc(N)cc(C(F)(F)F)c4)ncnc3n3cnnc23)CCOCC1] |
| 124 | 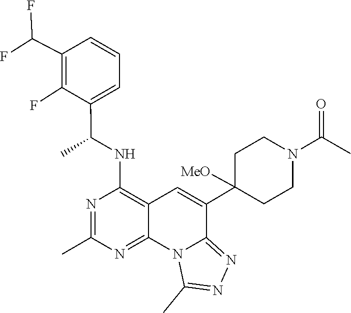 [SMILES: COC1(c2cc3c(N[C@H](C)c4cccc(C(F)F)c4F)nc(C)nc3n3c(C)nnc23)CCN(C(C)=O)CC1] |
| 125 | 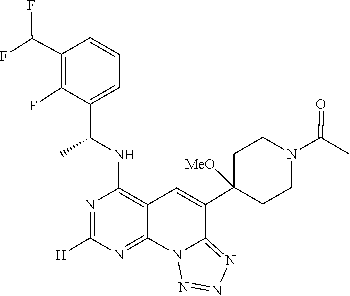 [SMILES: COC1(c2cc3c(N[C@H](C)c4cccc(C(F)F)c4F)ncnc3n3nnnc23)CCN(C(C)=O)CC1] |
| 126 | 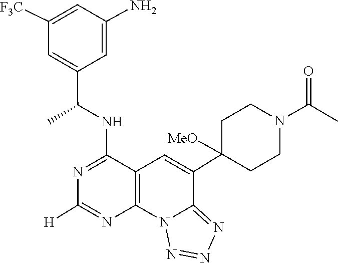 [SMILES: COC1(c2cc3c(N[C@H](C)c4cc(N)cc(C(F)(F)F)c4)ncnc3n3nnnc23)CCN(C(C)=O)CC1] |
| 127 | 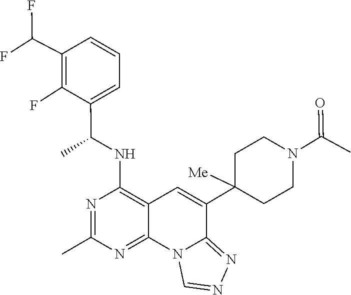 [SMILES: CC(=O)N1CCC(C)(c2cc3c(N[C@H](C)c4cccc(C(F)F)c4F)nc(C)nc3n3cnnc23)CC1] |
| 128 | 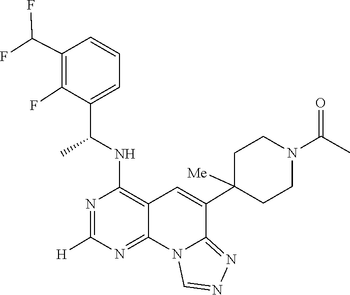 [SMILES: CC(=O)N1CCC(C)(c2cc3c(N[C@H](C)c4cccc(C(F)F)c4F)ncnc3n3cnnc23)CC1] |
| 129 | 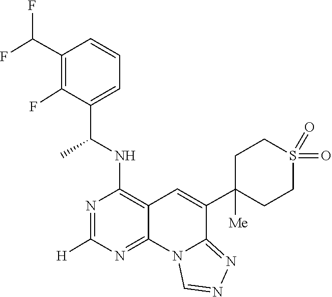 [SMILES: C[C@@H](Nc1ncnc2c1cc(C1(C)CCS(=O)(=O)CC1)c1nncn12)c1cccc(C(F)F)c1F] |
| 130 | 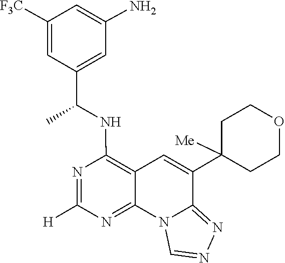 [SMILES: C[C@@H](Nc1ncnc2c1cc(C1(C)CCOCC1)c1nncn12)c1cc(N)cc(C(F)(F)F)c1] |
| 131 | 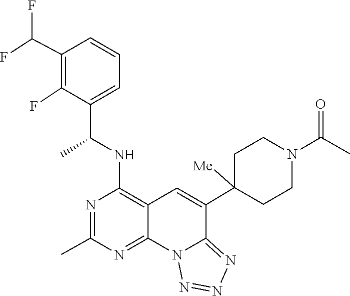 [SMILES: CC(=O)N1CCC(C)(c2cc3c(N[C@H](C)c4cccc(C(F)F)c4F)nc(C)nc3n3nnnc23)CC1] |
| 132 | 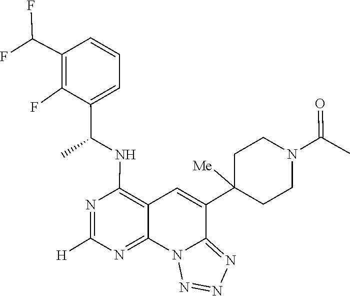 [SMILES: CC(=O)N1CCC(C)(c2cc3c(N[C@H](C)c4cccc(C(F)F)c4F)ncnc3n3nnnc23)CC1] |
| 133 | 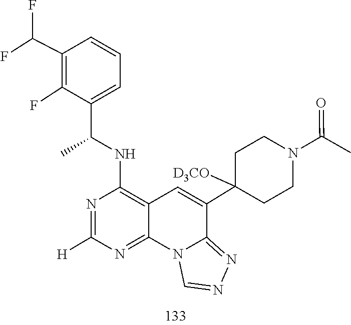 |
| 134 | 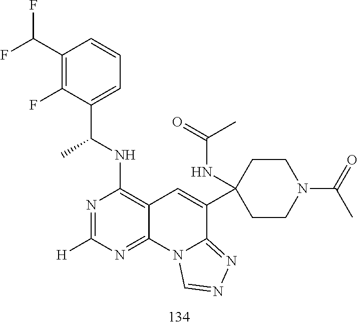 [SMILES: CC(=O)NC1(c2cc3c(N[C@H](C)c4cccc(C(F)F)c4F)ncnc3n3cnnc23)CCN(C(C)=O)CC1] |
| 135 | 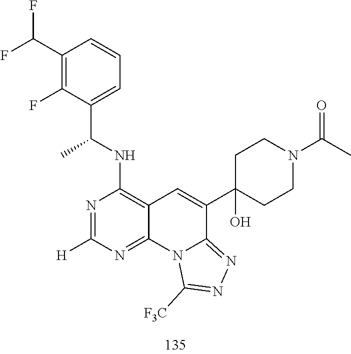 [SMILES: CC(=O)N1CCC(O)(c2cc3c(N[C@H](C)c4cccc(C(F)F)c4F)ncnc3n3c(C(F)(F)F)nnc23)CC1] |
| 136 | 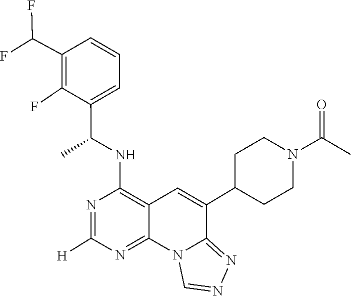 [SMILES: CC(=O)N1CCC(c2cc3c(N[C@H](C)c4cccc(C(F)F)c4F)ncnc3n3cnnc23)CC1] |
| 137 | 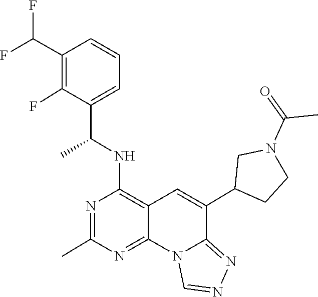 [SMILES: CC(=O)N1CCC(c2cc3c(N[C@H](C)c4cccc(C(F)F)c4F)nc(C)nc3n3cnnc23)C1] |
| 138 | 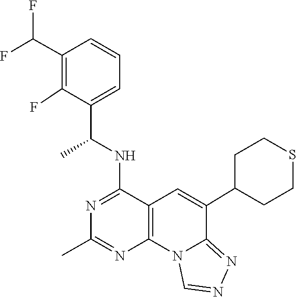 [SMILES: Cc1nc(N[C@H](C)c2cccc(C(F)F)c2F)c2cc(C3CCSCC3)c3nncn3c2n1] |
| 139 | 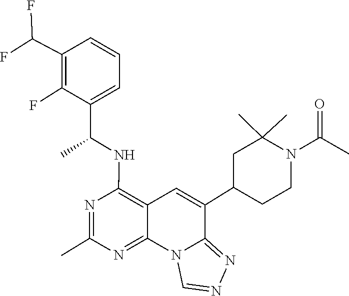 [SMILES: CC(=O)N1CCC(c2cc3c(N[C@H](C)c4cccc(C(F)F)c4F)nc(C)nc3n3cnnc23)CC1(C)C] |
| 140 | 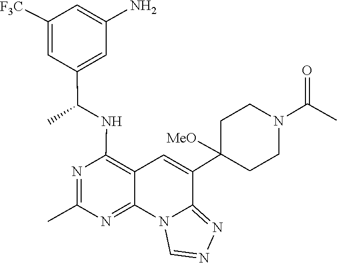 [SMILES: COC1(c2cc3c(N[C@H](C)c4cc(N)cc(C(F)(F)F)c4)nc(C)nc3n3cnnc23)CCN(C(C)=O)CC1] |
| 141 | 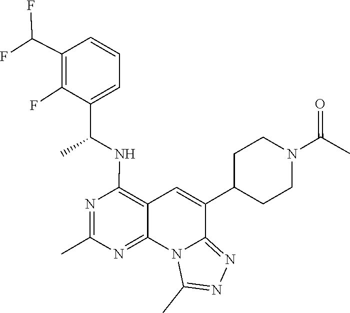 [SMILES: CC(=O)N1CCC(c2cc3c(N[C@H](C)c4cccc(C(F)F)c4F)nc(C)nc3n3c(C)nnc23)CC1] |
| 142 | 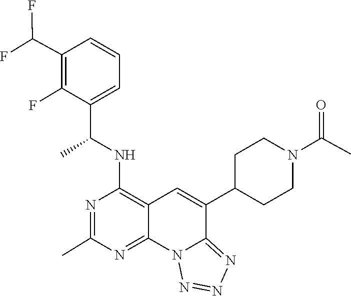 [SMILES: CC(=O)N1CCC(c2cc3c(N[C@H](C)c4cccc(C(F)F)c4F)nc(C)nc3n3nnnc23)CC1] |
| 143 | 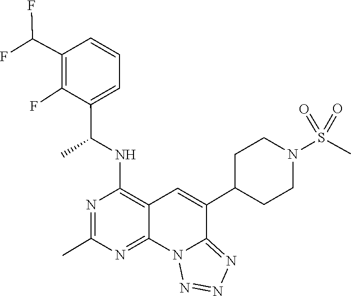 [SMILES: Cc1nc(N[C@H](C)c2cccc(C(F)F)c2F)c2cc(C3CCN(S(C)(=O)=O)CC3)c3nnnn3c2n1] |
| 144 | 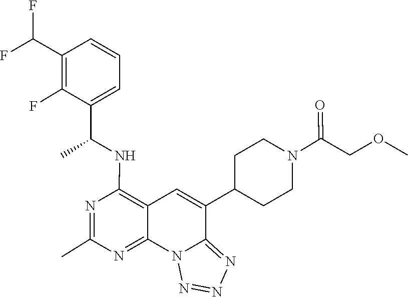 [SMILES: COCC(=O)N1CCC(c2cc3c(N[C@H](C)c4cccc(C(F)F)c4F)nc(C)nc3n3nnnc23)CC1] |
| 145 | 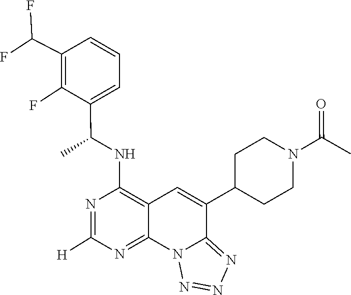 [SMILES: CC(=O)N1CCC(c2cc3c(N[C@H](C)c4cccc(C(F)F)c4F)ncnc3n3nnnc23)CC1] |
| 146 | 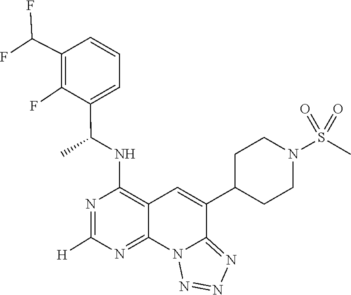 [SMILES: C[C@@H](Nc1ncnc2c1cc(C1CCN(S(C)(=O)=O)CC1)c1nnnn12)c1cccc(C(F)F)c1F] |
| 147 | 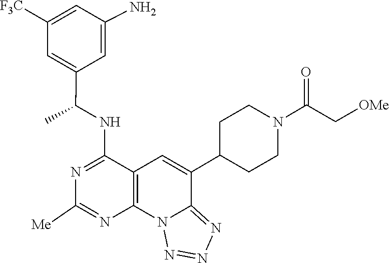 [SMILES: COCC(=O)N1CCC(c2cc3c(N[C@H](C)c4cc(N)cc(C(F)(F)F)c4)nc(C)nc3n3nnnc23)CC1] |
| 148 | 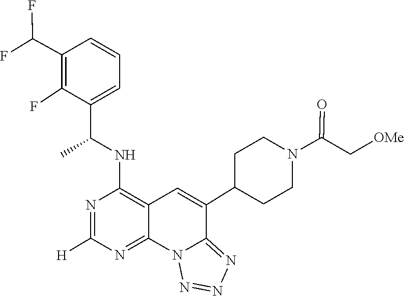 [SMILES: COCC(=O)N1CCC(c2cc3c(N[C@H](C)c4cccc(C(F)F)c4F)ncnc3n3nnnc23)CC1] |
| 149 | 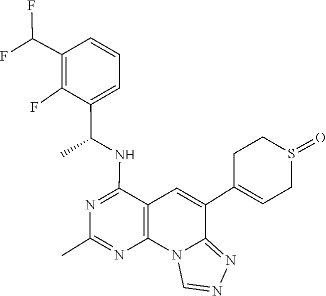 [SMILES: Cc1nc(N[C@H](C)c2cccc(C(F)F)c2F)c2cc(C3=CCS(=O)CC3)c3nncn3c2n1] |
| 150 | 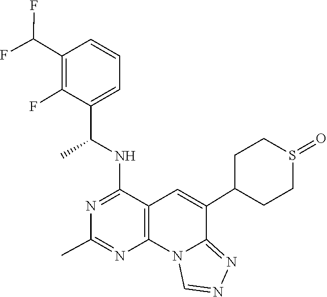 [SMILES: Cc1nc(N[C@H](C)c2cccc(C(F)F)c2F)c2cc(C3CCS(=O)CC3)c3nncn3c2n1] |
| 151 | 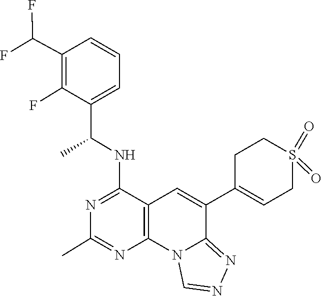 [SMILES: Cc1nc(N[C@H](C)c2cccc(C(F)F)c2F)c2cc(C3=CCS(=O)(=O)CC3)c3nncn3c2n1] |
| 152 | 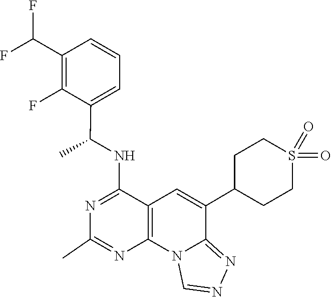 [SMILES: Cc1nc(N[C@H](C)c2cccc(C(F)F)c2F)c2cc(C3CCS(=O)(=O)CC3)c3nncn3c2n1] |
| 153 | 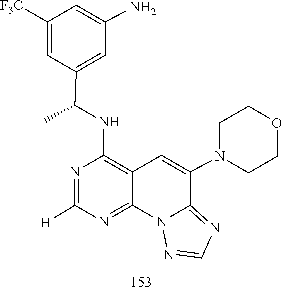 [SMILES: C[C@@H](Nc1ncnc2c1cc(N1CCOCC1)c1ncnn12)c1cc(N)cc(C(F)(F)F)c1] |
| 154 | 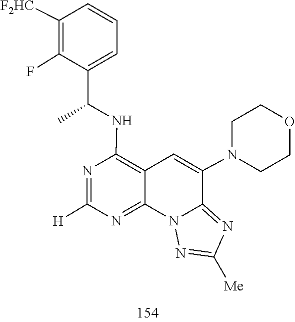 [SMILES: Cc1nc2c(N3CCOCC3)cc3c(N[C@H](C)c4cccc(C(F)F)c4F)ncnc3n2n1] |
| 155 | 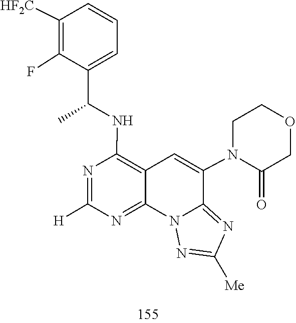 |
| 156 | 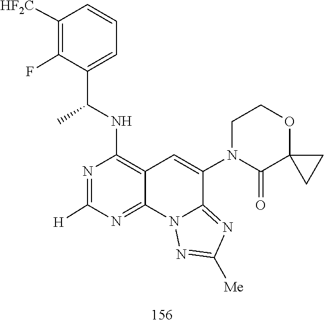 |
| 157 | 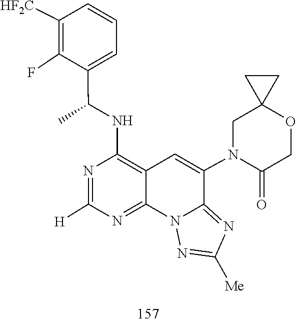 |
| 158 | 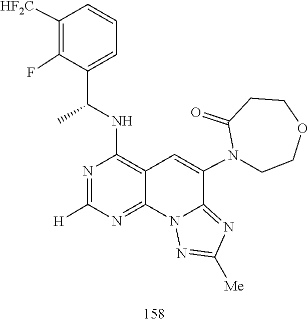 |
| 159 | 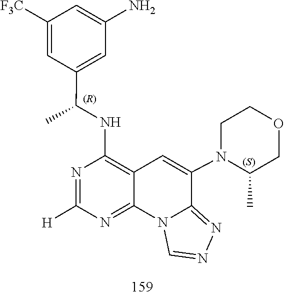 |
| 160 | 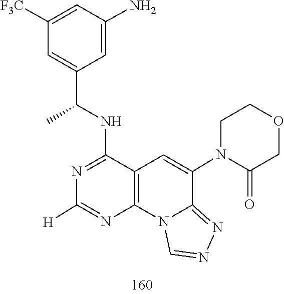 [SMILES: C[C@@H](Nc1ncnc2c1cc(N1CCOCC1=O)c1nncn12)c1cc(N)cc(C(F)(F)F)c1] |
| 161 | 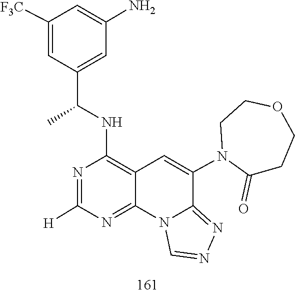 [SMILES: C[C@@H](Nc1ncnc2c1cc(N1CCOCCC1=O)c1nncn12)c1cc(N)cc(C(F)(F)F)c1] |
| 162 | 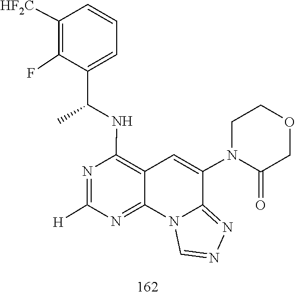 |
| 163 | 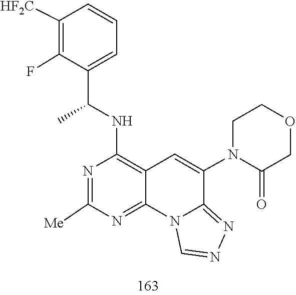 |
| 164 | 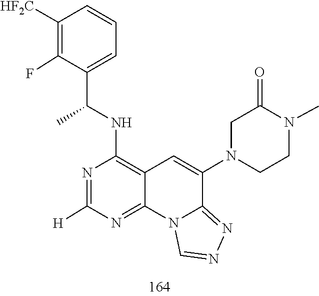 |
| 165 | 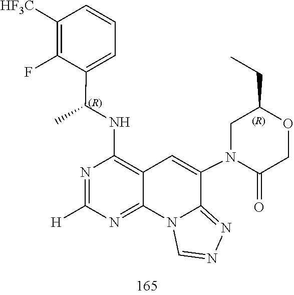 |
| 166 | 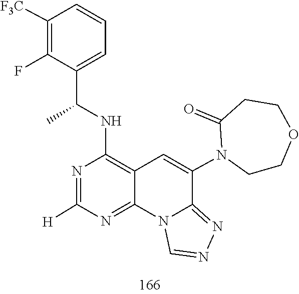 [SMILES: C[C@@H](Nc1ncnc2c1cc(N1CCOCCC1=O)c1nncn12)c1cccc(C(F)(F)F)c1F] |
| 167 | 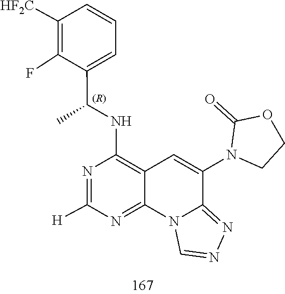 |
| 168 | 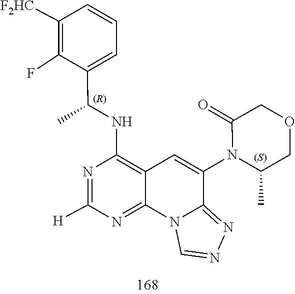 |
| 169 | 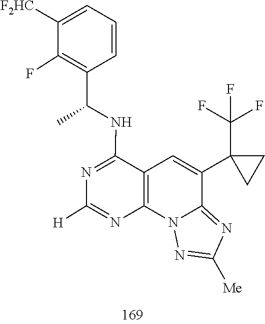 [SMILES: Cc1nc2c(C3(C(F)(F)F)CC3)cc3c(N[C@H](C)c4cccc(C(F)F)c4F)ncnc3n2n1] |
| 170 | 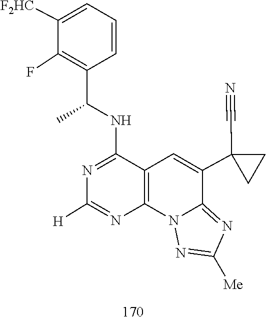 [SMILES: Cc1nc2c(C3(C#N)CC3)cc3c(N[C@H](C)c4cccc(C(F)F)c4F)ncnc3n2n1] |
| 171 | 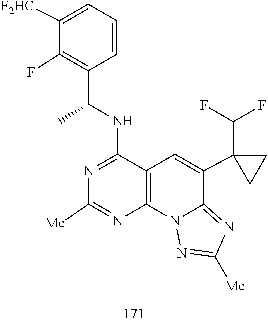 [SMILES: Cc1nc(N[C@H](C)c2cccc(C(F)F)c2F)c2cc(C3(C(F)F)CC3)c3nc(C)nn3c2n1] |
| 172 | 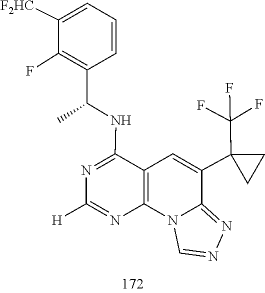 [SMILES: C[C@@H](Nc1ncnc2c1cc(C1(C(F)(F)F)CC1)c1nncn12)c1cccc(C(F)F)c1F] |
| 173 | 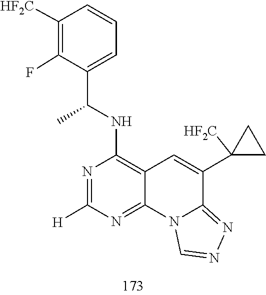 |
| 174 | 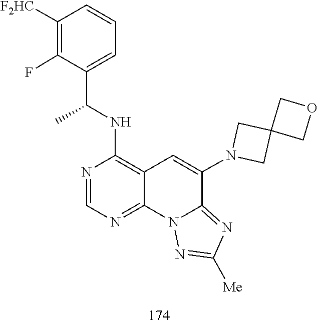 [SMILES: Cc1nc2c(N3CC4(COC4)C3)cc3c(N[C@H](C)c4cccc(C(F)F)c4F)ncnc3n2n1] |

In some embodiments, the preferred compounds are selected from the following:

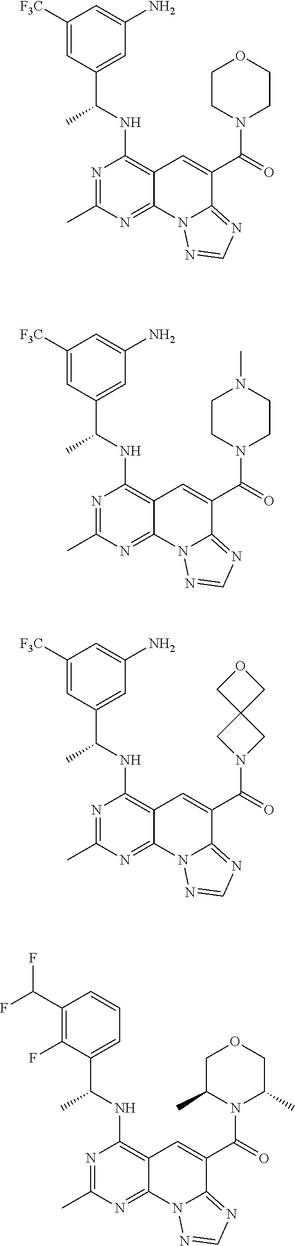 [SMILES: Cc1nc(N[C@H](C)c2cc(N)cc(C(F)(F)F)c2)c2cc(C(=O)N3CCOCC3)c3ncnn3c2n1 ; Cc1nc(N[C@H](C)c2cc(N)cc(C(F)(F)F)c2)c2cc(C(=O)N3CCN(C)CC3)c3ncnn3c2n1 ; Cc1nc(N[C@H](C)c2cc(N)cc(C(F)(F)F)c2)c2cc(C(=O)N3CC4(COC4)C3)c3ncnn3c2n1 ; Cc1nc(N[C@H](C)c2cccc(C(F)F)c2F)c2cc(C(=O)N3[C@@H](C)COC[C@@H]3C)c3ncnn3c2n1]

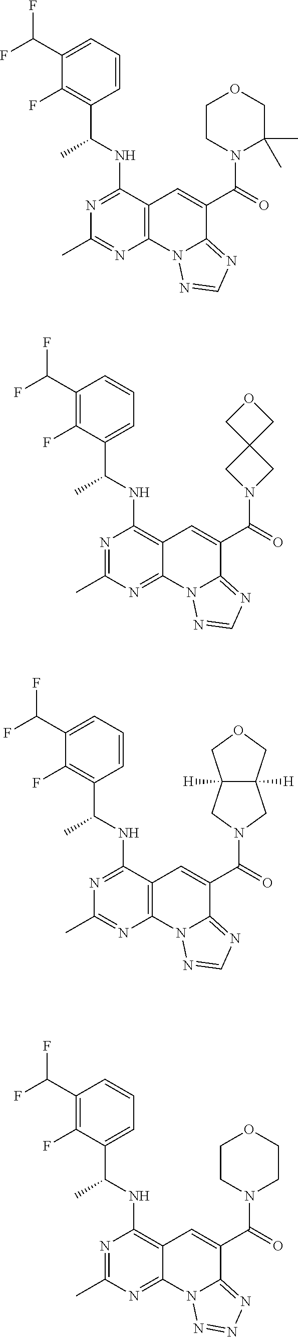 [SMILES: Cc1nc(N[C@H](C)c2cccc(C(F)F)c2F)c2cc(C(=O)N3CCOCC3(C)C)c3ncnn3c2n1 ; Cc1nc(N[C@H](C)c2cccc(C(F)F)c2F)c2cc(C(=O)N3CC4(COC4)C3)c3ncnn3c2n1 ; Cc1nc(N[C@H](C)c2cccc(C(F)F)c2F)c2cc(C(=O)N3C[C@H]4COC[C@H]4C3)c3ncnn3c2n1 ; Cc1nc(N[C@H](C)c2cccc(C(F)F)c2F)c2cc(C(=O)N3CCOCC3)c3nnnn3c2n1]

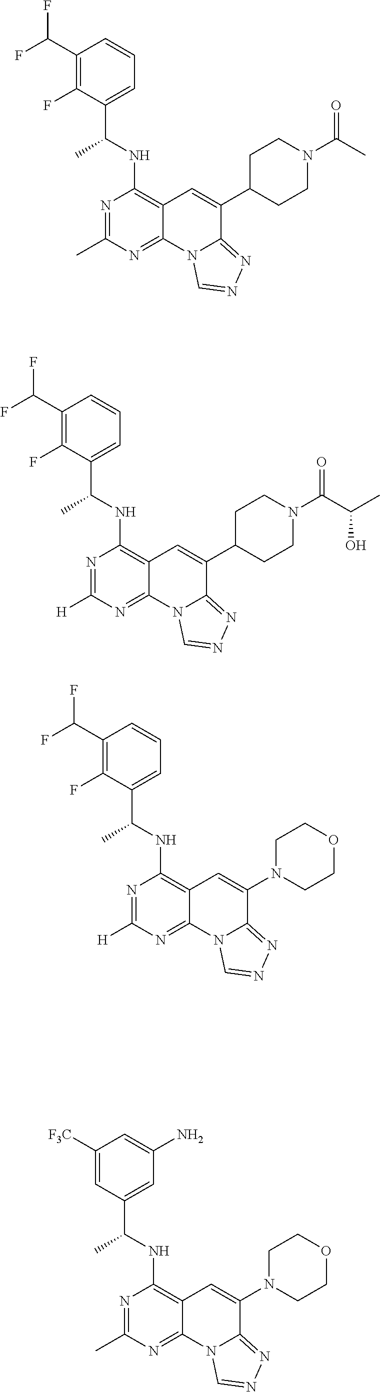 [SMILES: CC(=O)N1CCC(c2cc3c(N[C@H](C)c4cccc(C(F)F)c4F)nc(C)nc3n3cnnc23)CC1 ; C[C@H](O)C(=O)N1CCC(c2cc3c(N[C@H](C)c4cccc(C(F)F)c4F)ncnc3n3cnnc23)CC1 ; C[C@@H](Nc1ncnc2c1cc(N1CCOCC1)c1nncn12)c1cccc(C(F)F)c1F ; Cc1nc(N[C@H](C)c2cc(N)cc(C(F)(F)F)c2)c2cc(N3CCOCC3)c3nncn3c2n1]

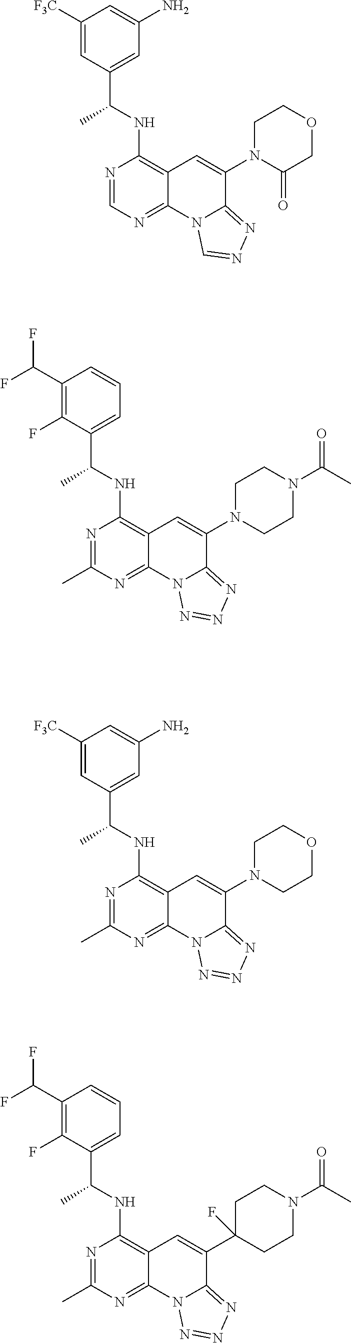 [SMILES: C[C@@H](Nc1ncnc2c1cc(N1CCOCC1=O)c1nncn12)c1cc(N)cc(C(F)(F)F)c1 ; CC(=O)N1CCN(c2cc3c(N[C@H](C)c4cccc(C(F)F)c4F)nc(C)nc3n3nnnc23)CC1 ; Cc1nc(N[C@H](C)c2cc(N)cc(C(F)(F)F)c2)c2cc(N3CCOCC3)c3nnnn3c2n1 ; CC(=O)N1CCC(F)(c2cc3c(N[C@H](C)c4cccc(C(F)F)c4F)nc(C)nc3n3nnnc23)CC1]

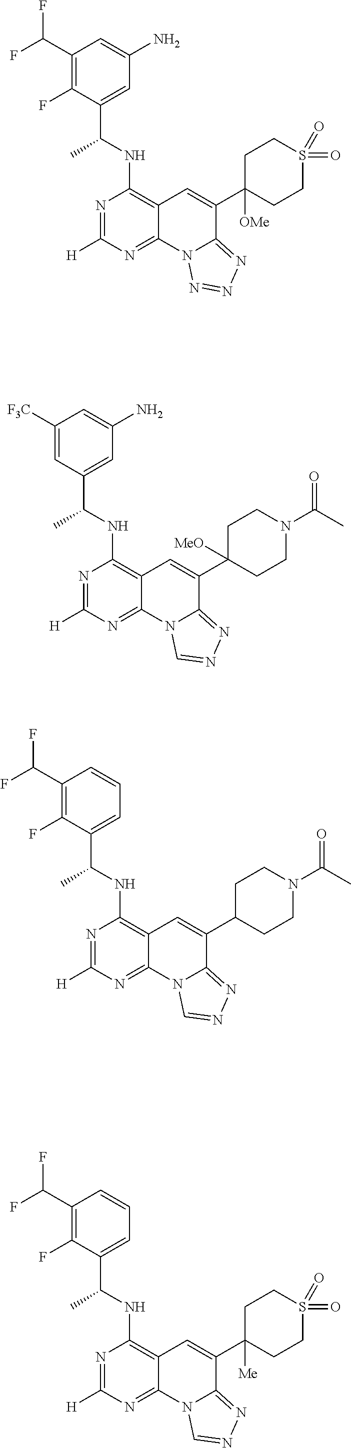 [SMILES: COC1(c2cc3c(N[C@H](C)c4cc(N)cc(C(F)F)c4F)ncnc3n3nnnc23)CCS(=O)(=O)CC1 ; COC1(c2cc3c(N[C@H](C)c4cc(N)cc(C(F)(F)F)c4)ncnc3n3cnnc23)CCN(C(C)=O)CC1 ; CC(=O)N1CCC(c2cc3c(N[C@H](C)c4cccc(C(F)F)c4F)ncnc3n3cnnc23)CC1 ; C[C@@H](Nc1ncnc2c1cc(C1(C)CCS(=O)(=O)CC1)c1nncn12)c1cccc(C(F)F)c1F]

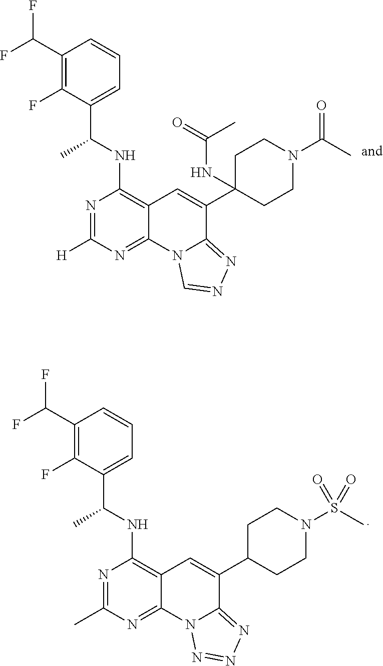

Another aspect of this invention includes pharmaceutical compositions each including a compound as described, and a pharmaceutically acceptable carrier or excipient.

Yet still another aspect of this invention provides for treating a disorder associated with RAS dysfunction in a subject in need thereof. The method includes administering to the subject a therapeutically effective amount of a compound or a pharmaceutical composition as described above. In some embodiments, the disorder is cancer. Cancer can refer to a tissue or organ type and can also spread from one tissue or organ to another tissue type or organ. Cancer can occur in any cell of any type including but not limited to breast, prostate, skin, lung, pancreatic, stomach, brain, kidney, uterine, ovarian, testicular, endothelial, colon, bladder, bone as well as cells of the blood to produce various forms of leukemia. Preferably, the cancer is lung cancer, e.g., non-small cell lung cancer (NSCLC), colorectal cancer (CRC), or pancreatic cancer.

Yet still another aspect of this invention provides uses of the compound or the pharmaceutical composition as described for the manufacture of a medicament for treating a disorder associated with RAS dysfunction.

### DETAILED DESCRIPTION OF THE INVENTION

The inventor of the present invention designs and synthesizes a new fused heterocyclic compound, and a tautomer, a stereoisomer, or a pharmaceutically acceptable salt thereof, as a modulator of RAS signaling. Compounds and compositions of the present invention are useful in the treatment of cancers and other diseases related with RAS dysfunction in a subject. The subject can be a mammal or a human.

The present invention can be understood more readily by reference to the following detailed description of the invention and the Examples included therein.

Unless the context indicates otherwise, references to Formula (I) in all sections of this document (including the uses, methods and other aspects of the invention) include references to all other sub-formula, sub-groups, preferences, embodiments and examples as defined herein.

### Definitions

Unless otherwise stated, the following terms used in the specification and claims have the meanings discussed below:

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used herein, the term “modulate” can refer to enhancement of activity or inhibition of activity.

As used herein, the term “subject” refers to a target of administration. In the present invention, the subject can be a mammal or a human.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intra aural administration, intracerebral administration, rectal administration, and parenteral administration, including injectable such as intravenous administration, intra-arterial administration, intramuscular administration, and subcutaneous administration. Administration can be continuous or intermittent.

Throughout this specification, “C(O)” is a short hand notation for a carbonyl group, i.e., C═O; and “SO₂” or “S(O)₂” is a short hand notation for

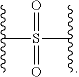 [SMILES: *S(*)(=O)=O]

Throughout this specification, “—SH” is thiol group. The term “alkylthio” means alkyl-S—.

Compounds described herein can contain one or more double bonds and, thus, potentially give rise to cis/trans isomers, as well as other conformational isomers. Unless stated to the contrary, the invention includes all such possible isomers, as well as mixtures of such isomers. In addition, the invention also includes all possible optical isomers, as well as mixtures of optical isomers.

As used herein, the term “Alkyl” refers to a saturated straight or branched hydrocarbon chain radical consisting of carbon and hydrogen atoms, containing no unsaturation, having the stated number of carbon atoms (e.g., C₁-C₁₀ or C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3 carbon atoms, 4 carbon atoms, etc., up to and including 10 carbon atoms, although the present definition also covers the occurrence of the term “alkyl” where no numerical range is designated. Examples include methyl, ethyl, propyl, 2-propyl, n-butyl, iso-butyl, tert-butyl, pentyl, hexyl, and the like. The term “substituted alkyl” refers to alkyl substituted with one or more substituents.

As used herein, the term “alkylene” by itself or as part of another molecule means a divalent radical derived from an alkane, which can be a straight chain or branched chain. In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, refers to an alkylene group having from 1 to 4 carbon atoms.

As used herein, the term “cyano” or formula “—CN” refers to —C═N, wherein the carbon and nitrogen atoms are bound together by a triple bond

As used herein, the term “Alkoxy” or “Alkoxyl” refers to a saturated straight or branched hydrocarbon linked to an oxygen atom. Examples include methoxy, ethoxy, propoxy, 2-propoxy, n-butoxy, iso-butoxy, tert-butoxy, pentoxy, hextoxy, and the like, preferably methoxy, ethoxy, propoxy or 2-propoxy. Representative saturated straight chain alkoxys include methoxy, ethoxyl, n-propoxy, n-butoxy, n-pentoxy, n-hextoxy, and the like; while saturated branched alkoxys include isopropoxyl, sec-butoxy, isobutoxy, tert-butoxy, isopentoxy, and the like. Cyclic alkoxy are referred to herein as a “cycloalkoxy”. Alkoxy can be linked to a molecule by one or two attachment points.

As used herein, the term “alkoxyalkyl,” as used herein, refers to an alkyl group substituted with one, two, or three alkoxy groups.

As used herein, the term “Alkenyl” refers to an unsaturated straight or branched hydrocarbon, and includes at least one C═C double bond. Example include ethenyl and propenyl. The alkenyl can also be substituted with one or more alkyl group(s). Alkenyl can be linked to a molecule by one or two attachment points.

As used herein, the term “carbonyl” refers to —C(═O)—.

As used herein, the term “alkoxycarbonyl” refers to a group of formula (alkoxy)(C═O)—, which is connected to parent molecular moiety through the carbonyl carbon.

As used herein, the term “alk” as a prefix (e.g., as in “alkoxy”) encompasses alkyl, alkenyl, and alkynyl—unless it is specifically defined or chemically required otherwise to be only, e.g., alkyl, alkenyl, or alkynyl.

As used herein, the term “hydroxyalkyl-carbonyl” refers to a group of formula (hydroxyalkyl)(C═O)—, which is connected to parent molecular moiety via carbonyl carbon.

As used herein, the term “aminocarbonyl”, as used herein, refers to the group —C(═O)—NRR′, in which, R and R′ are each independently H, alkyl, optionally substituted cycloalkyl or heterocycloalkyl. The term “alkylaminocarbonyl” refers to the group —C(═O)—NH-alkyl.

As used herein, the term “carboxyl” or “carboxy”, as used herein, refers to —C(O)—OH.

The term “acyl” refers to the groups H—C(O)—, alkyl-C(O)—, alkenyl-C(O)—, alkynyl-C(O)—, cycloalkyl-C(O)—, aryl-C(O)—, heteroaryl-C(O)—, heterocycloalkyl-C(O)—, wherein each of the alkyl, alkenyl, alkynyl, cycloalkyl, aryl, heteroaryl, and heterocycloalkyl is optionally further substituted.

As used herein, the term “amido” or “amide” includes both —C(═O)—NR₂ (i.e., aminocarbonyl) and R—C(═O)—NR—, with either the carbonyl or the amino group as the bonding atom.

As used herein, the term “amino” or “amine” refers to an —NR′R″ group in which each of R′ or R″ can be the same or different, and can be hydrogen (giving rise to —NH₂), alkyl (giving rise to alkylamino or dialkylamino) or another chemical moiety (such as optionally substituted heterocycloalkyl and cycloalkyl). The term “alkylamino” refers to —NH-alkyl, and the term “dialkylamino” refers to —NRR′, in which, R and R′ are the same or different alkyl.

As used herein, the term “alkamino” refers to an amino group that is attached to an alkylene. In general, if a compound is attached to an alkamino group, the alkylene portion of the alkamino is attached to the compound.

As used herein, the term “Cycloalkyl” by itself or as part of another substituent refers to a non-aromatic carbon-based ring composed of at least three carbon atoms. The term “cycloalkyl” includes monocyclic cycloalkyl, bicyclic cycloalkyl, polycyclic cycloalkyl, bridged cycloalkyl, fused cycloalkyl, and spiro cycloalkyl groups. In a bridged cycloalkyl, the rings share at least two common non-adjacent atoms. In a fused bicyclic cycloalkyl, two rings share a covalent bond. In a spirocyclic cycloalkyl group, one atom is common to two different rings.

As used herein, the term “fused phenyl” refers to a phenyl group fused to another ring, such as aryl, heteroaryl, cycloalkenyl, heterocycloalkenyl, cycloalkyl or heterocycloalkyl. The fused phenyl can be optionally substituted on any of the atoms within the fused system.

As used herein, the term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted.

In addition, ring-forming carbon atoms or heteroatoms of the cycloalkyl or heterocycloalkyl group can be optionally substituted with one or two oxo groups. For example, a ring-forming S atom can be substituted by 1 or 2 oxo (form a S(O) or S(O)₂). For another example, a ring-forming C atom can be substituted by oxo (form carbonyl).

As used herein, the term “Cycloalkenyl” refers to a partially unsaturated cyclic hydrocarbon group containing 1 to 4 rings and 3 to 8 carbons per ring. The cycloalkenyl does not belong to an aromatic series, and includes a monocyclic or polycyclic group. Exemplary such groups include cyclobutenyl, cyclopentenyl, cyclohexenyl, etc. The Cycloalkenyl group may be further substituted.

As used herein, the term “Heterocycloalkenyl” is a cycloalkenyl having at least one heteroatom in the ring. The heteroatom refers to a non-C atom. The heteroatom is preferably N, O, S, or P, etc. Heterocycloalkyl may be substituted or unsubstituted.

In addition, ring-forming carbon atoms or heteroatoms of the cycloalkenyl or heterocycloalkenyl group can be optionally substituted with one or two oxo groups.

As used herein, the term “Halogen” or “Halo” refers to chlorine (Cl), bromine (Br), fluorine (F) or iodine (I). The term “halogen” or “halo” in front of a group name refers to that the group is partially or completely halogenated, i.e., the group is substituted by F, Cl, Br or I in any combination.

As used herein, the term “Haloalkyl” refers to alkyl as defined above in which one or more of the hydrogen atoms have been replaced with a halogen independently selected from fluoro, chloro, bromo, and iodol. “Fluoroalkyl” means alkyl as defined above wherein one or more hydrogen atoms have been replaced by fluoro atoms. Unless otherwise specified with a number, a haloalkyl can include as many as chemically possible halo atoms as substituents on the alkyl group. For example, fluoroethyl can be —CH₂CF₃, —CHF—CH₃, or —CH₂CH₂F.

As used herein, the term “hydrogen” (or H) includes its isotopes of deuterium (D or ²H) and tritium (³H), meaning a or any hydrogen atom in the compounds of this invention can be replaced with either deuterium (D or ²H) and tritium (³H).

As used herein, the term “hydroxyl” or “hydroxy” refers to the group —OH.

As used herein, the term “hydroxyalkyl” by itself or as part of another substituent refers to an alkyl group in which one or more of the hydrogen atoms are replaced with a hydroxyl substituent. Thus, the term “hydroxyalkyl” is meant to include monohydroxyalkyls, dihydroxyalkyls, trihydroxyalkyls, etc.

As used herein, the term “sulfonyl” refers to —S(O)₂—.

As used herein, the term “alk-cycloalkyl” refers to a cycloalkyl group that is connected to an alkylene. In general, if a compound is attached to an alk-cycloalkyl group, the alkylene portion of the alk-cycloalkyl group is attached to the compound.

As used herein, the term “alk-S(O)₂—R” refers to a —S(O)₂—R group that is connected to an alkylene. In general, if a compound is attached to an alk-S(O)₂—R group, the alkylene portion of the alk-S(O)₂—R is attached to the compound. In some embodiments, R is H or alkyl.

As used herein, the term “aromatics” or “aromatic ring” or similar terms refer to planar rings having a delocalized pi-electron system containing 4n+2 pi-electrons, where n is a positive integer. Aromatic rings can be formed from five, six, seven, eight, nine, ten or more than ten atoms. Aromatics are optionally substituted. The term “aromatic” includes carbocyclic aryl (“aryl”, e.g., phenyl) and “heteroaryl” (or “heteroaromatic”) groups (e.g., pyridine). The term includes monocyclic or fused-ring polycyclic (i.e., rings that share adjacent pairs of carbon atoms) groups.

As used herein, the term “Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The Aryl group may be further substituted.

As used herein, the term “Heteroaryl” refers to a monocyclic or fused ring (i.e., rings which share an adjacent pair of atoms) of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, carbazole, benzimidazole, benzoxazole, benzthiazole, indazole and quinazoline. The heteroaryl group may be substituted or unsubstituted. Examples of substituents include, but not limited to, halo, alkyl, alkynyl, haloalkyl, cycloalkyl, heterocycloalkyl, hydroxyl, alkoxyl, amino, amine, cyano, alkenyl, cycloalkenyl, heterocycloalkenyl, aryl, heteroaryl, carbonyl, alkoxycarbonyl, aminocarbonyl, hydrocarboxyl, alkylthio, -D (deuterium), oxo, —C(O)—(CH₂)ₙ—OR, —(CH₂)ₙ—S(O)₂—R, —C(O)—R, —C(O)—NR, —(CH₂)ₙ—OR, —NR—C(O)—(CH₂)ₙ—, CD₃-O—, and —CN, in which, R is H or alkyl.

As used herein, the term “oxo” refers to an oxygen atom as a divalent substituent (i.e., ═O). It resulted in a carbonyl group when it is attached to a carbon atom, a sulfoxide or sulfone group when attached to a sulfur atom, or an N-oxide group attached to a nitrogen atom. In some embodiments, heterocyclic groups may be optionally substituted by 1 or 2 oxo substituents.

The above-defined groups may include prefixes and/or suffixes that are commonly used in the art to create additional well-recognized substituent groups. As examples, the term “haloalkoxy” or “haloalkyloxy” refers to a haloalkyl group attached to the parent molecular moiety through an oxygen atom. The term “(haloalkyl)oxyalkyl” refers to an alkyl group substituted with one, two, or three (haloalkyl)oxy groups. As another example, the term “hydroxyalkamino” refers to an amino group substituted with one or two hydroxyalkyl groups.

As used herein, the term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity. When a disclosed compound is named or depicted by structure without indicating stereochemistry, it is understood that the name or structure encompasses all possible stereoisomers, geometric isomers, including essentially pure stereo or geometric isomers, as well as combination thereof.

As used herein, the term “tautomer” or “tautomeric form” refers to structural isomers of different energies which are interconvertible via a low energy barrier. Where tautomerization is possible (e.g., in solution), a chemical equilibrium of tautomer can be reached. “Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the term “Pharmaceutically acceptable” means suitable for use in a human or other mammal.

As used herein, the term “a pharmaceutically acceptable salt” refers to a salt with known cations or anions, and may be applied to use in the field. Suitable salts with bases, for example, salts formed from compounds of Formula (I) containing carboxyl groups, include salts of alkali metals (such as sodium and potassium), alkali earth metals (such as calcium and magnesium), ammonium and amines. Suitable salts with acid radical additives, such as salts formed from compounds of Formula (I) containing amino, include salts formed with inorganic acids such as hydrochloride, sulphate, sulphite, phosphate, hydrogen phosphate and nitrate, and salts formed with organic acids such as acetic acid, malic acid, tartaric acid, citric acid, lactic acid, salicylic acid, oxalic acid, etc.

Moreover, if the compound is obtained as an acid addition salt, free alkalis can be obtained by alkalizing a solution of the acid salt. In contrast, if a product is a free alkali, the addition salt can be prepared by dissolving the free alkali in a suitable organic solvent and treating the solution with an acid, which is consistent with a conventional process of preparing an acid addition salt from an alkaline compound. Those skilled in the art should understand various synthesis methods that can be used to prepare acceptable addition salts without undue experimentation.

Furthermore, when a compound the present invention has a carboxyl group, it can be made to a pharmaceutically acceptable ester in an ordinary method (e.g., condensation reaction of a carboxylic acid with an alcohol), by reacting the compound with a corresponding alcohol (e.g., C₁₋₆ alcohol).

As used herein, the “a pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

As used herein, the term “a pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “a therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

The atom in a chemical group that is bonded to another atom is denoted with a wiggly line, for example as in

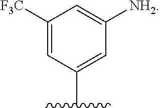 [SMILES: *c1cc(N)cc(C(F)(F)F)c1]

### EXAMPLES

The following examples are present to provide those of ordinary skill in the art with a more complete disclosure and description of how compounds of the present invention are made and used and are intended to be purely exemplary of the present invention and are not intended to limit the scope of what the inventors regard as their invention.

### Example 1: Ethyl (R)-6-((1-(3-amino-5-(trifluoromethyl)phenyl)ethyl)amino)-8-methyl-[1,2,4]triazolo[1′,5′:1,6]pyrido[2,3-d]pyrimidine-4-carboxylate

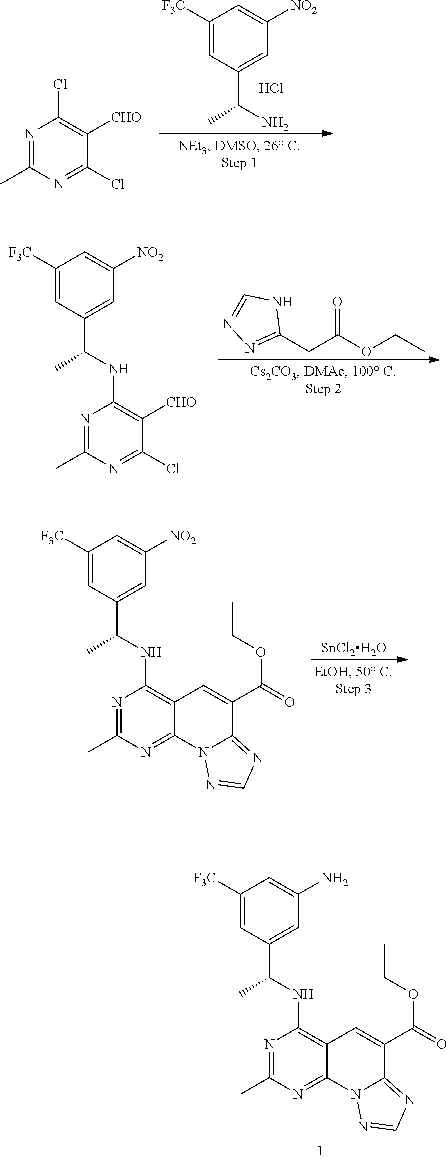

Step 1: To a solution of 4,6-dichloro-2-methylpyrimidine-5-carbaldehyde (300 mg, 1.57 mmol, 1.0 eq.) in DCM (2.5 mL) was added (R)-1-(3-nitro-5-(trifluoromethyl)phenyl)ethan-1-amine hydrochloride (367.8 mg, 1.57 mmol, 1.0 eq.) in DMSO (2.5 mL), then Et₃N (476.8 mg, 4.71 mmol, 3.0 eq.) was added at 0° C. The reaction was stirred at 26° C. for 10 min to give a red purple solution. The mixture was extracted with DCM (3×), the organic phase was concentrated under reduced pressure to dryness to afford crude product (R)-4-chloro-2-methyl-6-((1-(3-nitro-5-(trifluoromethyl)phenyl)ethyl)amino)pyrimidine-5-carbaldehyde.

Step 2: To a solution of (R)-4-chloro-2-methyl-6-((1-(3-nitro-5-(trifluoromethyl)phenyl)ethyl)amino)pyrimidine-5-carbaldehyde (400 mg, 1.03 mmol, 1.0 eq.) in DMAc (5.0 mL) was added Cs₂CO₃ (670.5 mg, 2.06 mmol, 2.0 eq.) and ethyl 2-(4H-1,2,4-triazol-3-yl)acetate (159.7 mg, 1.03 mmol, 1.0 eq.). The reaction was stirred at 100° C. for 0.5 h to give a red solution. The mixture was extracted with DCM (3×), the organic phase was concentrated under reduced pressure to dryness. The crude product was purified by flash column (SiO₂, eluted with 0˜100% EA in PE) to give compound ethyl (R)-8-methyl-6-((1-(3-nitro-5-(trifluoromethyl)phenyl)ethyl)amino)-[1,2,4]triazolo[1′,5′:1,6]pyrido[2,3-d]pyrimidine-4-carboxylate.

Step 3: To a solution of ethyl (R)-8-methyl-6-((1-(3-nitro-5-(trifluoromethyl)phenyl)ethyl)amino)-[1,2,4]triazolo[1′,5′:1,6]pyrido[2,3-d]pyrimidine-4-carboxylate (30 mg, 0.061 mmol, 1.0 eq.) in EtOH (1.0 mL) was added SnCl₂·2H₂O (69.2 mg, 0.31 mmol, 5.0 eq.). The reaction was stirred at 50° C. for 0.5 h to give a red solution. The reaction was concentrated under reduced pressure to dryness and purified by reversed column chromatography (MeCN/H₂O, from 0 to 100%) to give the product ethyl (R)-6-((1-(3-amino-5-(trifluoromethyl)phenyl)ethyl)amino)-8-methyl-[1,2,4]triazolo[1′,5′:1,6]pyrido[2,3-d]pyrimidine-4-carboxylate.

| Example # | Structure | Mass m/z (M + 1)⁺ |
|---|---|---|
| 1 | 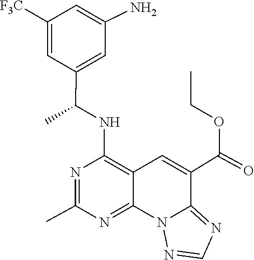 [SMILES: CCOC(=O)c1cc2c(N[C@H](C)c3cc(N)cc(C(F)(F)F)c3)nc(C)nc2n2ncnc12] | 460.2 |

### Example 2: Synthesis of (R)-(6-((1-(3-amino-5-(trifluoromethyl)phenyl)ethyl)amino)-8-methyl-[1,2,4]triazolo[1′,5′:1,6]pyrido[2,3-d]pyrimidin-4-yl)(morpholino)methanone

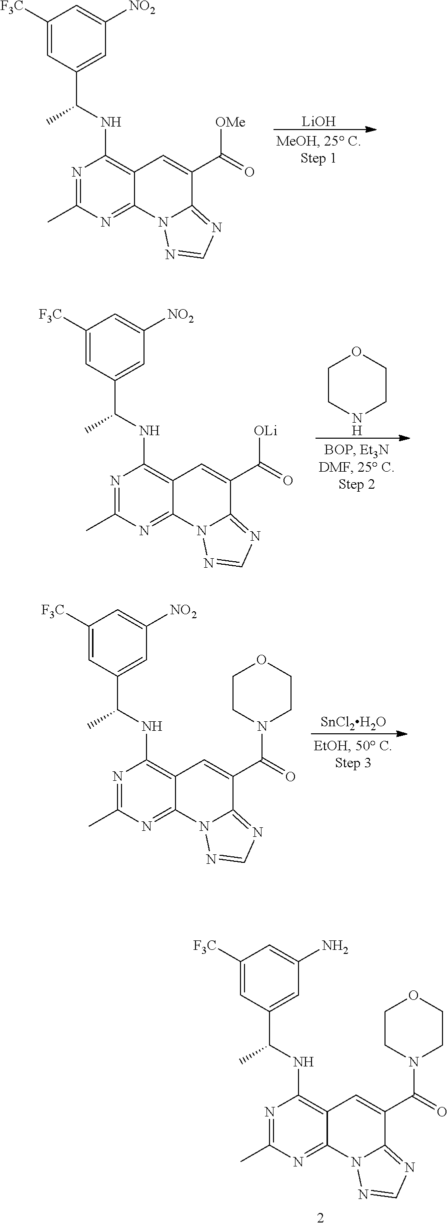

Step 1: To a solution of methyl (R)-8-methyl-6-((1-(3-nitro-5-(trifluoromethyl)phenyl)ethyl)amino)-[1,2,4]triazolo[1′,5′:1,6]pyrido[2,3-d]pyrimidine-4-carboxylate (200 mg, 0.4 mmol, 1.0 eq.) in MeOH (5.0 mL) was added 10% LiOH aq. (3.0 mL). The reaction was stirred at 25° C. for 10 mins to give a yellow solution. Then the mixture was added 1N HCl to adjust pH 3˜4 and extracted with DCM (3×). The organic phase was concentrated under reduced pressure to dryness to give the product lithium (R)-8-methyl-6-((1-(3-nitro-5-(trifluoromethyl)phenyl)ethyl)amino)-[1,2,4]triazolo[1′,5′:1,6]pyrido[2,3-d]pyrimidine-4-carboxylate.

Step 2: To a solution of lithium (R)-8-methyl-6-((1-(3-nitro-5-(trifluoromethyl)phenyl)ethyl)amino)-[1,2,4]triazolo[1′,5′:1,6]pyrido[2,3-d]pyrimidine-4-carboxylate (60 mg, 0.15 mmol, 1.0 eq.) in DMF (1.0 mL) was added BOP (127.5 mg, 0.3 mmol, 2.0 eq.), and stirred for 30 min, then Et₃N (0.04 mL) and morpholine (80.0 mg, 0.4 mmol, 3.0 eq.) was added. The mixture was stirred at 26° C. for 1.0 h to give a red solution. The mixture was concentrated under reduced pressure to dryness and purified by reversed column chromatography (MeCN/H₂O, from 0 to 100%) to give the product (R)-(8-methyl-6-((1-(3-nitro-5-(trifluoromethyl)phenyl)ethyl)amino)-[1,2,4]triazolo[1′,5′:1,6]pyrido[2,3-d]pyrimidin-4-yl)(morpholino)methanone.

Step 3: To a solution of (R)-(8-methyl-6-((1-(3-nitro-5-(trifluoromethyl)phenyl)ethyl)amino)-[1,2,4]triazolo[1′,5′:1,6]pyrido[2,3-d]pyrimidin-4-yl)(morpholino)methanone (20 mg, 0.03 mmol, 1.0 eq.) in EtOH (1.0 mL) was added SnCl₂·2H₂O (42.5 mg, 0.19 mmol, 5.0 eq.). The reaction was stirred at 50° C. in for 0.5 h to give a red solution. Then the mixture was added NaHCO₃ to adjust pH 7˜8 and extracted with DCM (3×). The organic phase was concentrated under reduced pressure to dryness and purified by reversed column chromatography (MeCN/H₂O, from 0 to 100%) to give the product (R)-(6-((1-(3-amino-5-(trifluoromethyl)phenyl)ethyl)amino)-8-methyl-[1,2,4]triazolo[1′,5′:1,6]pyrido[2,3-d]pyrimidin-4-yl)(morpholino)methanone.

| Example # | Structure | Mass m/z (M + 1)⁺ |
|---|---|---|
| 2 | 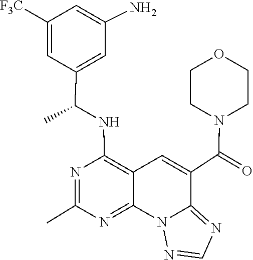 [SMILES: Cc1nc(N[C@H](C)c2cc(N)cc(C(F)(F)F)c2)c2cc(C(=O)N3CCOCC3)c3ncnn3c2n1] | 501.2 |
| 3 | 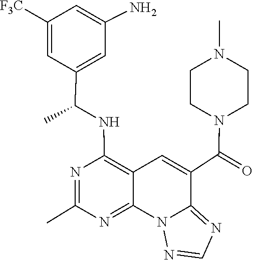 [SMILES: Cc1nc(N[C@H](C)c2cc(N)cc(C(F)(F)F)c2)c2cc(C(=O)N3CCN(C)CC3)c3ncnn3c2n1] | 514.2 |
| 4 | 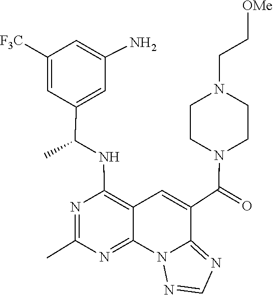 [SMILES: COCCN1CCN(C(=O)c2cc3c(N[C@H](C)c4cc(N)cc(C(F)(F)F)c4)nc(C)nc3n3ncnc23)CC1] | 558.2 |
| 5 | 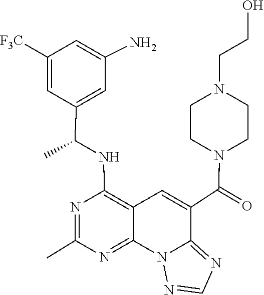 [SMILES: Cc1nc(N[C@H](C)c2cc(N)cc(C(F)(F)F)c2)c2cc(C(=O)N3CCN(CCO)CC3)c3ncnn3c2n1] | 544.2 |
| 6 | 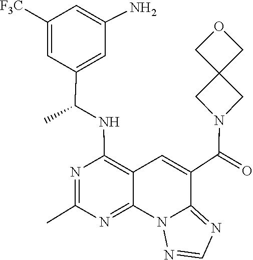 [SMILES: Cc1nc(N[C@H](C)c2cc(N)cc(C(F)(F)F)c2)c2cc(C(=O)N3CC4(COC4)C3)c3ncnn3c2n1] | 513.2 |
| 7 | 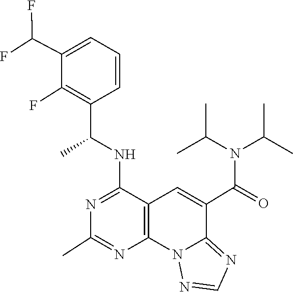 [SMILES: Cc1nc(N[C@H](C)c2cccc(C(F)F)c2F)c2cc(C(=O)N(C(C)C)C(C)C)c3ncnn3c2n1] | 500.2 |
| 8 | 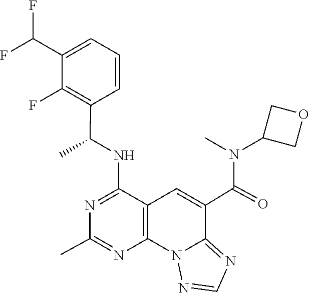 [SMILES: Cc1nc(N[C@H](C)c2cccc(C(F)F)c2F)c2cc(C(=O)N(C)C3COC3)c3ncnn3c2n1] | 486.5 |
| 9 | 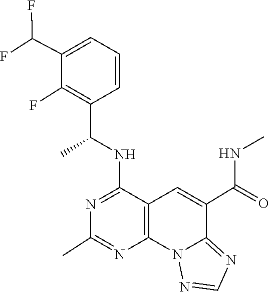 [SMILES: CNC(=O)c1cc2c(N[C@H](C)c3cccc(C(F)F)c3F)nc(C)nc2n2ncnc12] | 430.2 |
| 10 | 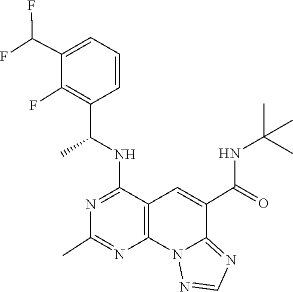 [SMILES: Cc1nc(N[C@H](C)c2cccc(C(F)F)c2F)c2cc(C(=O)NC(C)(C)C)c3ncnn3c2n1] | 472.2 |
| 11 | 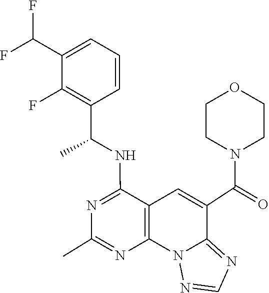 [SMILES: Cc1nc(N[C@H](C)c2cccc(C(F)F)c2F)c2cc(C(=O)N3CCOCC3)c3ncnn3c2n1] | 486.2 |
| 12 | 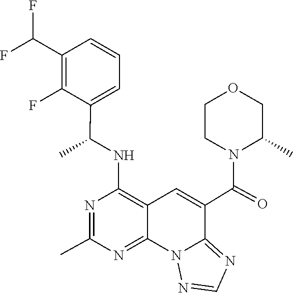 [SMILES: Cc1nc(N[C@H](C)c2cccc(C(F)F)c2F)c2cc(C(=O)N3CCOC[C@@H]3C)c3ncnn3c2n1] | 500.2 |
| 13 | 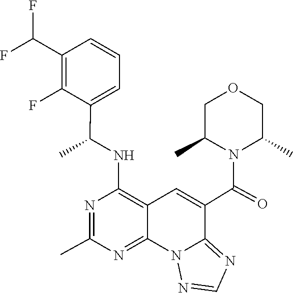 [SMILES: Cc1nc(N[C@H](C)c2cccc(C(F)F)c2F)c2cc(C(=O)N3[C@@H](C)COC[C@@H]3C)c3ncnn3c2n1] | 514.2 |
| 14 | 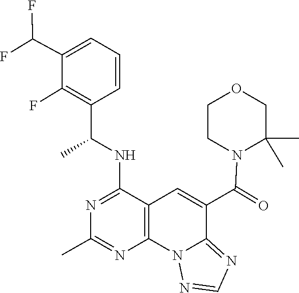 [SMILES: Cc1nc(N[C@H](C)c2cccc(C(F)F)c2F)c2cc(C(=O)N3CCOCC3(C)C)c3ncnn3c2n1] | 514.2 |
| 15 | 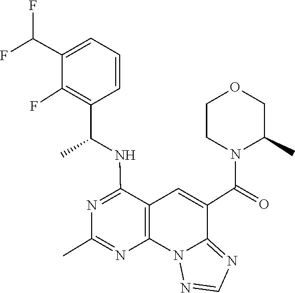 [SMILES: Cc1nc(N[C@H](C)c2cccc(C(F)F)c2F)c2cc(C(=O)N3CCOC[C@H]3C)c3ncnn3c2n1] | 500.2 |
| 16 | 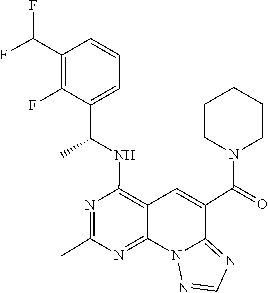 [SMILES: Cc1nc(N[C@H](C)c2cccc(C(F)F)c2F)c2cc(C(=O)N3CCCCC3)c3ncnn3c2n1] | 484.2 |
| 17 | 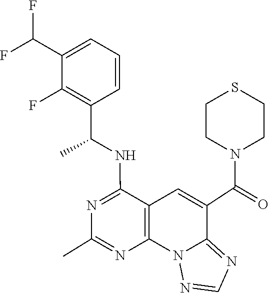 [SMILES: Cc1nc(N[C@H](C)c2cccc(C(F)F)c2F)c2cc(C(=O)N3CCSCC3)c3ncnn3c2n1] | 502.5 |
| 18 | 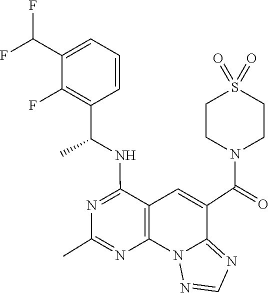 [SMILES: Cc1nc(N[C@H](C)c2cccc(C(F)F)c2F)c2cc(C(=O)N3CCS(=O)(=O)CC3)c3ncnn3c2n1] | 534.5 |
| 19 | 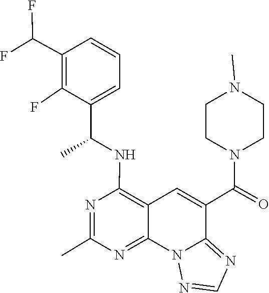 [SMILES: Cc1nc(N[C@H](C)c2cccc(C(F)F)c2F)c2cc(C(=O)N3CCN(C)CC3)c3ncnn3c2n1] | 499.2 |
| 20 | 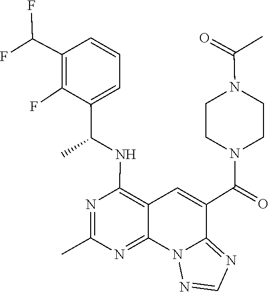 [SMILES: CC(=O)N1CCN(C(=O)c2cc3c(N[C@H](C)c4cccc(C(F)F)c4F)nc(C)nc3n3ncnc23)CC1] | 527.2 |
| 21 | 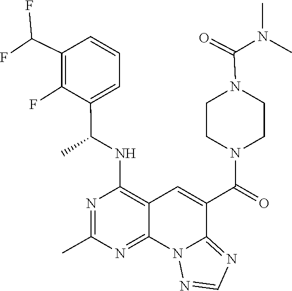 [SMILES: Cc1nc(N[C@H](C)c2cccc(C(F)F)c2F)c2cc(C(=O)N3CCN(C(=O)N(C)C)CC3)c3ncnn3c2n1] | 556.2 |
| 22 | 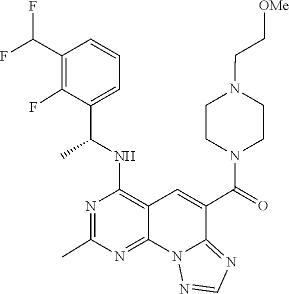 [SMILES: COCCN1CCN(C(=O)c2cc3c(N[C@H](C)c4cccc(C(F)F)c4F)nc(C)nc3n3ncnc23)CC1] | 556.2 |
| 23 | 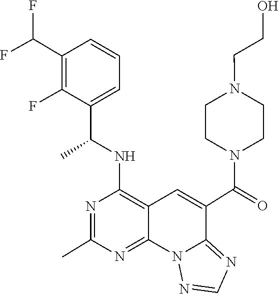 [SMILES: Cc1nc(N[C@H](C)c2cccc(C(F)F)c2F)c2cc(C(=O)N3CCN(CCO)CC3)c3ncnn3c2n1] | 529.2 |
| 24 | 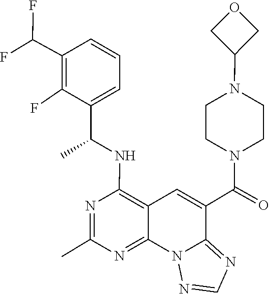 [SMILES: Cc1nc(N[C@H](C)c2cccc(C(F)F)c2F)c2cc(C(=O)N3CCN(C4COC4)CC3)c3ncnn3c2n1] | 541.2 |
| 25 | 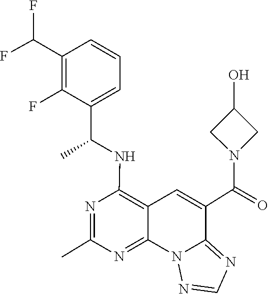 [SMILES: Cc1nc(N[C@H](C)c2cccc(C(F)F)c2F)c2cc(C(=O)N3CC(O)C3)c3ncnn3c2n1] | 472.2 |
| 26 | 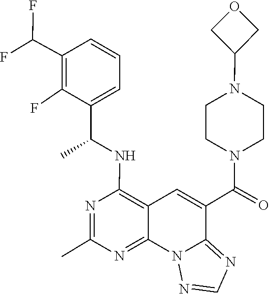 [SMILES: Cc1nc(N[C@H](C)c2cccc(C(F)F)c2F)c2cc(C(=O)N3CCN(C4COC4)CC3)c3ncnn3c2n1] | 541.2 |
| 27 | 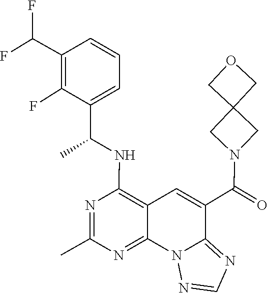 [SMILES: Cc1nc(N[C@H](C)c2cccc(C(F)F)c2F)c2cc(C(=O)N3CC4(COC4)C3)c3ncnn3c2n1] | 498.2 |
| 28 | 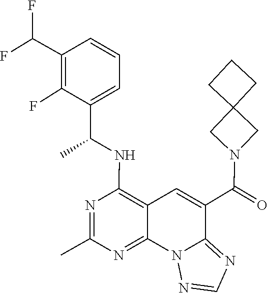 [SMILES: Cc1nc(N[C@H](C)c2cccc(C(F)F)c2F)c2cc(C(=O)N3CC4(CCC4)C3)c3ncnn3c2n1] | 496.5 |
| 29 | 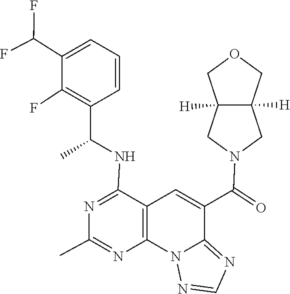 [SMILES: Cc1nc(N[C@H](C)c2cccc(C(F)F)c2F)c2cc(C(=O)N3C[C@H]4COC[C@H]4C3)c3ncnn3c2n1] | 512.5 |
| 30 | 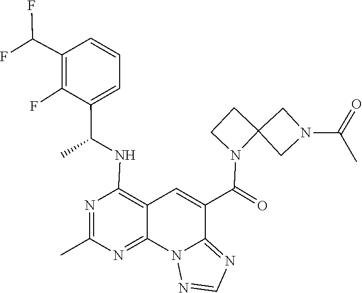 [SMILES: CC(=O)N1CC2(CCN2C(=O)c2cc3c(N[C@H](C)c4cccc(C(F)F)c4F)nc(C)nc3n3ncnc23)C1] | 539.2 |
| 31 | 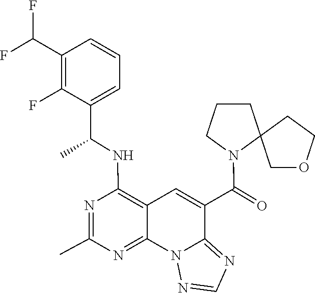 [SMILES: Cc1nc(N[C@H](C)c2cccc(C(F)F)c2F)c2cc(C(=O)N3CCCC34CCOC4)c3ncnn3c2n1] | 526.2 |
| 32 | 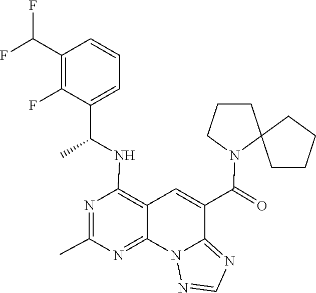 [SMILES: Cc1nc(N[C@H](C)c2cccc(C(F)F)c2F)c2cc(C(=O)N3CCCC34CCCC4)c3ncnn3c2n1] | 524.6 |
| 33 | 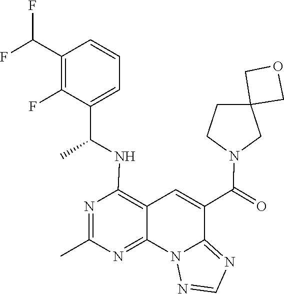 [SMILES: Cc1nc(N[C@H](C)c2cccc(C(F)F)c2F)c2cc(C(=O)N3CCC4(COC4)C3)c3ncnn3c2n1] | 512.5 |
| 34 | 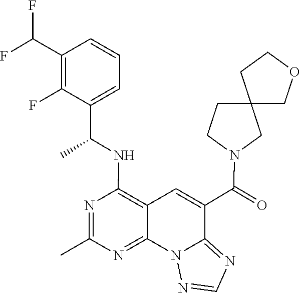 [SMILES: Cc1nc(N[C@H](C)c2cccc(C(F)F)c2F)c2cc(C(=O)N3CCC4(CCOC4)C3)c3ncnn3c2n1] | 526.5 |
| 35 | 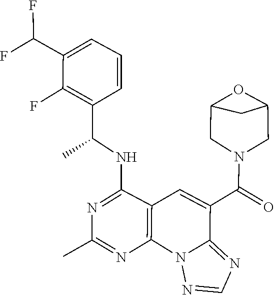 [SMILES: Cc1nc(N[C@H](C)c2cccc(C(F)F)c2F)c2cc(C(=O)N3CC4CC(C3)O4)c3ncnn3c2n1] | 498.5 |
| 36 | 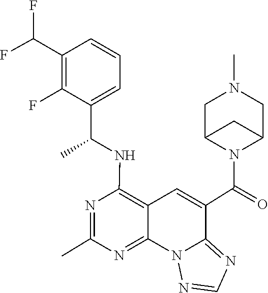 [SMILES: Cc1nc(N[C@H](C)c2cccc(C(F)F)c2F)c2cc(C(=O)N3C4CC3CN(C)C4)c3ncnn3c2n1] | 541.2 |
| 37 | 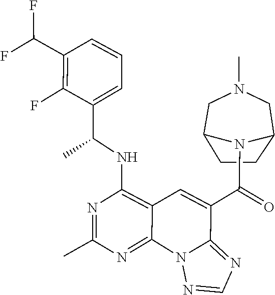 [SMILES: Cc1nc(N[C@H](C)c2cccc(C(F)F)c2F)c2cc(C(=O)N3C4CCC3CN(C)C4)c3ncnn3c2n1] | 525.6 |
| 38 | 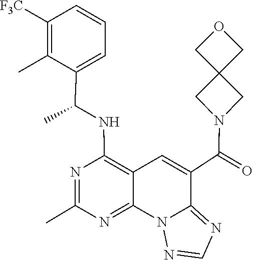 [SMILES: Cc1nc(N[C@H](C)c2cccc(C(F)(F)F)c2C)c2cc(C(=O)N3CC4(COC4)C3)c3ncnn3c2n1] | 512.2 |
| 39 | 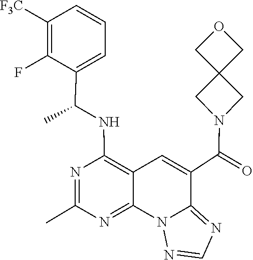 [SMILES: Cc1nc(N[C@H](C)c2cccc(C(F)(F)F)c2F)c2cc(C(=O)N3CC4(COC4)C3)c3ncnn3c2n1] | 516.2 |
| 40 | 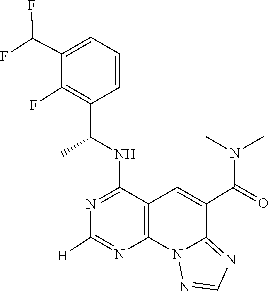 [SMILES: C[C@@H](Nc1ncnc2c1cc(C(=O)N(C)C)c1ncnn12)c1cccc(C(F)F)c1F] | 430.4 |
| 41 | 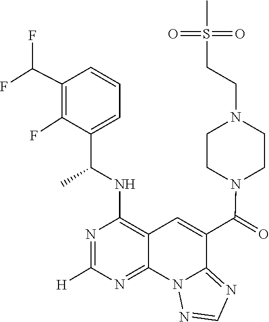 [SMILES: C[C@@H](Nc1ncnc2c1cc(C(=O)N1CCN(CCS(C)(=O)=O)CC1)c1ncnn12)c1cccc(C(F)F)c1F] | 577.6 |
| 42 | 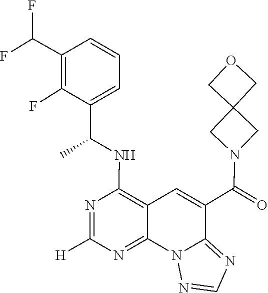 [SMILES: C[C@@H](Nc1ncnc2c1cc(C(=O)N1CC3(COC3)C1)c1ncnn12)c1cccc(C(F)F)c1F] | 484.5 |
| 43 | 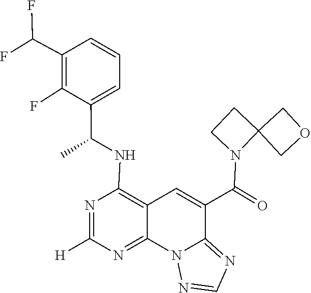 [SMILES: C[C@@H](Nc1ncnc2c1cc(C(=O)N1CCC13COC3)c1ncnn12)c1cccc(C(F)F)c1F] | 484.5 |
| 44 | 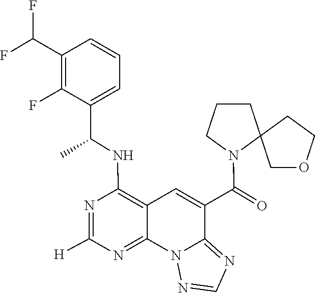 [SMILES: C[C@@H](Nc1ncnc2c1cc(C(=O)N1CCCC13CCOC3)c1ncnn12)c1cccc(C(F)F)c1F] | 512.5 |
| 45 | 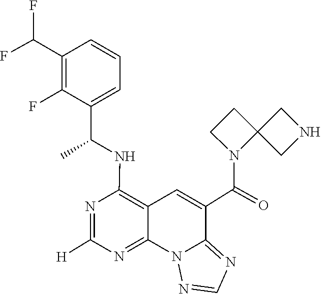 [SMILES: C[C@@H](Nc1ncnc2c1cc(C(=O)N1CCC13CNC3)c1ncnn12)c1cccc(C(F)F)c1F] | 483.5 |
| 46 | 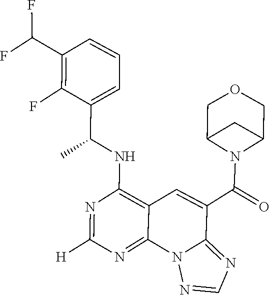 [SMILES: C[C@@H](Nc1ncnc2c1cc(C(=O)N1C3COCC1C3)c1ncnn12)c1cccc(C(F)F)c1F] | 484.4 |
| 47 | 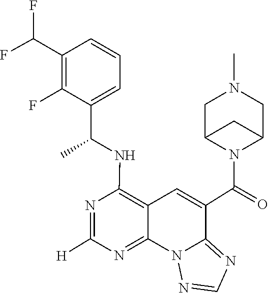 [SMILES: C[C@@H](Nc1ncnc2c1cc(C(=O)N1C3CC1CN(C)C3)c1ncnn12)c1cccc(C(F)F)c1F] | 497.5 |
| 48 | 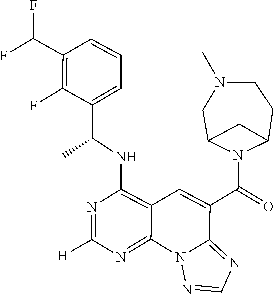 [SMILES: C[C@@H](Nc1ncnc2c1cc(C(=O)N1C3CCN(C)CC1C3)c1ncnn12)c1cccc(C(F)F)c1F] | 511.6 |
| 49 | 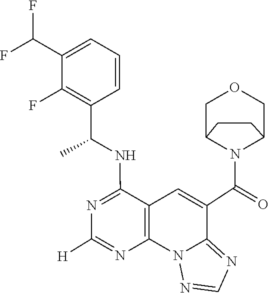 [SMILES: C[C@@H](Nc1ncnc2c1cc(C(=O)N1C3CCC1COC3)c1ncnn12)c1cccc(C(F)F)c1F] | 498.3 |

### Example 50: Synthesis of (R)-4-((1-(3-(difluoromethyl)-2-fluorophenyl)ethyl)amino)-N,N,2-trimethyl-[1,2,4]triazolo[4′,3′:1,6]pyrido[2,3-d]pyrimidine-6-carboxamide

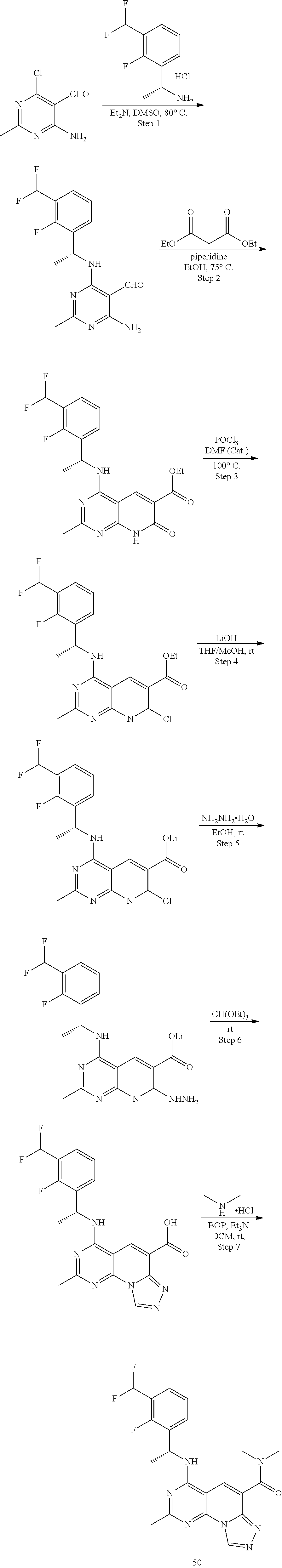

Step 1: To a stirred solution of 4-amino-6-chloro-2-methylpyrimidine-5-carbaldehyde (1.61 g, 9.38 mmol, 1.0 eq.) in dry DMSO (5 mL) at rt was added (R)-1-(3-(difluoromethyl)-2-fluorophenyl)ethan-1-amine hydrochloride (2.33 g, 10.32 mmol, 1.1 eq.) and Et₃N (2.85 g, 28.14 mmol, 3.0 eq.). The reaction was heated at 80° C. for 5 h to give a deep red solution. The reaction was diluted with H₂O (5 mL) and extracted with EtOAc (8×3 mL). The combined organic layers were washed with an aqueous solution of brine (10 mL×2) and dried over anhydrous Na₂SO₄, filtered and concentrated to the crude product which was used in the next step without further purification.

Step 2: To a suspension of 4-amino-6-chloro-2-methylpyrimidine-5-carbaldehyde (3.4 g, 9.38 mmol, 1.0 eq.) in EtOH (10 mL) was added diethyl malonate (3.0 g, 18.76 mmol, 2.0 eq.) and piperidine (0.80 g, 9.38 mmol, 1.0 eq) at RT. The mixture was stirred at 75° C. for 2 h leading to a light-yellow solution. The reaction was concentrated, and diluted with H₂O (10 mL), and extracted with EtOAc (10 mL×2). The combined organic layers were dried over anhydrous Na₂SO₄, filtered and concentrated to afford the crude product, which was purified by flash column (SiO₂, eluted with 0-80% PE in EA). The collected flows were concentrated to afford ethyl (R)-4-((1-(3-(difluoromethyl)-2-fluorophenyl)ethyl)amino)-2-methyl-7-oxo-7,8-dihydropyrido[2,3-d]pyrimidine-6-carboxylate.

Step 3: A vial was charged with ethyl (R)-4-((1-(3-(difluoromethyl)-2-fluorophenyl)ethyl)amino)-2-methyl-7-oxo-7,8-dihydropyrido[2,3-d]pyrimidine-6-carboxylate (221 mg, 0.53 mmol, 1.0 eq.) and POCl₃ (0.5 mL). To this mixture was added a catalytic amount of DMF (2 drops). The mixture was stirred at 90° C. for 3 h under N₂ atmosphere to give a deep red solution. The reaction was poured into ice-cold saturated NaHCO₃ solution (20 mL) at 0° C. The reaction was then extracted with EtOAc (10 mL×2). The combined organic layers were washed with brine (10 mL), dried over Na₂SO₄, filtered and concentrated to afford ethyl (R)-7-chloro-4-((1-(3-(difluoromethyl)-2-fluorophenyl)ethyl)amino)-2-methylpyrido[2,3-d]pyrimidine-6-carboxylate.

Step 4: To a solution of ethyl (R)-7-chloro-4-((1-(3-(difluoromethyl)-2-fluorophenyl)ethyl)amino)-2-methylpyrido[2,3-d]pyrimidine-6-carboxylate (90 mg, 0.51 mmol, 1.0 eq.) in MeOH (1.0 mL) and THF (1.5 mL) was added LiOH solution (51 mg dissolved in 0.5 mL H₂O, 0.54 mmol, 6.0 eq.). The mixture was stirred at RT for 4 h to give a light-yellow solution. The solvents were removed under reduced pressure to give 136 mg crude of lithium (R)-7-chloro-4-((1-(3-(difluoromethyl)-2-fluorophenyl)ethyl)amino)-2-methylpyrido[2,3-d]pyrimidine-6-carboxylate which was used in the next step without further purification.

Step 5: To a solution of lithium (R)-7-chloro-4-((1-(3-(difluoromethyl)-2-fluorophenyl)ethyl)amino)-2-methylpyrido[2,3-d]pyrimidine-6-carboxylate (136 mg, 0.51 mmol, 1.0 eq.) in EtOH (2.0 mL) was added NH₂NH₂·H₂O (0.5 mL). The mixture was stirred at RT for 4 min to give a red solution. The reaction concentrated to afford the crude of lithium (R)-4-((1-(3-(difluoromethyl)-2-fluorophenyl)ethyl)amino)-7-hydrazinyl-2-methylpyrido[2,3-d]pyrimidine-6-carboxylate which was used in the next step without further purification.

Step 6: To the above crude was added CH(OEt)₃ (2.0 mL), and the reaction was stirred at RT overnight to give a red solution. The mixture was neutralized at pH 7-8 with 2N HCl solution and concentrated to give (R)-4-((1-(3-(difluoromethyl)-2-fluorophenyl)ethyl)amino)-2-methyl-[1,2,4]triazolo[4′,3′:1,6]pyrido[2,3-d]pyrimidine-6-carboxylic acid.

Step 7: To the mixture of (R)-4-((1-(3-(difluoromethyl)-2-fluorophenyl)ethyl)amino)-2-methyl-[1,2,4]triazolo[4′,3′:1,6]pyrido[2,3-d]pyrimidine-6-carboxylic acid (20 mg, 0.048 mmol, 1.0 eq.) and dimethylamine hydrochloride (9.8 mg, 0.120 mmol, 2.5 eq.) in DCM (2.0 mL) was added Et₃N (14.6 mg, 0.144 mmol, 3.0 eq.) and BOP (21.4 mg, 0.048 mmol, 1.0 eq.). The mixture was stirred at RT overnight to give a deep red solution. The reaction was diluted with H₂O (4 mL), and extracted with DCM (12 mL). The organic layer was washed with brine (8 mL) and dried over anhydrous Na₂SO₄, filtered and concentrated to afford the crude, which was purified by flash column (C18, eluted with 0-80% MeCN in H₂O (0.5% HCO₂H)). The collected flows were concentrated and freeze-dried to afford (R)-4-((1-(3-(difluoromethyl)-2-fluorophenyl)ethyl)amino)-N,N,2-trimethyl-[1,2,4]triazolo[4′,3′:1,6]pyrido[2,3-d]pyrimidine-6-carboxamide.

| Example | | Mass m/z |
|---|---|---|
| # | Structure | (M + 1)⁺ |
| 50 | 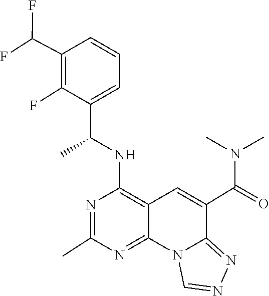 [SMILES: Cc1nc(N[C@H](C)c2cccc(C(F)F)c2F)c2cc(C(=O)N(C)C)c3nncn3c2n1] | 444.2 |
| 51 | 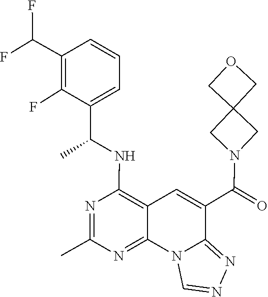 [SMILES: Cc1nc(N[C@H](C)c2cccc(C(F)F)c2F)c2cc(C(=O)N3CC4(COC4)C3)c3nncn3c2n1] | 498.2 |
| 52 | 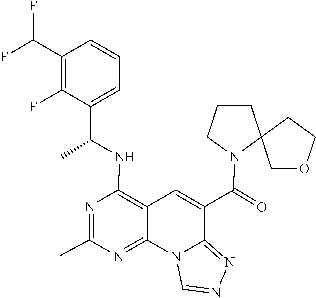 [SMILES: Cc1nc(N[C@H](C)c2cccc(C(F)F)c2F)c2cc(C(=O)N3CCCC34CCOC4)c3nncn3c2n1] | 526.3 |
| 53 | 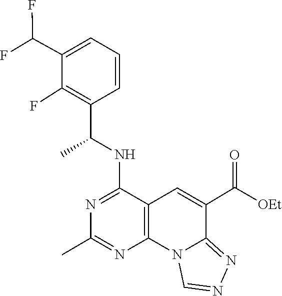 [SMILES: CCOC(=O)c1cc2c(N[C@H](C)c3cccc(C(F)F)c3F)nc(C)nc2n2cnnc12] | 445.2 |

### Example 54: Synthesis of (R)-(4-((1-(3-(difluoromethyl)-2-fluorophenyl)ethyl)amino)-2-methylimidazo[1′,2′:1,6]pyrido[2,3-d]pyrimidin-6-yl)(morpholino)methanone

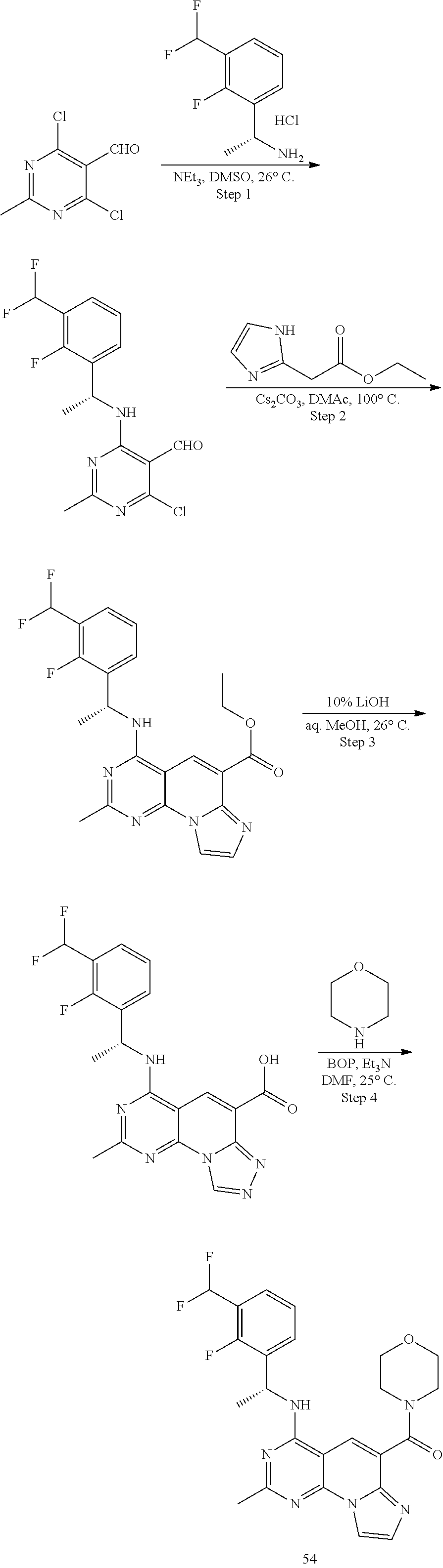

Step 1: To a solution of (R)-1-(3-(difluoromethyl)-2-fluorophenyl)ethan-1-amine hydrochloride (2.0 g, 8.7 mmol, 1.0 eq.) in DMSO (10 mL) was added 4,6-dichloro-2-methylpyrimidine-5-carbaldehyde (1.9 g, 9.8 mmol, 1.1 eq.) in DMSO (10 mL), then Et₃N (3.7 mL) was added in portions. The reaction was stirred at 26° C. for 10 min to give a crimson solution. The mixture was extracted with DCM (3×), the organic phase was concentrated under reduced pressure to dryness to give compound (R)-4-chloro-6-((1-(3-(difluoromethyl)-2-fluorophenyl)ethyl)amino)-2-methylpyrimidine-5-carbaldehyde.

Step 2: To a solution of (R)-4-chloro-6-((1-(3-(difluoromethyl)-2-fluorophenyl)ethyl)amino)-2-methylpyrimidine-5-carbaldehyde (1.2 g, 3.5 mmol, 1.0 eq.) in DMAc (12 mL) was added K₂CO₃ (1.0 g, 7.0 mmol, 2.0 eq.) and ethyl 2-(1H-imidazol-2-yl)acetate (0.5 g, 3.5 mmol, 1.0 eq.). The reaction was stirred at 100° C. for 2 h to give a red solution. The mixture was extracted with DCM (3×), the organic phase was concentrated under reduced pressure to dryness. The crude product was purified by flash column (SiO₂, eluted with 0˜100% EA in PE) to give ethyl (R)-4-((1-(3-(difluoromethyl)-2-fluorophenyl)ethyl)amino)-2-methylimidazo[1′,2′:1,6]pyrido[2,3-d]pyrimidine-6-carboxylate.

Step 3: To a solution of ethyl (R)-4-((1-(3-(difluoromethyl)-2-fluorophenyl)ethyl)amino)-2-methylimidazo[1′,2′:1,6]pyrido[2,3-d]pyrimidine-6-carboxylate (200 mg, 0.45 mmol, 1.0 eq.) in MeOH (5 mL) was added 10% LiOH aq. (3 mL). The reaction was stirred at 25° C. for 10 min to give a yellow solution. Then the mixture was added 1N HCl to adjust pH 3˜4 and extracted with DCM (3×). The organic phase was concentrated under reduced pressure to dryness to give (R)-4-((1-(3-(difluoromethyl)-2-fluorophenyl)ethyl)amino)-2-methylimidazo[1′,2′:1,6]pyrido[2,3-d]pyrimidine-6-carboxylic acid.

Step 4: To a solution of (R)-4-((1-(3-(difluoromethyl)-2-fluorophenyl)ethyl)amino)-2-methylimidazo[1′,2′:1,6]pyrido[2,3-d]pyrimidine-6-carboxylic acid (67.8 mg, 0.16 mmol, 1.0 eq.) in DMF (1 mL) was added BOP (144.4 mg, 0.33 mmol, 2.0 eq.) and stirred for 30 min, then Et₃N (0.045 mL) and morpholine (71.1 mg, 0.82 mmol, 2.0 eq.) was added. The mixture was stirred at 25° C. for 1 h to give a red solution. The mixture was concentrated under reduced pressure to dryness and purified by reversed column chromatography (MeCN/H₂O, from 0 to 100%) to give compound (R)-(4-((1-(3-(difluoromethyl)-2-fluorophenyl)ethyl)amino)-2-methylimidazo[1′,2′:1,6]pyrido[2,3-d]pyrimidin-6-yl)(morpholino)methanone.

| Example | | Mass m/z |
|---|---|---|
| # | Structure | (M + 1)⁺ |
| 54 | 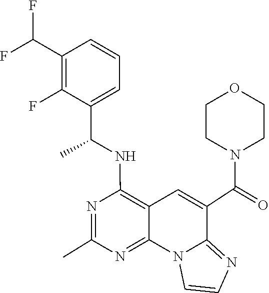 [SMILES: Cc1nc(N[C@H](C)c2cccc(C(F)F)c2F)c2cc(C(=O)N3CCOCC3)c3nccn3c2n1] | 485.2 |

### Example 55: (R)-6-((1-(3-(difluoromethyl)-2-fluorophenyl)ethyl)amino)-N,N,8-trimethyltetrazolo[1′,5′:1,6]pyrido[2,3-d]pyrimidine-4-carboxamide

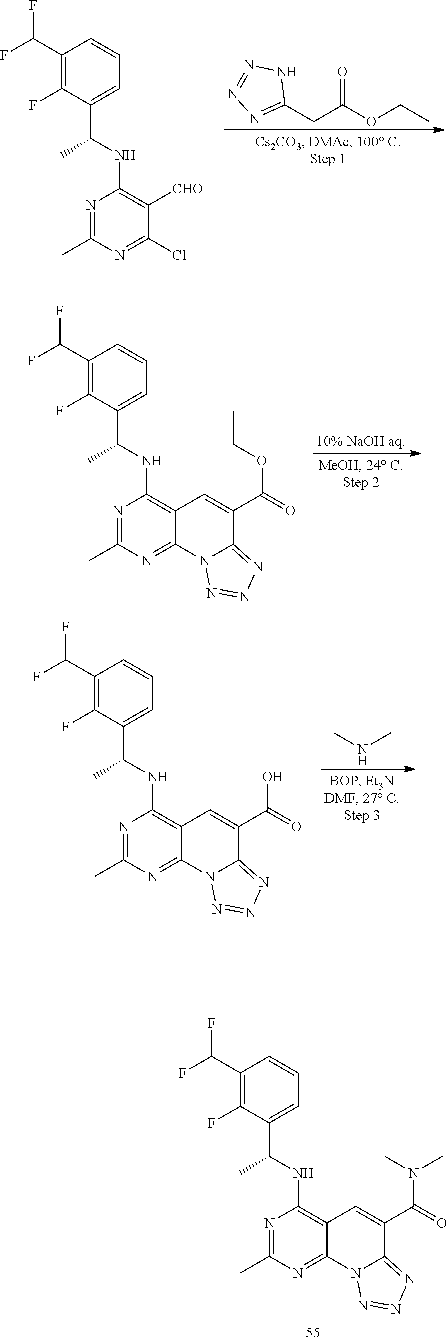

Step 1: To a solution of (R)-4-chloro-6-((1-(3-(difluoromethyl)-2-fluorophenyl)ethyl)amino)-2-methylpyrimidine-5-carbaldehyde (3.5 g, 9.5 mmol, 1.0 eq.) in ACN (30 mL) was added K₂CO₃ (2.6 g, 19.0 mmol, 2.0 eq.) and methyl 2-(1H-tetrazol-5-yl)acetate (1.5 g, 10.5 mmol, 1.0 eq.). The reaction was stirred at 100° C. overnight to give a red solution. The mixture was extracted with DCM (3×), the organic phase was concentrated under reduced pressure to dryness. The crude product was purified by flash column (SiO₂, eluted with 0˜100% EA in PE) to give compound methyl (R)-6-((1-(3-(difluoromethyl)-2-fluorophenyl)ethyl)amino)-8-methyl-[1,2,4]triazolo[1′,5′:1,6]pyrido[2,3-d]pyrimidine-4-carboxylate.

Step 2: To a solution of methyl (R)-6-((1-(3-(difluoromethyl)-2-fluorophenyl)ethyl)amino)-8-methyltetrazolo[1′,5′:1,6]pyrido[2,3-d]pyrimidine-4-carboxylate (732.7 mg, 1.7 mmol, 1.0 eq.) in MeOH (5 mL) was added 10% NaOH aq. (3 mL). The reaction was stirred at 24° C. for 10 min to give a yellow solution. Then the mixture was added 1N HCl to adjust pH 3˜4 and extracted with DCM (3×). The organic phase was concentrated under reduced pressure to dryness to give compound (R)-6-((1-(3-(difluoromethyl)-2-fluorophenyl)ethyl)amino)-8-methyltetrazolo[1′,5′:1,6]pyrido[2,3-d]pyrimidine-4-carboxylic acid.

Step 3: To a solution of (R)-6-((1-(3-(difluoromethyl)-2-fluorophenyl)ethyl)amino)-8-methyltetrazolo[1′,5′:1,6]pyrido[2,3-d]pyrimidine-4-carboxylic acid (100 mg, 0.24 mmol, 1.0 eq.) in DMF (2 mL) was added BOP (212 mg, 0.48 mmol, 2.0 eq.), and stirred for 30 min, then Et₃N (0.166 mL) and dimethylamine (108.0 mg, 2.4 mmol, 10 eq.) was added. The mixture was stirred at 26° C. for 1 h to give a red solution. The mixture was concentrated under reduced pressure to dryness and purified by reversed column chromatography (MeCN/H₂O, from 0 to 100%) to give (R)-6-((1-(3-(difluoromethyl)-2-fluorophenyl)ethyl)amino)-N,N,8-trimethyltetrazolo[1′,5′:1,6]pyrido[2,3-d]pyrimidine-4-carboxamide.

| Example # | Structure | Mass m/z (M + 1)⁺ |
|---|---|---|
| 55 | 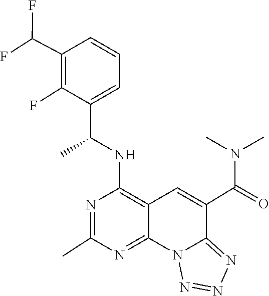 [SMILES: Cc1nc(N[C@H](C)c2cccc(C(F)F)c2F)c2cc(C(=O)N(C)C)c3nnnn3c2n1] | 445.2 |
| 56 | 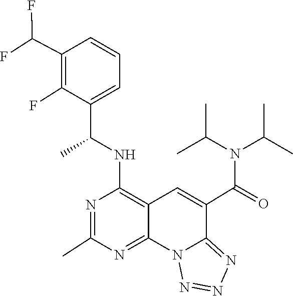 [SMILES: Cc1nc(N[C@H](C)c2cccc(C(F)F)c2F)c2cc(C(=O)N(C(C)C)C(C)C)c3nnnn3c2n1] | 501.2 |
| 57 | 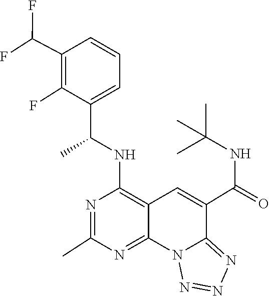 [SMILES: Cc1nc(N[C@H](C)c2cccc(C(F)F)c2F)c2cc(C(=O)NC(C)(C)C)c3nnnn3c2n1] | 473.2 |
| 58 | 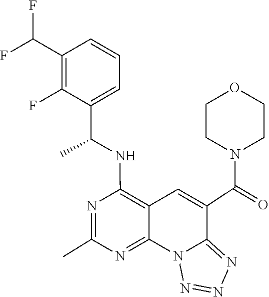 [SMILES: Cc1nc(N[C@H](C)c2cccc(C(F)F)c2F)c2cc(C(=O)N3CCOCC3)c3nnnn3c2n1] | 487.2 |
| 59 | 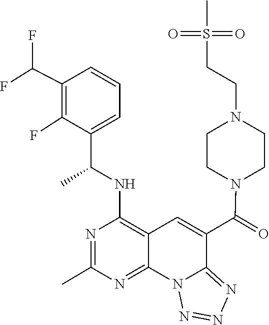 [SMILES: Cc1nc(N[C@H](C)c2cccc(C(F)F)c2F)c2cc(C(=O)N3CCN(CCS(C)(=O)=O)CC3)c3nnnn3c2n1] | 592.2 |
| 60 | 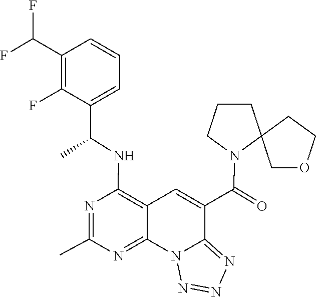 [SMILES: Cc1nc(N[C@H](C)c2cccc(C(F)F)c2F)c2cc(C(=O)N3CCCC34CCOC4)c3nnnn3c2n1] | 527.2 |
| 61 | 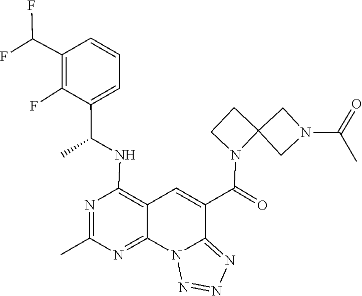 [SMILES: CC(=O)N1CC2(CCN2C(=O)c2cc3c(N[C@H](C)c4cccc(C(F)F)c4F)nc(C)nc3n3nnnc23)C1] | 540.2 |
| 62 | 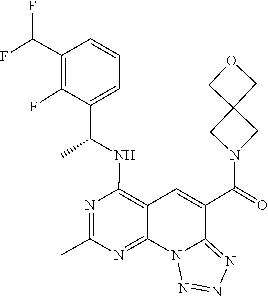 [SMILES: Cc1nc(N[C@H](C)c2cccc(C(F)F)c2F)c2cc(C(=O)N3CC4(COC4)C3)c3nnnn3c2n1] | 499.2 |
| 63 | 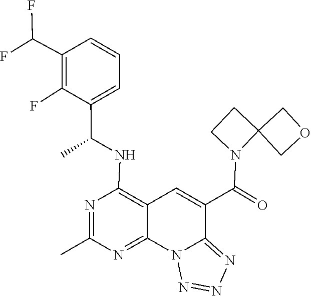 [SMILES: Cc1nc(N[C@H](C)c2cccc(C(F)F)c2F)c2cc(C(=O)N3CCC34COC4)c3nnnn3c2n1] | 499.2 |
| 64 | 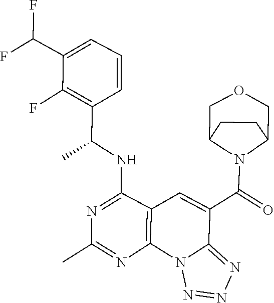 [SMILES: Cc1nc(N[C@H](C)c2cccc(C(F)F)c2F)c2cc(C(=O)N3C4CCC3COC4)c3nnnn3c2n1] | 513.2 |
| 65 | 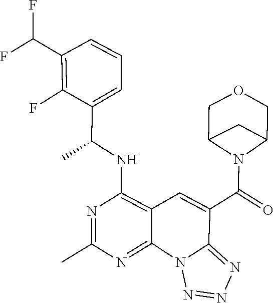 [SMILES: Cc1nc(N[C@H](C)c2cccc(C(F)F)c2F)c2cc(C(=O)N3C4COCC3C4)c3nnnn3c2n1] | 499.2 |
| 66 | 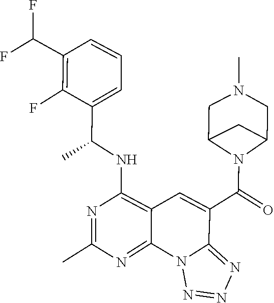 [SMILES: Cc1nc(N[C@H](C)c2cccc(C(F)F)c2F)c2cc(C(=O)N3C4CC3CN(C)C4)c3nnnn3c2n1] | 512.2 |

### Example 67: (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)piperidin-1-yl)ethan-1-one

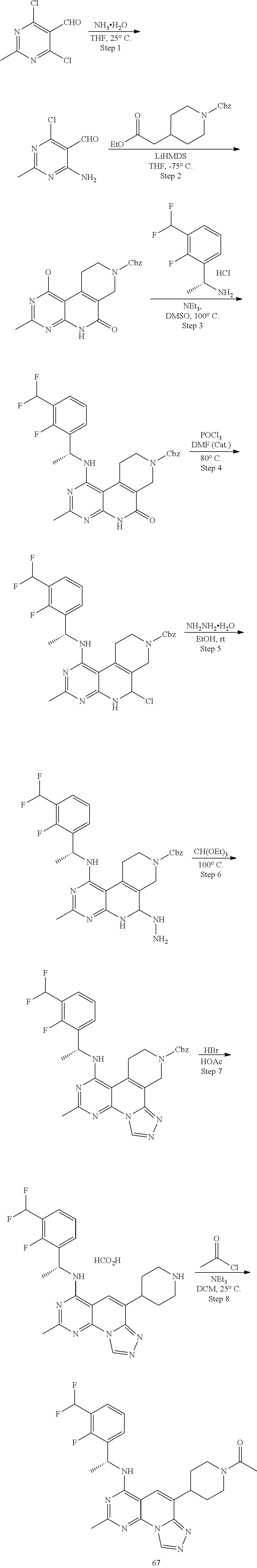

Step 1: The solution of 4,6-dichloro-2-methylpyrimidine-5-carbaldehyde (20.0 g, 105.8 mmol, 1.0 eq.) in THF (150 mL) was added drop wise into a solution of NH₃—H₂O (80 mL) at 0° C., then the reaction was stirred at room temperature for 1.0 h leading to an orange solution. The reaction was filtered to afford 4-amino-6-chloro-2-methylpyrimidine-5-carbaldehyde.

Step 2: A solution of LiHMDS (4.4 mL, 4.37 mmol, 2.5 eq., 1.0 M in THF) was added drop wise into a solution of benzyl 4-(2-ethoxy-2-oxoethyl)piperidine-1-carboxylate (764.1 mg, 2.62 mmol, 1.5 eq.) in THF (5 mL) at −78° C., the reaction was stirred at −78° C. for 30 min. A suspension of 4-amino-6-chloro-2-methylpyrimidine-5-carbaldehyde (300 mg, 1.75 mmol, 1.0 eq.) in THF (5 mL) was added to the above solution at −78° C. The reaction mixture was allowed to warm slowly to room temperature and stirred for 1 h leading to a yellow solution. Then the reaction was heated to 50° C. overnight. Water was added drop wise into the reaction mixture, and the residue was partitioned between ethyl acetate (20 mL). The separated organic layer was washed with water, dried over anhydrous Na₂SO₄ and evaporated to dryness. The crude product was purified by silica gel chromatography (PE/EA, from 0 to 50%) to give benzyl 4-(4-chloro-2-methyl-7-oxo-7,8-dihydropyrido[2,3-d]pyrimidin-6-yl)piperidine-1-carboxylate.

Step 3: To a solution of benzyl 4-(4-chloro-2-methyl-7-oxo-7,8-dihydropyrido[2,3-d]pyrimidin-6-yl)piperidine-1-carboxylate (550.0 mg, 1.33 mmol, 1.0 eq.) in DMSO (10 mL) was added (R)-1-(3-(difluoromethyl)-2-fluorophenyl)ethan-1-amine hydrochloride (360.7 mg, 1.60 mmol, 3.0 eq.) and Et₃N (0.56 mL, 4.0 mmol, 3.0 eq.) at rt, then the reaction mixture was heated at 90° C. for 2 h. The mixture was turned to a light-yellow solution. H₂O (10 mL) was added. The aqueous layer was extracted with EtOAc. The combined organic layers were washed with brine and dried over anhydrous Na₂SO₄. The crude product was purified by silica gel chromatography (PE/EA, from 0 to 80%) to give benzyl (R)-4-(4-((1-(3-(difluoromethyl)-2-fluorophenyl)ethyl)amino)-2-methyl-7-oxo-7,8-dihydropyrido[2,3-d]pyrimidin-6-yl)piperidine-1-carboxylate.

Step 4: benzyl (R)-4-(4-((1-(3-(difluoromethyl)-2-fluorophenyl)ethyl)amino)-2-methyl-7-oxo-7,8-dihydropyrido[2,3-d]pyrimidin-6-yl)piperidine-1-carboxylate (1.5 g, 2.65 mmol), POCl₃ (5.0 mL), and DMF (1 drop) were combined and the resulting mixture was heated at 80° C. in a sealed tube. After 3.0 h, the mixture was turned to a red brown solution. The reaction was concentrated under vacuum and the residue benzyl (R)-4-(7-chloro-4-((1-(3-(difluoromethyl)-2-fluorophenyl)ethyl)amino)-2-methylpyrido[2,3-d]pyrimidin-6-yl)piperidine-1-carboxylate was used for next step without further purification.

Step 5: To a solution of crude benzyl (R)-4-(7-chloro-4-((1-(3-(difluoromethyl)-2-fluorophenyl)ethyl)amino)-2-methylpyrido[2,3-d]pyrimidin-6-yl)piperidine-1-carboxylate (5.83 mmol) in EtOH (10.0 mL) was added hydrated hydrazine (2.0 mL) drop wise at RT and stirred for 1.0 h at 100° C. in a sealed tube. The mixture was turned to a dark red solution. Most of solvent was removed under reduced pressure and the residue benzyl (R)-4-(4-((1-(3-(difluoromethyl)-2-fluorophenyl)ethyl)amino)-7-hydrazineyl-2-methylpyrido[2,3-d]pyrimidin-6-yl)piperidine-1-carboxylate was used for next step without further purification.

Step 6: The solution of crude benzyl (R)-4-(4-((1-(3-(difluoromethyl)-2-fluorophenyl)ethyl)amino)-7-hydrazineyl-2-methylpyrido[2,3-d]pyrimidin-6-yl)piperidine-1-carboxylate (5.83 mmol) in CH(OEt)₃ (10.0 mL) was stirred for 12 h at 100° C. in a sealed tube leading to a dark red solution. The reaction mixture was cooled to rt and purified by reversed column chromatography (MeCN/H₂O, from 0 to 100%) to provide benzyl (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)piperidine-1-carboxylate

Step 7: To a solution of benzyl (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)piperidine-1-carboxylate (200 mg, 0.34 mmol, 1.0 eq.) in EtOH (2.0 mL) was added 12 M HCl (2.0 mL). The mixture was stirred for 24 h at rt to give a light brown solution. The reaction was poured into ice water and basified with Na₂CO₃ (aq.) to pH=10˜11, and then extracted with EA. The combined organic layers were washed with brine, dried over Na₂SO₄, filtered and concentrated to dryness. The residue was purified by flash column (C18, eluting a gradient of ACN/H₂O containing 0.5% HCCOH). The collected flows were lyophilized to afford (R)-N-(1-(3-(difluoromethyl)-2-fluorophenyl)ethyl)-2-methyl-6-(piperidin-4-yl)-[1,2,4]triazolo[4′,3′:1,6]pyrido[2,3-d]pyrimidin-4-amine formate.

Step 8: To a solution of (R)-N-(1-(3-(difluoromethyl)-2-fluorophenyl)ethyl)-2-methyl-6-(piperidin-4-yl)-[1,2,4]triazolo[4′,3′:1,6]pyrido[2,3-d]pyrimidin-4-amine formate (50 mg, 0.11 mmol, 1.0 eq.) in THF (1 mL) was added Et₃N (0.076 mL), then Acetyl chloride (8.6 mg, 0.11 mmol, 1.0 eq.) was added in dropwise. The reaction was stirred at 26° C. for 5 min to give a red solution. The mixture was concentrated under reduced pressure to dryness and purified by reversed column chromatography (MeCN/H₂O, from 0 to 100%) to give (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)piperidin-1-yl)ethan-1-one.

| Example # | Structure | Mass m/z (M + 1)⁺ |
|---|---|---|
| 67 | 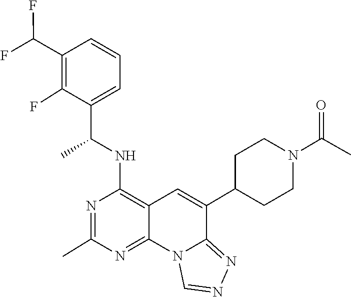 [SMILES: CC(=O)N1CCC(c2cc3c(N[C@H](C)c4cccc(C(F)F)c4F)nc(C)nc3n3cnnc23)CC1] | 498.2 |
| 68 | 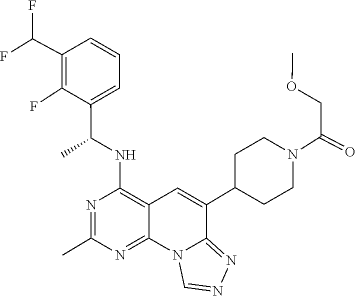 [SMILES: COCC(=O)N1CCC(c2cc3c(N[C@H](C)c4cccc(C(F)F)c4F)nc(C)nc3n3cnnc23)CC1] | 528.2 |
| 69 | 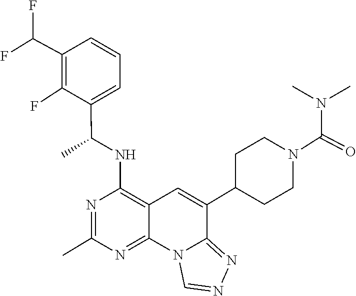 [SMILES: Cc1nc(N[C@H](C)c2cccc(C(F)F)c2F)c2cc(C3CCN(C(=O)N(C)C)CC3)c3nncn3c2n1] | 527.2 |
| 70 | 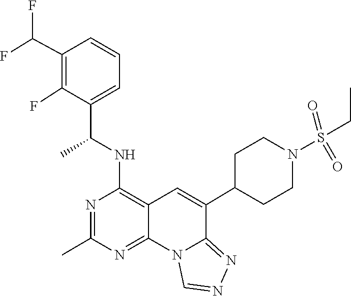 [SMILES: CCS(=O)(=O)N1CCC(c2cc3c(N[C@H](C)c4cccc(C(F)F)c4F)nc(C)nc3n3cnnc23)CC1] | 548.2 |
| 71 | 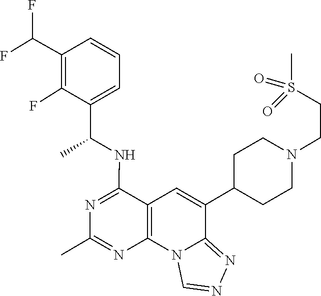 [SMILES: Cc1nc(N[C@H](C)c2cccc(C(F)F)c2F)c2cc(C3CCN(CCS(C)(=O)=O)CC3)c3nncn3c2n1] | 562.2 |
| 72 | 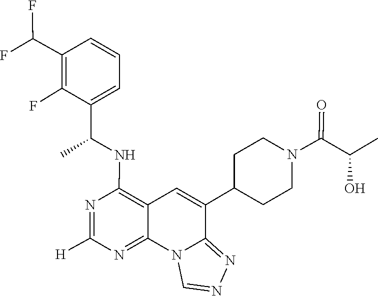 [SMILES: C[C@H](O)C(=O)N1CCC(c2cc3c(N[C@H](C)c4cccc(C(F)F)c4F)ncnc3n3cnnc23)CC1] | 514.2 |

### Example 73: (R)-2-(4-(4-((1-(3-(difluoromethyl)-2-fluorophenyl)ethyl)amino)-[1,2,4]triazolo[4′,3′:1,6]pyrido[2,3-d]pyrimidin-6-yl)piperidin-1-yl)oxazol-4(5H)-one

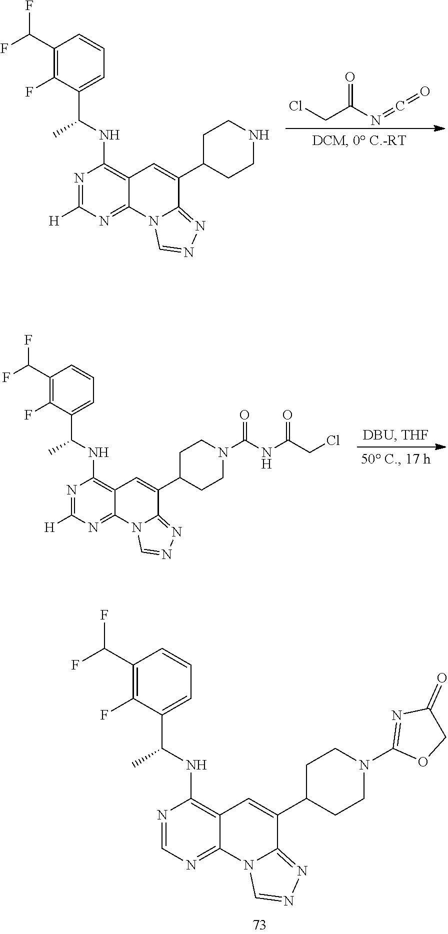 [SMILES: C[C@@H](Nc1ncnc2c1cc(C1CCNCC1)c1nncn12)c1cccc(C(F)F)c1F ; O=C=NC(=O)CCl ; C[C@@H](Nc1ncnc2c1cc(C1CCN(C(=O)NC(=O)CCl)CC1)c1nncn12)c1cccc(C(F)F)c1F ; C[C@@H](Nc1ncnc2c1cc(C1CCN(C3=NC(=O)CO3)CC1)c1nncn12)c1cccc(C(F)F)c1F]

Step 1: To a solution of (R)-N-(1-(3-(difluoromethyl)-2-fluorophenyl)ethyl)-6-(piperidin-4-yl)-[1,2,4]triazolo[4′,3′:1,6]pyrido[2,3-d]pyrimidin-4-amine (220 mg) in DCM (5.0 mL) was added 2-chloroacetyl isocyanate (60 mg, 0.50 mmol, 2.0 eq.) at 0° C. The mixture was then stirred at RT for 1 h to give a red-brown solution. The reaction was quenched with saturated NaHCO₃ solution (1 mL). The organic layer was washed with brine (5 mL), dried over anhydrous Na₂SO₄, filtered and concentrated to afford (R)-N-(2-chloroacetyl)-4-(4-((1-(3-(difluoromethyl)-2-fluorophenyl)ethyl)amino)-[1,2,4]triazolo[4′,3′:1,6]pyrido[2,3-d]pyrimidin-6-yl)piperidine-1-carboxamide which was used in the next step without further purification.

Step 2: To the above crude in THF (5.0 mL) was DBU (76 mg, 0.5 mmol, 2.0 eq). The was stirred at RT for 2 h to give a brown suspension. The volatiles were removed, and the crude was purified by flash column (C18, eluted with 0-80% MeCN in H₂O (0.5% HCO₂H)) to give (R)-2-(4-(4-((1-(3-(difluoromethyl)-2-fluorophenyl)ethyl)amino)-[1,2,4]triazolo[4′,3′:1,6]pyrido[2,3-d]pyrimidin-6-yl)piperidin-1-yl)oxazol-4(5H)-one.

LC/MS: 525.20 (M+1)⁺

¹H NMR (400 MHz, CD₃OD) δ 9.62 (s, 1H), 8.49 (s, 1H), 8.08 (d, J=3.7 Hz, 1H), 7.63 (t, J=7.4 Hz, 1H), 7.51 (t, J=7.0 Hz, 1H), 7.26 (t, J=7.7 Hz, 1H), 7.03 (t, J=54.8 Hz, 1H), 5.87 (q, J=6.9 Hz, 1H), 4.79 (d, J=13.2 Hz, 1H), 4.68 (q, J=6.6 Hz, 1H), 4.62 (s, 1H), 4.27 (d, J=13.6 Hz, 1H), 3.67-3.55 (m, 1H), 3.40 (d, J=13.6 Hz, 1H), 2.95 (t, J=12.8 Hz, 1H), 2.34-2.17 (m, 2H), 1.92 (tt, J=12.0, 6.3 Hz, 2H), 1.72 (d, J=7.0 Hz, 3H), 1.40 (dd, J=10.8, 6.6 Hz, 3H).

### Example 74: (R)—N-(1-(3-(difluoromethyl)-2-fluorophenyl)ethyl)-2-methyl-6-morpholino-[1,2,4]triazolo[4′,3′:1,6]pyrido[2,3-d]pyrimidin-4-amine

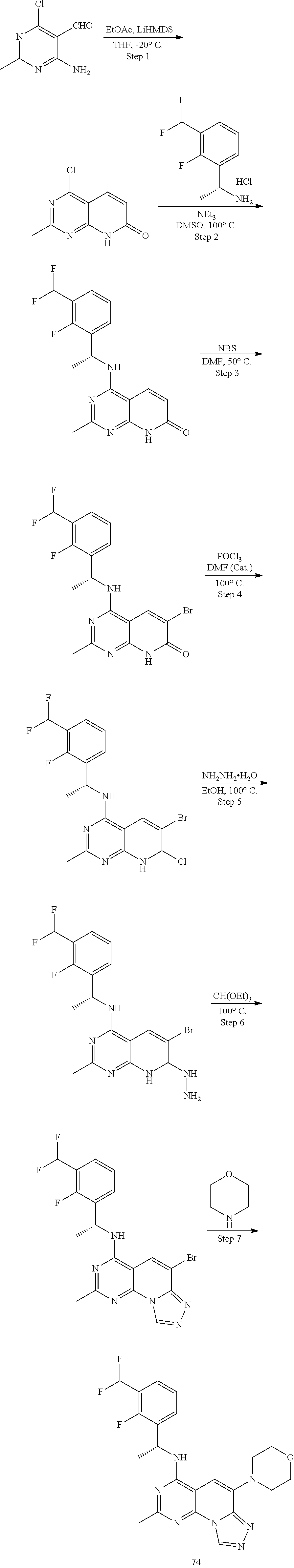

Step 1: To a mixture of 4-amino-6-chloro-2-methylpyrimidine-5-carbaldehyde (3.0 g, 17.48 mmol, 1.0 eq.) and EtOAc (2.56 mL, 26.23 mmol, 1.5 eq.) in THF (30 mL) was added LiHMDS (43.7 mL, 43.71 mmol, 2.5 eq.) at −20° C. and the resulting mixture was stirred at RT for 2 h leading to a red brown suspension. NH₄Cl (Sat.) was added dropwise to quench the reaction, and the suspension was concentrated to dryness. The residue was purified by FC(SiO₂) (PE/EA, 80%) to give 4-chloro-2-methylpyrido[2,3-d]pyrimidin-7(8H)-one.

Step 2: To a solution of 4-chloro-2-methylpyrido[2,3-d]pyrimidin-7(8H)-one (1.45 g, 7.41 mmol, 1.0 eq.) in DMSO (15.0 mL) was added (R)-1-(3-(difluoromethyl)-2-fluorophenyl)ethan-1-amine hydrochloride (1.67 g, 7.41 mmol, 1.0 eq.) and TEA (3.09 mL, 22.24 mmol, 3.0 eq.) at RT, the reaction mixture was heated at 90° C. for 2 h. The mixture was turned to a light-yellow solution. After cooling to room temperature, H₂O (10 mL) was added. The aqueous layer was extracted with EtOAc. The combined organic layers were washed with brine and dried over anhydrous Na₂SO₄ and concentrated under vacuum to (R)-4-((1-(3-(difluoromethyl)-2-fluorophenyl)ethyl)amino)-2-methylpyrido[2,3-d]pyrimidin-7(8H)-one which was used for the next step without purification.

Step 3: To a solution of (R)-4-((1-(3-(difluoromethyl)-2-fluorophenyl)ethyl)amino)-2-methylpyrido[2,3-d]pyrimidin-7(8H)-one (7.41 mmol) in DMF (5.0 mL) was added NBS (1.32 g, 7.41 mmol, 1.0 eq.) at rt, the reaction mixture was heated at 50° C. for 2 h. The mixture was turned to a light-yellow suspension. After cooling to room temperature, H₂O (20 mL) was added. The aqueous layer was extracted with EtOAc. The combined organic layers were washed with brine and dried over anhydrous Na₂SO₄, then concentrated under vacuum. The crude product was purified by silica gel chromatography (PE/EA, 40%) to give (R)-6-bromo-4-((1-(3-(difluoromethyl)-2-fluorophenyl)ethyl)amino)-2-methylpyrido[2,3-d]pyrimidin-7(8H)-one.

Step 4: (R)-6-bromo-4-((1-(3-(difluoromethyl)-2-fluorophenyl)ethyl)amino)-2-methylpyrido[2,3-d]pyrimidin-7(8H)-one (1.25 g, 2.93 mmol, 1.0 eq.), POCl₃ (5.0 mL), and DMF (1 drop) were combined and the resulting mixture was heated at 90° C. in a sealed tube. After 2 h, the mixture was turned to a red brown solution. The reaction was poured into ice water slowly, then extracted with EtOAc. The combined organic layers were washed with brine and dried over anhydrous Na₂SO₄, then concentrated under vacuum to the crude product (R)-6-bromo-7-chloro-N-(1-(3-(difluoromethyl)-2-fluorophenyl)ethyl)-2-methylpyrido[2,3-d]pyrimidin-4-amine which was used for the next step without purification.

Step 5: To a solution of crude (R)-6-bromo-7-chloro-N-(1-(3-(difluoromethyl)-2-fluorophenyl)ethyl)-2-methylpyrido[2,3-d]pyrimidin-4-amine (3.04 mmol) in EtOH (10.0 mL) was added hydrated hydrazine (1.0 mL) drop wise at RT and stirred for 1 h at 100° C. in a sealed tube. The mixture was turned to a dark red solution. Most of solvent was removed under reduced pressure and the residue (R)-6-bromo-N-(1-(3-(difluoromethyl)-2-fluorophenyl)ethyl)-7-hydrazineyl-2-methylpyrido[2,3-d]pyrimidin-4-amine was used for next step without further purification.

Step 6: A solution of crude (R)-6-bromo-N-(1-(3-(difluoromethyl)-2-fluorophenyl)ethyl)-7-hydrazineyl-2-methylpyrido[2,3-d]pyrimidin-4-amine (3.04 mmol) in CH(OEt)₃ (5.0 mL) was stirred for 12 h at 100° C. in a sealed tube leading to a dark red solution. The reaction mixture was cooled to RT, then extracted with EtOAc. The combined organic layers were washed with brine and dried over anhydrous Na₂SO₄, then concentrated under vacuum. The crude product was purified by silica gel chromatography (PE/EA, from 0 to 40% to 60%) to give (R)-6-bromo-N-(1-(3-(difluoromethyl)-2-fluorophenyl)ethyl)-2-methyl-[1,2,4]triazolo[4′,3′:1,6]pyrido[2,3-d]pyrimidin-4-amine.

Step 7: To a solution of (R)-6-bromo-N-(1-(3-(difluoromethyl)-2-fluorophenyl)ethyl)-2-methyl-[1,2,4]triazolo[4′,3′:1,6]pyrido[2,3-d]pyrimidin-4-amine (50 mg, 0.11 mmol, 1.0 eq) and morpholine (19.2 mg, 0.22 mmol, 2.0 eq.) in Dioxane (1.0 mL) was added XantPhos (12.8 mg, 0.022 mmol, 0.2 eq.), Cs₂CO₃ (108.3 mg, 0.33 mmol, 3.0 eq.) and Pd(OAc)₂ (2.5 mg, 0.011 mmol, 0.1 eq.) under N₂. The reaction was stirred at 110° C. by microwave for 2 h to give a black solution. The reaction was filtered through a pad of Celite. The filtrate was diluted with water and extracted with ethyl acetate. The combined organic layers were washed with brine, dried over sodium sulfate, filtered and concentrated to dryness. The residue was purified by flash column chromatography (C18, eluting a gradient of ACN/H₂O containing 0.5% HCCOH). The collected flows were concentrated to afford (R)—N-(1-(3-(difluoromethyl)-2-fluorophenyl)ethyl)-2-methyl-6-morpholino-[1,2,4]triazolo[4′,3′:1,6]pyrido[2,3-d]pyrimidin-4-amine.

| Example # | Structure | Mass m/z (M + 1)⁺ |
|---|---|---|
| 74 | 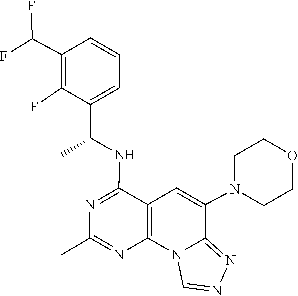 [SMILES: Cc1nc(N[C@H](C)c2cccc(C(F)F)c2F)c2cc(N3CCOCC3)c3nncn3c2n1] | 458.2 |
| 75 | 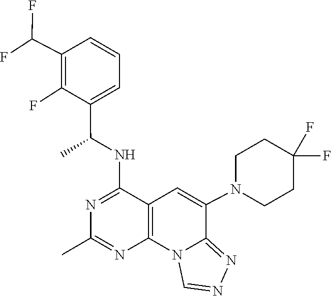 [SMILES: Cc1nc(N[C@H](C)c2cccc(C(F)F)c2F)c2cc(N3CCC(F)(F)CC3)c3nncn3c2n1] | 492.2 |
| 76 | 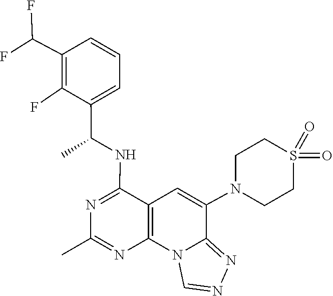 [SMILES: Cc1nc(N[C@H](C)c2cccc(C(F)F)c2F)c2cc(N3CCS(=O)(=O)CC3)c3nncn3c2n1] | 506.1 |
| 77 | 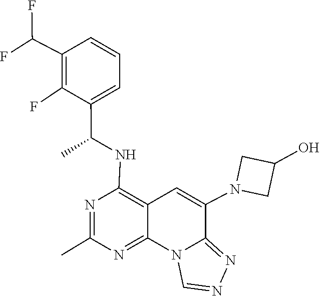 [SMILES: Cc1nc(N[C@H](C)c2cccc(C(F)F)c2F)c2cc(N3CC(O)C3)c3nncn3c2n1] | 444.3 |
| 78 | 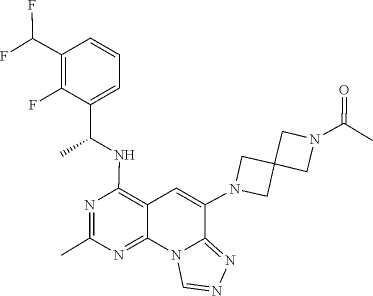 [SMILES: CC(=O)N1CC2(C1)CN(c1cc3c(N[C@H](C)c4cccc(C(F)F)c4F)nc(C)nc3n3cnnc13)C2] | 511.1 |
| 79 | 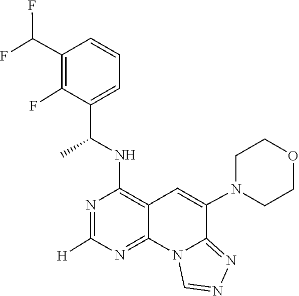 [SMILES: C[C@@H](Nc1ncnc2c1cc(N1CCOCC1)c1nncn12)c1cccc(C(F)F)c1F] | 444.2 |
| 80 | 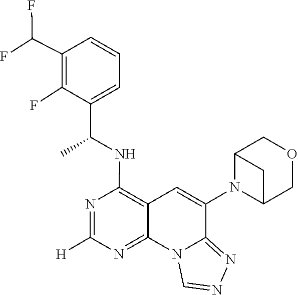 [SMILES: C[C@@H](Nc1ncnc2c1cc(N1C3COCC1C3)c1nncn12)c1cccc(C(F)F)c1F] | 456.2 |
| 81 | 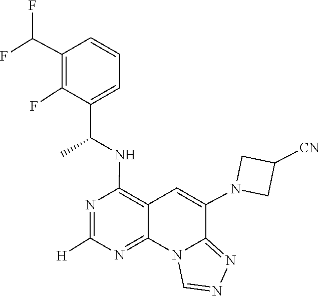 [SMILES: C[C@@H](Nc1ncnc2c1cc(N1CC(C#N)C1)c1nncn12)c1cccc(C(F)F)c1F] | 438.9 |
| 82 | 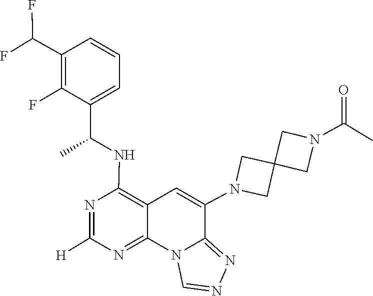 [SMILES: CC(=O)N1CC2(C1)CN(c1cc3c(N[C@H](C)c4cccc(C(F)F)c4F)ncnc3n3cnnc13)C2] | 497.1 |
| 83 | 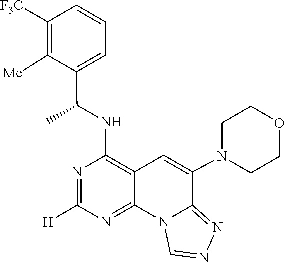 [SMILES: Cc1c([C@@H](C)Nc2ncnc3c2cc(N2CCOCC2)c2nncn23)cccc1C(F)(F)F] | 458.28 |
| 84 | 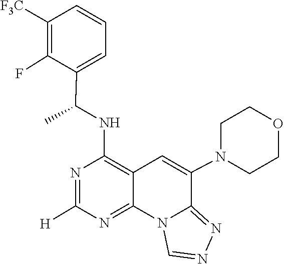 [SMILES: C[C@@H](Nc1ncnc2c1cc(N1CCOCC1)c1nncn12)c1cccc(C(F)(F)F)c1F] | 462.20 |
| 85 | 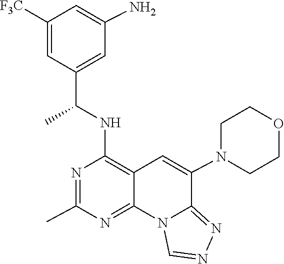 [SMILES: Cc1nc(N[C@H](C)c2cc(N)cc(C(F)(F)F)c2)c2cc(N3CCOCC3)c3nncn3c2n1] | 473.25 |
| 86 | 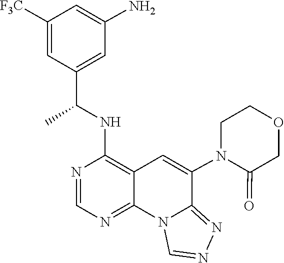 [SMILES: C[C@@H](Nc1ncnc2c1cc(N1CCOCC1=O)c1nncn12)c1cc(N)cc(C(F)(F)F)c1] | 473.20 |
| 87 | 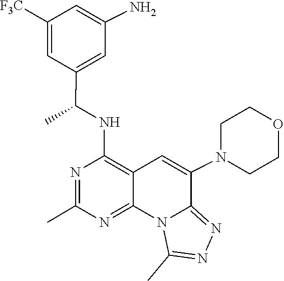 [SMILES: Cc1nc(N[C@H](C)c2cc(N)cc(C(F)(F)F)c2)c2cc(N3CCOCC3)c3nnc(C)n3c2n1] | 487.30 |
| 88 | 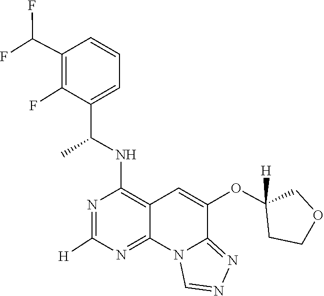 [SMILES: C[C@@H](Nc1ncnc2c1cc(O[C@H]1CCOC1)c1nncn12)c1cccc(C(F)F)c1F] | 445.3 |
| 89 | 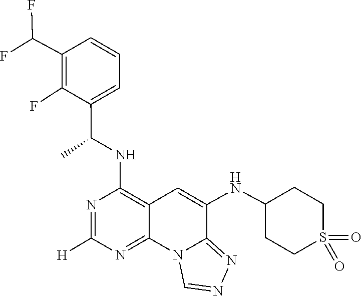 [SMILES: C[C@@H](Nc1ncnc2c1cc(NC1CCS(=O)(=O)CC1)c1nncn12)c1cccc(C(F)F)c1F] | 506.3 |

### Example 90: Cis-4-(4-(((R)-1-(3-(difluoromethyl)-2-fluorophenyl)ethyl)amino)-2-methyl-[1,2,4]triazolo[4′,3′:1,6]pyrido[2,3-d]pyrimidin-6-yl)cyclohexan-1-ol and trans-4-(4-(((R)-1-(3-(difluoromethyl)-2-fluorophenyl)ethyl)amino)-2-methyl-[1,2,4]triazolo[4′,3′:1,6]pyrido[2,3-d]pyrimidin-6-yl)cyclohexan-1-ol

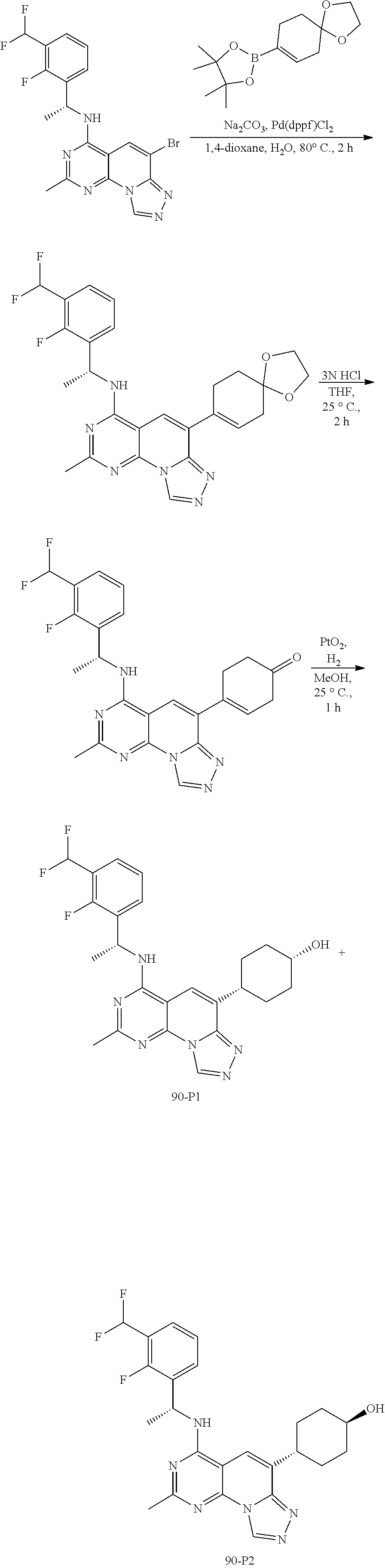

Step 1: A mixture (R)-6-bromo-N-(1-(3-(difluoromethyl)-2-fluorophenyl)ethyl)-2-methyl-[1,2,4]triazolo[4′,3′:1,6]pyrido[2,3-d]pyrimidin-4-amine (2.0 g, 4.4 mmol, 1.0 eq), 4,4,5,5-tetramethyl-2-(1,4-dioxaspiro[4.5]dec-7-en-8-yl)-1,3,2-dioxaborolane (2.63 g, 9.9 mmol, 2.25 eq), Pd(dppf)Cl₂ (0.64 mg, 0.88 mmol, 0.2 eq) and Na₂CO₃ (1.4 mg, 13.2 mmol, 3.0 eq) in 1,4-dioxane/H₂O (42 mL, 5:1) was stirred at 80° C. for 2.5 h. The reaction mixture was diluted with water (100 mL) and extracted with ethyl acetate (100 mL×3). The combined organic layers were washed with brine, dried, filtered and concentrated. The residue was purified by flash column chromatography (eluting with DCM:MeOH=97:3) to (R)—N-(1-(3-(difluoromethyl)-2-fluorophenyl)ethyl)-2-methyl-6-(1,4-dioxaspiro[4.5]dec-7-en-8-yl)-[1,2,4]triazolo[4′,3′:1,6]pyrido[2,3-d]pyrimidin-4-amine. LC-MS [M+H]+=511.2.

Step 2: To a solution of (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)cyclohex-3-en-1-one (1.8 g, 3.5 mmol) in THF (30 mL) was added HCl (10 mL, 3N) portionwise. The reaction mixture was stirred at 25° C. for 1.5 h. The reaction was quenched with NaHCO₃ (aq), then extracted with ethyl acetate (10 mL×3). The combined organic layers were washed with brine (100 mL), dried over sodium sulfate and filtered. The filtrate was concentrated and the residue was purified by recrystallization with MeOH to afford (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)cyclohex-3-en-1-one. LC-MS [M+H]+=467.15.

Step 3: To a solution of (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)cyclohex-3-en-1-one (1.2 g, 3.2 mmol) in MeOH (200 mL) was added PtO₂. The reaction mixture was stirred at 25° C. for 1.5 hours under hydrogen. The reaction was filtered. The filtrate was concentrated to afford (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)cyclohexan-1-ol (1.2 g, >100%) as a yellow solid. 100 mg of the crude product was purified with prep-HPLC (column: Gemini 5 um C18 150*20 mm; mobile phase: ACN-H₂O (0.1% TFA); gradient: 5-95) and lyophilized to afford cis-4-(4-(((R)-1-(3-(difluoromethyl)-2-fluorophenyl)ethyl)amino)-2-methyl-[1,2,4]triazolo[4′,3′:1,6]pyrido[2,3-d]pyrimidin-6-yl)cyclohexan-1-ol and trans-4-(4-(((R)-1-(3-(difluoromethyl)-2-fluorophenyl)ethyl)amino)-2-methyl-[1,2,4]triazolo[4′,3′:1,6]pyrido[2,3-d]pyrimidin-6-yl)cyclohexan-1-ol.

### Isomer 1: 90-P1

LC/MS: 471.15 (M+1)⁺.

¹H NMR (400 MHz, CD₃OD) δ 8.18 (s, 1H), 7.61 (t, J=7.2 Hz, 1H), 7.48 (t, J=7.2 Hz, 1H), 7.24 (t, J=8.0 Hz, 1H), 7.14-6.87 (t, J=54.4 Hz, 1H), 5.86-5.81 (m, 1H), 3.70 (s, 1H), 3.23 (s, 1H), 2.50 (s, 3H), 2.16-2.14 (m, 4H), 1.81 (s, 2H), 1.69 (d, J=7.2 Hz, 3H), 1.57-1.52 (m, 2H). LC/MS: 471.15 (M+1)⁺.

### Isomer 2: 90-P2

¹H NMR (400 MHz, CD₃OD) δ 9.67 (s, 1H), 8.19 (s, 1H), 7.63 (t, J=7.2 Hz, 1H), 7.48 (t, J=6.8 Hz, 1H), 7.24 (t, J=8.0 Hz, 1H), 7.14-6.87 (m, 1H), 5.86 (q, J=7.2 Hz, 1H), 4.15 (s, 1H), 3.24 (m, 1H), 2.51 (s, 3H), 2.12 (m, 2H), 1.99 (m, 2H), 1.91 (m, 2H), 1.82 (m, 2H), 1.71 (d, J=7.2 Hz, 3H).

### Example 91: Synthesis of (R)—N-(1-(3-(difluoromethyl)-2-fluorophenyl)ethyl)-8-methyl-4-morpholinotetrazolo[1′,5′:1,6]pyrido[2,3-d]pyrimidin-6-amine

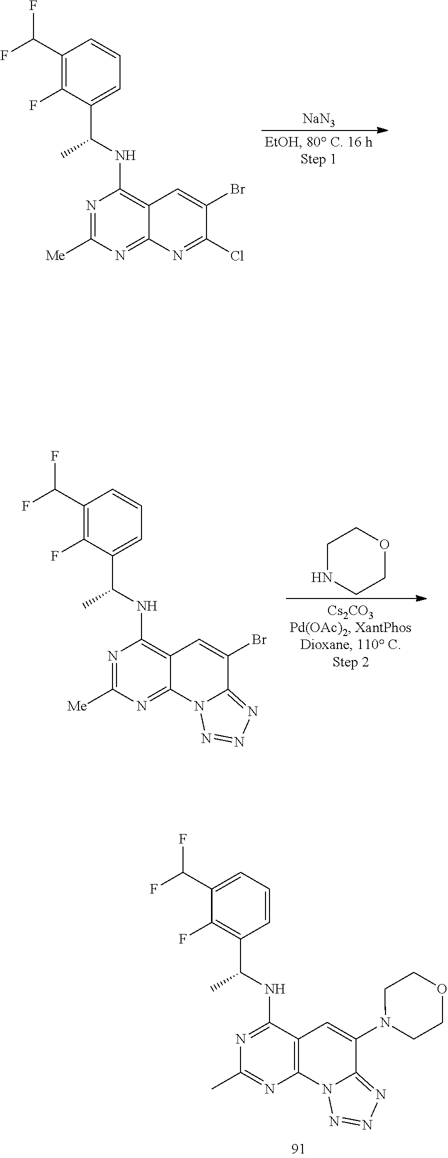 [SMILES: Cc1nc(N[C@H](C)c2cccc(C(F)F)c2F)c2cc(Br)c(Cl)nc2n1 ; Cc1nc(N[C@H](C)c2cccc(C(F)F)c2F)c2cc(Br)c3nnnn3c2n1 ; C1COCCN1 ; Cc1nc(N[C@H](C)c2cccc(C(F)F)c2F)c2cc(N3CCOCC3)c3nnnn3c2n1 ; "NaN3"]

Step 1: To a solution of (R)-6-bromo-7-chloro-N-(1-(3-(difluoromethyl)-2-fluorophenyl)ethyl)-2-methylpyrido[2,3-d]pyrimidin-4-amine (2 g, 4.5 mmol, 1.0 eq) in EtOH at 25° C. was added NaN₃ (2.05 g, 31.5 mmol, 7.0 eq) portion wise. The reaction mixture was stirred at 80° C. for 16 h. The reaction was quenched with water (50 mL), extracted with ethyl acetate (50 mL×3). The combined organic layers were washed with brine (50 mL), dried over sodium sulfate and filtered. The filtrate was concentrated and the residue was purified by flash column chromatography (eluting with DCM:MeOH=98:2) to afford (R)-4-bromo-N-(1-(3-(difluoromethyl)-2-fluorophenyl)ethyl)-8-methyltetrazolo[1′,5′:1,6]pyrido[2,3-d]pyrimidin-6-amine. LC-MS [M+H]⁺=452.5.

Step 2: To a solution of (R)-4-bromo-N-(1-(3-(difluoromethyl)-2-fluorophenyl)ethyl)-8-methyltetrazolo[1′,5′:1,6]pyrido[2,3-d]pyrimidin-6-amine (50 mg, 0.11 mmol, 1.0 eq) and morpholine (19.2 mg, 0.22 mmol, 2.0 eq.) in Dioxane (1.0 mL) was added XantPhos (12.8 mg, 0.022 mmol, 0.2 eq.), Cs₂CO₃ (108.3 mg, 0.33 mmol, 3.0 eq.) and Pd(OAc)₂ (2.5 mg, 0.011 mmol, 0.1 eq.) under N₂. The reaction was stirred at 110° C. by microwave for 2 h to give a black solution. The reaction was filtered through a pad of Celite. The filtrate was diluted with water and extracted with ethyl acetate. The combined organic layers were washed with brine, dried over sodium sulfate, filtered and concentrated to dryness. The residue was purified by flash column chromatography (C18, eluting a gradient of ACN/H₂O containing 0.5% HCCOH). The collected flows were concentrated to afford (R)—N-(1-(3-(difluoromethyl)-2-fluorophenyl)ethyl)-8-methyl-4-morpholinotetrazolo[1′,5′:1,6]pyrido[2,3-d]pyrimidin-6-amine.

| Example # | Structure | Mass m/z (M + 1)⁺ |
|---|---|---|
| 91 | 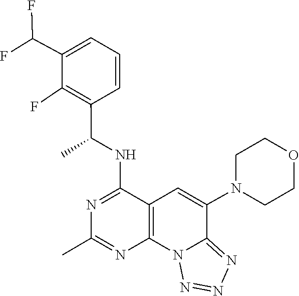 [SMILES: Cc1nc(N[C@H](C)c2cccc(C(F)F)c2F)c2cc(N3CCOCC3)c3nnnn3c2n1] | 459.2 |
| 92 | 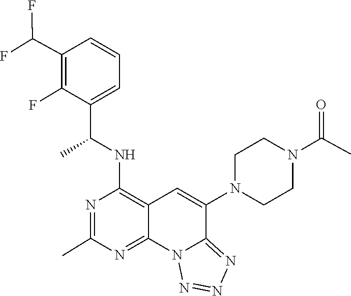 [SMILES: CC(=O)N1CCN(c2cc3c(N[C@H](C)c4cccc(C(F)F)c4F)nc(C)nc3n3nnnc23)CC1] | 500.3 |
| 93 | 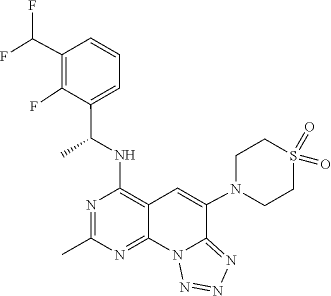 [SMILES: Cc1nc(N[C@H](C)c2cccc(C(F)F)c2F)c2cc(N3CCS(=O)(=O)CC3)c3nnnn3c2n1] | 507.1 |
| 94 | 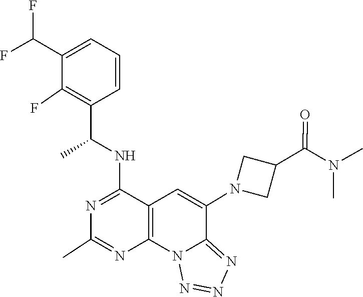 [SMILES: Cc1nc(N[C@H](C)c2cccc(C(F)F)c2F)c2cc(N3CC(C(=O)N(C)C)C3)c3nnnn3c2n1] | 500.2 |
| 95 | 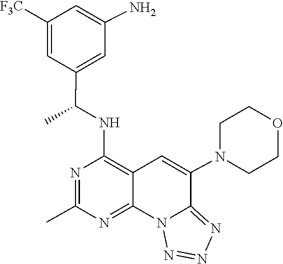 [SMILES: Cc1nc(N[C@H](C)c2cc(N)cc(C(F)(F)F)c2)c2cc(N3CCOCC3)c3nnnn3c2n1] | 474.2 |
| 96 | 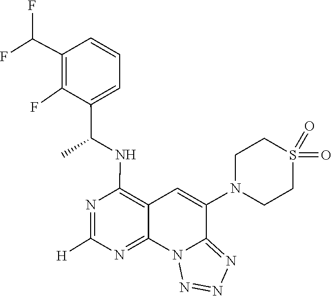 [SMILES: C[C@@H](Nc1ncnc2c1cc(N1CCS(=O)(=O)CC1)c1nnnn12)c1cccc(C(F)F)c1F] | 493.1 |
| 97 | 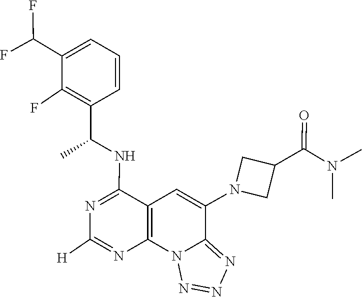 [SMILES: C[C@@H](Nc1ncnc2c1cc(N1CC(C(=O)N(C)C)C1)c1nnnn12)c1cccc(C(F)F)c1F] | 486.3 |
| 98 | 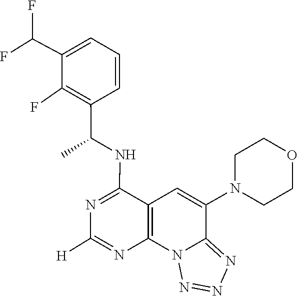 [SMILES: C[C@@H](Nc1ncnc2c1cc(N1CCOCC1)c1nnnn12)c1cccc(C(F)F)c1F] | 445.2 |
| 99 | 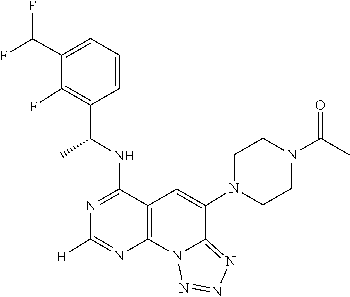 [SMILES: CC(=O)N1CCN(c2cc3c(N[C@H](C)c4cccc(C(F)F)c4F)ncnc3n3nnnc23)CC1] | 486.2 |

### Example 100: (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydropyridin-1(2H)-yl)ethan-1-one

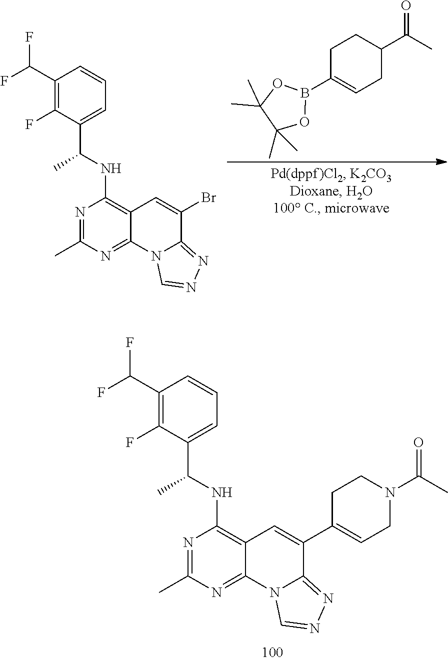 [SMILES: Cc1nc(N[C@H](C)c2cccc(C(F)F)c2F)c2cc(Br)c3nncn3c2n1 ; CC(=O)C1CC=C(B2OC(C)(C)C(C)(C)O2)CC1 ; CC(=O)N1CC=C(c2cc3c(N[C@H](C)c4cccc(C(F)F)c4F)nc(C)nc3n3cnnc23)CC1]

Step 1: To a solution of (R)-6-bromo-N-(1-(3-(difluoromethyl)-2-fluorophenyl)ethyl)-2-methyl-[1,2,4]triazolo[4′,3′:1,6]pyrido[2,3-d]pyrimidin-4-amine (1.0 g, 2.22 mmol, 1.0 eq.) and 1-(4-(4,4,5,5-tetramethyl-1,3,2-dioxaborolan-2-yl)cyclohex-3-en-1-yl)ethan-1-one (0.83 g, 3.32 mmol, 1.5 eq.) in dioxane (10.0 mL) and H₂O (2.0 mL) was added K₂CO₃ (0.92 g, 6.65 mmol, 3.0 eq.) and Pd(dppf)Cl₂ (81 mg, 0.11 mmol, 0.05 eq.) under N₂. The reaction was stirred for 0.5 hour at 100° C. by microwave to give a brown solution. The residue was diluted with H₂O and extracted with EA (30 mL×2). The combined organic layers were washed with brine, dried over Na₂SO₄, filtered and concentrated under vacuum to dryness. The crude product was purified by silica gel chromatography (DCM/MeOH, from 5%) to give (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydropyridin-1(2H)-yl)ethan-1-one.

| | | Mass |
|---|---|---|
| Example | | m/z |
| # | Structure | (M + 1)⁺ |
| 100 | 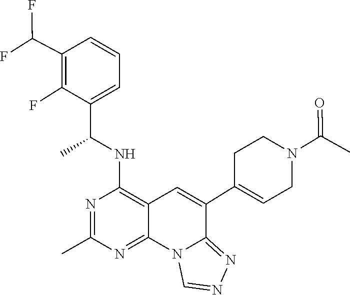 [SMILES: CC(=O)N1CC=C(c2cc3c(N[C@H](C)c4cccc(C(F)F)c4F)nc(C)nc3n3cnnc23)CC1] | 496.2 |
| 101 | 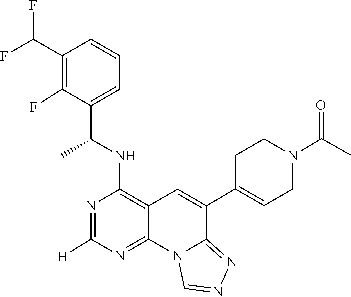 [SMILES: CC(=O)N1CC=C(c2cc3c(N[C@H](C)c4cccc(C(F)F)c4F)ncnc3n3cnnc23)CC1] | 482.2 |
| 102 | 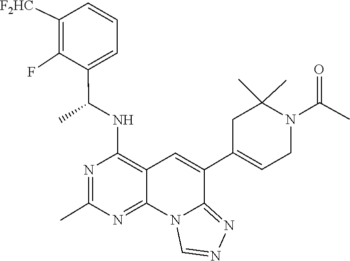 [SMILES: CC(=O)N1CC=C(c2cc3c(N[C@H](C)c4cccc(C(F)F)c4F)nc(C)nc3n3cnnc23)CC1(C)C] | 524.2 |
| 103 | 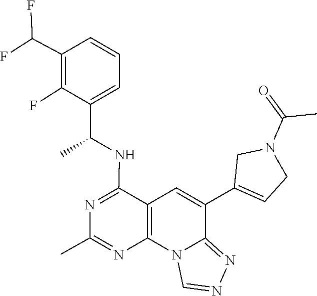 [SMILES: CC(=O)N1CC=C(c2cc3c(N[C@H](C)c4cccc(C(F)F)c4F)nc(C)nc3n3cnnc23)C1] | 482.2 |
| 104 | 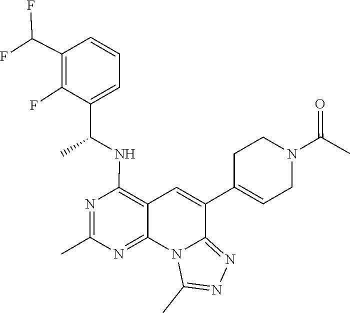 [SMILES: CC(=O)N1CC=C(c2cc3c(N[C@H](C)c4cccc(C(F)F)c4F)nc(C)nc3n3c(C)nnc23)CC1] | 510.2 |
| 105 | 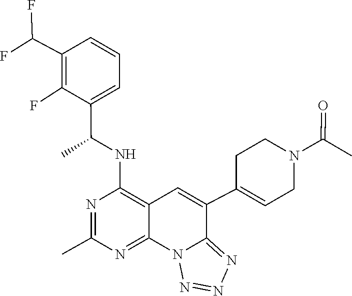 [SMILES: CC(=O)N1CC=C(c2cc3c(N[C@H](C)c4cccc(C(F)F)c4F)nc(C)nc3n3nnnc23)CC1] | 497.2 |
| 106 | 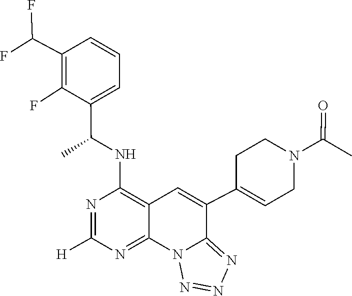 [SMILES: CC(=O)N1CC=C(c2cc3c(N[C@H](C)c4cccc(C(F)F)c4F)ncnc3n3nnnc23)CC1] | |

### Example 107: Synthesis of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-hydroxypiperidin-1-yl)ethan-1-one

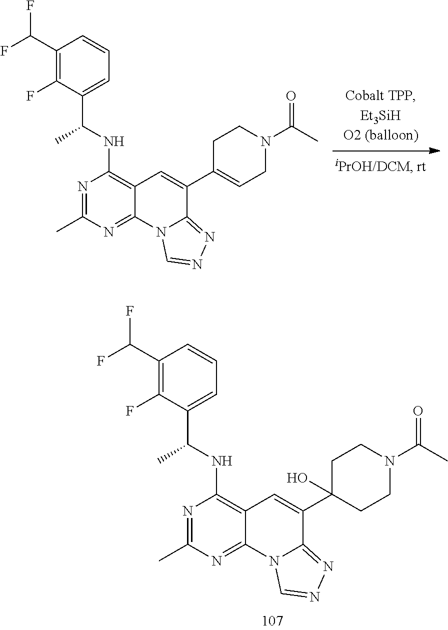 [SMILES: CC(=O)N1CC=C(c2cc3c(N[C@H](C)c4cccc(C(F)F)c4F)nc(C)nc3n3cnnc23)CC1 ; CC(=O)N1CCC(O)(c2cc3c(N[C@H](C)c4cccc(C(F)F)c4F)nc(C)nc3n3cnnc23)CC1]

Step 1: To a solution of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydropyridin-1(2H)-yl)ethan-1-one (500 mg, 1.01 mmol, 1.0 eq.) and Cobalt TPP (67.8 mg, 0.101 mmol, 0.1 eq.) in 8.0 mL of DCM/ⁱPrOH (1:1) was added Et₃SiH (0.48 mL, 3.027 mmol, 2.0 eq.) at 0° C. Then the reaction was stirred at RT under atmospheric pressure of O₂ (balloon) for 2 h leading to a brown solution. The reaction was concentrated under vacuum to dryness. The crude product was purified by silica gel chromatography (DCM/MeOH, from 0 to 5%) to give (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-hydroxypiperidin-1-yl)ethan-1-one.

| | | Mass |
|---|---|---|
| Example | | m/z |
| # | Structure | (M + 1)⁺ |
| 107 | 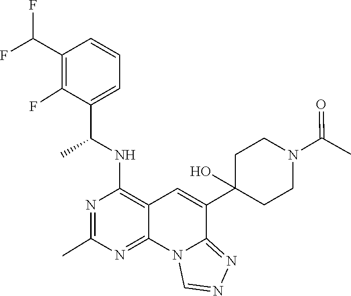 [SMILES: CC(=O)N1CCC(O)(c2cc3c(N[C@H](C)c4cccc(C(F)F)c4F)nc(C)nc3n3cnnc23)CC1] | 514.2 |
| 108 | 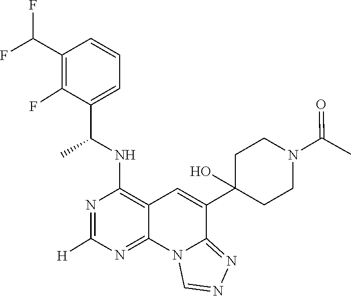 [SMILES: CC(=O)N1CCC(O)(c2cc3c(N[C@H](C)c4cccc(C(F)F)c4F)ncnc3n3cnnc23)CC1] | 500.2 |
| 109 | 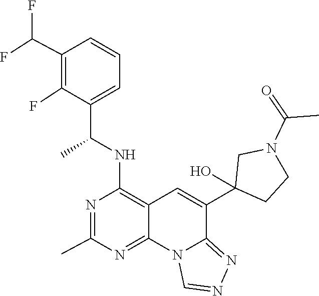 [SMILES: CC(=O)N1CCC(O)(c2cc3c(N[C@H](C)c4cccc(C(F)F)c4F)nc(C)nc3n3cnnc23)C1] | 500.2 |
| 110 | 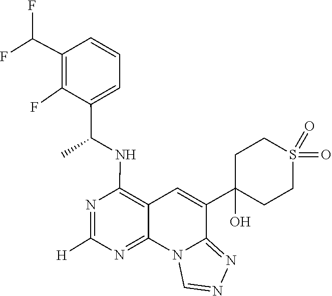 [SMILES: C[C@@H](Nc1ncnc2c1cc(C1(O)CCS(=O)(=O)CC1)c1nncn12)c1cccc(C(F)F)c1F] | 507.1 |
| 111 | 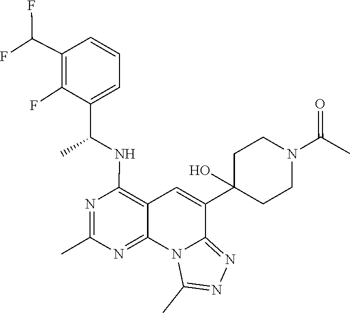 [SMILES: CC(=O)N1CCC(O)(c2cc3c(N[C@H](C)c4cccc(C(F)F)c4F)nc(C)nc3n3c(C)nnc23)CC1] | 528.2 |
| 112 | 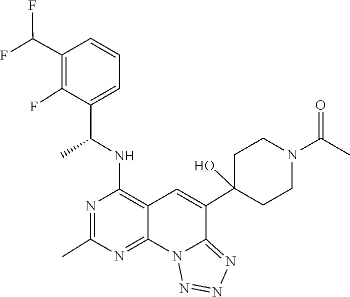 [SMILES: CC(=O)N1CCC(O)(c2cc3c(N[C@H](C)c4cccc(C(F)F)c4F)nc(C)nc3n3nnnc23)CC1] | 515.3 |
| 113 | 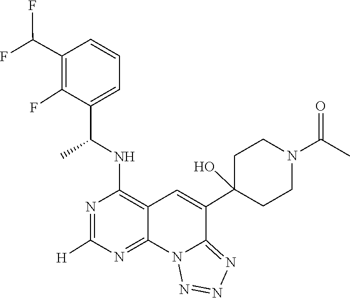 [SMILES: CC(=O)N1CCC(O)(c2cc3c(N[C@H](C)c4cccc(C(F)F)c4F)ncnc3n3nnnc23)CC1] | 501.1 |

### Example 114: Synthesis of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-fluoropiperidin-1-yl)ethan-1-one

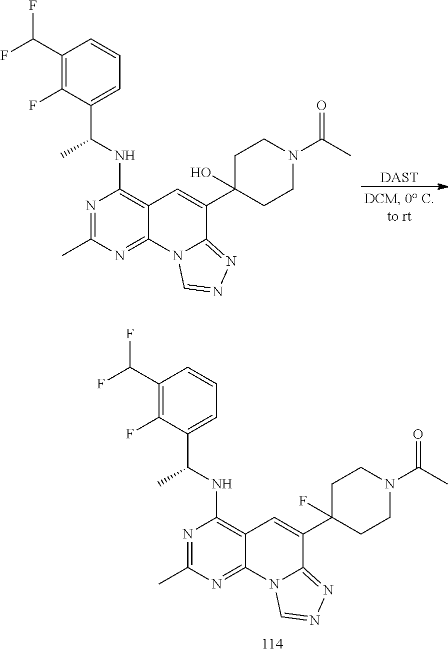

Step 1: To a solution of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-hydroxypiperidin-1-yl)ethan-1-one (300 mg, 0.584 mmol, 1.0 eq.) in 5.0 mL of DCM was added DAST (0.12 mL, 0.876 mmol, 1.5 eq.) at 0° C. After stirred at 0° C. for 0.5 h, the reaction was stirred at RT for 1 h leading to a brown solution. NaHCO₃ (Sat.) was added to quench the reaction and extracted with DCM. The combined organic layers were washed with brine, dried over Na₂SO₄, filtered and concentrated under vacuum to dryness. The crude product was purified by reversed column chromatography (MeCN/H₂O, from 0 to 50%) to provide (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-fluoropiperidin-1-yl)ethan-1-one

| | | Mass |
|---|---|---|
| Example | | m/z |
| # | Structure | (M + 1)⁺ |
| 114 | 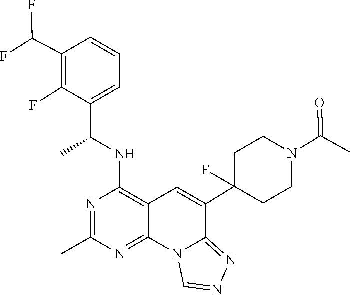 [SMILES: CC(=O)N1CCC(F)(c2cc3c(N[C@H](C)c4cccc(C(F)F)c4F)nc(C)nc3n3cnnc23)CC1] | 516.2 |
| 115 | 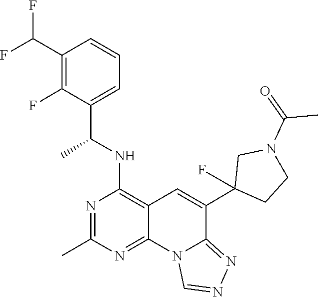 [SMILES: CC(=O)N1CCC(F)(c2cc3c(N[C@H](C)c4cccc(C(F)F)c4F)nc(C)nc3n3cnnc23)C1] | 502.2 |
| 116 | 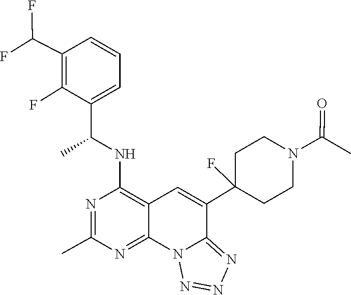 [SMILES: CC(=O)N1CCC(F)(c2cc3c(N[C@H](C)c4cccc(C(F)F)c4F)nc(C)nc3n3nnnc23)CC1] | 517.2 |
| 117 | 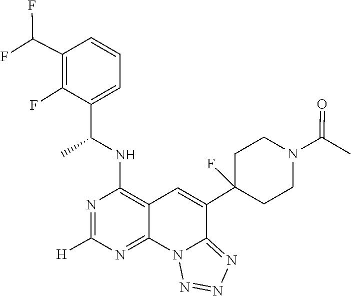 [SMILES: CC(=O)N1CCC(F)(c2cc3c(N[C@H](C)c4cccc(C(F)F)c4F)ncnc3n3nnnc23)CC1] | 503.2 |

### Example 118: Synthesis of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-methoxypiperidin-1-yl)ethan-1-one

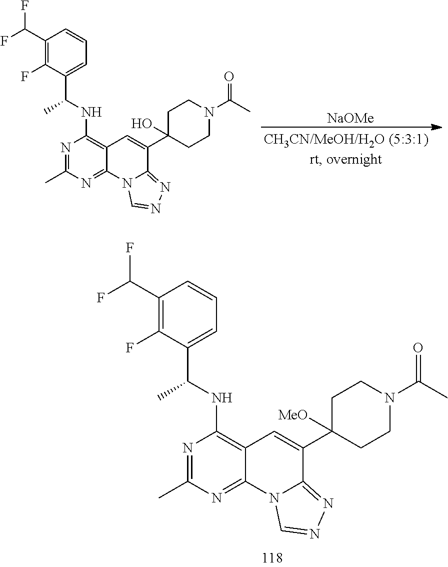 [SMILES: CC(=O)N1CCC(O)(c2cc3c(N[C@H](C)c4cccc(C(F)F)c4F)nc(C)nc3n3cnnc23)CC1 ; COC1(c2cc3c(N[C@H](C)c4cccc(C(F)F)c4F)nc(C)nc3n3cnnc23)CCN(C(C)=O)CC1 ; "NaOMe"]

Step 1: To a solution of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-fluoropiperidin-1-yl)ethan-1-one (24.5 mg, 0.047 mmol, 1.0 eq.) in 5.4 mL of CH₃CN/MeOH/H₂O (5:3:1) was added NaOMe (23.1 mg, 0.428 mmol, 9.0 eq.) at 0° C. The reaction was stirred at RT overnight leading to a yellow solution. NH₄Cl (Sat.) was added to quench the reaction and extracted with DCM. The combined organic layers were washed with brine, dried over Na₂SO₄, filtered and concentrated under vacuum to dryness. The crude product was purified by reversed column chromatography (MeCN/H₂O, from 0 to 50%) to provide (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-methoxypiperidin-1-yl)ethan-1-one.

| | | Mass |
|---|---|---|
| Example | | m/z |
| # | Structure | (M + 1)⁺ |
| 118 | 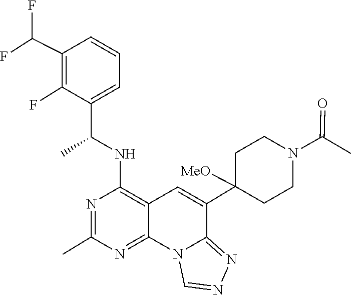 [SMILES: COC1(c2cc3c(N[C@H](C)c4cccc(C(F)F)c4F)nc(C)nc3n3cnnc23)CCN(C(C)=O)CC1] | 528.3 |
| 119 | 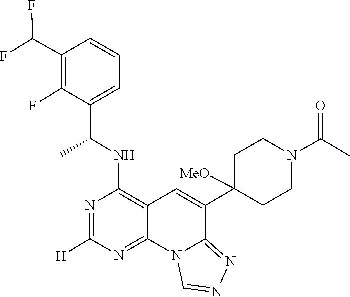 [SMILES: COC1(c2cc3c(N[C@H](C)c4cccc(C(F)F)c4F)ncnc3n3cnnc23)CCN(C(C)=O)CC1] | 514.2 |
| 120 | 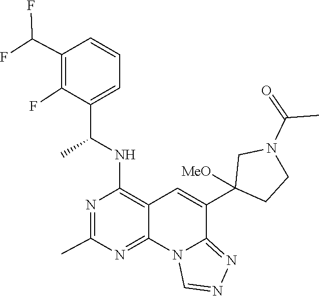 [SMILES: COC1(c2cc3c(N[C@H](C)c4cccc(C(F)F)c4F)nc(C)nc3n3cnnc23)CCN(C(C)=O)C1] | 514.5 |
| 121 | 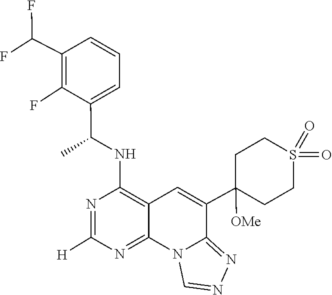 [SMILES: COC1(c2cc3c(N[C@H](C)c4cccc(C(F)F)c4F)ncnc3n3cnnc23)CCS(=O)(=O)CC1] | 521.1 |
| 122 | 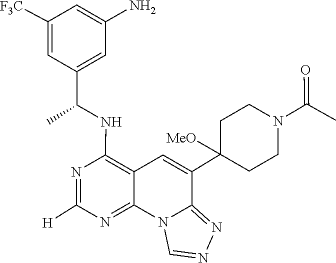 [SMILES: COC1(c2cc3c(N[C@H](C)c4cc(N)cc(C(F)(F)F)c4)ncnc3n3cnnc23)CCN(C(C)=O)CC1] | 529.2 |
| 123 | 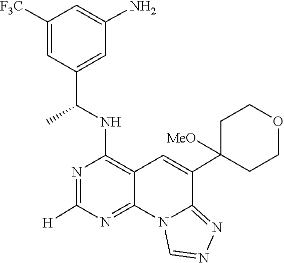 [SMILES: COC1(c2cc3c(N[C@H](C)c4cc(N)cc(C(F)(F)F)c4)ncnc3n3cnnc23)CCOCC1] | 488.3 |
| 124 | 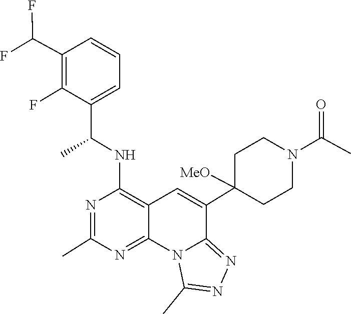 [SMILES: COC1(c2cc3c(N[C@H](C)c4cccc(C(F)F)c4F)nc(C)nc3n3c(C)nnc23)CCN(C(C)=O)CC1] | 542.2 |
| 125 | 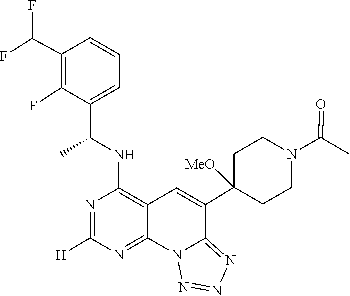 [SMILES: COC1(c2cc3c(N[C@H](C)c4cccc(C(F)F)c4F)ncnc3n3nnnc23)CCN(C(C)=O)CC1] | 515.2 |
| 126 | 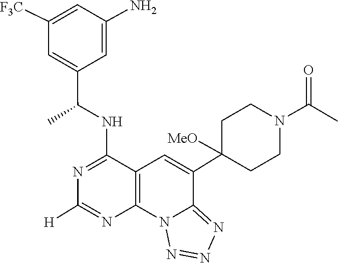 [SMILES: COC1(c2cc3c(N[C@H](C)c4cc(N)cc(C(F)(F)F)c4)ncnc3n3nnnc23)CCN(C(C)=O)CC1] | 530.2 |

### Example 127: (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-methylpiperidin-1-yl)ethan-1-one

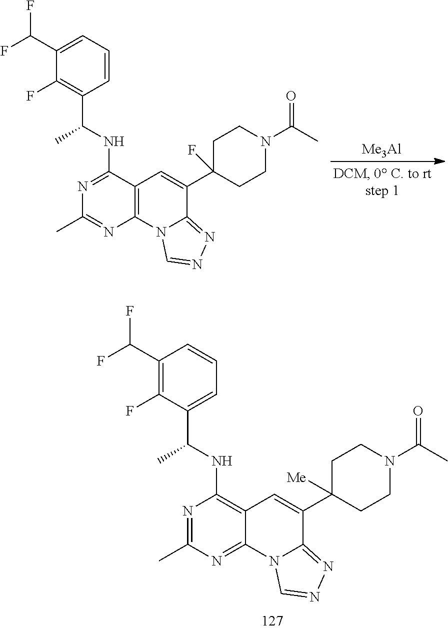 [SMILES: "Me3Al" ; CC(=O)N1CCC(F)(c2cc3c(N[C@H](C)c4cccc(C(F)F)c4F)nc(C)nc3n3cnnc23)CC1 ; CC(=O)N1CCC(C)(c2cc3c(N[C@H](C)c4cccc(C(F)F)c4F)nc(C)nc3n3cnnc23)CC1]

Step 1: To a solution of (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-fluoropiperidin-1-yl)ethan-1-one (30 mg, 0.058 mmol, 1 eq) in DCM (2 mL) was added Me₃Al (2M in hexane, 0.1 mL) by dropwise at 0° C. The resulting mixture was stirred at 25° C. for 2 h. The mixture was quenched with MeOH and filtrated. The filtrate was concentrated under reduced pressure and the residue was purified with prep-HPLC (Gemini 5 um C18 150*21.2 mm, mobile phase: ACN-H₂O (0.1% TFA); gradient: 5-95) and lyophilized to afford (R)-1-(4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-4-methylpiperidin-1-yl)ethan-1-one.

| | | Mass |
|---|---|---|
| Example | | m/z |
| # | Structure | (M + 1)⁺ |
| 127 | 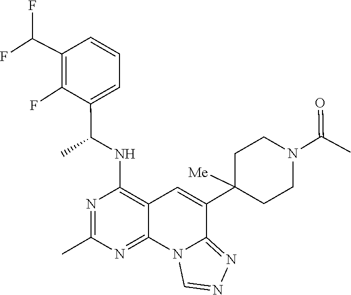 [SMILES: CC(=O)N1CCC(C)(c2cc3c(N[C@H](C)c4cccc(C(F)F)c4F)nc(C)nc3n3cnnc23)CC1] | 512.2 |
| 128 | 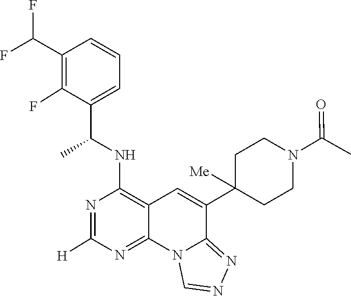 [SMILES: CC(=O)N1CCC(C)(c2cc3c(N[C@H](C)c4cccc(C(F)F)c4F)ncnc3n3cnnc23)CC1] | 498.1 |
| 129 | 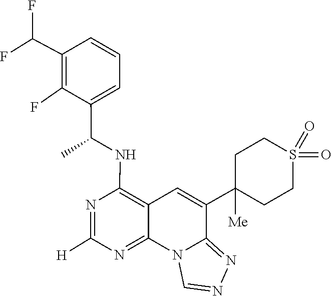 [SMILES: C[C@@H](Nc1ncnc2c1cc(C1(C)CCS(=O)(=O)CC1)c1nncn12)c1cccc(C(F)F)c1F] | 505.2 |
| 130 | 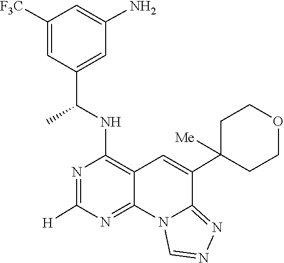 [SMILES: C[C@@H](Nc1ncnc2c1cc(C1(C)CCOCC1)c1nncn12)c1cc(N)cc(C(F)(F)F)c1] | 472.3 |
| 131 | 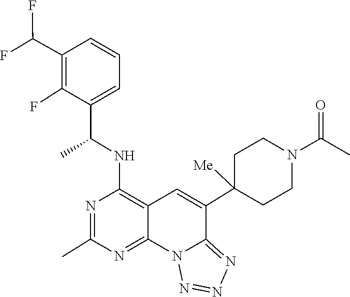 [SMILES: CC(=O)N1CCC(C)(c2cc3c(N[C@H](C)c4cccc(C(F)F)c4F)nc(C)nc3n3nnnc23)CC1] | 513.3 |
| 132 | 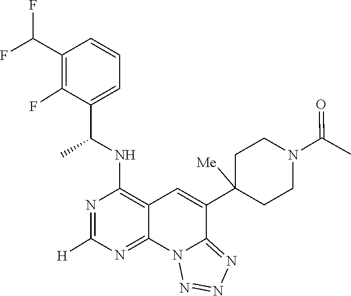 [SMILES: CC(=O)N1CCC(C)(c2cc3c(N[C@H](C)c4cccc(C(F)F)c4F)ncnc3n3nnnc23)CC1] | 499.3 |

### Example 133: (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)-4-(methoxy-d3)piperidin-1-yl)ethan-1-one

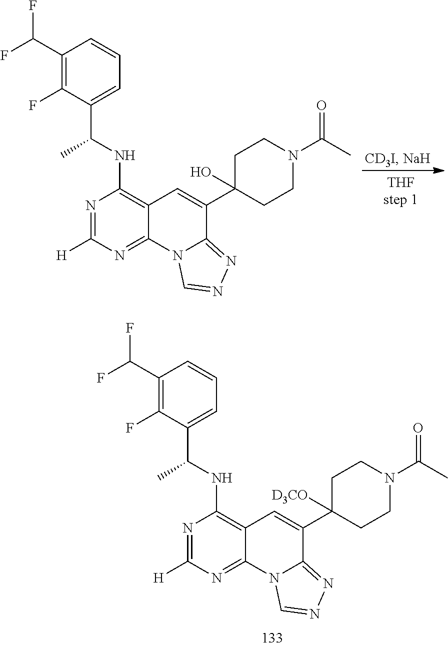

Step 1: To a solution of (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)-4-hydroxypiperidin-1-yl)ethan-1-one (150 mg, 0.3 mmol) in THF (2 mL) was added sodium hydride (10.8 mg, 0.45 mmol) portionwise at 0° C. The reaction mixture was stirred at 0° C. for 0.5 h and then CD₃I (42.6 mg, 0.3 mmol, 1.0 eq) was added. The reaction mixture was stirred at 50° C. for 2 hours. The reaction was quenched with water (5 mL) and extracted with EtOAc (10 mL×3). The combined organic layers were washed with brine (10 mL), dried over sodium sulfate, filtered, and concentrated. The residue was purified with prep-TLC and prep-HPLC (column: Gemini 5 um C18 150*21.2 mm; mobile phase ACN-H₂O (0.05% TFA); gradient: 20-95) and lyophilized to afford final compound.

LC/MS: 517.2 (M+1)⁺.

### Example 134: (R)—N-(1-acetyl-4-(4-((1-(3-(difluoromethyl)-2-fluorophenyl)ethyl)amino)-[1,2,4]triazolo[4′,3′:1,6]pyrido[2,3-d]pyrimidin-6-yl)piperidin-4-yl)acetamide

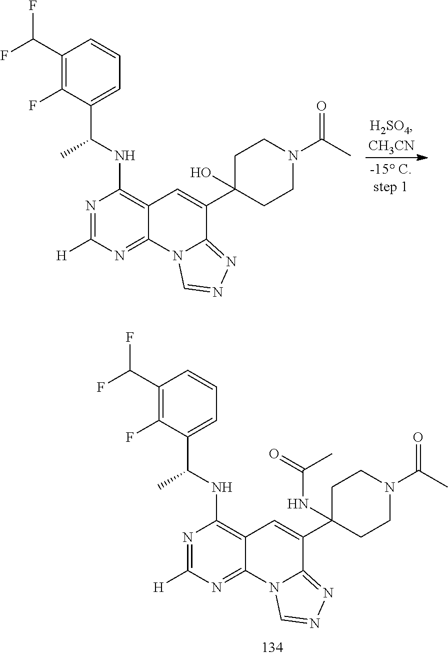

Step 1: To a solution of (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)-4-hydroxypiperidin-1-yl)ethan-1-one (100 mg, 0.2 mmol, 1 eq) in CH₃CN (1 mL) stirred under nitrogen at −15° C. was added H₂SO₄ (2 mL) dropwise. The reaction mixture was stirred at −15° C. for 8 hours. The mixture was quenched with NaHCO₃ (20 mL), followed by extraction with DCM (3×10 mL). The combined organic layers were washed with brine, dried, filtered and concentrated. The residue was purified with prep-HPLC (column: Shim-pack Velox C18, 3.0×50 mm, 2.7 um; mobile phase: ACN-H₂O (0.05% HCOOH); gradient: 5-95) and lyophilized to afford final compound.

LC/MS: 541.4 (M+1)⁺.

### Example 135: (R)-1-(4-(4-((1-(3-(difluoromethyl)-2-fluorophenyl)ethyl)amino)-9-(trifluoromethyl)-[1,2,4]triazolo[4′,3′:1,6]pyrido[2,3-d]pyrimidin-6-yl)-4-hydroxypiperidin-1-yl)ethan-1-one

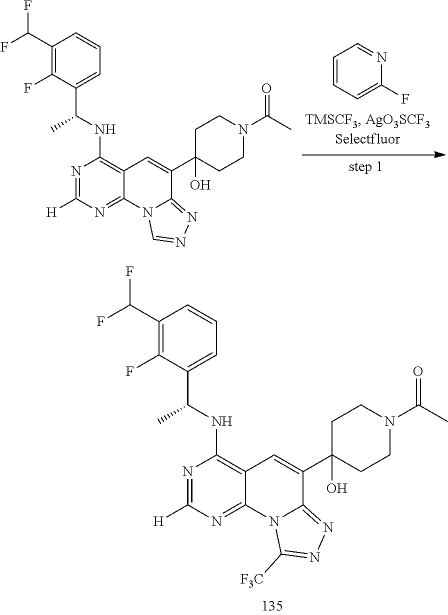 [SMILES: CC(=O)N1CCC(O)(c2cc3c(N[C@H](C)c4cccc(C(F)F)c4F)ncnc3n3cnnc23)CC1 ; Fc1ccccn1 ; CC(=O)N1CCC(O)(c2cc3c(N[C@H](C)c4cccc(C(F)F)c4F)ncnc3n3c(C(F)(F)F)nnc23)CC1]

Step 1: To a reaction tube that was equipped with a stirring bar, AgOTf (77 mg, 0.3 mmol, 3 eq), selectfluor (53 mg, 0.15 mmol, 1.5 eq), KF (23 mg, 0.4 mmol, 4 eq), (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)-4-hydroxypiperidin-1-yl)ethan-1-one (50 mg, 0.1 mmol, 1 eq) were added successively in a nitrogen-filled glovebox. Then ethyl acetate (2 mL), 2-fluoropyridine (29 mg, 0.3 mmol, 3.0 eq) and CF₃TMS (43 mg, 0.3 mmol, 3.0 eq) were added successively under N₂ atmosphere. The reaction mixture was stirred at 25° C. for 12 hours. The mixture was filtered through a plug of silica. The filtrate was concentrated and the residue was purified with prep-HPLC (column: Shim-pack Velox C18, 3.0×50 mm, 2.7 um; mobile phase: ACN-H₂O (0.05% NH₃·H₂O); gradient: 5-95) and lyophilized to afford final compound LC/MS: 568.3 (M+1)⁺.

### Example 136: (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)piperidin-1-yl)ethan-1-one

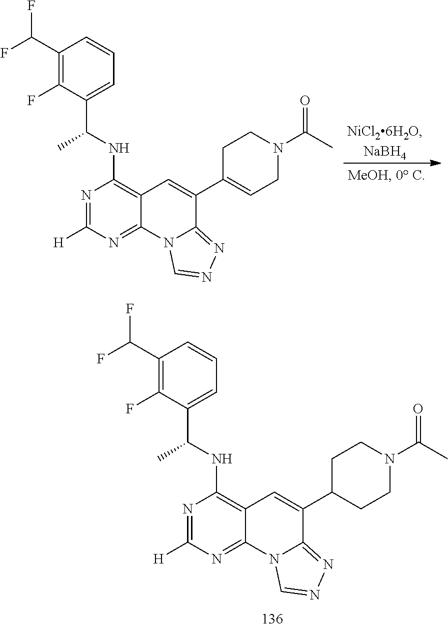 [SMILES: CC(=O)N1CC=C(c2cc3c(N[C@H](C)c4cccc(C(F)F)c4F)ncnc3n3cnnc23)CC1 ; "NiCl2·6H2O, NaBH4" ; CC(=O)N1CCC(c2cc3c(N[C@H](C)c4cccc(C(F)F)c4F)ncnc3n3cnnc23)CC1]

Step 1: To a solution of (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)-3,6-dihydropyridin-1(2H)-yl)ethan-1-one (40 mg, 83.1 μmol, 1.0 eq.) and NiCl₂·6H₂O (2.0 mg, 8.31 μmol, 0.1 eq.) in MeOH (2 mL) was added NaBH₄ (9.4 mg, 249.2 μmol, 3.0 eq.) in portions. The reaction was stirred for 0.5 h at rt to give a black brown solution. The reaction was diluted with H₂O and extracted with EA (10 mL×3). The combined organic layers were washed with brine, dried over Na₂SO₄, filtered and concentrated to dryness. The residue was purified by flash column (C18 column, eluting a gradient of ACN/H₂O containing 0.5% HCOOH, at flow rate of 30 mL/min). The collected flows were lyophilized to afford (R)-1-(4-(4-((1-(3-(difluoromethyl)-2-fluorophenyl)ethyl)amino)-[1,2,4]triazolo[4′,3′:1,6]pyrido[2,3-d]pyrimidin-6-yl)piperidin-1-yl)ethan-1-one.

| | | Mass |
|---|---|---|
| Example | | m/z |
| # | Structure | (M + 1)⁺ |
| 136 | 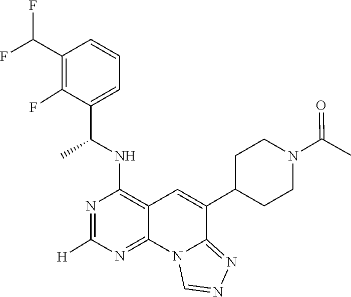 [SMILES: CC(=O)N1CCC(c2cc3c(N[C@H](C)c4cccc(C(F)F)c4F)ncnc3n3cnnc23)CC1] | 484.2 |
| 137 | 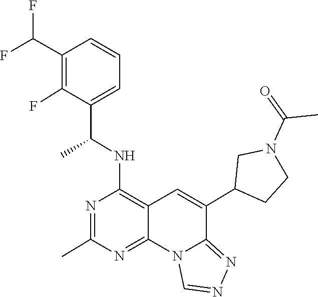 [SMILES: CC(=O)N1CCC(c2cc3c(N[C@H](C)c4cccc(C(F)F)c4F)nc(C)nc3n3cnnc23)C1] | 484.5 |
| 138 | 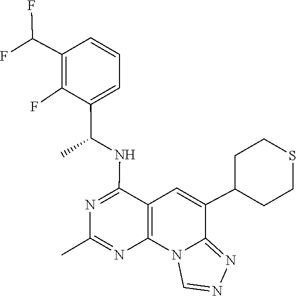 [SMILES: Cc1nc(N[C@H](C)c2cccc(C(F)F)c2F)c2cc(C3CCSCC3)c3nncn3c2n1] | 473.2 |
| 139 | 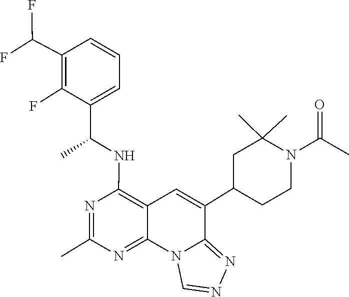 [SMILES: CC(=O)N1CCC(c2cc3c(N[C@H](C)c4cccc(C(F)F)c4F)nc(C)nc3n3cnnc23)CC1(C)C] | 526.2 |
| 140 | 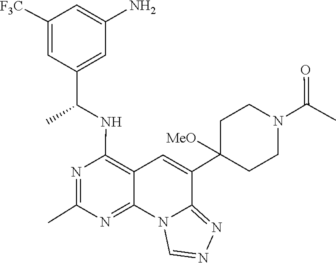 [SMILES: COC1(c2cc3c(N[C@H](C)c4cc(N)cc(C(F)(F)F)c4)nc(C)nc3n3cnnc23)CCN(C(C)=O)CC1] | 512.2 |
| 141 | 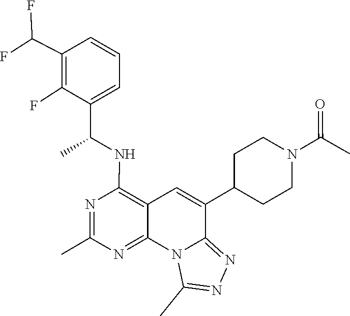 [SMILES: CC(=O)N1CCC(c2cc3c(N[C@H](C)c4cccc(C(F)F)c4F)nc(C)nc3n3c(C)nnc23)CC1] | 512.2 |
| 142 | 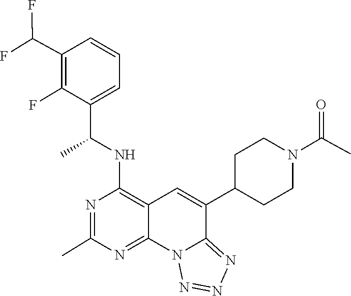 [SMILES: CC(=O)N1CCC(c2cc3c(N[C@H](C)c4cccc(C(F)F)c4F)nc(C)nc3n3nnnc23)CC1] | 499.3 |
| 143 | 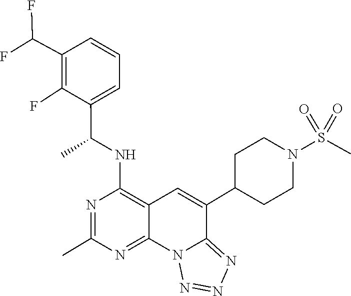 [SMILES: Cc1nc(N[C@H](C)c2cccc(C(F)F)c2F)c2cc(C3CCN(S(C)(=O)=O)CC3)c3nnnn3c2n1] | 535.3 |
| 144 | 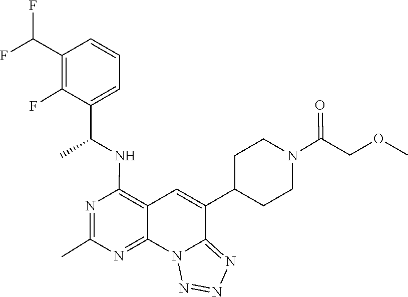 [SMILES: COCC(=O)N1CCC(c2cc3c(N[C@H](C)c4cccc(C(F)F)c4F)nc(C)nc3n3nnnc23)CC1] | 529.6 |
| 145 | 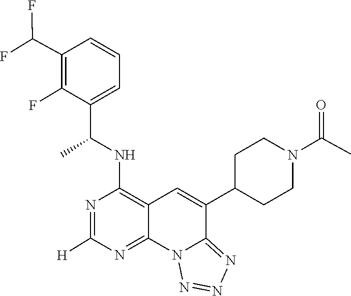 [SMILES: CC(=O)N1CCC(c2cc3c(N[C@H](C)c4cccc(C(F)F)c4F)ncnc3n3nnnc23)CC1] | 485.3 |
| 146 | 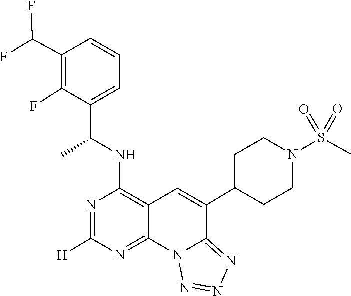 [SMILES: C[C@@H](Nc1ncnc2c1cc(C1CCN(S(C)(=O)=O)CC1)c1nnnn12)c1cccc(C(F)F)c1F] | 521.3 |
| 147 | 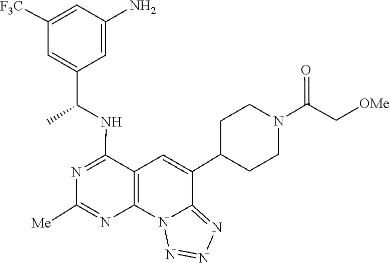 [SMILES: COCC(=O)N1CCC(c2cc3c(N[C@H](C)c4cc(N)cc(C(F)(F)F)c4)nc(C)nc3n3nnnc23)CC1] | 544.3 |
| 148 | 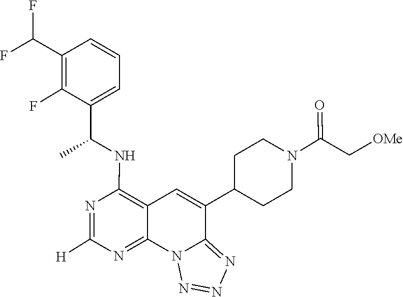 [SMILES: COCC(=O)N1CCC(c2cc3c(N[C@H](C)c4cccc(C(F)F)c4F)ncnc3n3nnnc23)CC1] | 515.2 |

### Example 149: 4-(4-(((R)-1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydro-2H-thiopyran 1-oxide

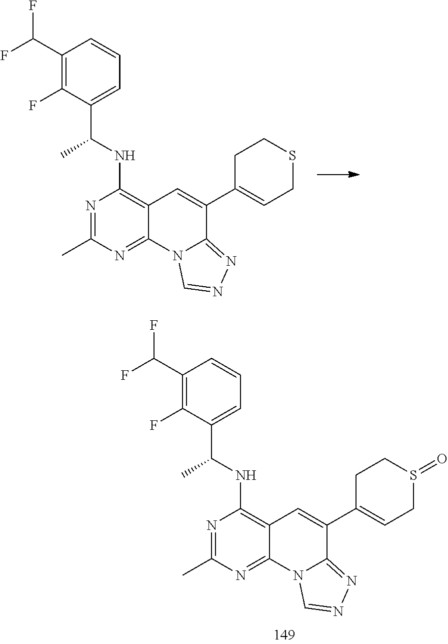 [SMILES: Cc1nc(N[C@H](C)c2cccc(C(F)F)c2F)c2cc(C3=CCSCC3)c3nncn3c2n1 ; Cc1nc(N[C@H](C)c2cccc(C(F)F)c2F)c2cc(C3=CCS(=O)CC3)c3nncn3c2n1]

Step 1: To a solution of (R)—N-(1-(3-(difluoromethyl)-2-fluorophenyl)ethyl)-6-(3,6-dihydro-2H-thiopyran-4-yl)-2-methyl-[1,2,4]triazolo[4′,3′:1,6]pyrido[2,3-d]pyrimidin-4-amine (80 mg, 170.0 μmol, 1 eq.) in THF (2 mL) and H₂O (0.5 mL) was added Oxone (52.3 mg, 85.0 μmol, 0.5 eq.) at 0˜5° C. The reaction was stirred for 0.5 h at rt to give an off-white suspension. The residue was quenched with Na₂SO₃ aq. and extracted with EA (10 mL×3). The combined organic layers were washed with brine, dried over Na₂SO₄, filtered and concentrated to dryness. The residue was purified by flash column (C18 column, eluting a gradient of ACN/H₂O containing 0.5% HCOOH) to afford 4-(4-(((R)-1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydro-2H-thiopyran 1-oxide.

| | | Mass |
|---|---|---|
| Example | | m/z |
| # | Structure | (M + 1)⁺ |
| 149 | 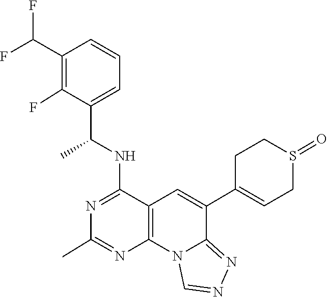 [SMILES: Cc1nc(N[C@H](C)c2cccc(C(F)F)c2F)c2cc(C3=CCS(=O)CC3)c3nncn3c2n1] | 487.14 |
| 150 | 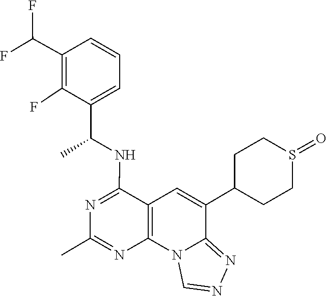 [SMILES: Cc1nc(N[C@H](C)c2cccc(C(F)F)c2F)c2cc(C3CCS(=O)CC3)c3nncn3c2n1] | 489.2 |

### Example 151: (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydro-2H-thiopyran 1,1-dioxide

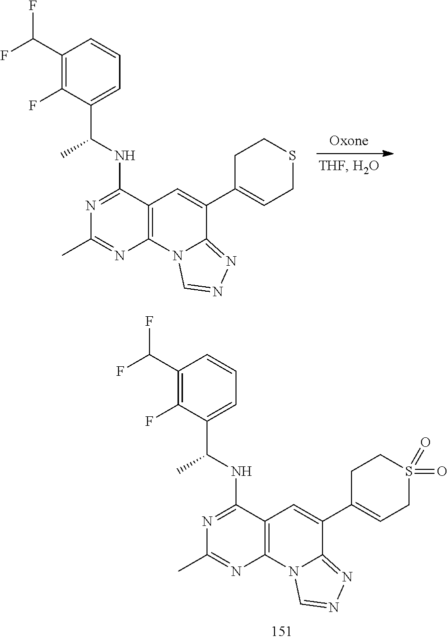 [SMILES: Cc1nc(N[C@H](C)c2cccc(C(F)F)c2F)c2cc(C3=CCSCC3)c3nncn3c2n1 ; Cc1nc(N[C@H](C)c2cccc(C(F)F)c2F)c2cc(C3=CCS(=O)(=O)CC3)c3nncn3c2n1]

Step 1: To a solution of (R)—N-(1-(3-(difluoromethyl)-2-fluorophenyl)ethyl)-6-(3,6-dihydro-2H-thiopyran-4-yl)-2-methyl-[1,2,4]triazolo[4′,3′:1,6]pyrido[2,3-d]pyrimidin-4-amine (10 mg, 21.3 μmol, 1.0 eq.) in THF (2 mL) and H₂O (0.5 mL) was added Oxone (13.1 mg, 21.3 μmol, 1.0 eq.) at 0˜5° C. The reaction was stirred at rt for 0.5 h to give an off-white suspension. The residue was quenched with Na₂SO₃ aq. and extracted with EA (10 mL×3). The combined organic layers were washed with brine, dried over Na₂SO₄, filtered and concentrated to dryness. The residue was purified by flash column (C18 column, eluting a gradient of ACN/H₂O containing 0.5% HCOOH) to afford (R)-4-(4-((1-(3-(difluoromethyl)-2-fluorophenyl)ethyl)amino)-2-methyl-[1,2,4]triazolo[4′,3′:1,6]pyrido[2,3-d]pyrimidin-6-yl)-3,6-dihydro-2H-thiopyran 1,1-dioxide.

| | | Mass |
|---|---|---|
| Example | | m/z |
| # | Structure | (M + 1)⁺ |
| 151 | 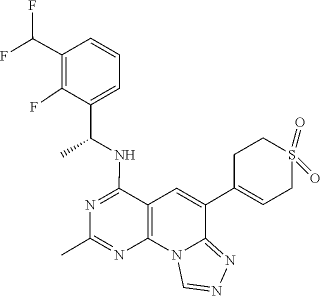 [SMILES: Cc1nc(N[C@H](C)c2cccc(C(F)F)c2F)c2cc(C3=CCS(=O)(=O)CC3)c3nncn3c2n1] | 503.1 |
| 152 | 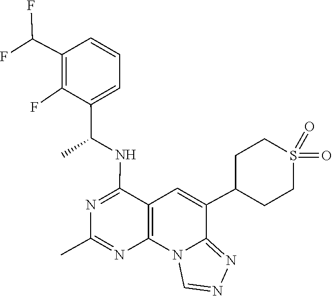 [SMILES: Cc1nc(N[C@H](C)c2cccc(C(F)F)c2F)c2cc(C3CCS(=O)(=O)CC3)c3nncn3c2n1] | 505.2 |

### Example 153: (R)—N-(1-(3-amino-5-(trifluoromethyl)phenyl)ethyl)-4-morpholino-[1,2,4]triazolo[1′,5′:1,6]pyrido[2,3-d]pyrimidin-6-amine

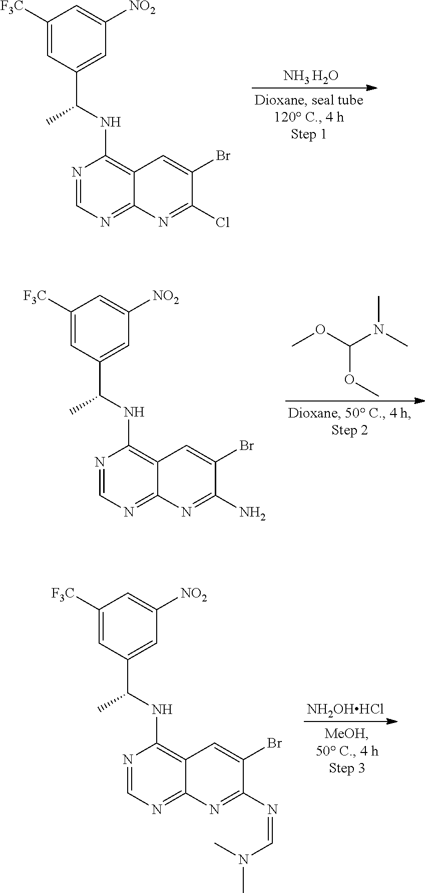

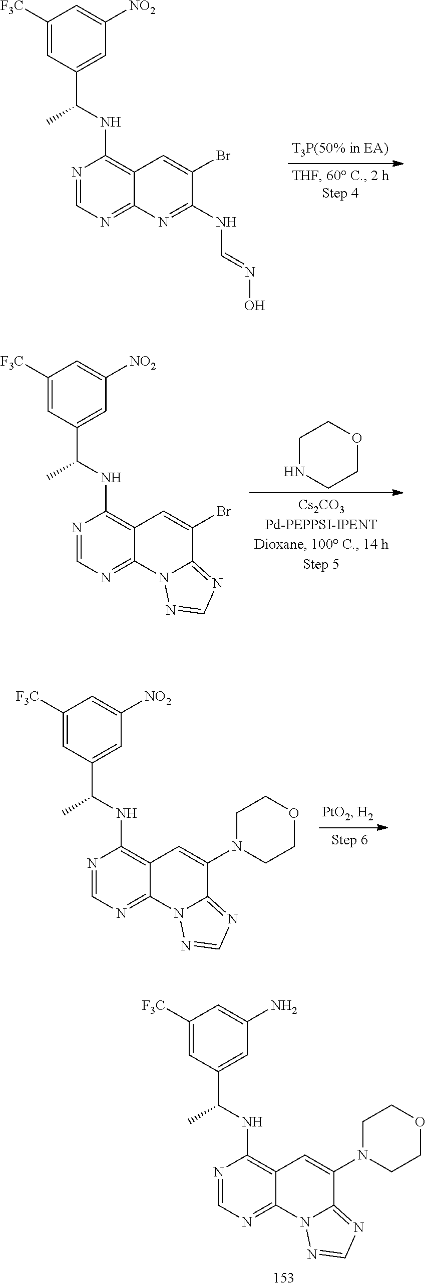 [SMILES: C[C@@H](Nc1ncnc2nc(N/C=N/O)c(Br)cc12)c1cc([N+](=O)[O-])cc(C(F)(F)F)c1 ; C[C@@H](Nc1ncnc2c1cc(Br)c1ncnn12)c1cc([N+](=O)[O-])cc(C(F)(F)F)c1 ; C1COCCN1 ; C[C@@H](Nc1ncnc2c1cc(N1CCOCC1)c1ncnn12)c1cc([N+](=O)[O-])cc(C(F)(F)F)c1 ; C[C@@H](Nc1ncnc2c1cc(N1CCOCC1)c1ncnn12)c1cc(N)cc(C(F)(F)F)c1]

Step 1: A solution of (R)-6-bromo-7-chloro-N-(1-(3-nitro-5-(trifluoromethyl)phenyl)ethyl)pyrido[2,3-d]pyrimidin-4-amine (3.6 g, 8.3 mmol, 1 eq. synthesized following procedures for example 74) in 1,4-dioxane (40 mL) and NH₃·H₂O (30 mL) in sealed tube was stirred at 120° C. for 4 hours. The solution was concentrated under reduced pressure and purified by flash column chromatography (DCM:MeOH=95:5) to afford (R)-6-bromo-N4-(1-(3-nitro-5-(trifluoromethyl)phenyl)ethyl)pyrido[2,3-d]pyrimidine-4,7-diamine as a light brown solid.

LC-MS [M+H]⁺: 457.0; 459.1

Step 2: A solution of (R)-6-bromo-N4-(1-(3-nitro-5-(trifluoromethyl)phenyl)ethyl)pyrido[2,3-d]pyrimidine-4,7-diamine (3 g, 9.1 mmol, 1 eq) and dimethylformamide dimethyl acetal (5.78 g, 48.5 mmol, 5 eq) in 1,4-dioxane (40 mL) was stirred at 50° C. for 4 hours. The solution was concentrated under reduced pressure to afford crude (R,Z)-N′-(6-bromo-4-((1-(3-nitro-5-(trifluoromethyl)phenyl)ethyl)amino)pyrido[2,3-d]pyrimidin-7-yl)-N,N-dimethylformimidamide as a black solid which was used for next step directly.

LC-MS [M+H]+: 512.2; 514.2

Step 3: To a solution of (R,Z)-N′-(6-bromo-4-((1-(3-(difluoromethyl)-2-fluorophenyl)ethyl)amino)-2-methylpyrido[2,3-d]pyrimidin-7-yl)-N,N-dimethylformimidamide (3.00 g, 8.6 mmol, 1 eq) in MeOH (50 mL) was added hydroxylamine hydrochloride (2.99 g, 4.3 mmol, 5 eq). The mixture was stirred at 50° C. for 4 hours. The mixture was concentrated under reduced pressure. The residue was dissolved in THF and filtered, and the filtrate was concentrated under reduced pressure to afford crude (R,E)-N-(6-bromo-4-((1-(3-nitro-5-(trifluoromethyl)phenyl)ethyl)amino)pyrido[2,3-d]pyrimidin-7-yl)-N′-hydroxyformimidamide which was used for next step directly.

LC-MS [M+H]+: 499.9; 501.9

Step 4: To a solution of (R,E)-N-(6-bromo-4-((1-(3-nitro-5-(trifluoromethyl)phenyl)ethyl)amino)pyrido[2,3-d]pyrimidin-7-yl)-N′-hydroxyformimidamide (3.00 g, 6.6 mmol, 1 eq) in THF (50 mL) was added propylphosphonic anhydride (50% in EtOAc, 4.2 g, 13.2 mmol, 2 eq). The mixture was stirred at 60° C. for 2 hours. The reaction mixture was quenched with aqueous NaHCO₃ to adjust pH=8 and extracted with ethyl acetate (3×200 mL). The combined organic phase was washed with brine (500 mL), dried over anhydrous sodium sulfate, filtrated and concentrated. The residue was purified by flash column chromatography (DCM:MeOH=97:3) to afford (R)-4-bromo-N-(1-(3-nitro-5-(trifluoromethyl)phenyl)ethyl)-[1,2,4]triazolo[1′,5′:1,6]pyrido[2,3-d]pyrimidin-6-amine as a light brown solid.

LC-MS [M+H]+: 481.9; 483.9

¹H NMR (400 MHz, DMSO) δ 13.90 (s, 1H), 9.38 (d, J=7.4 Hz, 1H), 8.75 (s, 1H), 8.60 (s, 1H), 8.51 (s, 1H), 8.40 (s, 1H), 8.32 (s, 1H), 5.86-5.69 (m, 1H), 1.65 (d, J=7.0 Hz, 3H).

Step 5: To a solution of 7-bromo-N-[(1R)-1-{3-[(difluoromethyl)-$l\hat{ }{2}-fluoranyl]-5-nitrophenyl}ethyl]-2,3,5,11,13-pentaazatricyclo[7.4.0.0\hat{ }{2,6}]trideca-1(13),3,5,7,9,11-hexaen-10-amine [100 mg, 0.20 mmol, 1.0 eq], morpholine [36.1 mg, 0.41 mmol, 2.05 eq], Cs₂CO₃ [203.5 mg, 0.62 mmol, 3.1 eq] and Pd-PEPPSI-IPENT [7.9 mg, 0.01 mmol, 0.05 eq] in 1,4-dioxane stirred under nitrogen at 100° C. for 14 hours. The reaction was quenched with water (10 mL) and the mixture was extracted with ethyl acetate (3×10 mL). The separated organic layer was washed with brine, dried over anhydrous Na₂SO₄ and filtered. The filtrate was concentrated and the residue was purified by flash column chromatography (DCM/MeOH=4%) to afford product as a yellow solid.

LC-MS [M+H]⁺ 489.4

Step 6: To a solution of 7-(morpholin-4-yl)-N-[(1R)-1-[3-nitro-5-(trifluoromethyl)phenyl]ethyl]-2,3,5,11,13-pentaazatricyclo[7.4.0.0\hat{ }{2,6}]trideca-1(13),3,5,7,9,11-hexaen-10-amine [63.4 mg, 0.13 mmol] and PtO₂ [2.9 mg, 0.01 mmol] in MeOH stirred under H₂ at 25° C. The reaction mixture was stirred at 25° C. for 2 hours. The reaction was filtered. The filtrate was concentrated and the residue was purified by Genal-Prep-HPLC to afford product as a white solid.

LC-MS [M+H]⁺=459.4

¹H NMR (400 MHz, DMSO) δ 8.59 (s, 1H), 8.30 (s, 1H), 7.52 (s, 1H), 6.82-6.79 (m, 3H), 6.71 (s, 1H), 5.58 (s, 2H), 5.51 (q, J=7.7 Hz, 1H), 3.75 (t, J=4.2 Hz, 4H), 3.17 (s, 4H), 1.55 (d, J=7.0 Hz, 3H).

| | | Mass |
|---|---|---|
| Example | | m/z |
| # | Structure | (M + 1)⁺ |
| 153 | 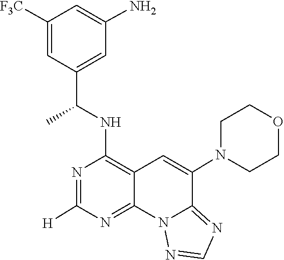 [SMILES: C[C@@H](Nc1ncnc2c1cc(N1CCOCC1)c1ncnn12)c1cc(N)cc(C(F)(F)F)c1] | 459.4 |
| 154 | 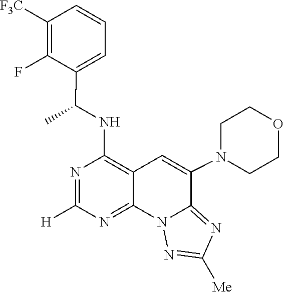 [SMILES: Cc1nc2c(N3CCOCC3)cc3c(N[C@H](C)c4cccc(C(F)(F)F)c4F)ncnc3n2n1] | 458.4 |
| 155 | 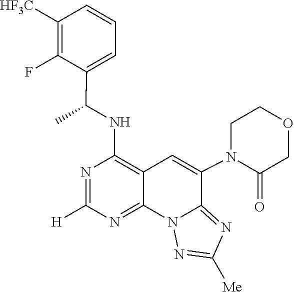 | 472.5 |
| 156 | 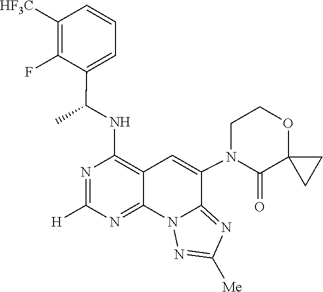 | 498.1 |
| 157 | 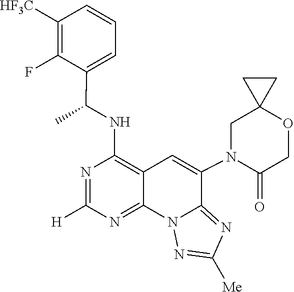 | 498.2 |
| 158 | 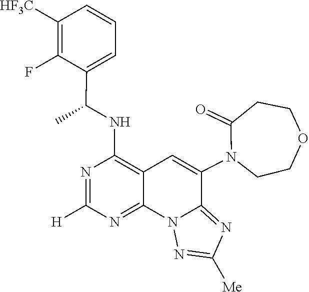 | 485.8 |
| 159 | 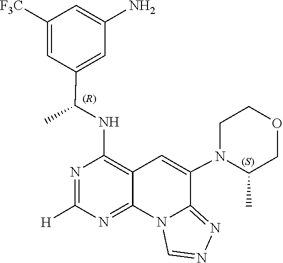 | 473.4 |
| 160 | 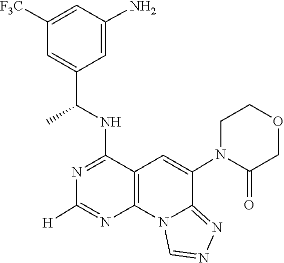 [SMILES: C[C@@H](Nc1ncnc2c1cc(N1CCOCC1=O)c1nncn12)c1cc(N)cc(C(F)(F)F)c1] | 502.4 |
| 161 | 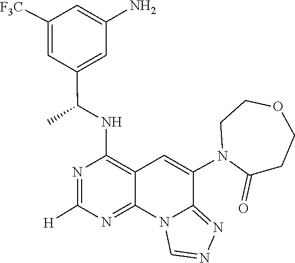 [SMILES: C[C@@H](Nc1ncnc2c1cc(N1CCOCCC1=O)c1nncn12)c1cc(N)cc(C(F)(F)F)c1] | 501.1 |
| 162 | 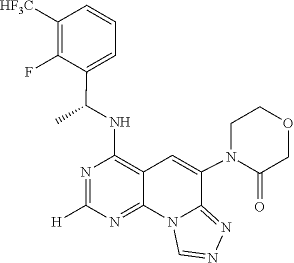 | 458.2 |
| 163 | 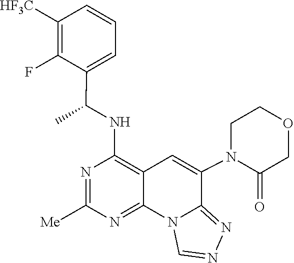 | 472.3 |
| 164 | 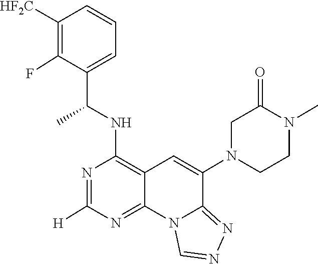 | 471.3 |
| 165 | 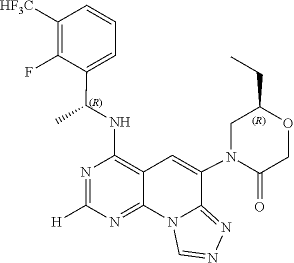 | 486.3 |
| 166 | 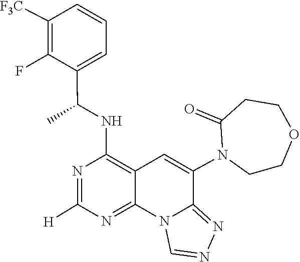 [SMILES: C[C@@H](Nc1ncnc2c1cc(N1CCOCCC1=O)c1nncn12)c1cccc(C(F)(F)F)c1F] | 472.4 |
| 167 | 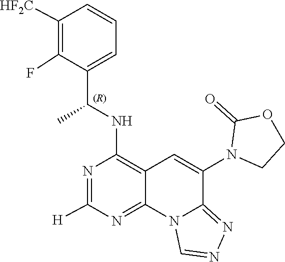 | 444.1 |
| 168 | 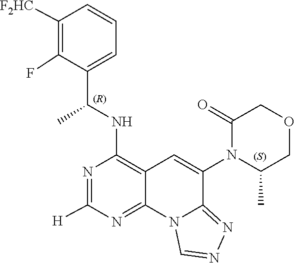 | 472.2 |

### Example 169: (R)—N-(1-(3-(difluoromethyl)-2-fluorophenyl)ethyl)-2-methyl-4-(1-(trifluoromethyl)cyclopropyl)-[1,2,4]triazolo[1′,5′:1,6]pyrido[2,3-d]pyrimidin-6-amine

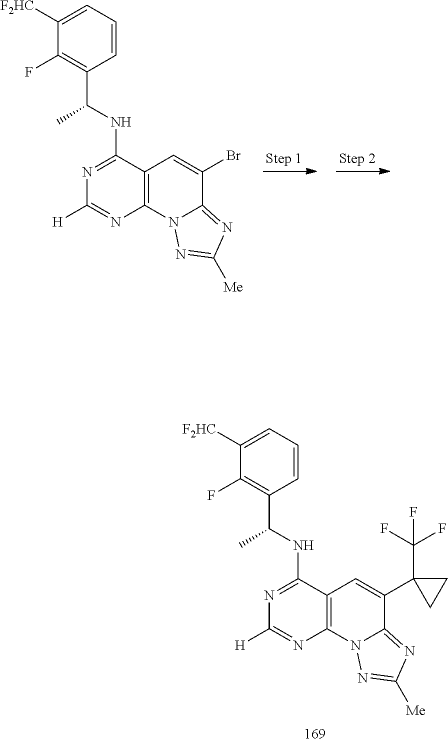 [SMILES: Cc1nc2c(Br)cc3c(N[C@H](C)c4cccc(C(F)F)c4F)ncnc3n2n1 ; Cc1nc2c(C3(C(F)(F)F)CC3)cc3c(N[C@H](C)c4cccc(C(F)F)c4F)ncnc3n2n1]

Step 1: To a mixture of 4-bromo-6-{[(1R)-1-[3-(difluoromethyl)-2-fluorophenyl]ethyl]amino}-2-methyl[1,2,4]triazolo[5′,1′:6,1]pyrido[2,3-d]pyrimidine (100.0 mg, 0.22 mmol) and 4,4,6-trimethyl-2-(3,3,3-trifluoroprop-1-en-2-yl)-1,3,2-dioxaborinane (59.0 mg, 0.27 mmol) in dioxane (1 mL) and water (0.1 mL) was added Potassium carbonate (92.0 mg, 0.666 mmol) and 1,1′-bis(diphenylphosphino)ferrocene-palladium(II)dichloride dichloromethane complex (18.0 mg, 0.02 mmol). The reaction mixture was stirred at 90° C. for overnight leading to a dark red suspension. LCMS showed desired mass was observed. The reaction mixture was cooled to rt and filtered through celite. The filtration was concentrated and the residue was purified by silica column chromatography (DCM/MeOH: from 0 to 6%) to provide 6-{[(1R)-1-[3-(difluoromethyl)-2-fluorophenyl]ethyl]amino}-2-methyl-4-(3,3,3-trifluoroprop-1-en-2-yl)[1,2,4]triazolo[5′,1′:6,1]pyrido[2,3-d]pyrimidine as a yellow oil.

LCMS m/z (M+1)⁺: 467.2.

Step 2: To a solution of 6-{[(1R)-1-[3-(difluoromethyl)-2-fluorophenyl]ethyl]amino}-2-methyl-4-(3,3,3-trifluoroprop-1-en-2-yl)[1,2,4]triazolo[5′,1′:6,1]pyrido[2,3-d]pyrimidine (93.0 mg, 0.20 mmol) and Methyl(diphenyl)sulfoniumfluoroborate (149.1 mg, 0.52 mmol) in THF (2 mL) was added Sodium bis(trimethylsilyl)amide (0.80 mL, 0.80 mmol) at 0° C. The reaction mixture was stirred at the same temperature for 10 min and rt for overnight leading to a dark red suspension. LCMS showed desired mass was observed. The mixture was quenched with MeOH and water, extracted with EA, dried over sodium sulfonate and concentrated under reduced pressure. The residue was purified by Pre-TLC (DCM:MeOH=20:1) to provide 6-{[(1R)-1-[3-(difluoromethyl)-2-fluorophenyl]ethyl]amino}-2-methyl-4-[(trifluoromethyl)cyclopropyl][1,2,4]triazolo[5′,1′:6,1]pyrido[2,3-d]pyrimidine as a white solid.

¹H NMR (400 MHz, Methanol-d₄) δ 8.75 (s, 1H), 8.56 (s, 1H), 7.62 (t, J=7.5 Hz, 1H), 7.50 (t, J=7.2 Hz, 1H), 7.26 (t, J=7.8 Hz, 1H), 7.02 (t, J=54.8 Hz, 1H), 5.89 (q, J=7.0 Hz, 1H), 2.66 (s, 3H), 1.72 (d, J=7.1 Hz, 3H), 1.66-1.58 (m, 2H), 1.40-1.33 (m, 2H).

### Example 170: (R)-1-(6-((1-(3-(difluoromethyl)-2-fluorophenyl)ethyl)amino)-2-methyl-[1,2,4]triazolo[1′,5′:1,6]pyrido[2,3-d]pyrimidin-4-yl)cyclopropane-1-carbonitrile

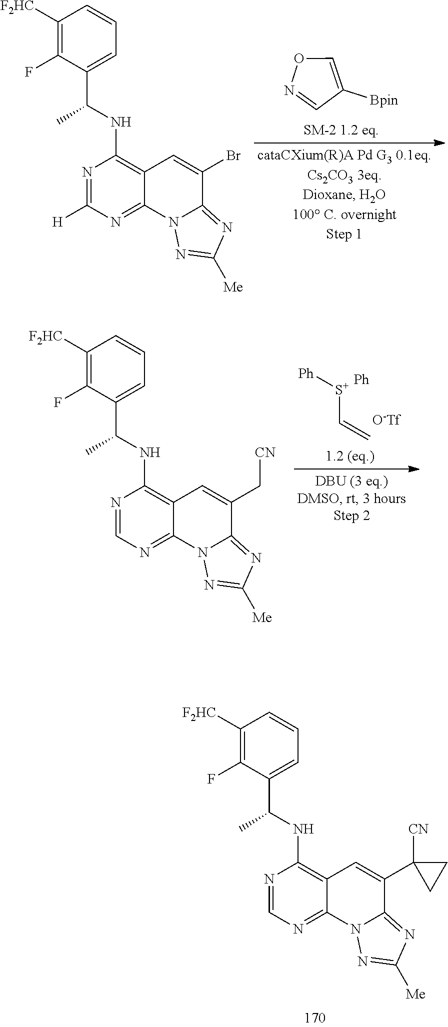

Step 1: To a suspension of (R)-4-bromo-N-(1-(3-(difluoromethyl)-2-fluorophenyl)ethyl)-2-methyl-[1,2,4]triazolo[1′,5′:1,6]pyrido[2,3-d]pyrimidin-6-amine [500.0 mg, 1.1 mmol, 1.0 eq], 4-(4,4,5,5-tetramethyl-1,3,2-dioxaborolan-2-yl)isoxazole [322.0 mg, 1.65 mmol, 1.2 eq] and Cs₂CO₃ [1070.0 mg, 3.3 mmol, 3.0 eq] in dioxane [10 mL]/H₂O (2 ml) was added cataCXium(R)A Pd G3 [80.0 mg, 0.1 eq] under N₂. The reaction mixture was stirred at 100° C. for 16 hours, then heated at 120° C. for 6 hours. The mixture was filtered. The filtrate was extracted with EA (30 ml) and washed with brine (20 ml), dried by Na₂SO₄, filtered and concentrated under reduced pressure and the residue was purified with flash column chromatography (mobile phase: MeOH/CH₂Cl₂, volume gradient: 0-10%) to afford the desired product (R)-2-(6-((1-(3-(difluoromethyl)-2-fluorophenyl)ethyl)amino)-2-methyl-[1,2,4]triazolo[1′,5′:1,6]pyrido[2,3-d]pyrimidin-4-yl)acetonitrile as a brown solid.

LCMS m/z (M+1)⁺: 412.0

Step 2: To a solution of (R)-2-(6-((1-(3-(difluoromethyl)-2-fluorophenyl)ethyl)amino)-2-methyl-[1,2,4]triazolo[1′,5′:1,6]pyrido[2,3-d]pyrimidin-4-yl)acetonitrile [100.0 mg, 0.24 mmol, 1.0 eq] in DMSO [5 mL] was added DBU [0.11 ml, 3.0 eq] and diphenyl(vinyl)sulfonium trifluoromethanesulfonate (108.0 mg, 1.2 eq). The reaction mixture was stirred at rt for 3 hours. Water (20 ml) was added. The solution was extracted with EA (30 ml) and washed with brine (20 ml), dried by Na₂SO₄, filtered and concentrated under reduced pressure and the residue was purified with reversed column chromatography (mobile phase: MeOH/0.1% NH₄HCO₃ solution, volume gradient: 40-80%), then purified with Prep-HPLC to afford the product (R)-1-(6-((1-(3-(difluoromethyl)-2-fluorophenyl)ethyl)amino)-2-methyl-[1,2,4]triazolo[1′,5′:1,6]pyrido[2,3-d]pyrimidin-4-yl)cyclopropane-1-carbonitrile as a brown solid.

LCMS m/z (M+1)⁺: 437.8

The following examples were synthesized using the above methods except example 174, which used the same method for example 159.

| | | Mass |
|---|---|---|
| Example | | m/z |
| # | Structure | (M + 1)⁺ |
| 169 | 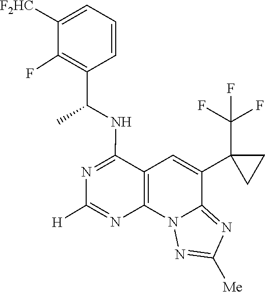 [SMILES: Cc1nc2c(C3(C(F)(F)F)CC3)cc3c(N[C@H](C)c4cccc(C(F)F)c4F)ncnc3n2n1] | 481.3 |
| 170 | 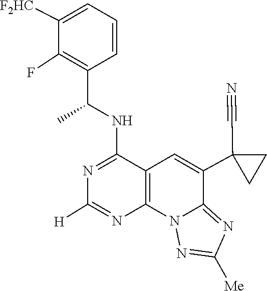 [SMILES: Cc1nc2c(C3(C#N)CC3)cc3c(N[C@H](C)c4cccc(C(F)F)c4F)ncnc3n2n1] | 437.8 |
| 171 | 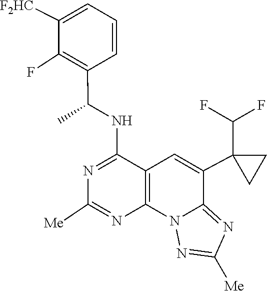 [SMILES: Cc1nc(N[C@H](C)c2cccc(C(F)F)c2F)c2cc(C3(C(F)F)CC3)c3nc(C)nn3c2n1] | 477.2 |
| 172 | 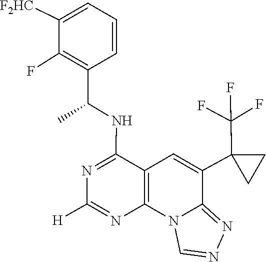 [SMILES: C[C@@H](Nc1ncnc2c1cc(C1(C(F)(F)F)CC1)c1nncn12)c1cccc(C(F)F)c1F] | 467.3 |
| 173 | 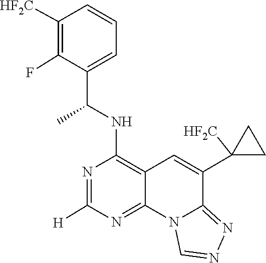 | 448.8 |
| 174 | 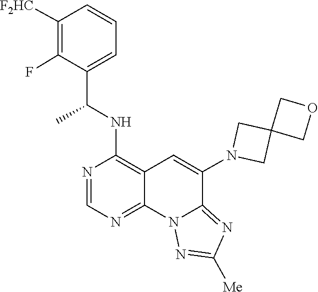 [SMILES: Cc1nc2c(N3CC4(COC4)C3)cc3c(N[C@H](C)c4cccc(C(F)F)c4F)ncnc3n2n1] | 469.8 |

### BIOLOGICAL EXAMPLES

### Example A. Protein-Protein Interaction (PPI) Assay

This Example illustrates that exemplary compounds of the present invention blockade the interaction of SOS1 to KRAS protein.

The ability of a compound of Formula (I) to block the interaction of SOS1 to KRAS was measured using a SOS1-KRAS binding assay kit (CAT #63ADK000CB16PEG, Cisbio). Utilizing HTRF (Homogeneous Time-resolved Fluorescence) technology, the assay enables simple and rapid characterization of compound and antibody blockers in a high throughput format. The interaction between Tag1-SOS1 and Tag2-KRAS-G12C is detected by using anti-Tag1-Terbium antibody (HTRF donor) and anti-Tag2-XL665 antibody (HTRF acceptor). When the donor and acceptor antibodies are brought into close proximity due to SOS1 and KRAS-G12C binding, excitation of the donor antibody triggers fluorescent resonance energy transfer (FRET) towards the acceptor antibody, which in turn emits specifically at 665 nm. This specific signal is directly proportional to the extent of KRAS-G12C/SOS1 interaction.

The assay was carried according to the manufacturer's instructions. Compounds at an initial concentration of 10 mM in DMSO were first serially diluted on a 384-well plate using assay buffer at a ratio of 1:3 for a total of 9 concentrations with each of a volume of 20 μL. Blank buffer was included as the negative control. The final compound solution was in the buffer solution with 1% DMSO. Then, in each well of a 384-well plate, a solution of 2 μL compound solution, 4 μL tagged SOS1 and KRAS (G12C) protein stocks, respectively, and 10 μM GTP was incubated for 15 min. 10 μL mixture of anti-Tag1X665 antibody and anti-Tag1-Terbium antibody was introduced to each individual well. After 2 hours incubation at room temperature, fluorescence readout was measured with a Pelkin Elmer multiple plate reader VICTOR Nivo.

Excitation was from over a range of 245-395 nm, and emission from the donor (emission 1) was detected at (657.5-672.5) nm and emission from the acceptor (emission 2) was detected at (606.5-623.5) nm. The HTRF ratio was calculated using the formula: (emission 1 @665 nm/emission 2 @620 nm)×10000. Thus, compounds blocking KRAS-G12C/SOS1 interaction will cause a reduction in HTRF signal ratio. The IC₅₀ values were then calculated with the Graph pad Prism software. Results are listed in Tables 1 and 2.

### Example B: Phospho-ERK (p-ERK) Assay

This Example illustrates that exemplary compounds of the present invention prevent KRas-mediated GTP nucleotide exchange mediated by SOS1 to inhibit KRas activity, thereby inhibiting the generation of the downstream effector pERK.

Hela cells (23,000/w) were seeded in a black clear flat bottom 96-well cell culture plate (Corning, #3904) and incubated at 37° C. overnight. At day 2, cells were dosed with compounds of Formula (I) with a 10 μM starting concentration and serially diluted 3× for a total of 9 concentrations. The cells were incubated for 1 hour with the compounds solubilized in DMSO at 37° C. Cells were immediately fixed by adding 100 μL of 8% formaldehyde to all wells in a fume hood and the plates were incubated for 1 hr at room temperature. The formaldehyde was discarded from the plates and washed 5 times and each time for 5 min with 200 μL of Triton Washing Solution (0.1% Triton in PBS) each time to permeabilize the cells. Cells were then blocked with 150 μL of Odyssey blocking buffer (LI-COR Biosciences #927-50010) at room temperature on a shaker. The blocking buffer was discarded and 50 μL of primary antibodies pERK (cell signaling Technology #4370; Rabbit, 1:500) and β-actin (AF5001; Mouse, 1:2000) diluted in Odyssey blocking buffer was added. The plates were incubated overnight at 4° C. on a shaker.

On day 3, the primary antibody solution was removed. Each plate was washed 5× times with 200 μL of 1×PBST (PBS+0.1% Tween 20) and incubated with 50 μL of secondary antibodies: Anti-Rabbit (LI-COR Biosciences #926-32211) and Anti-Mouse (LI-COR Biosciences #68070) at 1:1000 dilution in Odyssey blocking buffer at room temperature on a shaker for 1 hour (protected from light). The secondary antibody solution as removed and each plate was washed with PBST 5× times. Any liquid remaining was discarded and the plate was imaged using the Licor Odyssey machine according to the manufacturer's instruction, using a set focus length at 3.5 mm and both 800 nm and 700 nm filters. The 3-actin normalized scan values for each well were divided by the average of vehicle wells to get the % of p-ERK inhibition. The IC₅₀ values were then calculated with the Graph pad Prism software. Results are listed in Tables 1 and 2. In general, p-ERK IC₅₀ data are in line with the PPI IC₅₀ data.

TABLE 1
PPI IC₅₀ < 100 nM

| | PPI Assay | p-ERK Assay |
|---|---|---|
| Example # | IC₅₀ (nM) | IC₅₀ (nM) |
| 2 | <100 | >100 |
| 3 | <100 | <100 |
| 4 | <100 | >100 |
| 5 | <100 | >100 |
| 6 | <100 | <100 |
| 11 | <100 | >100 |
| 12 | <100 | N/A |
| 13 | <100 | <100 |
| 14 | <100 | N/A |
| 16 | <100 | N/A |
| 17 | <100 | >100 |
| 18 | <100 | >100 |
| 19 | <100 | >100 |
| 20 | <100 | >100 |
| 21 | <100 | >100 |
| 22 | <100 | <100 |
| 23 | <100 | >100 |
| 24 | <100 | >100 |
| 25 | <100 | N/A |
| 26 | <100 | >100 |
| 27 | <100 | <100 |
| 29 | <100 | <100 |
| 30 | <100 | N/A |
| 31 | <100 | >100 |
| 32 | <100 | N/A |
| 33 | <100 | >100 |
| 34 | <100 | N/A |
| 35 | <100 | >100 |
| 36 | <100 | >100 |
| 37 | <100 | >100 |
| 38 | <100 | >100 |
| 39 | <100 | >100 |
| 40 | <100 | N/A |
| 41 | <100 | N/A |
| 42 | <100 | >100 |
| 43 | <100 | N/A |
| 44 | <100 | N/A |
| 45 | <100 | N/A |
| 46 | <100 | N/A |
| 47 | <100 | >100 |
| 48 | <100 | N/A |
| 49 | <100 | N/A |
| 50 | <100 | >100 |
| 51 | <100 | >100 |
| 52 | <100 | N/A |
| 54 | <100 | N/A |
| 55 | <100 | >100 |
| 58 | <100 | <100 |
| 59 | <100 | N/A |
| 60 | <100 | >100 |
| 61 | <100 | N/A |
| 62 | <100 | >100 |
| 63 | <100 | N/A |
| 64 | <100 | N/A |
| 66 | <100 | >100 |
| 67 | <100 | >100 |
| 68 | <100 | >100 |
| 69 | <100 | >100 |
| 71 | <100 | >100 |
| 72 | <100 | NA |
| 74 | <100 | >100 |
| 76 | <100 | >100 |
| 78 | <100 | N/A |
| 79 | <100 | <100 |
| 85 | <100 | <100 |
| 86 | <100 | <100 |
| 88 | <100 | <100 |
| 90-P1 | <100 | N/A |
| 90-P2 | <100 | N/A |
| 91 | <100 | <100 |
| 92 | <100 | <100 |
| 93 | <100 | >100 |
| 94 | <100 | N/A |
| 95 | <100 | <100 |
| 96 | <100 | >100 |
| 97 | <100 | >100 |
| 98 | <100 | <100 |
| 99 | <100 | <100 |
| 100 | <100 | >100 |
| 101 | <100 | <100 |
| 104 | <100 | N/A |
| 105 | <100 | <100 |
| 106 | <100 | <100 |
| 107 | <100 | <100 |
| 108 | <100 | <100 |
| 109 | <100 | >100 |
| 110 | <100 | N/A |
| 111 | <100 | N/A |
| 112 | <100 | >100 |
| 113 | <100 | >100 |
| 114 | <100 | <100 |
| 115 | <100 | >100 |
| 116 | <100 | <100 |
| 117 | <100 | <100 |
| 118 | <100 | <100 |
| 119 | <100 | <100 |
| 120 | <100 | <100 |
| 121 | <100 | <100 |
| 122 | <100 | <100 |
| 123 | <100 | <100 |
| 124 | <100 | <100 |
| 125 | <100 | <100 |
| 126 | <100 | N/A |
| 127 | <100 | <100 |
| 128 | <100 | N/A |
| 129 | <100 | <100 |
| 130 | <100 | >100 |
| 131 | <100 | <100 |
| 132 | <100 | <100 |
| 133 | <100 | N/A |
| 134 | <100 | N/A |
| 135 | <100 | >100 |
| 136 | <100 | >100 |
| 137 | <100 | >100 |
| 138 | <100 | N/A |
| 139 | <100 | N/A |
| 140 | <100 | >100 |
| 142 | <100 | >100 |
| 143 | <100 | N/A |
| 144 | <100 | N/A |
| 145 | <100 | <100 |
| 146 | <100 | <100 |
| 147 | <100 | >100 |
| 148 | <100 | >100 |
| 150 | <100 | >100 |
| 151 | <100 | >100 |
| 152 | <100 | >100 |
| 153 | <100 | >100 |
| 154 | <100 | <100 |
| 155 | <100 | <100 |
| 156 | <100 | <100 |
| 157 | <100 | <100 |
| 158 | <100 | <100 |
| 159 | <100 | >100 |
| 160 | <100 | <100 |
| 161 | <100 | <100 |
| 162 | <100 | <100 |
| 163 | <100 | <100 |
| 164 | <100 | <100 |
| 165 | <100 | <100 |
| 166 | <100 | <100 |
| 167 | <100 | <100 |
| 168 | <100 | <100 |
| 169 | <100 | <100 |
| 170 | <100 | <100 |
| 171 | <100 | <100 |
| 172 | <100 | <100 |
| 173 | <100 | <100 |
| 174 | <100 | <100 |

TABLE 2
PPI IC₅₀ > 100 nM

| | PPI Assay | p-ERK Assay |
|---|---|---|
| Example # | IC₅₀ (100 nM) | IC₅₀ (nM) |
| 1 | >100 | >100 |
| 7 | >100 | N/A |
| 8 | >100 | N/A |
| 9 | >100 | N/A |
| 10 | >100 | N/A |
| 15 | >100 | N/A |
| 28 | >100 | N/A |
| 53 | >100 | N/A |
| 56 | >100 | N/A |
| 57 | >100 | N/A |
| 65 | >100 | N/A |
| 70 | >100 | >100 |
| 75 | >100 | N/A |
| 77 | >100 | N/A |
| 80 | >100 | N/A |
| 81 | >100 | N/A |
| 82 | >100 | N/A |
| 83 | >100 | N/A |
| 84 | >100 | N/A |
| 87 | >100 | N/A |
| 89 | >100 | N/A |
| 102 | >100 | N/A |
| 103 | >100 | N/A |
| 141 | >100 | N/A |
| 149 | >100 | >100 |

Finally, it should be noted that the above embodiments are only used to illustrate the technical solutions of the present invention, but are not intended to limit the scope of protection of the present invention. Those of ordinary skill in the art can make other different forms of transformations or changes based on the above description and ideas. All the embodiments need not and cannot be exhaustive here. Any modifications, equivalent substitutions, and improvements made within the spirit and principle of the present invention shall all fall within the scope of protection of the claims of the present invention.

1. A fused heterocyclic compound of Formula (I), 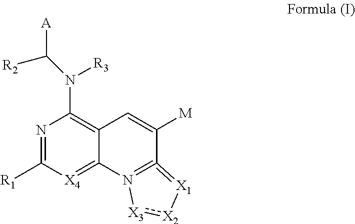
wherein
X₁ is N or CR₁₁;
X₂ and X₃ are each independently N, CR₁₁ or O, but are not both O at the same time; and
when one of X₂ and X₃ is O, X₂ and X₃ are bonded together by a single bond; R₁₁ is H, halo, alkyl, haloalkyl, cycloalkyl, heterocycloalkyl, hydroxyl, hydroxyalkyl, alkoxyl, alkoxyalkyl, amine, cyano, alkynyl or alkenyl;
X₄ is N or CR₁₂; R₁₂ is H, halo, haloalkyl or alkyl;
R₁ is H, halo, alkyl, haloalkyl, cycloalkyl, heterocycloalkyl, hydroxyl, hydroxyalkyl, alkoxyl, alkoxyalkyl, amine, cyano, alkynyl or alkenyl;
R₂ and R₃ are each independently H, halo, alkyl, haloalkyl, alkoxyl, alkoxyalkyl, hydroxyl, hydroxyalkyl, acyl, alkoxycarbonyl, or amine;
A is cycloalkyl, heterocycloalkyl, cycloalkenyl, heterocycloalkenyl, aryl, heteroaryl, or 8- to 13-membered fused bicyclic ring, and A is optionally substituted with one or more substituents each of which is independently halo, alkyl, haloalkyl, alkoxy, alkoxyalkyl, hydroxy, hydroxyalkyl, acyl, alkoxycarbonyl, alkoxyalkcarbonyl, cyano, amine, alkylamino, or dialkylamino; the 8- to 13-membered fused bicyclic ring contains at least one aromatic ring;
M is amine, alkyl, halo, haloalkyl, hydroxy, hydroxyalkyl, alkoxy, alkoxyalkyl, acyl, alkoxycarbonyl, alkoxyalkcarbonyl, aminocarbonyl, carboxyl, alkenyl, alkynyl, alkylthio, aryl, heteroaryl, cycloalkyl, heterocycloalkyl, cycloalkenyl, heterocycloalkenyl, fused bicyclic cycloalkyl or heterocyloalkyl, spirocyclic cycloalkyl or heterocyloalkyl, or bridged cycloalkyl or heterocyloalkyl; and M is optionally substituted with 1-5 R₁₀ groups; each R₁₀ group is independently oxo, acyl, hydroxylcarbonyl, hydroxyalkyl-carbonyl, alkoxycarbonyl, alkoxyalkcarbonyl, aminocarbonyl, alkylaminocarbonyl, dialkylaminocarbonyl, —C(O)—(CH₂)ₙ—OR, —S(O)₂—R, alk-S(O)₂—R, alk-C(O)—R, alk-C(O)—NRR′, CD₃-O—, cyano, alkoxyl, alkoxyalkyl, hydroxyl, hydroxyalkyl, alkyl, halo, haloalkyl, cycloalkyl, heterocycloalkyl, cycloalkenyl, or heterocycloalkenyl; optionally, two R₁₀ groups together with the atom to which they are both bonded form cycloalkyl or heterocycloalkyl;
optionally, M is connected to the fused tri-heterocyclic moiety in Formula (I) via a linker which is —CH₂—, —(CH₂)ₙ—O—(CH₂)ₙ—, —(CH₂)ₙ—NR—(CH₂)ₙ—, —(CH₂)ₙ—C(O)—O—(CH₂)ₙ—, —(CH₂)ₙ—O—C(O)—(CH₂)ₙ—, —(CH₂)ₙ—C(O)—(CH₂)ₙ—, —NR—C(O)— or —C(O)—NR—;
each R is independently H or alkyl;
each R′ is independently H or alkyl;
n is 0, 1, 2, or 3;
a ring-forming carbon atom or heteroatom in cycloalkyl, heterocycloalkyl, cycloalkenyl and heterocycloalkenyl is optionally substituted by one or two oxo groups;
any hydrogen (H) can be optionally replaced by deuterium (D);
or its tautomer, stereoisomer, or pharmaceutically acceptable salt thereof.

2. The compound of claim 1, wherein A is aryl, heteroaryl, or 8- to 13-membered fused bicyclic ring, and is optionally substituted with one or more substituents each of which is independently halo, alkyl, haloalkyl, alkoxy, alkoxyalkyl, hydroxy, hydroxyalkyl, acyl, alkoxycarbonyl, alkoxyalkcarbonyl, cyano, amine, alkylamino, or dialkylamino.

3. The compound of claim 1, wherein A is phenyl or fused phenyl, and A is optionally substituted with 1-5 substituents selected from halo, haloalkyl, alkyl, and amine.

4. The compound of claim 1, wherein halo is F.

5. The compound of claim 1, wherein A is

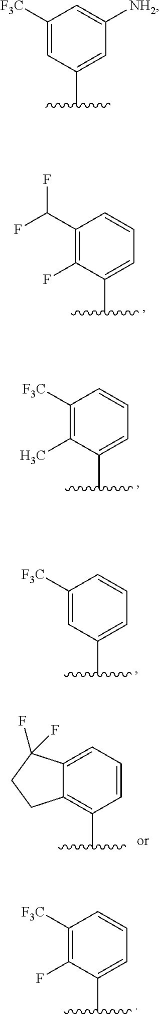 [SMILES: *c1cc(N)cc(C(F)(F)F)c1 ; *c1cccc(C(F)F)c1F ; *c1cccc(C(F)(F)F)c1C ; *c1cccc(C(F)(F)F)c1 ; *c1cccc2c1CCC2(F)F ; *c1cccc(C(F)(F)F)c1F]

6. The compound of claim 1, wherein X₁ is N.

7. The compound of claim 1, wherein X₄ is N.

8. The compound of claim 1, wherein X₂ and X₃ are each independently N or CH.

9. The compound of claim 1, wherein M is alkoxycarbonyl, alkoxyalkyl-carbonyl, aminocarbonyl, alkylaminocarbonyl, dialkylaminocarbonyl, acyl, 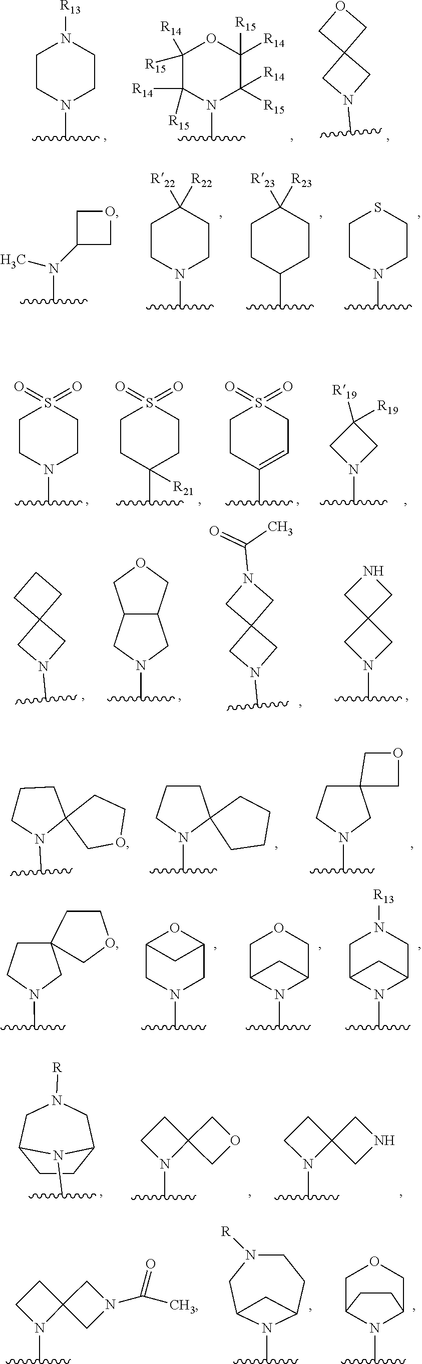 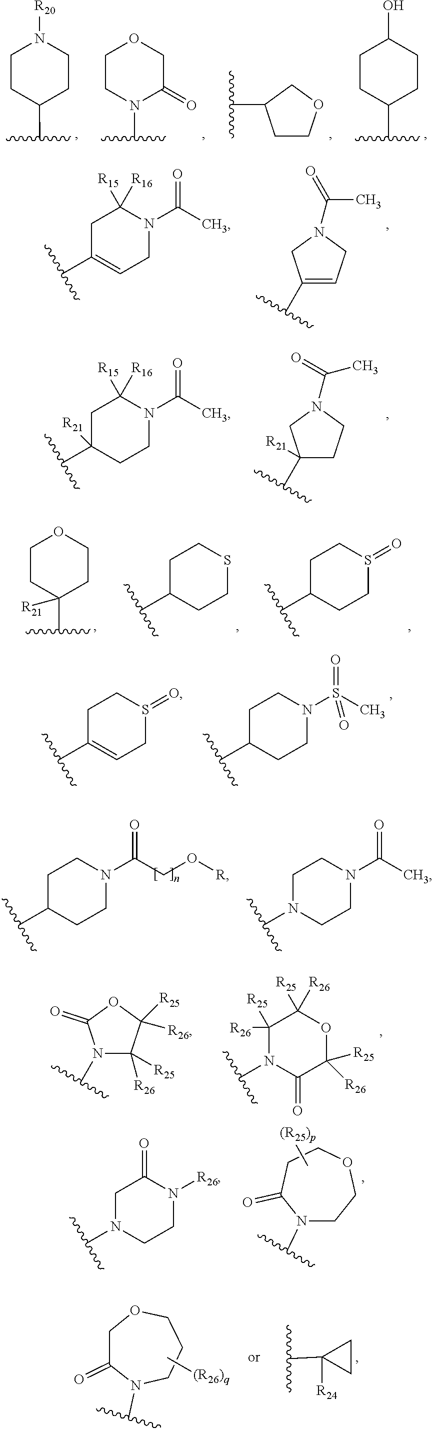
R₁₃ is H, hydroxyl, hydroxyalkyl, alkoxyl, alkoxyalkyl, acyl, —C(O)—NR₁₇R₁₈, —S(O)₂—R, -alk-S(O)₂—R, hydroxycarbonyl, alkoxycarbonyl, hydroxyalkyl-carbonyl, alkoxyalkyl-carbonyl, oxo, alkyl, cycloalkenyl, heterocycloalkenyl, cycloalkyl or heterocycloalkyl; and R₁₃ is optionally further substituted with one or more halo, alkyl, hydroxyl, haloalkyl, or alkoxy;
each of R₁₄, R₁₅, and R₁₆ is, independently, H, halo, hydroxyl, hydroxyalkyl, haloalkyl, alkyl, amine, alkoxy, or alkoxyalkyl; optionally, R₁₄ and R₁₅ together with the atom to which they are both bonded form cycloalkyl or heterocycloalkyl; or optionally, R₁₅ and R₁₆ together with the atom to which they are both bonded form cycloalkyl or heterocycloalkyl;
each of R₁₇ and R₁₈ is, independently, H, cycloalkyl, heterocycloalkyl, or alkyl; optionally, R₁₇ and R₁₈ together with the nitrogen atom (N) to which they are both bonded form heterocycloalkyl;
each of R₁₉ and R′₁₉ is, independently, H, alkyl, alkoxy, alkoxyalkyl, hydroxyl, hydroxyalkyl, halo, haloalkyl, oxo, acyl, hydroxylcarbonyl, alkoxycarbonyl, hydroxyalkyl-carbonyl, alkoxyalkyl-carbonyl, cyano, —S(O)₂—R, alk-S(O)₂—R, —C(O)—NR₁₇R₁₈, cycloalkenyl, heterocycloalkenyl, cycloalkyl or heterocycloalkyl; optionally, R₁₉ and R′₁₉ together with the atom to which they are both bonded form cycloalkyl or heterocycloalkyl;
R₂₀ is H, alkyl, halo, haloalkyl, alkoxyl, alkoxyalkyl, acyl, oxo, —S(O)₂—R, -alk-S(O)₂—R, hydroxylcarbonyl, alkoxycarbonyl, hydroxyalkyl-carbonyl, alkoxyalkyl-carbonyl, —C(O)—NR₁₇R₁₈—, cycloalkenyl, heterocycloalkenyl, cycloalkyl, heterocycloalkyl, or 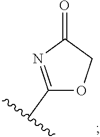 [SMILES: *C1=NC(=O)CO1]
R₂₁ is H, alkyl, hydroxyl, hydroxyalkyl, alkoxyl, alkoxyalkyl, halo, haloalkyl, haloalkyloxy, CD₃-O—, —NR—C(O)—R, or —(CH₂)ₙ—C(O)—NRR′;
each of R₂₂, R′₂₂, R₂₃ and R′₂₃ is, independently, H, alkyl, alkoxy, alkoxyalkyl, hydroxyl, hydroxyalkyl, halo, haloalkyl, acyl, hydroxylcarbonyl, alkoxycarbonyl, hydroxyalkyl-carbonyl, alkoxyalkyl-carbonyl, amine, oxo, —S(O)₂—R, alk-S(O)₂—R, —NR—C(O)—R, —C(O)—NR₁₇R₁₈, or —(CH₂)ₙ—C(O)—NRR′; optionally, R₂₂ and R′₂₂ together with the atom to which they are both bonded form cycloalkyl or heterocycloalkyl; or optionally, R₂₃ and R′₂₃ together with the atom to which they are both bonded form cycloalkyl or heterocycloalkyl;
R₂₄ is H, alkyl, haloalkyl, haloalkyloxy, hydroxy, hydroxyalkyl, alkoxyl, alkoxyalkyl, or cyano;
each of R₂₅ and R₂₆ is, independently, H, alkyl, halo, haloalkyl, alkoxyalkyl, alkoxy, hydroxyl, hydroxyalkyl, cycloalkyl, or heterocycloalkyl; optionally, R₂₅ and R₂₆ together with the atom to which they are both bonded form cycloalkyl or heterocycloalkyl;
each R is independently H or alkyl;
each R′ is independently H or alkyl;
n is 0, 1, 2, or 3;
p is 0, 1, 2, 3, or 4;
q is 0, 1, 2, 3, 4, 5, or 6;
optionally, M is connected to the fused tri-heterocyclic moiety in Formula (I) via the linker —C(O)—.

10. The compound of claim 1, wherein M is 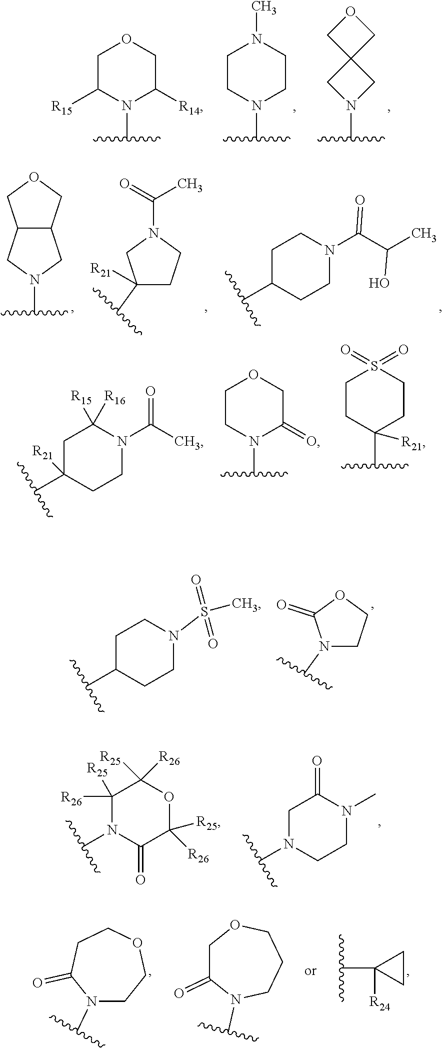
and M is connected to the fused tri-heterocyclic moiety in Formula (I) optionally via the linker —C(O)—.

11. The compound of claim 1, wherein R₃ is H.

12. The compound of claim 1, wherein R₂ is alkyl.

13. The compound of claim 1, wherein R₁ is H or alkyl.

14. The compound of claim 1, wherein the compound is selected from the following:

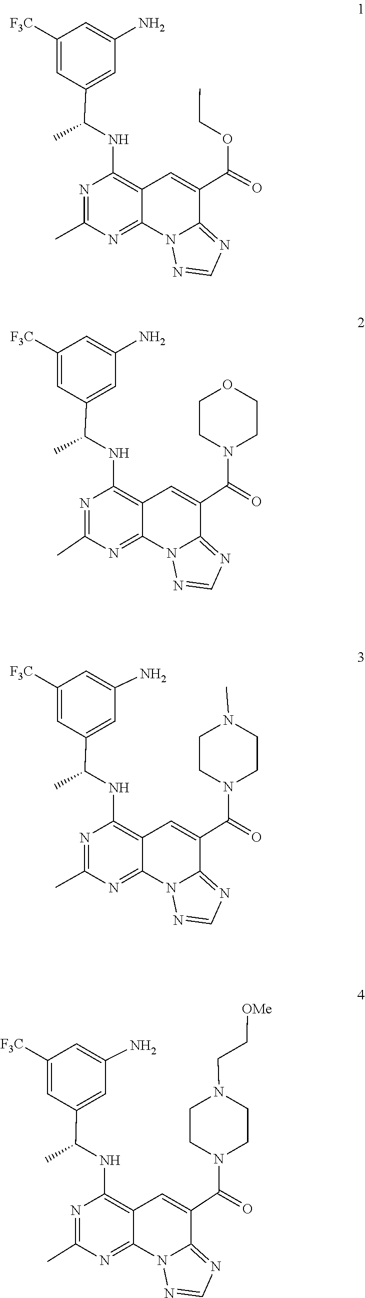 [SMILES: CCOC(=O)c1cc2c(N[C@H](C)c3cc(N)cc(C(F)(F)F)c3)nc(C)nc2n2ncnc12 ; Cc1nc(N[C@H](C)c2cc(N)cc(C(F)(F)F)c2)c2cc(C(=O)N3CCOCC3)c3ncnn3c2n1 ; Cc1nc(N[C@H](C)c2cc(N)cc(C(F)(F)F)c2)c2cc(C(=O)N3CCN(C)CC3)c3ncnn3c2n1 ; COCCN1CCN(C(=O)c2cc3c(N[C@H](C)c4cc(N)cc(C(F)(F)F)c4)nc(C)nc3n3ncnc23)CC1]

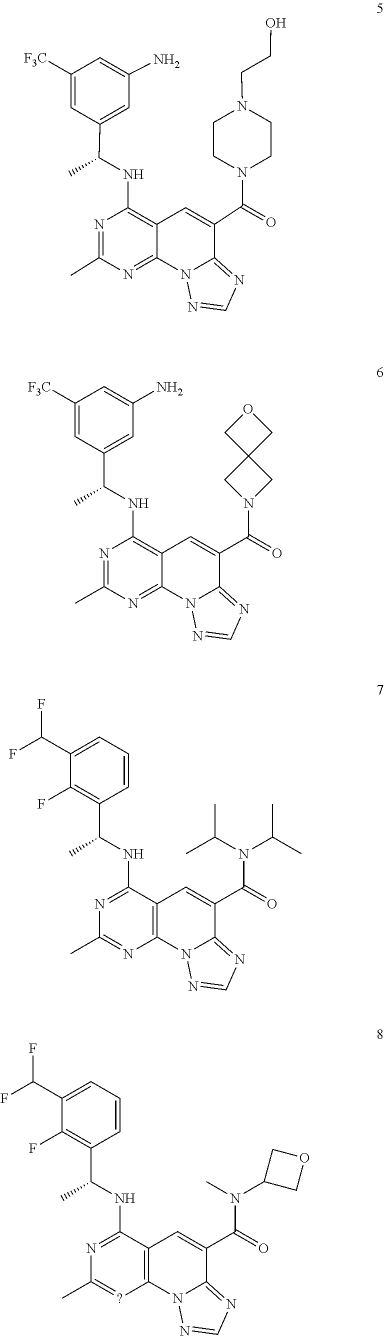

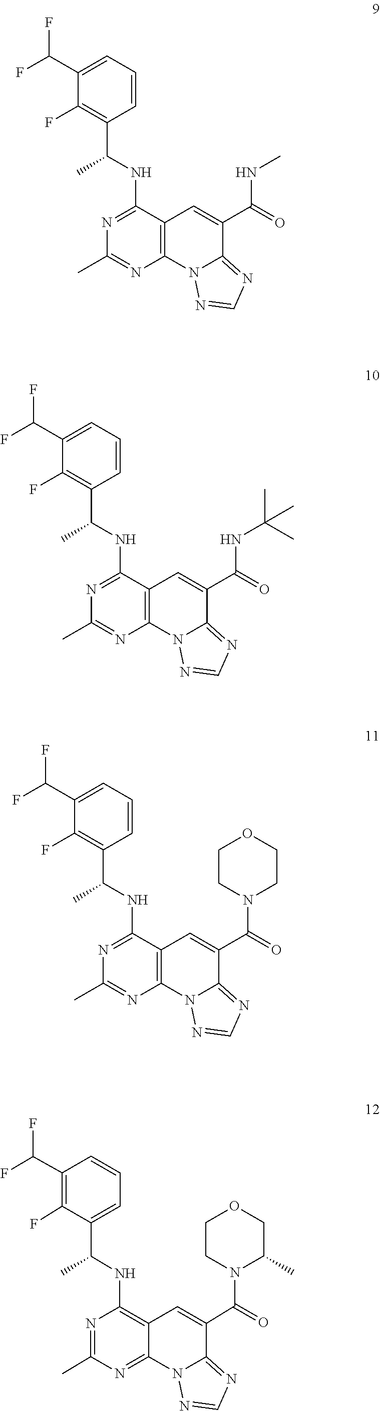 [SMILES: CNC(=O)c1cc2c(N[C@H](C)c3cccc(C(F)F)c3F)nc(C)nc2n2ncnc12 ; Cc1nc(N[C@H](C)c2cccc(C(F)F)c2F)c2cc(C(=O)NC(C)(C)C)c3ncnn3c2n1 ; Cc1nc(N[C@H](C)c2cccc(C(F)F)c2F)c2cc(C(=O)N3CCOCC3)c3ncnn3c2n1 ; Cc1nc(N[C@H](C)c2cccc(C(F)F)c2F)c2cc(C(=O)N3CCOC[C@@H]3C)c3ncnn3c2n1]

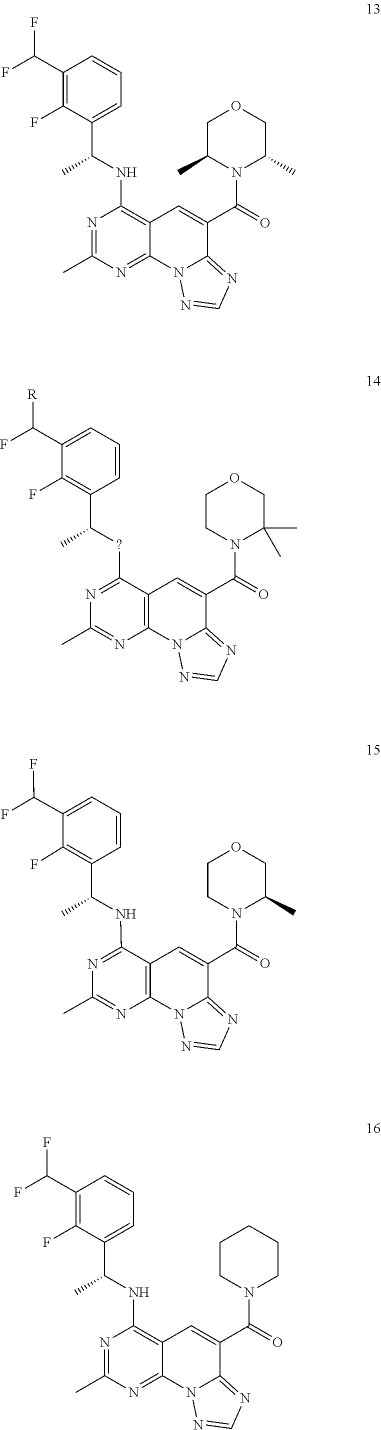

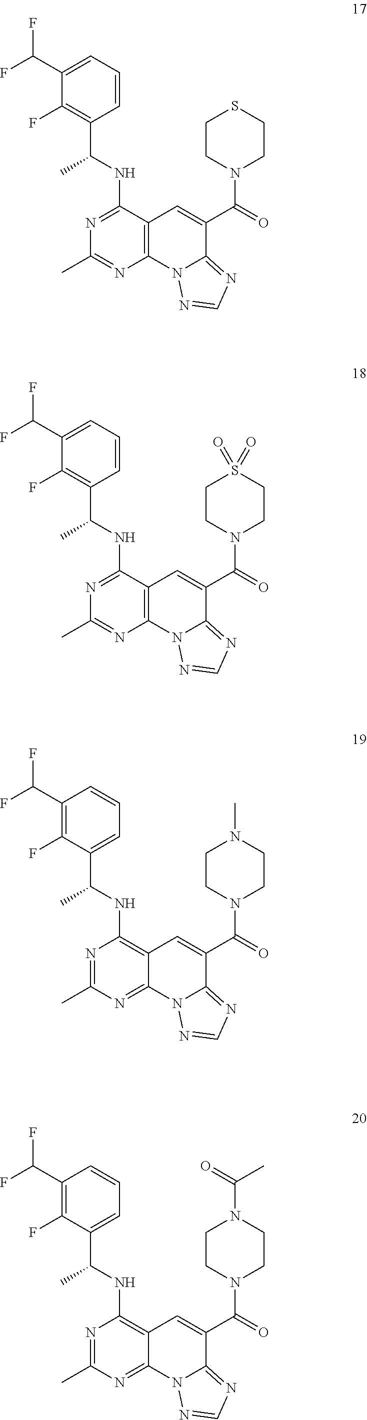 [SMILES: Cc1nc(N[C@H](C)c2cccc(C(F)F)c2F)c2cc(C(=O)N3CCSCC3)c3ncnn3c2n1 ; Cc1nc(N[C@H](C)c2cccc(C(F)F)c2F)c2cc(C(=O)N3CCS(=O)(=O)CC3)c3ncnn3c2n1 ; Cc1nc(N[C@H](C)c2cccc(C(F)F)c2F)c2cc(C(=O)N3CCN(C)CC3)c3ncnn3c2n1 ; CC(=O)N1CCN(C(=O)c2cc3c(N[C@H](C)c4cccc(C(F)F)c4F)nc(C)nc3n3ncnc23)CC1]

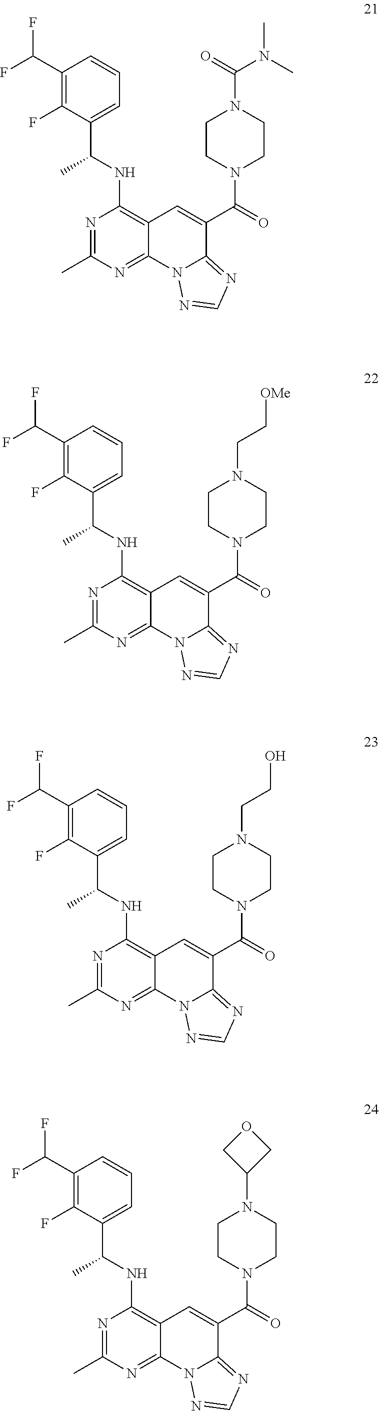 [SMILES: Cc1nc(N[C@H](C)c2cccc(C(F)F)c2F)c2cc(C(=O)N3CCN(C(=O)N(C)C)CC3)c3ncnn3c2n1 ; COCCN1CCN(C(=O)c2cc3c(N[C@H](C)c4cccc(C(F)F)c4F)nc(C)nc3n3ncnc23)CC1 ; Cc1nc(N[C@H](C)c2cccc(C(F)F)c2F)c2cc(C(=O)N3CCN(CCO)CC3)c3ncnn3c2n1 ; Cc1nc(N[C@H](C)c2cccc(C(F)F)c2F)c2cc(C(=O)N3CCN(C4COC4)CC3)c3ncnn3c2n1]

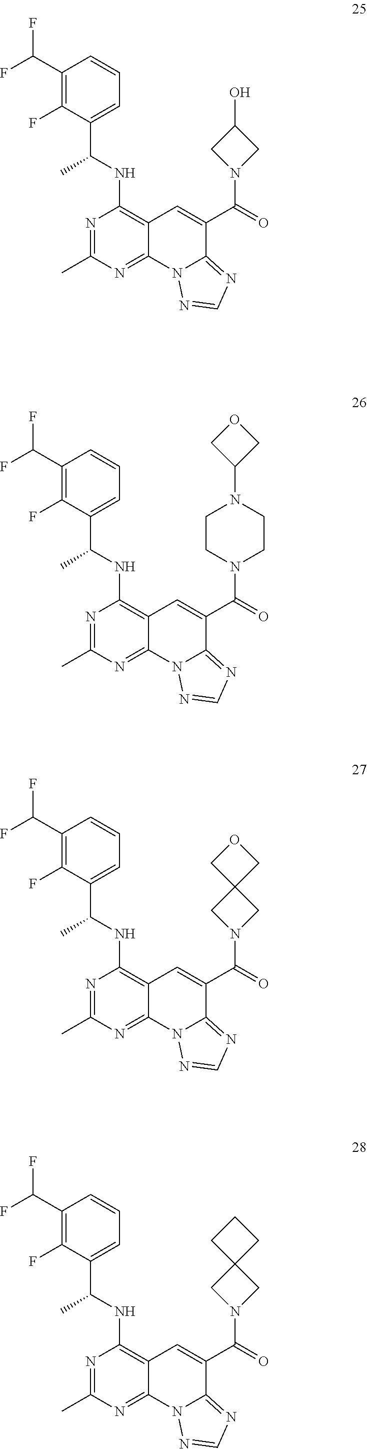 [SMILES: Cc1nc(N[C@H](C)c2cccc(C(F)F)c2F)c2cc(C(=O)N3CC(O)C3)c3ncnn3c2n1 ; Cc1nc(N[C@H](C)c2cccc(C(F)F)c2F)c2cc(C(=O)N3CCN(C4COC4)CC3)c3ncnn3c2n1 ; Cc1nc(N[C@H](C)c2cccc(C(F)F)c2F)c2cc(C(=O)N3CC4(COC4)C3)c3ncnn3c2n1 ; Cc1nc(N[C@H](C)c2cccc(C(F)F)c2F)c2cc(C(=O)N3CC4(CCC4)C3)c3ncnn3c2n1]

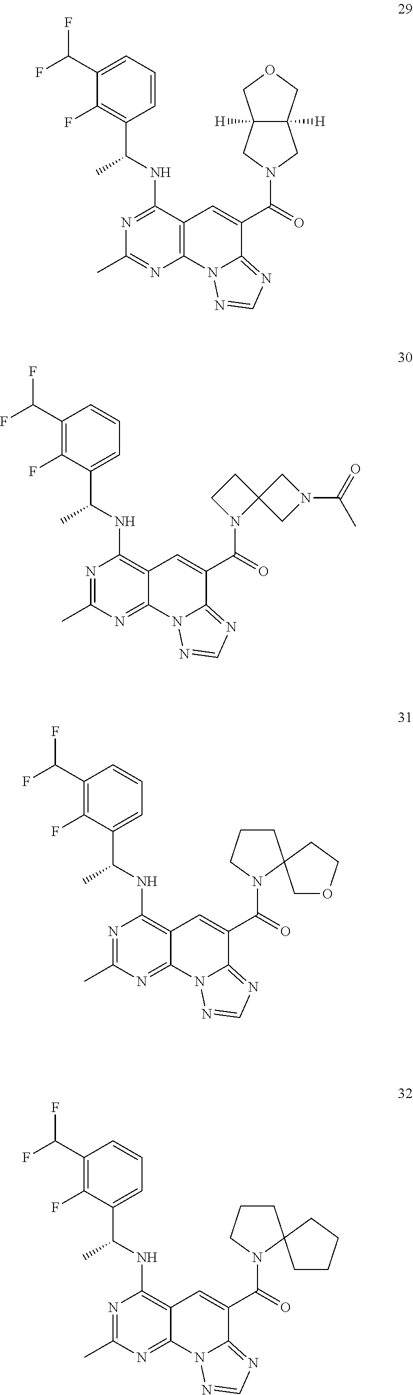 [SMILES: Cc1nc(N[C@H](C)c2cccc(C(F)F)c2F)c2cc(C(=O)N3C[C@H]4COC[C@H]4C3)c3ncnn3c2n1 ; CC(=O)N1CC2(CCN2C(=O)c2cc3c(N[C@H](C)c4cccc(C(F)F)c4F)nc(C)nc3n3ncnc23)C1 ; Cc1nc(N[C@H](C)c2cccc(C(F)F)c2F)c2cc(C(=O)N3CCCC34CCOC4)c3ncnn3c2n1 ; Cc1nc(N[C@H](C)c2cccc(C(F)F)c2F)c2cc(C(=O)N3CCCC34CCCC4)c3ncnn3c2n1]

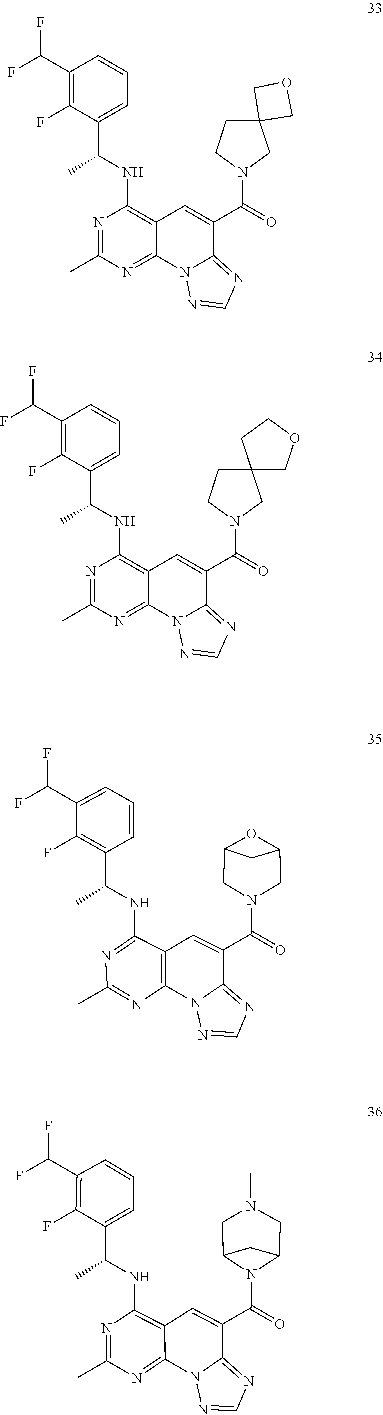 [SMILES: Cc1nc(N[C@H](C)c2cccc(C(F)F)c2F)c2cc(C(=O)N3CCC4(COC4)C3)c3ncnn3c2n1 ; Cc1nc(N[C@H](C)c2cccc(C(F)F)c2F)c2cc(C(=O)N3CCC4(CCOC4)C3)c3ncnn3c2n1 ; Cc1nc(N[C@H](C)c2cccc(C(F)F)c2F)c2cc(C(=O)N3CC4CC(C3)O4)c3ncnn3c2n1 ; Cc1nc(N[C@H](C)c2cccc(C(F)F)c2F)c2cc(C(=O)N3C4CC3CN(C)C4)c3ncnn3c2n1]

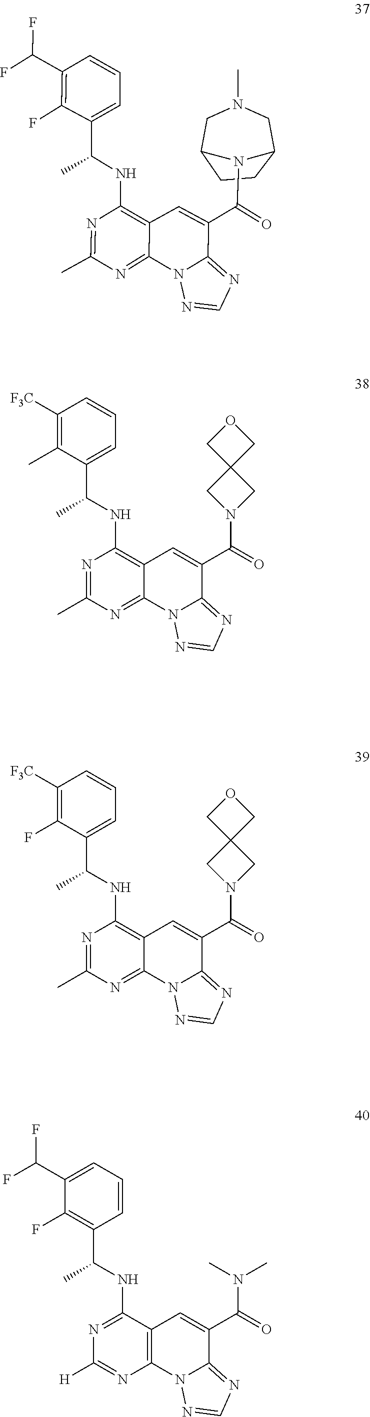 [SMILES: Cc1nc(N[C@H](C)c2cccc(C(F)F)c2F)c2cc(C(=O)N3C4CCC3CN(C)C4)c3ncnn3c2n1 ; Cc1nc(N[C@H](C)c2cccc(C(F)(F)F)c2C)c2cc(C(=O)N3CC4(COC4)C3)c3ncnn3c2n1 ; Cc1nc(N[C@H](C)c2cccc(C(F)(F)F)c2F)c2cc(C(=O)N3CC4(COC4)C3)c3ncnn3c2n1 ; C[C@@H](Nc1ncnc2c1cc(C(=O)N(C)C)c1ncnn12)c1cccc(C(F)F)c1F]

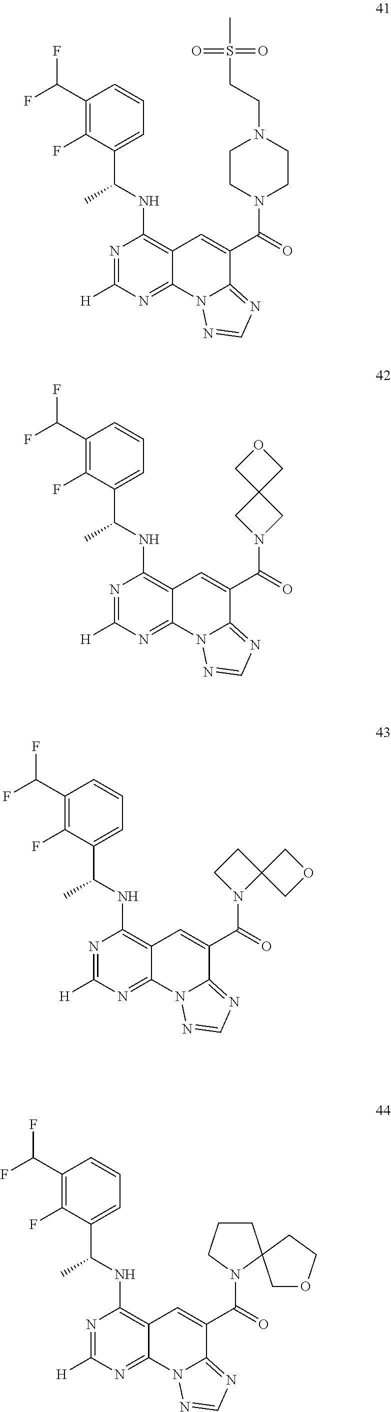 [SMILES: C[C@@H](Nc1ncnc2c1cc(C(=O)N1CCN(CCS(C)(=O)=O)CC1)c1ncnn12)c1cccc(C(F)F)c1F ; C[C@@H](Nc1ncnc2c1cc(C(=O)N1CC3(COC3)C1)c1ncnn12)c1cccc(C(F)F)c1F ; C[C@@H](Nc1ncnc2c1cc(C(=O)N1CCC13COC3)c1ncnn12)c1cccc(C(F)F)c1F ; C[C@@H](Nc1ncnc2c1cc(C(=O)N1CCCC13CCOC3)c1ncnn12)c1cccc(C(F)F)c1F]

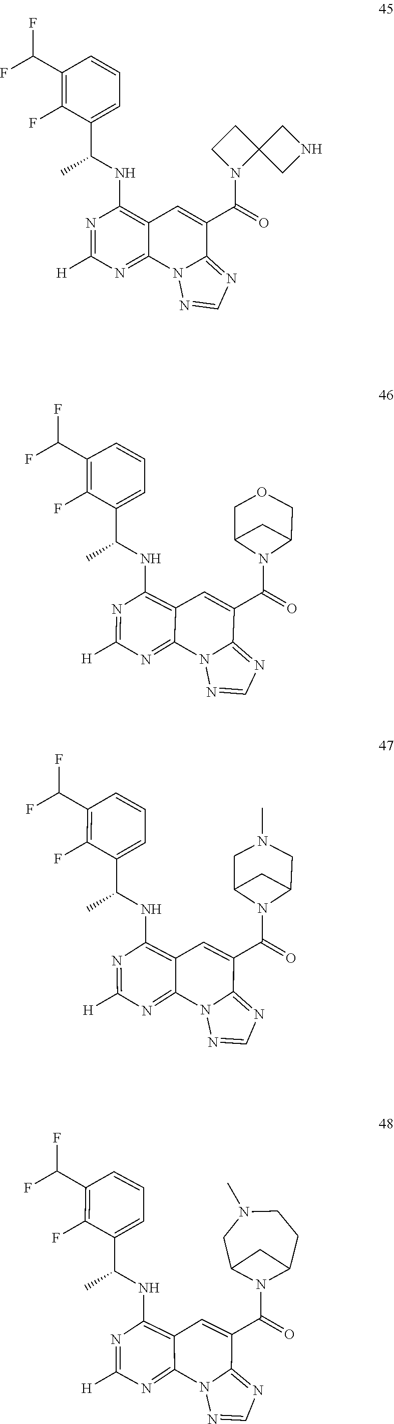 [SMILES: C[C@@H](Nc1ncnc2c1cc(C(=O)N1CCC13CNC3)c1ncnn12)c1cccc(C(F)F)c1F ; C[C@@H](Nc1ncnc2c1cc(C(=O)N1C3COCC1C3)c1ncnn12)c1cccc(C(F)F)c1F ; C[C@@H](Nc1ncnc2c1cc(C(=O)N1C3CC1CN(C)C3)c1ncnn12)c1cccc(C(F)F)c1F ; C[C@@H](Nc1ncnc2c1cc(C(=O)N1C3CCN(C)CC1C3)c1ncnn12)c1cccc(C(F)F)c1F]

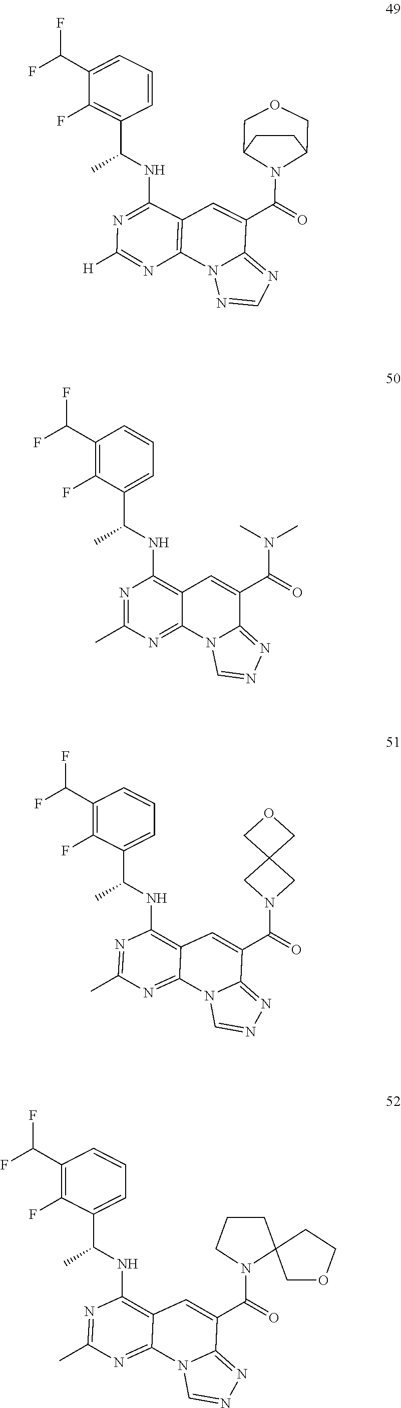 [SMILES: C[C@@H](Nc1ncnc2c1cc(C(=O)N1C3CCC1COC3)c1ncnn12)c1cccc(C(F)F)c1F ; Cc1nc(N[C@H](C)c2cccc(C(F)F)c2F)c2cc(C(=O)N(C)C)c3nncn3c2n1 ; Cc1nc(N[C@H](C)c2cccc(C(F)F)c2F)c2cc(C(=O)N3CC4(COC4)C3)c3nncn3c2n1 ; Cc1nc(N[C@H](C)c2cccc(C(F)F)c2F)c2cc(C(=O)N3CCCC34CCOC4)c3nncn3c2n1]

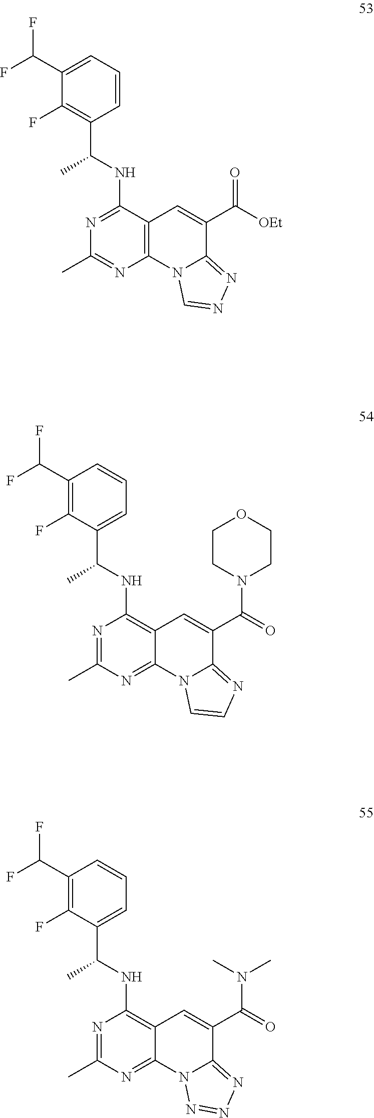 [SMILES: CCOC(=O)c1cc2c(N[C@H](C)c3cccc(C(F)F)c3F)nc(C)nc2n2cnnc12 ; Cc1nc(N[C@H](C)c2cccc(C(F)F)c2F)c2cc(C(=O)N3CCOCC3)c3nccn3c2n1 ; Cc1nc(N[C@H](C)c2cccc(C(F)F)c2F)c2cc(C(=O)N(C)C)c3nnnn3c2n1]

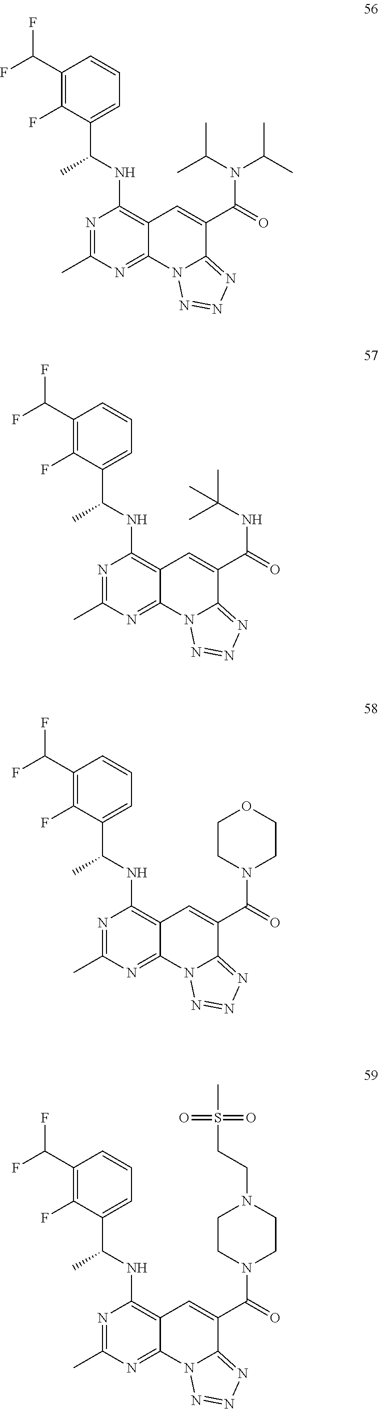 [SMILES: Cc1nc(N[C@H](C)c2cccc(C(F)F)c2F)c2cc(C(=O)N(C(C)C)C(C)C)c3nnnn3c2n1 ; Cc1nc(N[C@H](C)c2cccc(C(F)F)c2F)c2cc(C(=O)NC(C)(C)C)c3nnnn3c2n1 ; Cc1nc(N[C@H](C)c2cccc(C(F)F)c2F)c2cc(C(=O)N3CCOCC3)c3nnnn3c2n1 ; Cc1nc(N[C@H](C)c2cccc(C(F)F)c2F)c2cc(C(=O)N3CCN(CCS(C)(=O)=O)CC3)c3nnnn3c2n1]

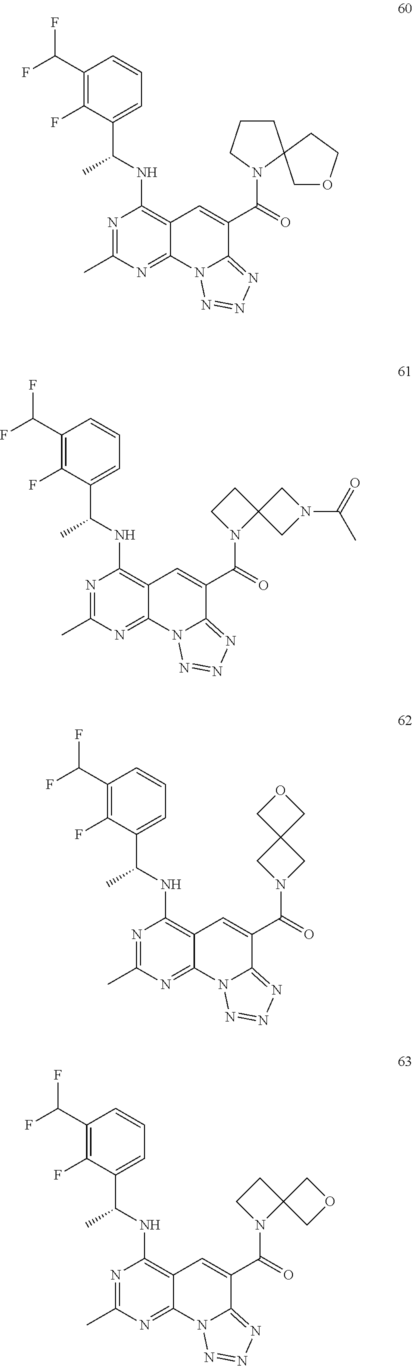 [SMILES: Cc1nc(N[C@H](C)c2cccc(C(F)F)c2F)c2cc(C(=O)N3CCCC34CCOC4)c3nnnn3c2n1 ; CC(=O)N1CC2(CCN2C(=O)c2cc3c(N[C@H](C)c4cccc(C(F)F)c4F)nc(C)nc3n3nnnc23)C1 ; Cc1nc(N[C@H](C)c2cccc(C(F)F)c2F)c2cc(C(=O)N3CC4(COC4)C3)c3nnnn3c2n1 ; Cc1nc(N[C@H](C)c2cccc(C(F)F)c2F)c2cc(C(=O)N3CCC34COC4)c3nnnn3c2n1]

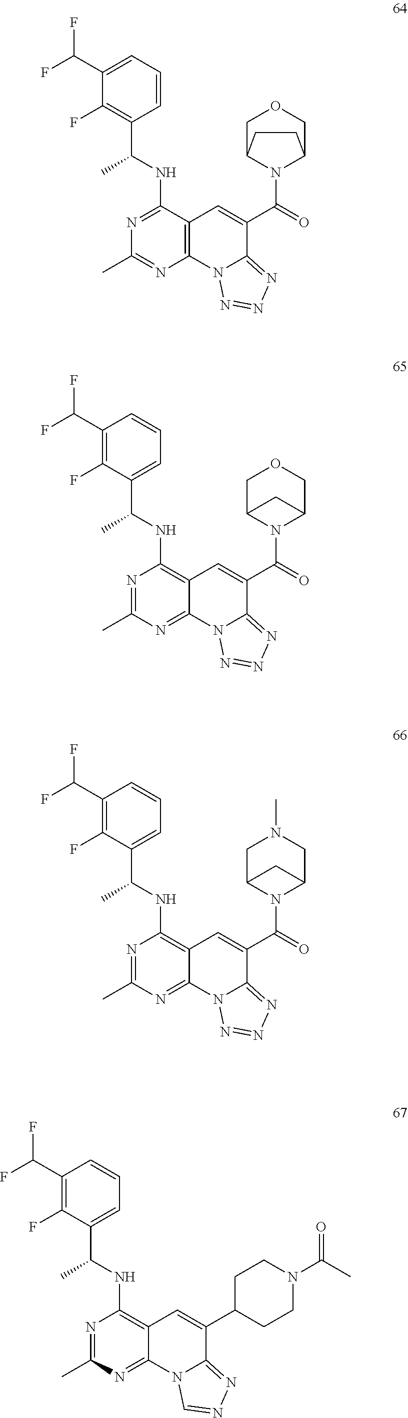

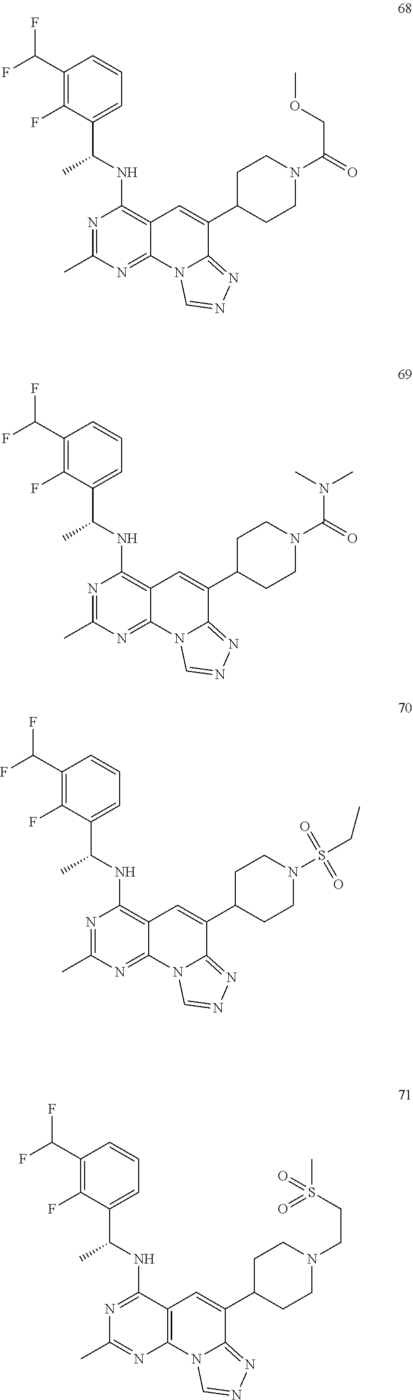 [SMILES: COCC(=O)N1CCC(c2cc3c(N[C@H](C)c4cccc(C(F)F)c4F)nc(C)nc3n3cnnc23)CC1 ; Cc1nc(N[C@H](C)c2cccc(C(F)F)c2F)c2cc(C3CCN(C(=O)N(C)C)CC3)c3nncn3c2n1 ; CCS(=O)(=O)N1CCC(c2cc3c(N[C@H](C)c4cccc(C(F)F)c4F)nc(C)nc3n3cnnc23)CC1 ; Cc1nc(N[C@H](C)c2cccc(C(F)F)c2F)c2cc(C3CCN(CCS(C)(=O)=O)CC3)c3nncn3c2n1]

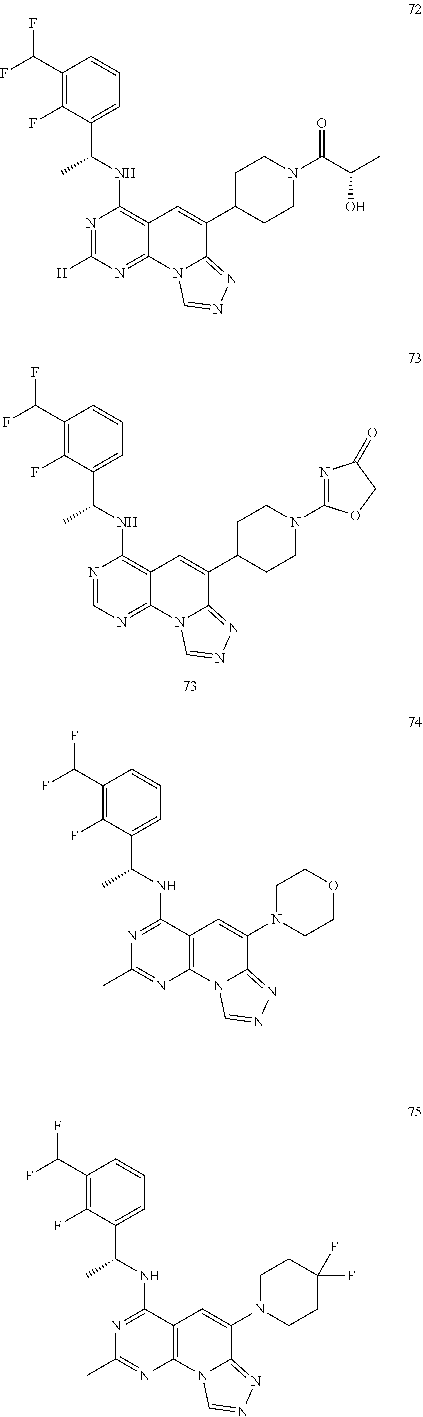 [SMILES: C[C@H](O)C(=O)N1CCC(c2cc3c(N[C@H](C)c4cccc(C(F)F)c4F)ncnc3n3cnnc23)CC1 ; C[C@@H](Nc1ncnc2c1cc(C1CCN(C3=NC(=O)CO3)CC1)c1nncn12)c1cccc(C(F)F)c1F ; Cc1nc(N[C@H](C)c2cccc(C(F)F)c2F)c2cc(N3CCOCC3)c3nncn3c2n1 ; Cc1nc(N[C@H](C)c2cccc(C(F)F)c2F)c2cc(N3CCC(F)(F)CC3)c3nncn3c2n1]

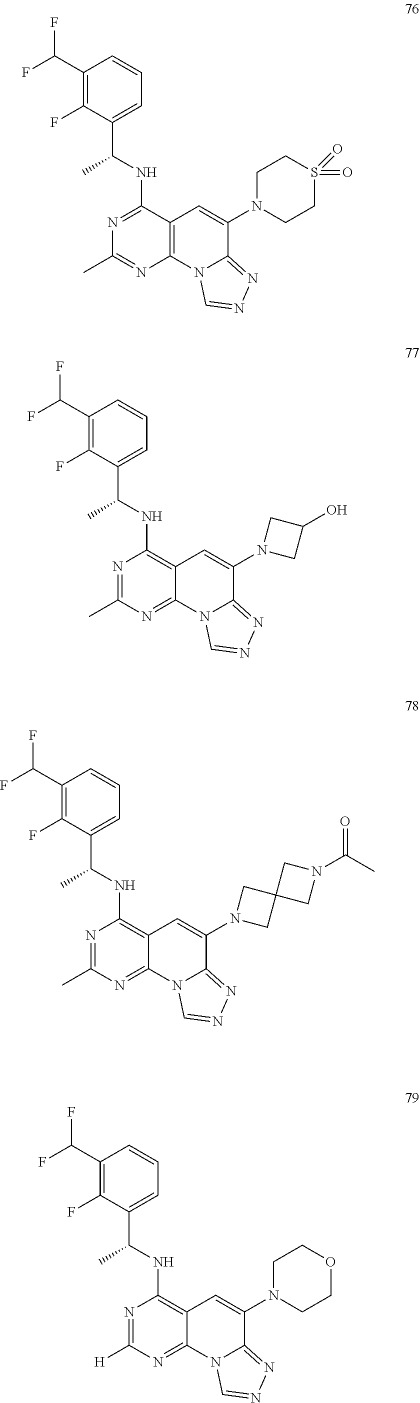 [SMILES: Cc1nc(N[C@H](C)c2cccc(C(F)F)c2F)c2cc(N3CCS(=O)(=O)CC3)c3nncn3c2n1 ; Cc1nc(N[C@H](C)c2cccc(C(F)F)c2F)c2cc(N3CC(O)C3)c3nncn3c2n1 ; CC(=O)N1CC2(C1)CN(c1cc3c(N[C@H](C)c4cccc(C(F)F)c4F)nc(C)nc3n3cnnc13)C2 ; C[C@@H](Nc1ncnc2c1cc(N1CCOCC1)c1nncn12)c1cccc(C(F)F)c1F]

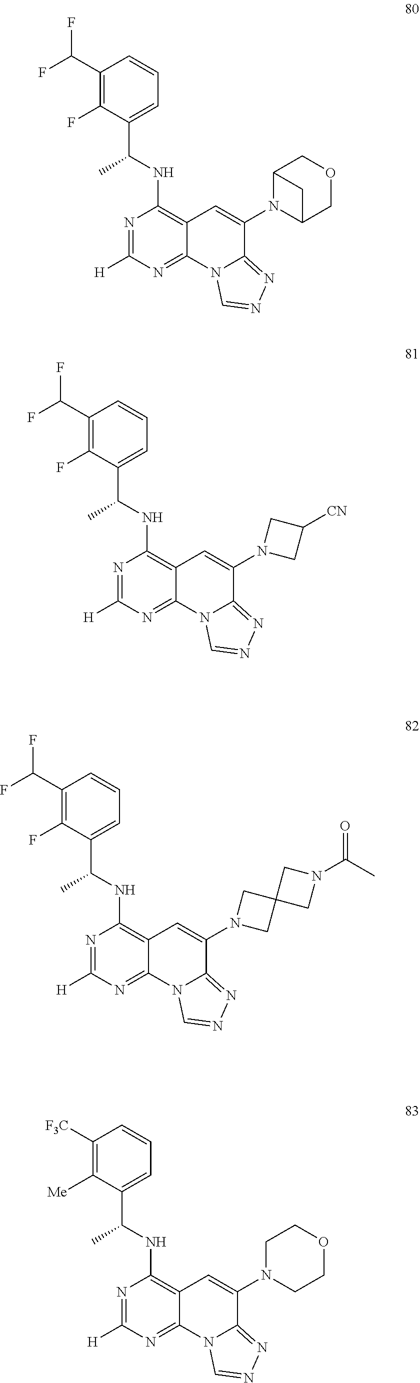 [SMILES: C[C@@H](Nc1ncnc2c1cc(N1C3COCC1C3)c1nncn12)c1cccc(C(F)F)c1F ; C[C@@H](Nc1ncnc2c1cc(N1CC(C#N)C1)c1nncn12)c1cccc(C(F)F)c1F ; CC(=O)N1CC2(C1)CN(c1cc3c(N[C@H](C)c4cccc(C(F)F)c4F)ncnc3n3cnnc13)C2 ; Cc1c([C@@H](C)Nc2ncnc3c2cc(N2CCOCC2)c2nncn23)cccc1C(F)(F)F]

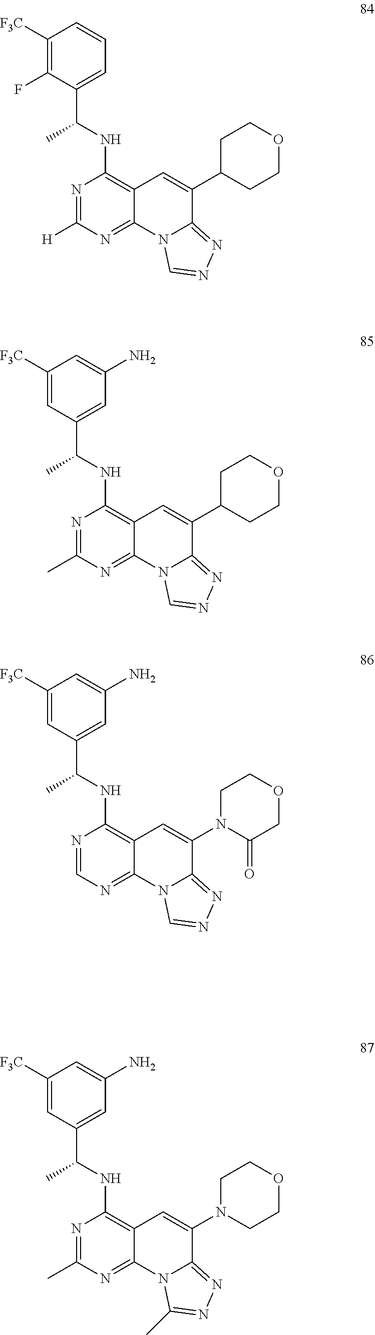 [SMILES: C[C@@H](Nc1ncnc2c1cc(C1CCOCC1)c1nncn12)c1cccc(C(F)(F)F)c1F ; Cc1nc(N[C@H](C)c2cc(N)cc(C(F)(F)F)c2)c2cc(C3CCOCC3)c3nncn3c2n1 ; C[C@@H](Nc1ncnc2c1cc(N1CCOCC1=O)c1nncn12)c1cc(N)cc(C(F)(F)F)c1 ; Cc1nc(N[C@H](C)c2cc(N)cc(C(F)(F)F)c2)c2cc(N3CCOCC3)c3nnc(C)n3c2n1]

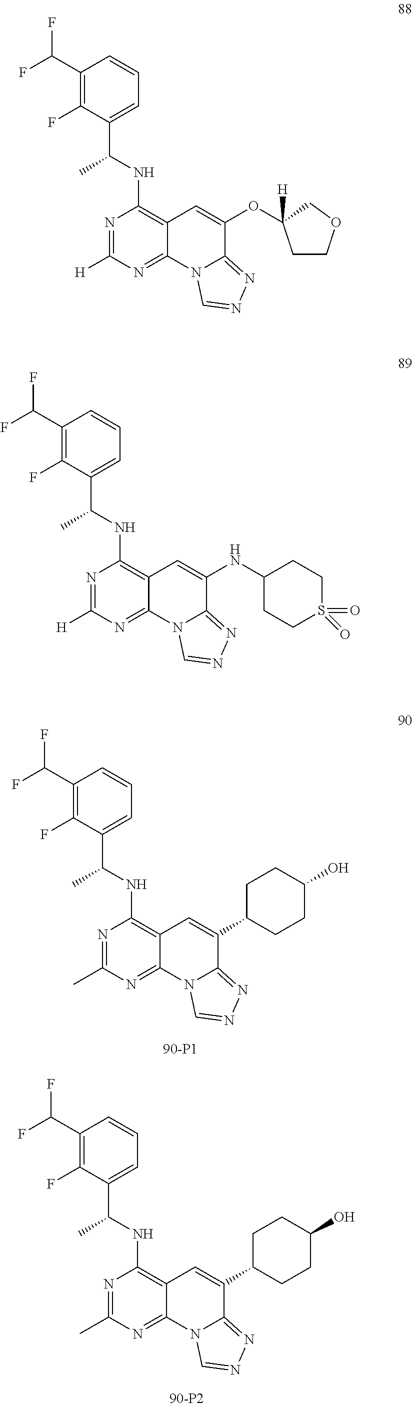 [SMILES: C[C@@H](Nc1ncnc2c1cc(O[C@H]1CCOC1)c1nncn12)c1cccc(C(F)F)c1F ; C[C@@H](Nc1ncnc2c1cc(NC1CCS(=O)(=O)CC1)c1nncn12)c1cccc(C(F)F)c1F ; Cc1nc(N[C@H](C)c2cccc(C(F)F)c2F)c2cc([C@H]3CC[C@@H](O)CC3)c3nncn3c2n1 ; Cc1nc(N[C@H](C)c2cccc(C(F)F)c2F)c2cc([C@H]3CC[C@H](O)CC3)c3nncn3c2n1]

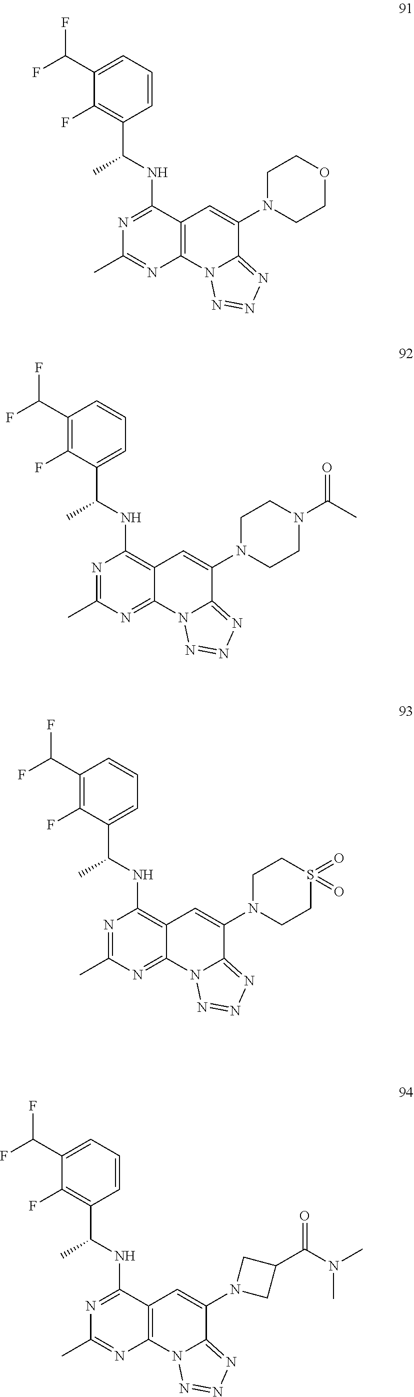 [SMILES: Cc1nc(N[C@H](C)c2cccc(C(F)F)c2F)c2cc(N3CCOCC3)c3nnnn3c2n1 ; CC(=O)N1CCN(c2cc3c(N[C@H](C)c4cccc(C(F)F)c4F)nc(C)nc3n3nnnc23)CC1 ; Cc1nc(N[C@H](C)c2cccc(C(F)F)c2F)c2cc(N3CCS(=O)(=O)CC3)c3nnnn3c2n1 ; Cc1nc(N[C@H](C)c2cccc(C(F)F)c2F)c2cc(N3CC(C(=O)N(C)C)C3)c3nnnn3c2n1]

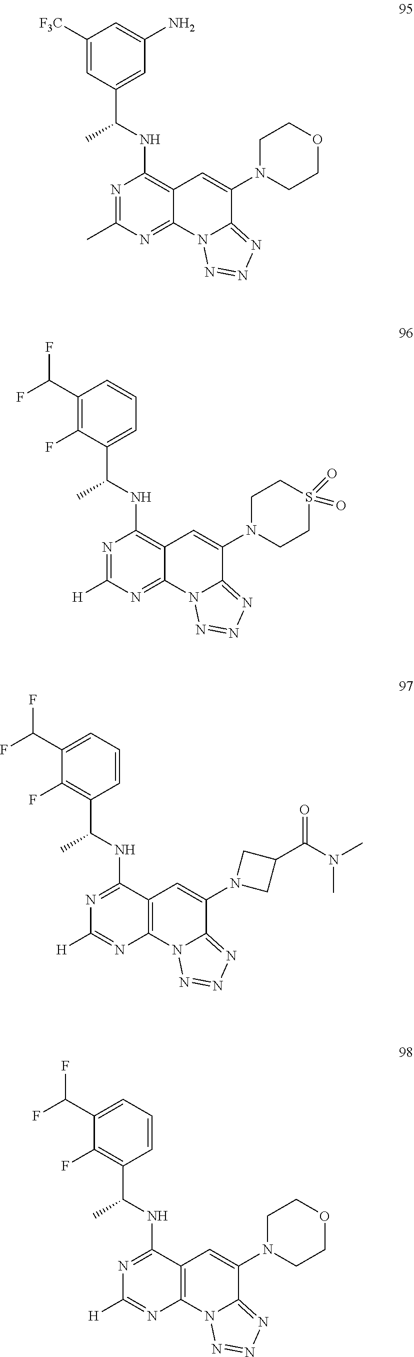 [SMILES: Cc1nc(N[C@H](C)c2cc(N)cc(C(F)(F)F)c2)c2cc(N3CCOCC3)c3nnnn3c2n1 ; C[C@@H](Nc1ncnc2c1cc(N1CCS(=O)(=O)CC1)c1nnnn12)c1cccc(C(F)F)c1F ; C[C@@H](Nc1ncnc2c1cc(N1CC(C(=O)N(C)C)C1)c1nnnn12)c1cccc(C(F)F)c1F ; C[C@@H](Nc1ncnc2c1cc(N1CCOCC1)c1nnnn12)c1cccc(C(F)F)c1F]

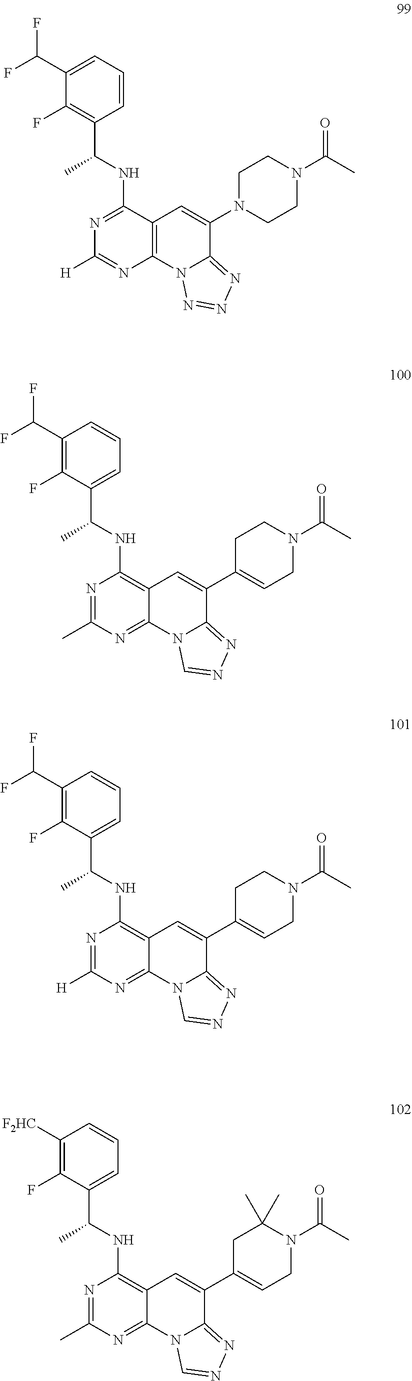 [SMILES: CC(=O)N1CCN(c2cc3c(N[C@H](C)c4cccc(C(F)F)c4F)ncnc3n3nnnc23)CC1 ; CC(=O)N1CC=C(c2cc3c(N[C@H](C)c4cccc(C(F)F)c4F)nc(C)nc3n3cnnc23)CC1 ; CC(=O)N1CC=C(c2cc3c(N[C@H](C)c4cccc(C(F)F)c4F)ncnc3n3cnnc23)CC1 ; CC(=O)N1CC=C(c2cc3c(N[C@H](C)c4cccc(C(F)F)c4F)nc(C)nc3n3cnnc23)CC1(C)C]

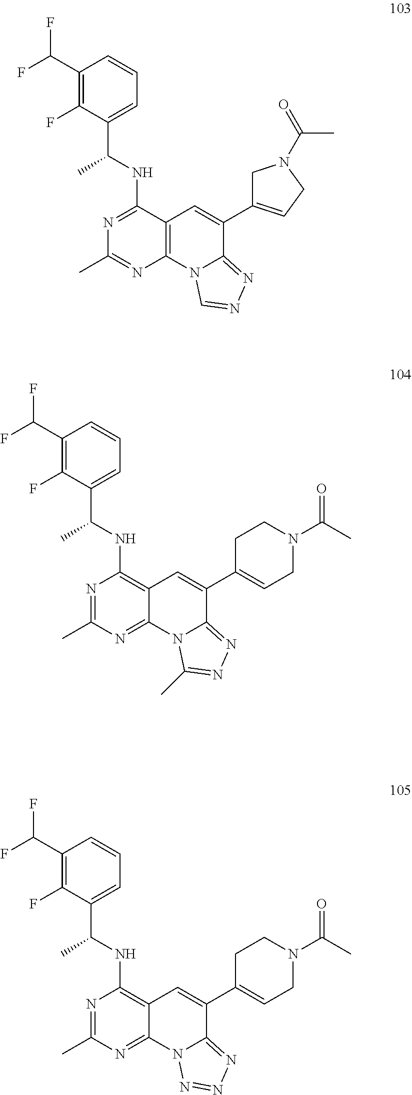 [SMILES: CC(=O)N1CC=C(c2cc3c(N[C@H](C)c4cccc(C(F)F)c4F)nc(C)nc3n3cnnc23)C1 ; CC(=O)N1CC=C(c2cc3c(N[C@H](C)c4cccc(C(F)F)c4F)nc(C)nc3n3c(C)nnc23)CC1 ; CC(=O)N1CC=C(c2cc3c(N[C@H](C)c4cccc(C(F)F)c4F)nc(C)nc3n3nnnc23)CC1]

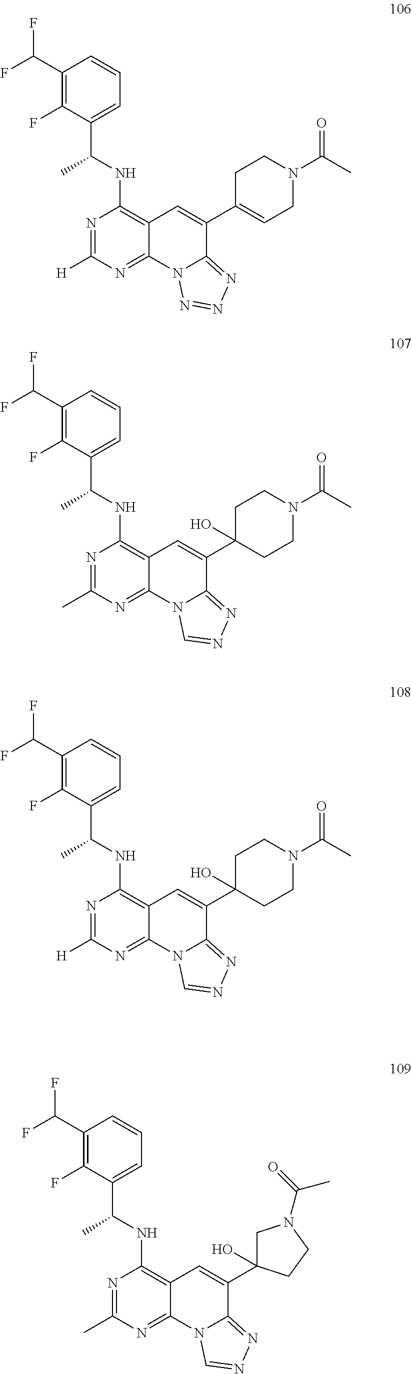 [SMILES: CC(=O)N1CC=C(c2cc3c(N[C@H](C)c4cccc(C(F)F)c4F)ncnc3n3nnnc23)CC1 ; CC(=O)N1CCC(O)(c2cc3c(N[C@H](C)c4cccc(C(F)F)c4F)nc(C)nc3n3cnnc23)CC1 ; CC(=O)N1CCC(O)(c2cc3c(N[C@H](C)c4cccc(C(F)F)c4F)ncnc3n3cnnc23)CC1 ; CC(=O)N1CCC(O)(c2cc3c(N[C@H](C)c4cccc(C(F)F)c4F)nc(C)nc3n3cnnc23)C1]

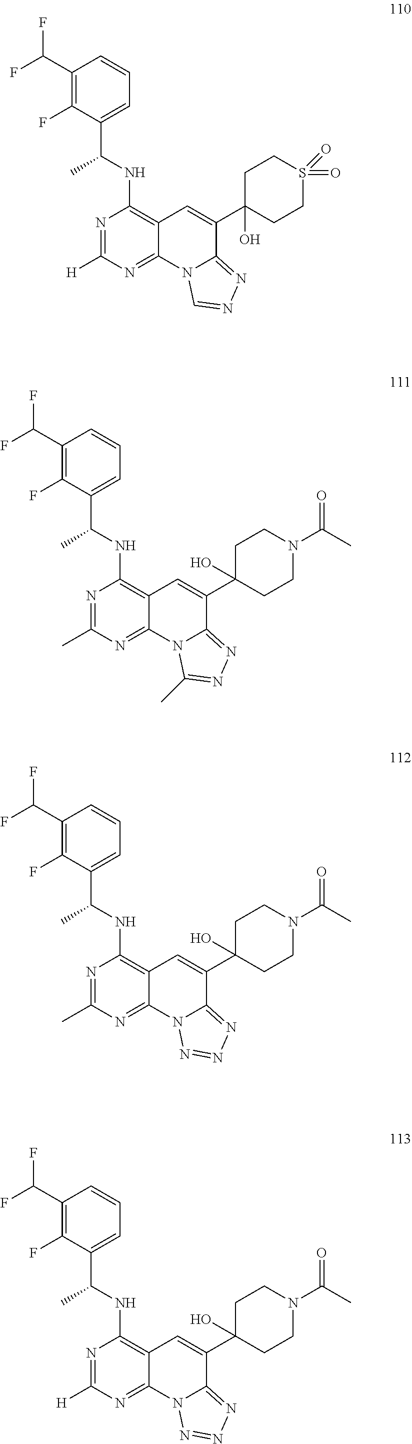 [SMILES: C[C@@H](Nc1ncnc2c1cc(C1(O)CCS(=O)(=O)CC1)c1nncn12)c1cccc(C(F)F)c1F ; CC(=O)N1CCC(O)(c2cc3c(N[C@H](C)c4cccc(C(F)F)c4F)nc(C)nc3n3c(C)nnc23)CC1 ; CC(=O)N1CCC(O)(c2cc3c(N[C@H](C)c4cccc(C(F)F)c4F)nc(C)nc3n3nnnc23)CC1 ; CC(=O)N1CCC(O)(c2cc3c(N[C@H](C)c4cccc(C(F)F)c4F)ncnc3n3nnnc23)CC1]

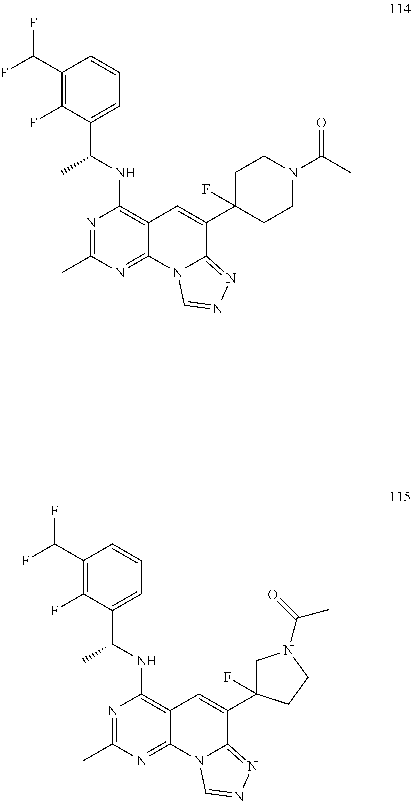 [SMILES: CC(=O)N1CCC(F)(c2cc3c(N[C@H](C)c4cccc(C(F)F)c4F)nc(C)nc3n3cnnc23)CC1 ; CC(=O)N1CCC(F)(c2cc3c(N[C@H](C)c4cccc(C(F)F)c4F)nc(C)nc3n3cnnc23)C1]

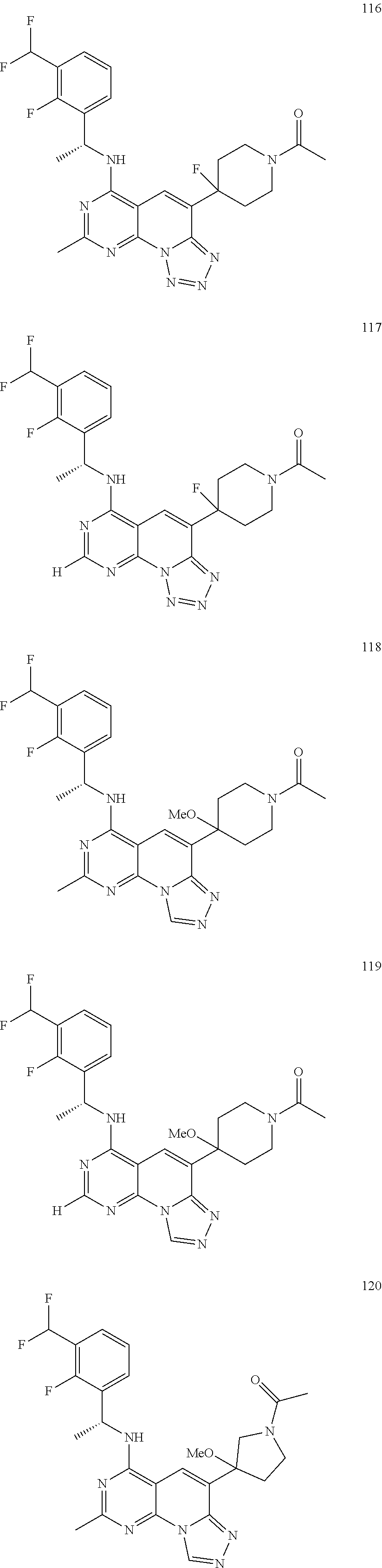 [SMILES: CC(=O)N1CCC(F)(c2cc3c(N[C@H](C)c4cccc(C(F)F)c4F)nc(C)nc3n3nnnc23)CC1 ; CC(=O)N1CCC(F)(c2cc3c(N[C@H](C)c4cccc(C(F)F)c4F)ncnc3n3nnnc23)CC1 ; COC1(c2cc3c(N[C@H](C)c4cccc(C(F)F)c4F)nc(C)nc3n3cnnc23)CCN(C(C)=O)CC1 ; COC1(c2cc3c(N[C@H](C)c4cccc(C(F)F)c4F)ncnc3n3cnnc23)CCN(C(C)=O)CC1 ; COC1(c2cc3c(N[C@H](C)c4cccc(C(F)F)c4F)nc(C)nc3n3cnnc23)CCN(C(C)=O)C1]

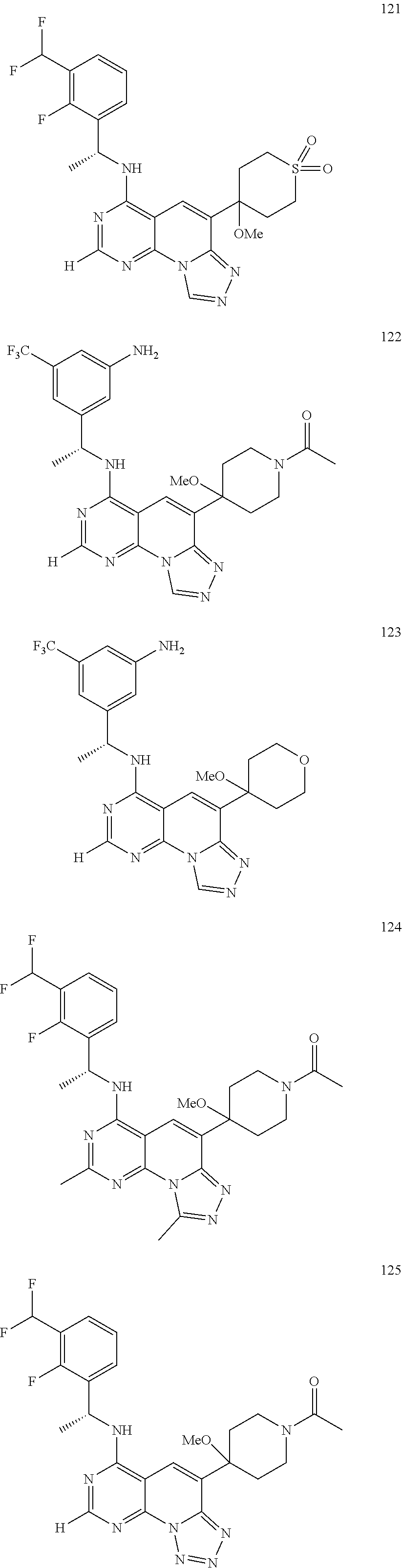 [SMILES: COC1(c2cc3c(N[C@H](C)c4cccc(C(F)F)c4F)ncnc3n3cnnc23)CCS(=O)(=O)CC1 ; COC1(c2cc3c(N[C@H](C)c4cc(N)cc(C(F)(F)F)c4)ncnc3n3cnnc23)CCN(C(C)=O)CC1 ; COC1(c2cc3c(N[C@H](C)c4cc(N)cc(C(F)(F)F)c4)ncnc3n3cnnc23)CCOCC1 ; COC1(c2cc3c(N[C@H](C)c4cccc(C(F)F)c4F)nc(C)nc3n3c(C)nnc23)CCN(C(C)=O)CC1 ; COC1(c2cc3c(N[C@H](C)c4cccc(C(F)F)c4F)ncnc3n3nnnc23)CCN(C(C)=O)CC1]

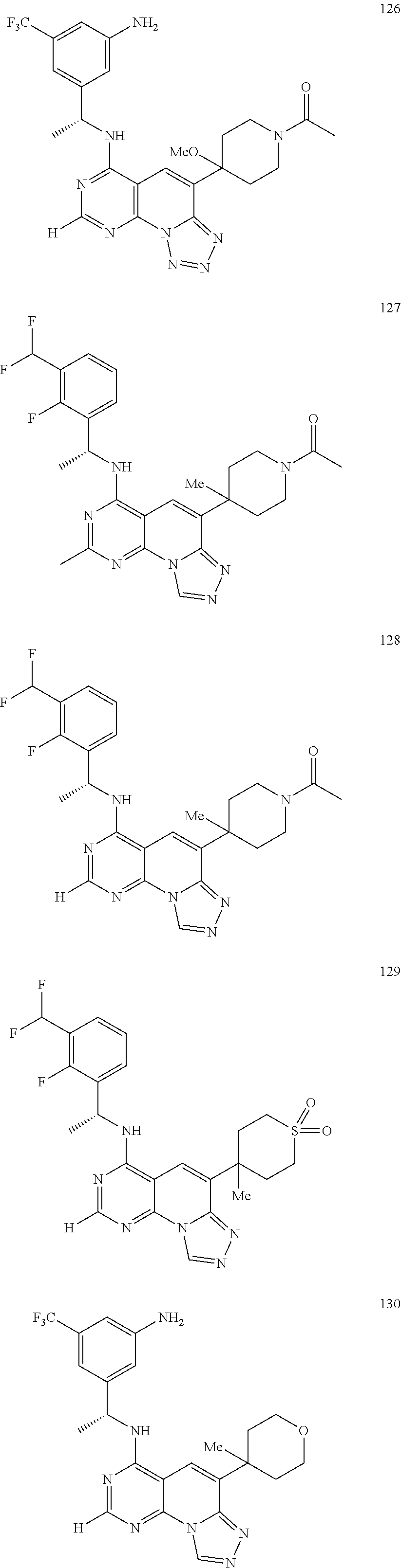 [SMILES: COC1(c2cc3c(N[C@H](C)c4cc(N)cc(C(F)(F)F)c4)ncnc3n3nnnc23)CCN(C(C)=O)CC1 ; CC(=O)N1CCC(C)(c2cc3c(N[C@H](C)c4cccc(C(F)F)c4F)nc(C)nc3n3cnnc23)CC1 ; CC(=O)N1CCC(C)(c2cc3c(N[C@H](C)c4cccc(C(F)F)c4F)ncnc3n3cnnc23)CC1 ; C[C@@H](Nc1ncnc2c1cc(C1(C)CCS(=O)(=O)CC1)c1nncn12)c1cccc(C(F)F)c1F ; C[C@@H](Nc1ncnc2c1cc(C1(C)CCOCC1)c1nncn12)c1cc(N)cc(C(F)(F)F)c1]

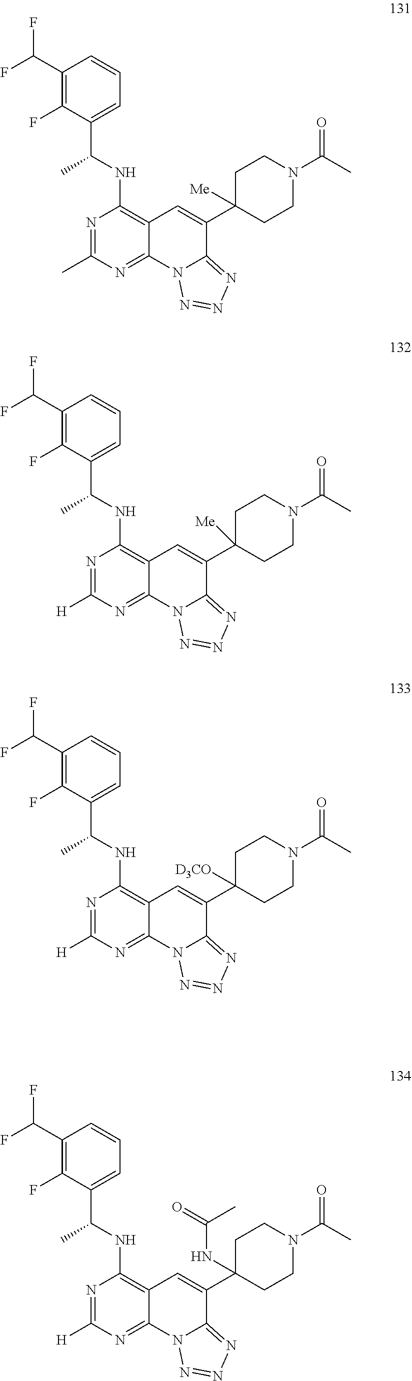

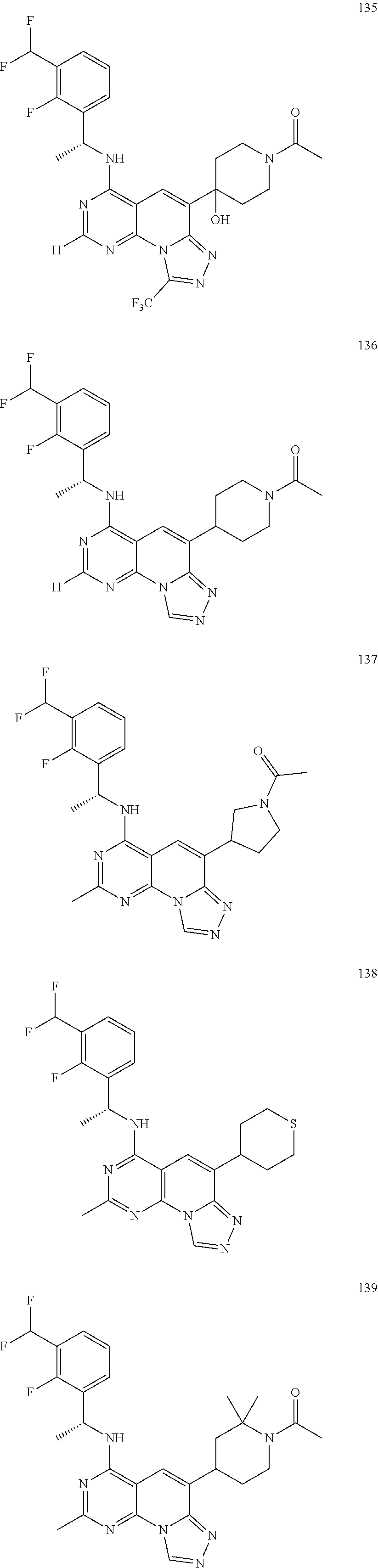 [SMILES: CC(=O)N1CCC(O)(c2cc3c(N[C@H](C)c4cccc(C(F)F)c4F)ncnc3n3c(C(F)(F)F)nnc23)CC1 ; CC(=O)N1CCC(c2cc3c(N[C@H](C)c4cccc(C(F)F)c4F)ncnc3n3cnnc23)CC1 ; CC(=O)N1CCC(c2cc3c(N[C@H](C)c4cccc(C(F)F)c4F)nc(C)nc3n3cnnc23)C1 ; Cc1nc(N[C@H](C)c2cccc(C(F)F)c2F)c2cc(C3CCSCC3)c3nncn3c2n1 ; CC(=O)N1CCC(c2cc3c(N[C@H](C)c4cccc(C(F)F)c4F)nc(C)nc3n3cnnc23)CC1(C)C]

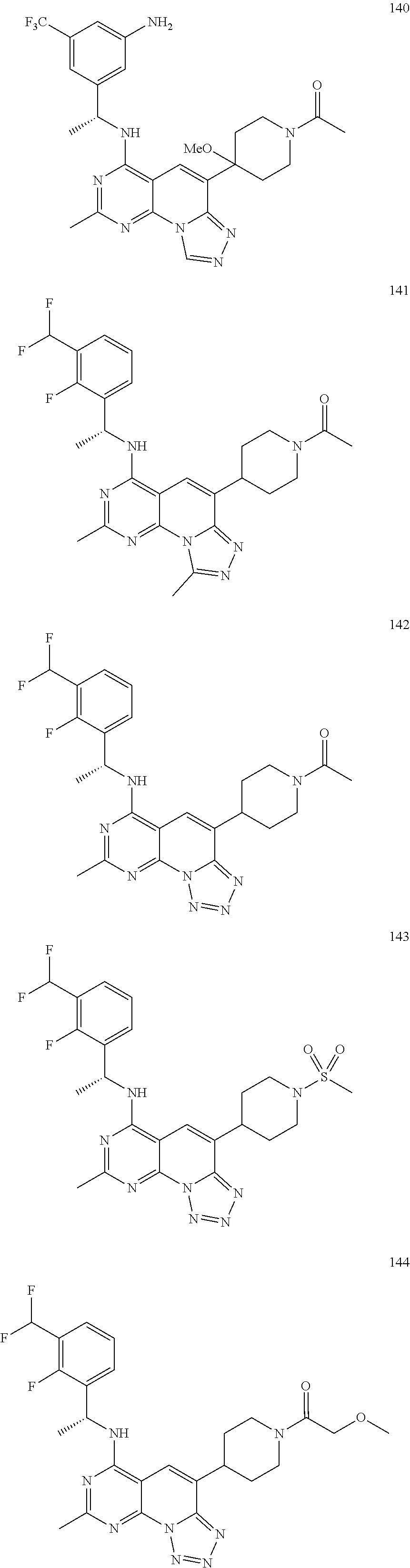 [SMILES: COC1(c2cc3c(N[C@H](C)c4cc(N)cc(C(F)(F)F)c4)nc(C)nc3n3cnnc23)CCN(C(C)=O)CC1 ; CC(=O)N1CCC(c2cc3c(N[C@H](C)c4cccc(C(F)F)c4F)nc(C)nc3n3c(C)nnc23)CC1 ; CC(=O)N1CCC(c2cc3c(N[C@H](C)c4cccc(C(F)F)c4F)nc(C)nc3n3nnnc23)CC1 ; Cc1nc(N[C@H](C)c2cccc(C(F)F)c2F)c2cc(C3CCN(S(C)(=O)=O)CC3)c3nnnn3c2n1 ; COCC(=O)N1CCC(c2cc3c(N[C@H](C)c4cccc(C(F)F)c4F)nc(C)nc3n3nnnc23)CC1]

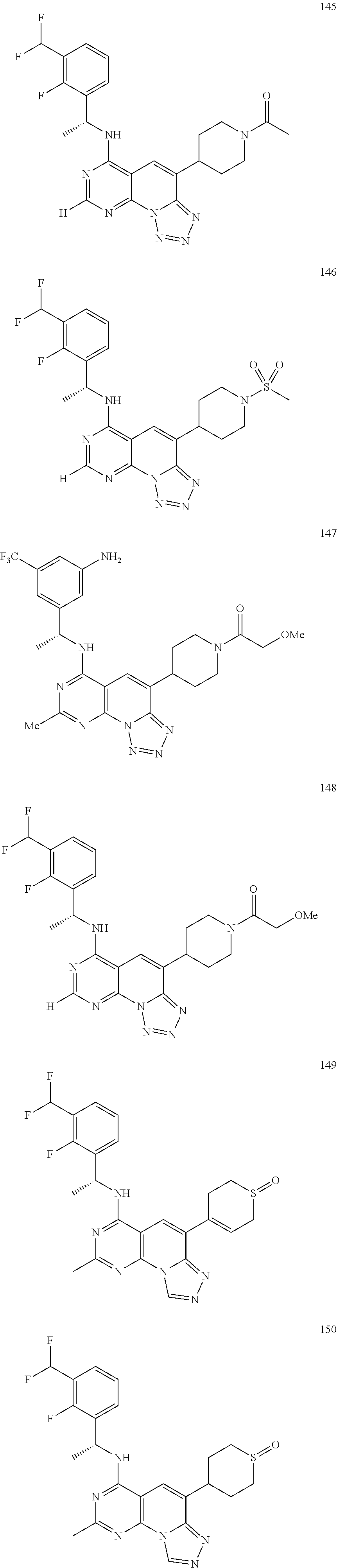 [SMILES: CC(=O)N1CCC(c2cc3c(N[C@H](C)c4cccc(C(F)F)c4F)ncnc3n3nnnc23)CC1 ; C[C@@H](Nc1ncnc2c1cc(C1CCN(S(C)(=O)=O)CC1)c1nnnn12)c1cccc(C(F)F)c1F ; COCC(=O)N1CCC(c2cc3c(N[C@H](C)c4cc(N)cc(C(F)(F)F)c4)nc(C)nc3n3nnnc23)CC1 ; COCC(=O)N1CCC(c2cc3c(N[C@H](C)c4cccc(C(F)F)c4F)ncnc3n3nnnc23)CC1 ; Cc1nc(N[C@H](C)c2cccc(C(F)F)c2F)c2cc(C3=CCS(=O)CC3)c3nncn3c2n1 ; Cc1nc(N[C@H](C)c2cccc(C(F)F)c2F)c2cc(C3CCS(=O)CC3)c3nncn3c2n1]

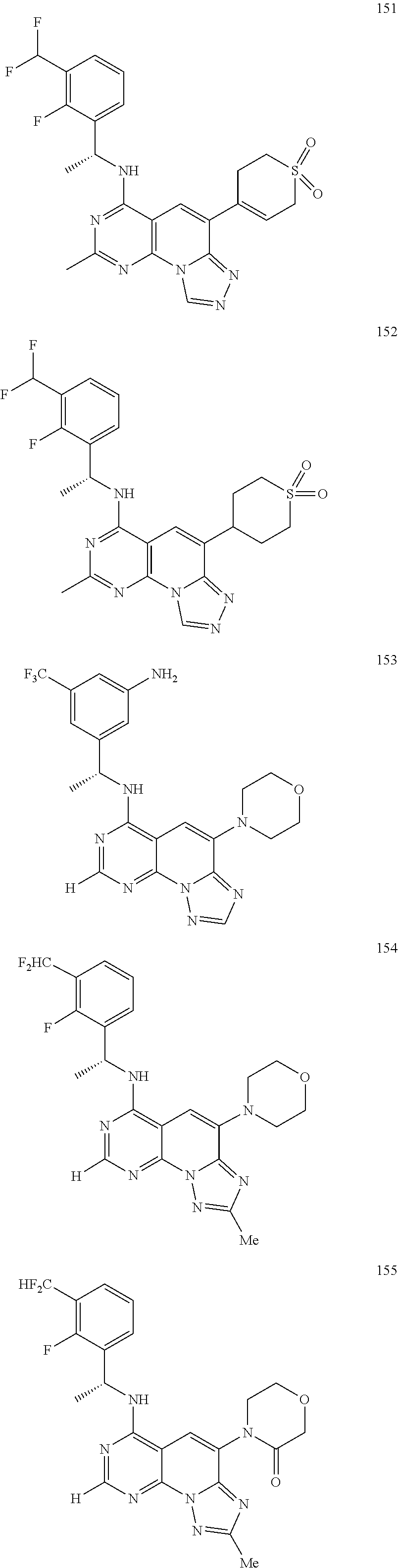

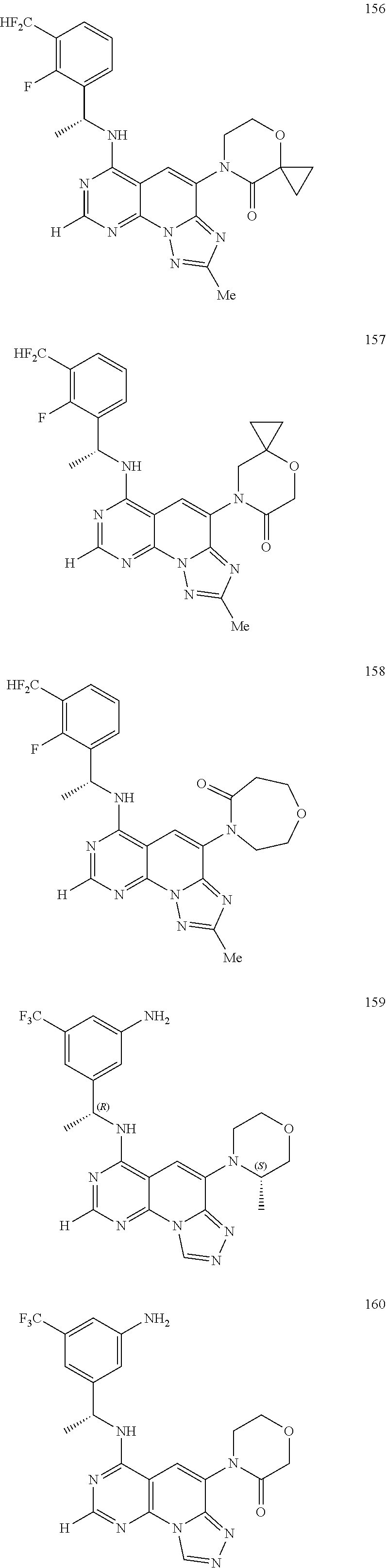

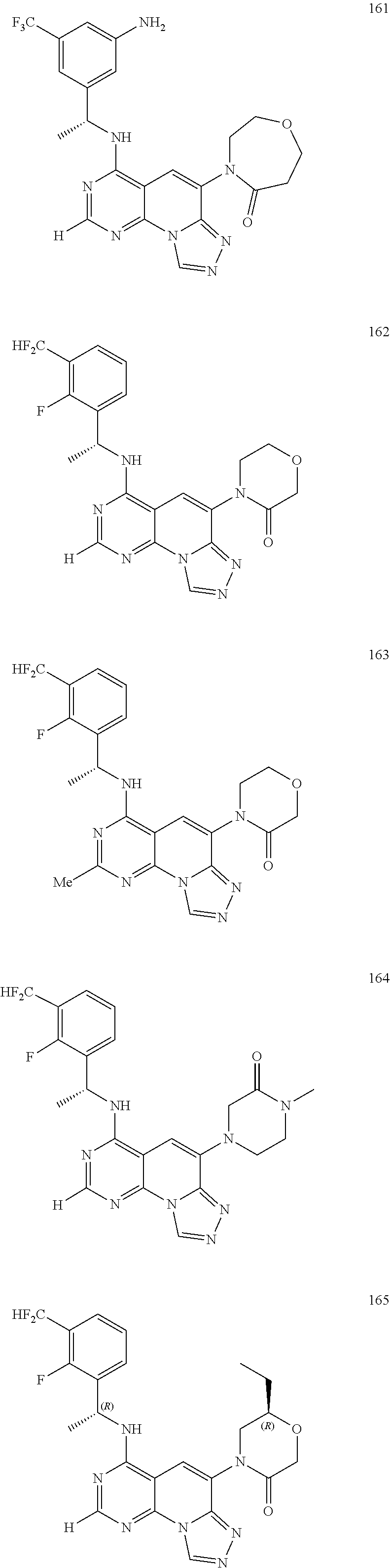

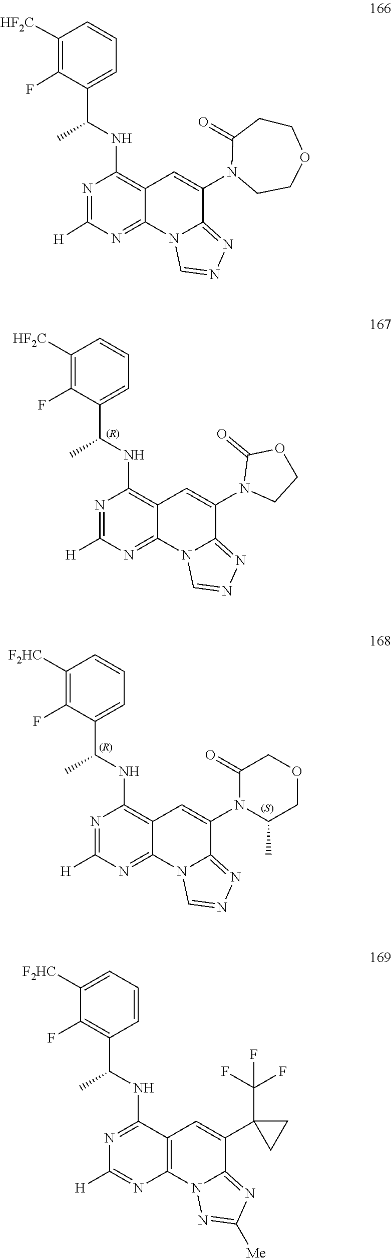

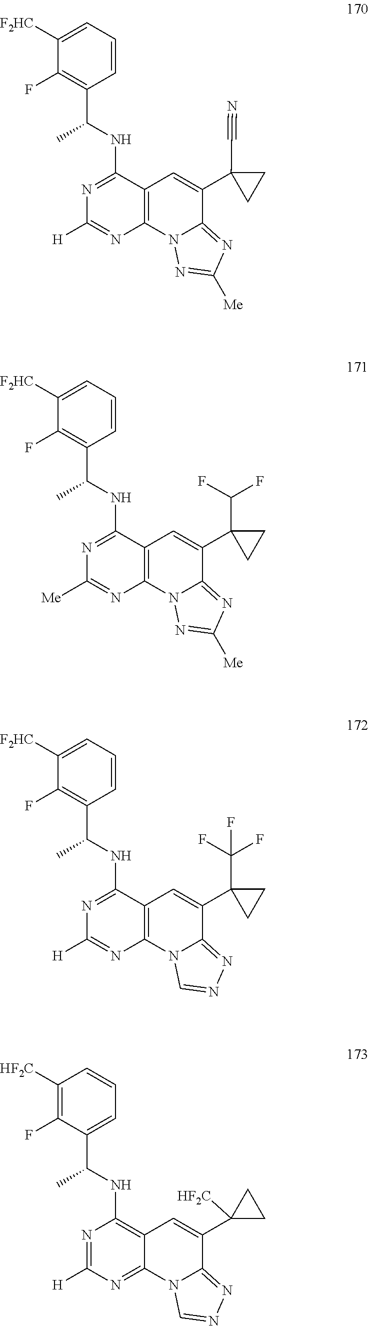

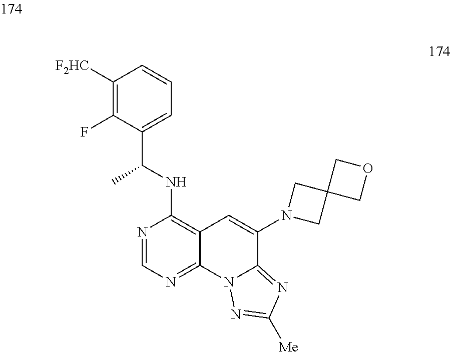 [SMILES: Cc1nc2c(N3CC4(COC4)C3)cc3c(N[C@H](C)c4cccc(C(F)F)c4F)ncnc3n2n1]

15. The compound of claim 1, wherein the compound is

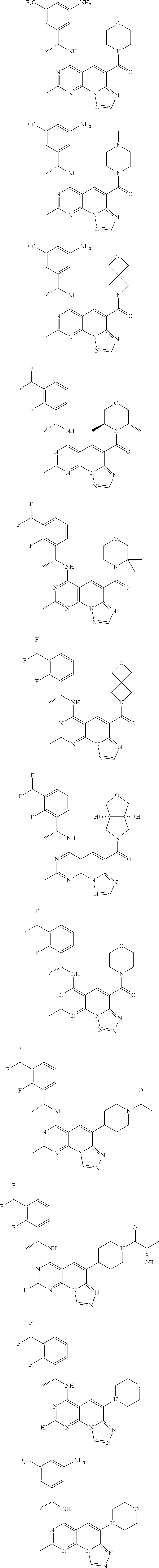

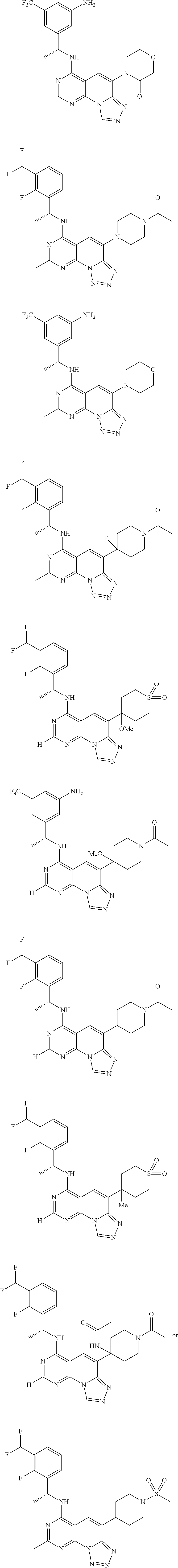

16. A pharmaceutical composition comprising a compound of claim 1, and a pharmaceutically acceptable carrier or excipient.

17. A method of treating a disorder associated with RAS dysfunction in a subject in need thereof, comprising administering to the subject a therapeutically effective amount of a compound of claim 1.

18. The method of claim 17, wherein the disorder is a cancer.

19. The method of claim 18, wherein the cancer occurs in breast, prostate, skin, lung, pancreatic, stomach, brain, kidney, uterine, ovarian, testicular, endothelial, colon, bladder, bone or blood.

20. The method of claim 19, wherein the cancer is lung cancer, colorectal cancer, or pancreatic cancer.

21. (canceled)

22. (canceled)
